SEQUENCE LISTING (1 sequence)

SEQ ID NO: 1 - DNA, 230966 bp, synthetic construct
  misc_feature 1..230966: note = rdCMV - Destabilized IE 1/2 and UL51
  source 1..230966: mol_type = other DNA
gggccgcgtggtgggtcctcgaggggcgggggggtgtttttagcgggggggtgaaacttggagttgcgtgtgtggacggcgactagttgcgtgtggtgcggaggacggcgacggcgaataaaagcgacgtgcggcgcgcacggcgaaaagaagacgcgtgtctgtgtctgtgtgattccccggggaaaagaggaagttcccgggggacggcagcatgggtccctggggacacacgaaaagcaacgcccgggggcgagggacgacggccctggggaccgcgggggaaataacggccgcgaggccacacactcgttcctgcgaagccgcacaccccgaggccgcgcacaccgccgacacaccccgccaccacaccccgccggcacacccgccacacgcccgcgacacacccggcacgacacacccggcacacgcccgcgacacaccctgacacaccctgccaacacacccccgacacacccaacacacgcccgcgacacacccggcacacacccacccggccgcgccccgacacacccaaaacaccgccggtgcggggccgcgtggtgggtcctcgagggagtgttgagggccgtaagcgtgttgtgtccgacgctgcctgcgcactgccggtgcgtgtcgtcccacggtatttgttgtcggcaccgggcttcgggacggtgtttcggcgcgctgccggtgcgttccacggtccttgcctgtgtcgtttcggcgctgcgcttgtcgggggttttcgagcgttctggccgccggcgatgccggggtgttgcggagacggggggtgtgcgggacggtgttggggccggggacgggggttgcgctggggccggggctgttcgcgccgcgtaggggaggttacgttggggacggggacagtttgcggcgcggaccagggaacccacctcacctatttaacctccacccactacaacacacacatgccgcacaatcatgccagccacagacacaaacagcacccacaccacgccgcttcacccagacgcccaacacacgttacccttacaccacagcaacacacaaccgcatgtccaaacctcggacaaacacgccgacgaagaacaccgcacacagatggagctcgacgccgcagactacgctgcttgcgcacaggcccgccaacacctctacgatcaaacacaacccctactactcgcataccccaacaccaacccacaggacagcgctcattttcccacagagaatcaacatcaactcacgcatccacttcacaacattggcgagggcgcagcactcggctaccccgtcccccgcgcggaaatccgccgcggcggtggcgactgggccgacagcgcaagcgactttgacgccgactgctggtgcatgtggggacgcttcggaaccatgggccgccaacctgtcgtcaccttactgttggcgcgccaacgcgacggcctcgctgactggaacgtcgtacgctgccgcggcacaggctttcgcgcacacgattccgaggacggcgtctctgtctggcgtcagcacctggtttttttactcggaggccacggccgccgtgtacagttagaacgtccatccgcgggagaagcccaagctcgaggcctcttgccacgcatccggatcacccccatctccacatctccacgtcggaaaccgccgcaccccgccacatccaccgcatcgcaccacccacatgcttcgcctcggtcagatcacacgctttttcctgtcccatctacaccctcagccacggttcacaatccccgaaactacgccgtccaacttcacgccgaaacgacccgcacatggcgctgggcacaacgcggtgaacgtggcgcgtggatgccggccgagacatttacgtgtccaaaggataaacgtccctggtagacggggtagggggatctaccagcccagggatcgcgtctttcgccgccacgctgcttcaccgatatccaataaacccatcccctcgccacgacgtctccgcgtatctttgtagcctcaagaatccgtccccacgtccacccatcccgagcactccacacgccataacaaaccacggacacgacaaatgcatgcaaacttctcatttattgtgtctactactctgtgttgctacagggagtgaagagggtgaaggcaaagaaaaaaaaaaggaacaaaataatagattagcagaaggaataatccgtgcgaccgagcttgtgcttcttttcttataaggaggcaaatatactagggaaaacataagaataggaagaaaccgaggtttgggagaaaagctgagataaaatagcgcattttccatacagaggttgttgtttttgtggatcctaagaggtttcaagtgcgaatctcaaagttctcacgagaatattgtcttcaagtatcgacaactgtggtccaagatttttttttggtctttttaggttctgcgagggacatcacgatggatcgttgcgatgaagtcacgcgtacgcctctggtgtggcgcggtgtcgtgacaggagagtgtgttttcagtgcagagctgtcttgattcctatatccgagtatctgttttctcgtaaggacggtaatcttctttggtgtaagtacatctaaaagctgcaaactatattttaagggctgtctctaggtgtactttgatgctggagtttttcgctgtgttgatgtgaataaatctactactactattatatgcagaaagagtgattatgccgagacaagattgcattggctgaactgtttcaaaaacgcctacactctacttatccgtaaacctaaggtaatactatgtgtaagttgtttttttttctttttgtagtaaaatggtgatacgtgcaattaaaactgtattccatgtttccatcctttcatttcaactttaaaggcggctttgagagcgaagaagtgcgaggataaaaatggatgactccttcgtgtccagggagtcgactactgcaacgctgattgattaaaagatggtctccgatgatgatgttgttattgatcgaatcatggtgcagaacggcgacggagaggagcgtgtccgccgccgggaaggtggtctctttctcttttcttttttcaagaaatcttccatgtgtttatcgtagtgatcgaaatcgactgatctcgggttctttttgttggtttcttttcggttaatcatgtattgttttctttttttacagaaagatactttttttcatgagcaattcctcgcccggcgccggcatgccgaggtggggccactgcgatcagcggcatgccgacgccgacccggggatcttggattcaccgttttctctcttctctctctacatacagaccgggtggcaggagcggtaaggaatcatcgtcgtctttcattcttcgatgattatggtaatactaaatcttatctaggagcatatacatctaagattggagtactagtagtcgtttgtggtttctatttttttttatatttatctatgacagtttttctgtttttcgttttgataataatataataaaaactcatggacgtgaaatctggcttggttgtggtgatttcattctcattattgttgttttctttccgtcttgcggatgaagatgttgcgatgcggttgttgttggtgttgctatacaccgagagagatgatctttttgttcttctggttcatttcctatgattgtttggctgctgaccgacgcgtcaggatgtgcagggcatgcggggaatcaggaccggacacgggataatttcatctacctatacggagatcgcggtcctcgccatgaggatcgcgacaggcgcgtcgagggggcaggaacacccttgcggattgacattcttggtggtgtttcgttgttgtcggtagttgttgttgacgatgaggataaataaaaatgaccttgtttttgttctgttttctcttgttgggaatcgtcgactttgaattcttcgagttatcggaaagctgaggtacccaaatgtctgtagcttttttctttttaccctcttgtttatcatctgcgattcgtggtaggtaggagagggaaatgataatccgagattaaggaaaggagaagataaaaaataaaaaaaaataataaaacagaagccgaccggccgccgacccgttccccaggaccagcctacgaggaacggataacgcggtggcgacggcagcggtggtggcgctgggggtggcggcagtggtactgctgatggtagtcgggacggaggagaggcgatgcatacatacacgcgtgcatgctgcatgggtggatggtacggccgggagacgcggaagagaaactcacataaaaaggtgacaaaaagagcggttgaaaaaagaaaacaagattcgaccagacagaagagaaggaccggggcttggcgacccttccacgactgctgttgtcatctcggctcctccgtcttctcccggccacgggcggctaagtcaccgccgttctccccatccgtccgagcgccgaccgaccagccggccgattcgcccgccggggcttctggagaacgccggggcagcagcgatctggggaagctgctaaacccctgcgtttttatatggtagctctgccgagcgcgggctgacgcgttgggtaagcggaaagacgtgtgtgacgaaaaggggtcccatggtatttcacgtgacgatgaggagatacggtttggagcacatacggtttagaaaaagggagttgtcgtgacaagggctgagggacctctgtctccatgtgtgtataaaaagcaaggcacgttcataatgtaaaaaagaacacgttgtaaacaagctattgctgtatcattcggctgactatgcttcattcggactgattttcttttcctaacggcgtaacttaaagtgattaacgtatgatatttgttccccagagttatactatagtcatcatcctaaaattcagatataaatgaacacatgtcgtatgggattattaagaaaccgaaactctccacagttcaccatcttcttcgtcattcaaccgatgacccactccgtacaacgaatcagtctgctgcgtcatattgcaaagcacaagcgacgtatgcgaacaacttgaaacacaggctgttgtattgacgaccgttgtaccattattagtcaccaccgttatcccatgtttcccacccgatggaaaaccgtcttctatcatcaactgtggtaagatttcgaccctgcgaggtattcagtttcctcatatccataacctggattttatcattaaaccccaatattaaacacttttttagtaccccccacccaccaaaaaatgtgactggaccggttcctagcagctctgggagccatgttcaggttgaaccacagctacagcgaaaccgagtccagtgaccggtaaccacgtccagcccctgcgtatgtaccagtccaagcacgtccggtcattgttctacacaggaaatctaactaggtcaacgcaattttattccaccgttacgcagaatactaacaaaaaaacacacaaatttaacgaattacacgtagtttattacatgaaaactgtaagaacaccaattcactaagcgatacaacatttagctgacttccaagtgccacacatcaccactgtattcatccatgttttcaccgaaccaacgagacagatcgaagaagccagaatctcccgactttaaattacataaatccaacgtattatgaccacagctcgacacacaaatagttgcgttactattcacagtagcattacctatacccgtaacgttgcacaaccactgatcaccattgttaccaaaaacggttttccacttagttgtcaacggatctttcctatgcgtaatggtaaaattactaccagtcgtcgcttttagctcattacgagtattatccgcatccacatatatcaacgtcatagctaggcacgctataagtaccccccccccacaatggaatgttgccaaaccggttctttcccgttatagccatagcgttcccaggcaaaagcaaacgccaaacctaatgcagtgaaaagcgcttgcagccagaaccagcttatgtaccagccacaatcacatccggttattgtttccacaggaaatcctaccaggcaaagccccgcttgttttgttcctatcttgtttagcaattcgtaaactgtcagcctagcgacgtccgtttagatcaaaagtcacgtatatagcgacgctgtttccatccgtttccccgtcccgccgtttccgaacaacccacccgggttcagacaaccgaccaccaacagaaatatacacacagaccactgggagttcagttaaagatttcatcaggtttattttggctgctgctagtcttttgcttcttagaaaaaaaatacccatatagagaaataatgatagtttgacaacacatatggcagggatttcttcttcatcaataagatatgcaattcccccagggagagactttcaacaattgaatttacaaaaacaaaattacatcaggagaaagagaggatacattaataaatatattatatctggtgtatatactgaatgctgctggttcataaggtaacgatgctactttttttaattccaagatggtttttctttgttagtcttttgttgacttgctggttcctaaaagttcgcaaaaacgattgtgtgaagattttatgacgttggttgactagttcatgagattctgctgtacgtgtgatggttattcgctggttcgttctaagatgagtatcgtactgtgtctgcgatggtcgtctcttactggcattctctcggctgcctcttgctttcatgattgaaaaggaaaaaaggactccgagggcgcggtcatcttttacttttcggttttctcgttggcgggtcagaggtagtcagatcatgagactgtcgtggtcgatgaaactgtgtctgctcaagtgacgtccatttcttgtacggagaaaaaagtcatcgggataaataaggctatacaaggcgttgtcaagcgtgcggctctaaacaaattaagcgatacaaaattacagtaatacgaataataaattacccccctccccctgtggtcccccgagacgagagccacccatcgtgtactctcgcaccacccacgaccacagagggagacgggacgaagagacgacgcacagcgccatctcctcctggaggccggcgacgttaactgctacagctgcggcggcgaagacagctgcgatttgtcggccgacatgccgatggtatgggcggcggcggcaatggccgcggcagcggggaggagaggagagagaagaggagcggggcgtccgaaggcgaggatggcatggtctcgccggagcgcccggcttttatggaacactcgcgtccggttgggtatcacccacaggaagatgagtcacaacttccaaaccatcttgagacccgagtaacggtttacaggtcgcacgccagtcagctaaaaacagcggacagtcccacgctgtttctgttgtggctctctccagtttcctcatcaccgtcccggtctccgtcgtcatcggaagaataccacccgctctcatgcggcagtcgatcggcctcgacgaacgagacgcggcgacgcctctccacggccgactggttgtggtggtgaaagaagagcaccagcaatcccaggaggagcaacaagccctcacatgtccaggaggtcggggagagggcctgtcggagatggccgtgaggcatcacgtacggcagctgaggagaaacggagaagaaaggaaaattaccgtcaggggccggggttcttattagagaaacagcacgtaggtcaggatccagatgctaatggcaatcatgatgacgatgatcatgcaggccaagacgcggcgcaccaatgccgaatccaatagccgccgtgcctccggttggtggccggcggcatctagagacatgatttgggggggaccggcggcgcaaaaagacagggagatggacagtgtcacggtgttttgttataattaggacatggggaccggaagccgagacagagtactacagggtgttgaagggtaacgtgagggagatcatgtcatgggcgggctgaagaccgtgcggggaggattgacgtgtgcggtgcttgtggaacacggtgttttaatatgtatccgcgtgtaatgcacgcggtgtgctttctggcactcagcttggtaagctatgtggccgtctgcgccgaaaccaaagtcgccaccaactgtctcgtgaaatcagaagatacccatttgacgtgcaagtgcagtccgaataacacatcatctaataccggcaatggcagcaagtgccacgcgatgtgcaaatgccggatcacagaacccattaccatgctaggcgcatactcggcctggggcgcgggctcgttcgtggctacgctgatagtcctgctggtggtcttctttgtaatttacgcgcgcgaggaggagaaaaacaacacgggcaccgaggtagatcaatgtctggcctatcggagcctgacacgcaaaaagctggaacaacacgcggctaaaaagcagaacatctacgaacggattccataccgaccctccagacagaaagataactccccgttgatcgaaccgacgggcacagacgacgaagaggacgaggacgacaacgtctgataaggaaggcgagaacgtgttttgcaccatgcagacctacagcacccccctcacgcttgtcatagtcacgtcgctgtttttgttcacaactcagggaagttcatcgaacgccgtcgaaccaaccaaaaaacccctaaagctcgccaactaccgtgccacctgcgaggaccgtacacgcacgctggttaccaggcttaacactagccatcacagcgtagtctggcagcgttatgatatctacagcagatacatgcgtcgtatgccgccactttgtatcattacagacgcctataaagaaaccacgcgtcagggcggtgcggcgttcgcgtgcacgcgccaaaatctgacgctgtacaatctcacggttaaagatacgggagtctacctcctgcaggatcagtataccggcgatgtcgaggctttctacctcatcatccacccacgtagcttctgccgagccttggaaacgcgtcgatgcttttatccgggaccagggagagttgtggttacggattcccaagaggcagaccgggcaattatctcggatttaaaacgccagtggtccggcctctcactccattgcgcctgggtttcgggaatgatgatctttgttggcgcgctggtcatctgcttcctgcgatcgcaacgaatcggggaacaggacgctgaacatctgcggacggacctagatacggaacctttgttgttgacggtggacggggatttacagtaaaagatgcgtgtcgcctgccgaagacctcaccatctcacgtacaggcatacggcgtatacaatcataatattctatattctgcatagagttacatgcaacagtactactaccaatactgcatccatcacatcacccaacactgcttctaccacctttgtgaccagcgtattttctactccgaataacaacacatcaacgacgccacacacatctgtcacctcacaagcgtcaaccattggcaacatcaccaacgttacctccgacttgagtactttcacaaccgtatattctacattcaatacatcatatgctaatatatccaatacggctgccactacagaattgatttcaacaaataccaacactatattatcttttaccaacgtaacagcaaacgctacatcatcttataacacaacaatcaccgtaactatcacgtcagatgaaacttcgcacaacgtatccactaatactgcacttataagcacgccatggcttacaaattgcagcgccacaacgtacaccacgtacaaccgtactaactcttccaacgcttgtcacacagagacaacaatcatacgtttcaaagaaactaatacaacaggaatagaagggagtaatgtcaccataaaaggtaattctacgtgggattgtctttcagtcgcctggatacgacattacaatcgatccacacacggacatcatctaggtcatcgtaagaacgcacatacccaatcttggtattggttacgcatccttacctctcatactgtatgtcattctcaacatgaaagaccttcactgtaccatgacttatgtcgttcgtgcaacaacacagaactacatctgtacgatctaaatatcaccaattccggcaggtacagcagacgttgttttaaagaaaattacttcacaggacatcacgaagatgaaaatttctacctattagtaacaccaaaaaatcatactgaagctattaatgctactttcgtttgccctagatacaacaccgatatcgaaaatgaagatagagagaaaggaagtcaacatactaacaatacacatcaccacaaacgtaatctctatcatagctcgcaaagaagccgcaccgtatggaccatcgtgttggtttgtatggcctgcatagttctgttttttgcacgacgagcctttaacaaaaagtaccatatgttgcaagacaccgtcagtgaatcagaattcattgttcgatatcacacagaacatgaagattgagctacgtttccgggcagacatcttatgaagctgaacaataaactaaaacattctgtaaggctcagcgttcaaaggaatattaatgcccattgagcgagaactaatattgcaatggactggcgatttacggttatgtggacgatactaatatccgcgttatcagaaagctgcaatcaaacctgttcctgtcaatgtccctgtagtactaccgttaactattccactagtactgagacagccacatcaacatacagtacaacagttatcagcaataaaagcacttcagaatctataaattgctctactgcaactgcaccagcaaccaccgtttctacaaaaccgtcgaaaacaaccacacagatatccacaacgacaaatacaaacgttgagactaccacatgtaccaacaccaccacgaccgttacttgtgatggtttcaattatacagtccataaaagatgcgaccgcagttacgaggtaatcaacgtaacaggatacgttggtggcaacataactctaaaaaatgcaatcagactgagaaatggcacaatgtagactggattcattatgagtaccccacgcataaaatgtgcgaattaggcaactatcaccaaacaacaccacggcacgacatatgttttgactgcaacgacacctccctaactatctacaacttaaccacaagaaacgctggaaaatataccaggcatcaccgtgataacggtcaagaagaaaattactacgtaacggtgttaattggagacacaacgttatccactcttggcacatgccctgtaagatataaagaatctaggaacactgaaaacaccattggaagtaacatcataaaaaccattgagaaagctaacattcccctgggaattcatgctgtatgggcaggcgtagtggtatcagtggcgcttatagcgttgtacatgggtagccatcgcattcccaaaaaaccgcattacaccaaacttcccaaatatgatccagatgaattttggactaaggcttaacatgcacatcaataaactttttttaaccaataacatgtctctgtttttttttgttaacaacctatgatataaagcggtatattcaatcattactaaacaaaaaaacatgggcatgcaatgcaacactaaattgttattgccagtcgcactaataccggttgtaatcatcctaattggtactctagtgcccatacttttacatgaacaaaaaaaggcgttttactggcgactttttctgcaaagtcaacatgtagaagcacccattacagtaacgcagggagacacagtctacctagatgctagcaataatccctgtaattattccagcttttggtaccacggtaattgcgaactttgtggatggaacggatatctacgcaatgttacacattactacacaaacacatcgtgttccccgcaattcatgtgcataaacgaaactaaaggtctgcagttatataatgtaacattaaacgattcaggtgcttatactgaacacgtttacgaatgtgatctttcatgtaacattactacttataacgaatatgaaatactcaattacttcgataactgtaactacaccataaatagcaccaagcatattatcaccgtggtgtcttcacgtcattctaaacaaacaaattcccacgtatccactcacgctggttgggcagccgccgtggtgacggtaattatgatctacgttttgatccactttaacgttccggcaactctgagacacaaactacgaactagaaacaacgtaaatcgcatagcgtgattacaaagtatcgacactaatttatccaagataaaatttgattactccgtgcggttctcaaaaactgtaaggtcccgcttttctactccatcatgaaggatcgcaatagaatactgctatgtatcatctttatttgcatcatgtgcctcatttgtatttactttaaacgtcgttgtgttcttactccgtctccagacaaagcggatctgcgagtggaatttccctcgttacccccgtgtatcggcatacaatgtgctgcatgagaacacgcgtgacacatagcgtacccctggacggtacagtttatgataacgtcattcaggggaagtatacattactatcgacgtgttatcacagaacacacagattttctgcgtgttttataaaagagcgtctcgaagcagcttgagccacactacggtccagatgacgagcgtaatcaaaaatatgccgcgcagtagtcgaaagccgtactgagcgtgcgaggcgggtagggtgccgaacgacggatatgcgtcgttgtcatcttcgactataaggatcgcgaccgagtcttcggccatggtaaacgtcaccctgtgtggctggtatgtagcgtatccggtttggaattgttctgctccagctcgggggatagtgaggaattctcaagggatacgggacccaatgactggataagagaagggtttttccccgtaagatgatcctcgtatcacatgaggtctggatatgtataaatgaagagtgaaataggcacagggaatcagatgccagcctcgtgatgcagccgctggttctctcggcgaagaaattgtcgtctctgttggcttgcaaatacatcccaccttaagcgatgagtccataaagcaccgttgtccgggtacggtgaaagtgactcggattgtagcacgtcccttttttttgtttttgtatcgcttatcgccactgacagtgcaatattttgatcgtgaggctgagtatggttatgatgcttagaacgtggagattattaccaatggtactacttgccgcgtactgttattgtgtttttgggacttgttcaatcggcacgacgactgctcccgtggaatggaagtctcccgaccgtcagattcctaagaatattacttgcgctaactactcagggaccatcaacggcaacgttacatttcgaggtcttcagaacaaaacggaagactttttgcactggttgttagggtggggtcataagtccatctgttcgttcttcccgaaactccagggcaactataacgaacaacattacagatatgaagtagcgaacctgacgtataactgcacctataaccgcttgacgttgctaaatctgacgacggaaaacagcggaaagtactattttaaaagggaagatgcgaatttcaccttttattactcttgttacaacctgaccgtgtcctaaagaacgcacgtgaagttccacagagccgcgtggctgtagctattgtgtttacgttgcttttgaaatgttaagcgtccctacggcgctaacatgtttctaggctactctgactgtgtagatcccggccttgctgtgtatcgtgtatctagatcacgcttaaagctcgtgttgtcttttgtgtggttggtcggtttgcgtctccatgattgtgccgcgttcgagtcctgctgttacgacatcaccgaggcggagagtaacaaggctatatcaagggacaaagcagcattcacctccagcgtgagcacccgtacaccgtccctggcgatcgcgcctcctcctgatcgatcgatgctgttgtcgcgggaggaagaactcgttccgtggagtcgtctcatcatcactaagcagttctacggaggcctgattttccacaccacctgggtcaccggcttcgtcttactaggacttttgacgcttttcgccagcctgtttcgcgtaccgcaatccatctgtcgtttctgcatagaccgtctccgggacatcgcccgtcctctgaaataccgctatcaacgtctcgtcgctaccgtgtagctagttagccagctgtgtatagtttgttgtgttttgcttttgcatatttgttttcagtcagagagtctgaaacggggtgggagggacttttacgggtaatgcatgctaagatgaacgggtgggctggggtgcgcttggtaactcactgtttgaatacgcgctcacgcacatatgtagcactcaacatgttagcttttgcccgcacgccccggggcgtgccgagctgcctttttaataaagtctgggtttccagatacgcgctggttctgattttgatggtttgtgcctctgaaagctctacgagctgggccgtgacatccaatcgactgcctaactgtagcacgataactacaacagcgggtcaagacgctgaattgcacggtccggcaccgttaagctgtaatgtgacccagtggggacgttacgagaatggaagcacacccgtattatggtgcactttatggggatcacgcacgcgagtctcattaggacaccgtgtagcgtttggctgttcttggaaaacattttttatttataacgtttctgaaagtagtggtggcacttattatcaaaaaggttacaactgcaccgacaaacatataacactatcttgtttcaacctaacggtggttcctcgagcggttcaaagcacaaccaccgtaatgacacccacggtggttacaaactccacattcagtgtgtcacttgttgcgtcgagactgacgacaaattccagcgcgtttagacacgctagttatcaacggcaacagcgtgtcggaaacgggacgttatccaagaacataactaacttggcattcacctacggcagctggggcgtcgcgatgctgctgttcgccgccgtgatggtgctcgttgatttgggtttgcctcaatcggcttggcgacgctggcgaagccacgtggacgatgaagaacgtggtttgttaatgtaggaaataaaaggcactgtttgagcatgactgtttccaaaccgtaacgtggtaaataaatcatggcttccgacgtgagctcccatcttctaacggttacacaatcccgttggacaatacatcatatgtacaataaactgttgattttggcgttgtttacccccgtgattctggaatccatcatctacgtgtctgggccacagggagggaacgttaccctggtatccaacttcacttcaaacatcagcgcacggtggtttcgctgggacggcaacgatagtcatctcatttgcttttacaaacgtggagagggtctttctacgccctatgtgggtttaagcctaagttgtgcggctaaccagatcactatcttcaacctcacgttaaacgactccggtcgttacggagcagaaggttttacgagaagcggcgaaaatgaaacgttcctgtggtataatttgaccgtgaaaccgaaacctttggaaactactacagctagtaacgtaacaaccatcgtcacgacgacaccaacggtgatcggcacgaaaagtaacgttacggggaacgccagtttagcaccacaactacgtgccgtcgctggattcttaaatcagacgcctcgggaaaacaacacgcacctggccttggtaggtgttatcgtatttatagctctaatagttgtttgtattatgggatggtggaagttgttatgtagtaaaccaaagttatagtgatgtgctttttatcagggagaaggttttgtgccaacaatgactaaccctgggctatatgcatcggaaaattataacggaaattatgaacttacggaagccgccaatacagcacgtacaaatagcagtgactgggtaacgttaggaaccagtgcgtcgctgttgagaagcacggagactgcggttaaccctagcaacgcgactacggttactccacaacctgtggaatacccagctggggaagtacaatatcaaagaacgaaaacacattattcttggatgctaattattgccataattctcatcatttttattatcatctgtctgcgagcacctcaaaaagtctacgatcgctggaaagacaataaacagtacggacaagtatttatgacggacacggagctgtgatgaactacaatgtatagatacacgtggctgctttggtggataacaatattgcttcgtatacaacagttctatcaatggtggaaaccagatacaacgtcatgcattcagaaaacgggatatgaaggtcaaaacctcagtctgcctcctagtaatgcattatcatctaaagactatactttttcatggtataaagattcacttaaagcccttaacatgttatgttattatactgaaaaacttgaagaaatagatagcaagccagatactatacgacgatgttttttgaatcatacattgtttcttattaatttaacaagtcactatagcgggatttactacttcgattctctatacacatatggttgggtattacggacacctctatgttacaatgtcactgtatattccatatatcaaacacacatccacacaactatattgctctatccgcctacgtccacatataattcattaactatatcatcatttacctcaaccaacttaacacataccgcggtccactatgccgccggtaacgttgaagcacaacacgatactgccaccccacatacaatgtggatcatacccttagttatcgttacaacaattatcgttttaatttgtttcaaatttccccagaaagcttggaataaattcacacaataccgatacaacagtatgctcaccgccgcttaaagaatcaccgtcgagaaaactaaaacgtaaaaagaatggccatgtacgtttatttttcagctcactgtttgaataccgtaaacataatgacgtacatatacgtgattatacaacaggtgtttgtgttatgcggcgactgattaaccatatcgtgaaccatgatcttttccgatggtccgtcatgaccgcaatgatattttacaggtattccgaaacctgtatggaggtcactgtcagagtaggtgatccagttaccctcggtagtggacatggttatcatccaggacaaaaagtacactggtataaccagtcatgcgtcggcattagcaacggcgaaaatacgcatcctatctgcacctacgaccctcctaaacctggtagacgaaagacaatgaaaaccactccgttaccatcaccactgttgtacgagtgtcacaattccacattaagcattcttcatgtaaacgtctcagatcccaaaaactattgcaggcgaaaatgtccaccaaacggtaactgtgaatttcccacatgttttacgttatcactgatttccagaacgacaaccaccagaaaacccggacaaaaaactacgttgtcgcgattaaaaaccacgccaaataaacatacgcagcacaaaagatccacgcgaagaacgtcacctagagattacaatgtaacgggtctgccgaaaggctttgcggactcgtttaccggtaacgtagaggcacatagagccaaagatgccgcacacagcgcatggatcctcattgtcatcatcattatcatagtcgtcattttatttttcttcaagattcctcaaagactcagagagaaatgggacaccagaggatacctttacaaaggaaccgacggcctgcccactacggactacttatcgtgagcggacggatatctccggtttcaaacccactgtttgaatatagggacagtccctacggaacctgagaacatgtggaaatcacctgtggtagaatgctgctcaggtacattacctttcatcgtgaaaaggtactttacctagcgatcgcatgcttctttggtatctacatcagtttccacgacgcatgcattctggtacctgctaaagtaggtactaacgtcacattgaacgcggtacatgtgcatgacggtgactatgtgtactggtcttttggtggaggtggagctaatagattaatgtgtcgctatacaccaaggctagacgaaattcacaaaaacaccaatcgaagtttttcatgtcttacaaatcacagtctccttctcatcaatgtaacggaagaatatactgattactatcgcaccatgaccacattcgtacatcagtcccataattggcacaaccacggcaacaaatggactttagacacatgttattatgtatacgttacccaaaacggaacacttcccactaccaccaccaaaaaacccactacgaccacgagaacgacaactaccaccacaacaaagaaaacaaccaccacgagcacgacaacgaccaccactaccaccaagaagacgacgacaagcactacccatcatcgacactccaatcccaaagaatccaccacccctaaaacccacgtagaacttcacgtcggtttaggagccacagcagcggaaacaccgttacaaccaagcccacagtaccaacacgtggctacacacgccctctgggttttagcggtcgtaatcgttattatcatcattatcattttctactttcgaataccgcaaaagctgtggctgctctggcagcatgacaagcacggcatcgtgctcatccctcaaaccgatctgtgagcaagtcgcgtaggaaatgattgcatgaaatcactgtgaaacgccaactccgtgccagctggcgcggcggacaggcctttgacgtatttgaagccaggcgcgctctcgataccgaaaggatccgagggggctttccaaagccgacgtccctgattcccttcataaagctgttgaccggccctagaaagaccaagagcatgctgtgggcccactgcggtcgcttcttgcgttatcatctgctcccgctgctgctgtgtagactgccattcttactccttttccagcggccgcagtgggcccacggcttggacattgtcgaggaggacgagtggctacgggagatacaaggagcgacgtaccagctgtccatagtgcgccaagccatgcagcacgccggattccaagtcagagcagcgtcggtcatgacgcggcgaaacgccgttgacctggaccgaccgccgctttggtcgggatcgctcccgcatttgcccgtctacgatgtgcgttccccgcggccgttgagaccgccgtcatcacagcatcacgccgtatcacccgaactgccgtcgcgagacgggatacgttggcagtaccaagagctgcagtatctggtggaagaacaacggcggcgaaatcagtcgcgcaatgcgattccgagaccctcgttcccccctccggatccaccatcgcagccggcagaggatgcacgagacgcggacgcagaacgtaccgaatcaccacatagtgcagaaagcaccgtcaggcacgacgcgagtgagaacgcagtgcggcgacggcacgaaagacggcgctataacgctctgacggtccgcagccgggactcgctgctcctgacgcgaatacgcttctccaaccaacggtgtttcggacgcgggcgtctgagacatcccgcgggaagcggtcccaacaccggcggaccgcgacccggcggtgcgggactccgtcaactacgccaacaactgacggtccgctggcagctgttccgcctacggtgccacggttggacacagcaggtctctagccagatcagaacccgctgggaggaaagcaacgtcgtgagccagacggccacgcgagtacgtacgtggttcgtggaaagaaccacgttttggcgtcgcacgtgggttccgagacagaacccggcggccgaagcgcaagaactggccgtcataccgccggcacccacggtgctccggcagaacgaggaaccacgtcaacagcttacgggagaggagacaagaaattcaacgcacactcaacgtgaagaagtggaggacgtttcgagagagggcgcgagagaagggaatgatgggagccgagcaagtggaaacgacgagagaaggaataatgcgggaagatatgatgatgatcatgaggttcaagagccgcaggtcacttatccagcgggacaaggagaactgaataggaggtcacaggaggagaacgaggaaggtggaccgtgtgaatcgccgccaatgacgacaaatacgctgaccgtggcctgtccgccccgagaacccccgcatcgtgccctgtttcgtctatgcttaggactgtgggtctcgagctacctggttcgacggcccatgacgatttagaatacaccgagccattcctttatttccccccatccccggtcgcttatgcgtgttaaacactaccaataaagataatctgccaatcgcaccttatatataatatgtggtcgcgtgtggtctttttaaggagctctgaaacacagacaggtatgggcggtggtcggctgccgccgctgtggctgccgctactgatcgcctggagcgagtggggcaactgctgcctcgatgcgcctccggtggtgcgttcgccctgtctgcagccggtgcgcgaccgcaaccgcgagcggaacccgggctcaccgcagttgctgccttacggcgaccgtctggaggtggcctgcatcttccccgcgcacgactggccagaggtctctatccgagtccacctctgctactggcccgagatcgtgcgttcgctggtggtggacgcacgcagcggtcaggtgttacacaacgacgccagctgttacatcgccggcgggcgctggcgcttcgaggacggcggcgcggcgcagcggctgagcctctcgtttcggctcatcaccgagaccgcgggcacctacacctgcgtgctgggcaacgagacccacagcctggcgaccgagaccacggcgctggtggccgacgtgcacgacctgcgccactcggaccgctcctgcgacctggctttcggatcgcgctcacagacgcggtacctgtggacgcccgatccctccaggttgcgcagtataaactgcggttgggagggtgaacggcaccgcgtagtccactacatccccggcacctcgggtttgctgccctcgtgcgaggaggacgagcgcgaactgtgcgtgcccttcatcagccagagcattgcggacaacaactgcagccgccggcatcgagttgacggcgctaggcggcgctatcatctacggagggattactggctgacggatccgaagatcgggttgctggccgcgggatcggtggccctgacctccctctgccacctgctgtgctactggtgttccgaatcgtaccggcgtctgaacaccgaggaggaaagcgaggcggcggaggaaactgccgcgggagaagcctctgcggtagcggcggcggccgtctctgaggaagagcagcggcgggagtaaacgaggagagccatgaagcggatgattcgcagtcacggcaggaaaacggaatgtcagatgacgagcgccggcgagcgacgcggctccgccgtcggtgcgcccatctgcggcagcggtacccgacgcggcagcggcgccaacgaacgccgcgactccgacgtcggtcccatcgcccacagtagcggtaccagacgcggttcggcgaatgaaacgtccgcctgtacgcggaccgatcaccagaaggcggacattgggctgtggttcatgtttctggtttttggactgtgttcgtggttggcgatgcggtatcgcgcacaataaattttgaatcgatgtcaaggaacgcgtgttttgtattttattgggaatattggcggggataaacctgtttcggatgtttacccttaatcttaccggggacctcgttgtcctctcctccttcttcctcggacaccgggctccatgctgacgtaggtaccgactggggtcaaaagcctgggtacttatgaggagcgcgcacaaaggaccgttaggcgccggcatggagcgtcgccgaggtacggtaccgctgggatgggtgttttttgttctttgcttatctgcctcttcctcgtgtgctgttgacctgggtagcaagtcctccaactcgacctgccgcttgaatgtgacggagttggcctcgatccatcctggggaaacgtggacgttacacgggatgtgtatttctatctgctactacgagaatgtgaccgaggacgagatcatcggcgtggcttttacttggcagcataacgagtctgtggttgacctgtggttgtaccagaacgacacggtgatccgcaatttcagcgacatcaccactaacatcttgcaagacggactgaaaatgcgaaccgtccctgtgactaaactgtacaccagccgcatggtcactaatcttaccgtgggccgctatgactgtttacgctgcgagaacggtacgacgaaaataatcgagcgcctctacgtccgattgggctcgctatatccgagaccgcccggatccgggctcgccaaacacccctccgtaagcgccgacgaggaactgtccgcgaccttggcgagagacatcgtgttggtctcagccatcactctgttcttcttcttgttggccctacggatcccccagcgactgtgtcagcggctgcgcattcgcctgccgcatcgataccagcggttacgcaccgaggactgaacggataaccgcaaaggccacgtgcaacgttcacgctgctataagaaggccatgtcccccgtggacgggtctctttgacacgagcgcggcacgccgttgccacgagcatggatcacgcgctcttcacacacttcgtcggccgaccccgtcactgtcggttggaaatgttgattctggacgaacaggtgtctaagagatcctgggacaccacggtttaccacaggcgccgcaaacatctacctcgacgtcgcgctccgtgcggcccccagaggcccgccgagattcccaaaagaagaaaaaaggcggccgtccttctattttggcacgatttgtgctggctgtttcgacgacttttctttcctcgggaggactcagagccactgatgtcggatccggcacggtctcccgaagaggaggagtaaacaacacacggctaagaggatacatcatcaaagaagataggaggggtcaaaacgcggactgaaagtatataacgccgatcatgtccgaggaactgttaataaaacgccatgatgacaatgtggtgtctgacgttgtttgtgctgtggatgttgagagtggtgggaatgcacgtgttgcgttacgggtacacggggattttcgatgatacatcgcatatgacgttgaccgttgtggggatttttgacgggcaacacttttttacctatcacgttaattccagcgataaagcgtcaagtcgggccaacggtaccatttcttggatggctaacgtctcggcggcctaccccacctacctggacggggaaagagccaaaggtgaccttatttttaaccaaaccgagcaaaacctgttagagctggaaattgcgttgggttaccggtcacagagcgtgctgacgtggacgcacgagtgtaataccacggaaaacggtagttttgtagccggttacgagggatttgggtgggacggggaaactttaatggagctcaaggataacctgacactatggacgggccccaattacgaaattagttggttgaagcaaaacaaaacgtacatcgacggtaaaattaaaaacatcagcgagggggatactacaatacaaaggaactatctcaagggtaattgcactcaatggtccgtcatttatagcgggtttcaaccccccgtcacccacccagtggtaaagggcggtgtccgaaaccagaatgacaacagagctgaagcattctgtacatcttacgggttctttccaggggaaattaatattacttttattcattacggtgataaggtgcccgaggatagcgagcctcaatgcaatccgctacttcccaccttggatgggactttccatcagggatgttacgtagccatcttttgcaatcaaaactacacctgccgcgttacacacggtaattggacggtggaaatccccatcagcgttacctcacctgacgacagttcctcgggggaggtccctgatcacccgacagctaacaaacgctataacaccatgaccatcagcagtgtcctcctagccctgcttttatgcgctttgctattcgcgttcctgcactactttaccaccttgaaacaatacctacgtaacctggcctttgcgtggcgctatcgcaaggtccggtcgtcatgaccagcaacgccctgtatgagctgtttcgacgtaggttaccgcgtgcccccgtcaacacggtcatgtttctcacgcgacgcactcgtgatgggttctgcggtcggttgacgtccatcgccacgaattcccactacactatgttcgtgttagatcacgggtccgtgcgcatcgagcgaccgagtcagtcagaagtggattgcgccagtttaatggaaacgctgaagcggattcggttacgaaattcgtgggtagcgtcagaagacgagctagatgtgagtcgcggggacgcgtgacacaaaacgcgttcaggattaacgtaggttttcgaaataacctacgtccgtgagtgacgcggtttcgtgttgaaacccgcgccggttctcacggtggtttatgatgaaaccggcgttggggatctacgcgggttcctcattcaacctgcgaaaagaggaagttgcggtaaaaccacgtcaataaagacgtcaatgacacctcaatgttgcgttggaacggtctttatatatacaaacgccgttatgttcagtgtccggcaagatgctcgggatacgggctatgctggtgatgctggattactactggatacagttgataacaaacaatgacactcgaagcaacaataccgataccatctttgtatctctccttaccggggccaacggagttactcgcacagccatcgggggtctgcattcaaactacaccaacttaaccgaggcattcagattcactccagcaaacacaacaactaactcttccacggagggtaattggagcgtgactaacctaacggagagttgcatcaaccgcggtgagtcctatctaactaccatctggcttctgaactgcgctgacaacaatacttattggtactctggaaatgcctataaccatacaattgacacttgtaaaaatacagtttcgggatatctcttcttcggcatgtgccagctatggaaagattgggttactaatgcttctcacgacactgtcagaattcagtcgttgggaaatgaaatacgctgcatgctgctccctagacagtataccctcaacgccacggtggaatggtacaacaaatctgaaggtgacgtaccagaagaatttatggactatgttatcctgacccccttggcagtgcttacatgcggactgcaggaagcttatatactcgacaagggtcgtagatacatgtatttgttttccgtgtcctgcgcgggaatcacaggtaccgtatctattatactcgtctccctatcgctgctcatcctcatctgttactatcgctgtggccggcttctgatatgcccacgcggctttgaactcttgccagaattcactgaggaagaggaggaaaaagaaaaattgttaacgtacaaggacattgaagtccaggtgcctatccgcacgcggcggctgctcgtcccttggatccgggagagcaaaatgtgggtactaccacccccgctgcctccacgacctccccacttaatagaattcccgccgtctcctccgccgtcgcctgggcccatgcacatggtggtctgcatgccagcatgacggactttgaactttgagccccaagcggtacggactacatattttccataaatctacactgaacttgagcacaaaaatactgacaatggactgaatatacagacttttatatgatccttgtacagatgtaaataaaatgtttttatttaaaactggtcccaatgttcttcgggaatcatggggtggggacgggggacgcggtaaggagcaaaaccgggtacatgggggggaacatcgtccagcagtagcaccagcggattgggtaggggttgctgcggaggtcggtcgatgacgatgtcgatctccatcggcagatccggcaacatctcttcgtctccctcaccgaccagcactcggcgctgttctggatgtatatgattttggaaaagcctccgacgagctcgcggcgcgtagaaagccaagcggcgcaagggccggcgagcccgaaagtccatgcgcacagatggcatgagtccttgagtgacggtggtgagctggggaacagggctacctcccatcgcgacggtgacagtggatccatgagagaggcgccgcacgctgcatggctaaataccgtgaatcccctgacgtcgtctttcgtcccgaacgcgtcatgttgggggcgaggcgtaaaccgtcgaggttgaaaaaccgcgtatctgcgacccgtccggactacgttgtttttcagaagcggccacatgacctcgagatgtcgtcacccaaggtatttaacggcacacagccagacgcgttcgtcagcagcgacgccgacaagacctcagcatggctcggaggctatggatcttgagcttactagccgtgaccttgacggtggctttggcggcaccttctcagaaatcgaagcgcaggtaaacggaatctggggaattcaacacaggtaagaaatacaaaaaataacgtgattgtgaacgcggttatcgtgtttttgcagcgtgacggtggaacaacccagtaccagcgctgatggtagtaataccacccccagcaagaacgtaactctcagtcagggggggtccaccaccgacggagacgaagattactccggggagtatgacgttttgattacagacggagatggcagcgaacatcagcaaccacaaaagactgatgaacacaaagaaaatcaagccaaagaaaatgaaaagaagattcagtaacagcagaccccaagggttaacgattatgttgactaccttgttttttattaaaaagctgtaaggttttgctctaaaaacaccccgcctccggtcttttttcttttgtattcggcacgcgaaacacggtttcttcccatagcctgtctaactagccttcccgtgagagtttatgaacatgtatctcaccagaatgctagtttgtagaggctatgcgggatgctgcggcggcgcgaccttccctctccacccagccccgtcaaaacacacgcgactcgagcggttcgtatgaaaaataaaaaacagctttttatttacaggaacggggaaaaaaaaggcacacggtccgtgggagacgcgggttcacgcgtcgtcaaaaagttggtggtccactccgtaaggacaggtaggcttatttagcttccgcatgctcctggttccgtaataaatgccgttttcgtggcagcgtgtcatgccgcgagtcacaaactccatcaaactgtcggccacgatgcaaacgtgctgattgttggcagcaaagacgcgcatacagtcgtccacgaagaggttgatcacgtcgtaggggctcaccaaccagcctaaaggttccacgtggttactgccgaccatgaccctccagtcgttaatctcgctccagtcgtacagccgaatcgtggagacgcgaatgacgctgtaatcacccatgaccatgagtcggccgcgatacgtagcacgccactgcgcgaacgcgtggatgtgcatgcagccggccagcgctctaagcgaggcggtgtgcggcagctcctctgggacggtgatgaagttgcagcgtcgcaaaccgatgttgagaaattcagtgatgctctcggccacaaaggtcaacgagtcagagtagatgtggtcggtccacaggtacatggcgcccgaggcgcccaggtacagttcagacggcacgttgtgatcgcccttgtgtttaagaaagttgtaggtgcagatgctgccgacgaaacgcagcggctcggggcagcagaggtagctggccagacgctgtgcatcccgtccttcgtcgcgcaccaagcgccagcgacgccggataacgaggcagcggtctttgggccagaccagggccacgcgttgcccgggtttccacggtcgcgacgtcttaggaggcctccagcggtcgagcagattgagaaaacagtccttgattaccgacatcgcggtcgcgcgtcggtggacaaaaagaaatcgggccgatccggaaaaaaaaaacgacggcaaaacaccgccgtgctcgagcgaagggtggcggagggccagaagaggcggccttgacggcgttggcagcgaaaaaattggcacgcgagtcaaacgggaagtagcgtcggtgttttatgccccaagcagcgtcgtcgtcactcgtggcgtcacagtcaacggtgctgacgtcctttggggcagtcgggcacgcgatcgtagatgccgttgtggccgctgaaacgtcggttttcaaacagcaggttaagtcccagacacatgaacgtgttgagattatctcccacccggatgtagcggtcgtcgcgcacgtcgcaggcgtagacggccccggtataggcgacgacgatggggataaggtcgacgggccagcgcaagtgaggaaagggcgcgttctcgcccttgaggctgacggttcccaggccgagaacgcgcattccgaaagcggttttgatgttgcgcagcaagtgaccgccttccacgctgttttcgaaacacctgaggttgcatagacgcagttccgttcccggcgggtacgtcaacggcatgaactgcccgtggtggcggatgatgaatcgcgccatggtatccaaaccgaggctccaggcgcgcaacagcgggcgaaagtagcgcttaaccaacgacgaggtcaggtagcgcatgcagtgcagggtctcgacggcgcgcagcccgacgcgcgcaaactccatgaggttgcgggccaggtagtagacggcggtgtcctcgcgtacatagcaaaaaacatagccctcgtccgagatgaggcacacagcggtcttcttctgctgatccggcgacaacacgccctcgttcacgaagcgacccacgaaggccaggcgcgtctggcaacacaggtagtgactccaagccttcacgtcctccggtttgaagtcctcgtccgtctcgatctcctgcagcactaggttccagcccggcggccagaccacgggcaacacctggcctgcgttgatgcgcacgtaagcttccagacagcccaggccgaactcggccgtgagcgccaggctagccagatcgctcatgtgacgcgccgagtcagtgggcgagcccgggggcccgtcgcacaccacgctccgtcttcttgtcctcaccgcggccagcgtggcgaggacactttccgcgcccgaggctgtatcttcggtttgcccgccggagccggccctcactatataacgtcccgcccgggtctcctccatgtatgcaggtaagcaactgagccgaacgcacctcagcagacgagaggatgtcgtcgcggcgtcgcagctcgtcacgtcgctctggcgaaccctcgacggtgatttatatcccctcgagcaacgaggacacgccggcggatgaggaggcggaggacagcgttttcacgagcacgcgggcgcgcagcgccacggaagatctggatcgcatggaggccggtttgtcgccctacagcgtctcctcggacgctccgtcgtccttcgagctcgtgcgcgagaccggcggcaccggcgccgccaagaaaccgagcgaaaagaaacgatcgtcgtcgcgtcggcaaccgcagatcgcagcgggcgcgcctcggggctcgccggcgacacccaaggccggcaagtcgcctaaagtctcgcgaccgcctagtgtgccctcgctgcccgagaacggcgccggcggcggtggcgacgataacagcagcagcggcggtagcagcagtcgcaccaccagtaacagtagcagaagtaccagtcccgtggcgccaggtgagccgtccgctgccgagggcgatgagttttccttctgcgacagcgacatcgaagactttgagcgcgaatgttaccgggtcagcgtggccgacaatctgggcttcgagcccagcgtggtcgcgccgcagcacgtcgagtatctcaaattcgtgctgcaagactttgacgtgcagcacctccgccgcctcaacgaatgcatacccatgccggccttcgcgctcaccagcctcgtcgaccccgtcttaaacaacgtagcgcctggcgagcgcgatctcacgcgtcggataatcacgcacgcggtgatcatcaactattactacgtggcgcaaaagaaagcgcgccacatggtggaggccatacggaccaccgtgcggggcgacacggtacgccgggtagccgcgcaggtcaacaaccagagccgttcggggcgtgcggccgcgctagcgcttcattttctcacgtcacgaaaaggagtgacggacggccagtacgccacgtctctgcggcggctggacgaagagctgcggcatcgcggcacgcccgaatcgccgcggctcaccgaggtttaccagacgctacgcgattacaacgtgctcttctataccgcccactacacctcgcgcggcgcgctctacctctatcggcaaaacctgcagcggctcaacgagaaccaccggggcatgctccggctgctttcggtcgaagagatatgcgaagagcacacgctcaacgatctggcgttcctagtaggcgtcgagcttatgatcacgcactttcaacgcaccattcgcgtgctgcgctgctatctccagcaccagctgcagagcatctcggagctgtgttacctcatctatgtacaactgccgtcgttgcgcgaagactacgcgcagcttagtgacgtgatctactgggccgtcagtcaaaactacgactacgcgctctacgcgagcacgccggcgttgtttgactttttacgcgtcgtgcgtcagcaggacgccttcatttgcaccgactacgtgtactgcgccctgcgtctgctggcctgtcccgacagacctattatcggtgacaccggcggcagcagtagctcccaacgcctcgtaggcgagtttatggtgcgcgatccgctgttgcgcgacccgcgcgccacccacctgcgccagaaactcatcacccgcgacatatgcgtggcgcggttgcaagcgcagccctcgagtcgacacattccggtcgaacacacgggtgtctcctccgtcaccctgctcaaaatctttagccaagtcccccccgacgaacgcgaagaagacacgttacgcgagatggctcttaaagcgtttatggaagcgaacggtaatcaccccgaacaaatctgccgatccccaccacccccgctgccaccgcgcgactatcctcaacgcgacgagcgggaccgtcaccgtcgcgaccgccgcgacagcggggaatactgttgctgatggtgggacgaaacagcagggcggaacagtttatgatagaaagtcacaggaaagtatgtgttgttttttttttaatgtaccaagaataaaaagtgcgtctacgaccaaagcggtgtgtggacgctcgtcctctctgtcttctccgggtttttttttcacgtgttttttccttcctattttgttacggcaacagcgctgatggcacgttgccggcttcgaacatcgcgtcggtgatttcttgcttgcccggcgtcacacggtgacgtagcagcacgcggctcacgtagcaggccgactcgcggatgacctggccgtcggcgtcgcgtcgcaggcccgagcggttgccgtgacgcagtctgccctgcgcagcgcgctccacgtcttcaaagtagctgtgtagcaggccgcgctccagcagctgcggcagcgagtcggcggcgcgcactacaaagttctcacggctgatctcgtagcacagcacgctgccgtcggccgccacgccggccacgctgcggtcccaactgaaaaggttggcgagtccgatggtgccgatgacgcgcaactgaccctgggtcaccaccagcagcttccagtattctacgtcgcgcggggtgaggatggtctcctccacgtcgcagacaaacaacgtgtagccgcgcggatagggcagatccaggtggcgaccgcgctggcggcgcataaaatcgtctaaattcaaaccgccgtcgggtgcgcgcctactcgtcatcgccgcgccttgtcggtcgatgaccccacggtgcttataacgcgccgccgcggcttcatgtggcgtgacctccgacctcgtgaggccgaaaacggcgtacatgaagacgctcaaacttttgaatgtgggcccggtagcgcaccgagggccccggggcggcgacgacggcgggcccgagttccagcggggccttgcggcggcagcggttggcgtggttgctcagctcggcgtccgagagcgccgagctgaactgcggcagccgcgtgcgatcctgcggcgcgtccccgtgtcgcagcgagtgccagagcaggcgctggacgcgcgccgtctcgggcgtcggcggcgcgcgacagccccggcgcagcttgaaaacgtgcaggcacagcagctcgcgcttgatgcgcagcgacacgctgcggtagtcgggaatccgctgcaccagctcgagaaagtcgcagaaggtctccacgaacgtgtcctcggtgaagcgaatgcgcttcagatcgtggacgtgtttgcgaaaccgcgacagttctcgacgttgcacggggttctgagcgagtcccttgcgcagcagcgcagcctcgcctttaaacagcctgatgagccgctgcacgtccccgctcaacatacgtatacacgccgtgtactcgtgacgtatactggcgcgcagcagccgaatgatacgcagggccagcacggcgttagaggccaggtacatggcgtagccgcgacgcgggttggcacaggcccagcccgcggggagcagaaagtagtcgtcgatcagcgtctgcgaccagtcggcgaagcccaggtcacgtgatacgctgtcctggacgcgggccacgtcgccggccgtgaggtggcggatcgccggcaggtgaaacgcgcccaggtgtcggttgcgctccagcctcagctcggcgtgctccaaacgggaatggtgggacgccaccgcggagggcgacaaagaggagtggtcattgccgtcgtggttaccgtcgtggttaccgccgttgtcgcgcccgtcgccgcactcgcaaaaggccgcgtagaggtccttcaatgccgcttcggctcgcgccataaacgtggcgtggaaaaaaacggcggcgcggtgcgtccggtacttgacgggcaacccgcggcacagggccgccggcaggcagcggccgatgagttcgcgctcctcgggctccagaaacaggcacagggtgccgtccaggcgcaggtacagctcctcggtcatcgagcatagctgccgcaagtaatgggtgcgcgtcccaaaggtcttgtaatcgagcaacgtgcacaccacgtattgccccgtggccacggccagagcgatgcgtttggcggcacgactgatctctggcaagtactgcgcctcgtgcaccagacggcggaaagcgccggcgttgagccagcgaaaatgctgcggatcgggcggcaagggcacgcctcgaagcgcggcccagacagcgaggtccgactcgagcgtcagaccgcggatgtcgtacttgccgtgcgccgtagcgcaggctgaatggaccagacagctgcggcgaatgtacaccatggcgtgcttgggatgtttgggcgccggcgttttctttttctgaccgccggcggccgccagatcctcgggcgtgcgacacaacaggccggcgcgcacagcctcctgtcgattacgaatcggcgtcaggtaggcgcgcaggaactggtgacaaaactcctcatcatcacgacagtcgtcgagatactcgtacgtggtgagcggatcgcgaaataggcgctcgtcaccgtcgtcatggtcttctttagcctgctcctccggctgctgggttggcagtggaggcggcggctgatccacggggttcatgactgagaggaagaagaaggtggcggcgaagcgacgcggagcgacggcggtaaagccagacaccggctatatagctagtcatcacagtctcctccttcacgacgcccccgtgccgctcacgctatccagcacgctacggcccgaaaacacgtactcgctgacgtcgtacgcgggcgatgtatggctgctcaccggtttcgcggcgacggttgcgctcgagtccaacggcgagaagcaaaaacgccgtgggcaacgaaaccagaaggagccctgacggataaaaccgcgcagcgtctcggccaacttaaccagcatcgtaccgtacagcagtacgtgaatgccgccatgcgcgtccataaatacggctttgttcacgggttccatccatccgatgactacaaaatgggcctgttctagcacgccgatcacgaaattgttggcctcgtcggcctcggccacgttccacgagccgaaagtgaaagtacaagcgggcgagccgcccaggcggatcttgctaccggcgtggagctgacatacgcgcagcagattggcgcggtcgtgcagtatctgggagagttcgtacatgcccgcaaaggtgtgcttaaaccacgcgccctctacgatctcatccacgtagtcgcgctcaaagaagctgtacacggcaaagaggccgttctcaaaaaactcgccgaacgagagccccagcacgtacaccttgtcctcgccgggcaggtacgcaaaggcgtgcccgtgcccggagacccagatctcgggcgccgtgtttgcgtccggcacgcattcgtacacactgacgaggccgataaagtacaagcggccagcctggcgcaggcacgagaagcgccggtaggtcttgtgatcgcgcaccaccccaaagtactgagtgtcgcccagcatgatgccgtgcagcggcggccagcacagcgggagccaacgacccgccgtggcgcgcacgtagcgctgcaggtgaaccccgctcgcacgctcgcgcggcttcgggcgcttgtgggtccaggcatcacgcagaccgcgccagatgctgctgaacttgggctgcccgcgcagatagagcgacgagagcgagtcaaagtagcccacgacgagcctgtcgggagacacaagagcgcgaaaatcaaacctagagcgacgacggtgaaaaaaccgaccagaagcgcgtgtctcaaacacgctactttcggttataaaaacaccgtcgccctatttctgggcgcgtgtacactgatgactcacctacgctttttgaacggcagtctcagctcgggattggcctcgtacagcgagctgcggtccacggggccgatgctctcgtagcgaaagtcgtcgatgagcagcgccagccccacgcgcacgaagcccctgaggtcgcgcgccagccgcaccaacttatcctgccccaccagcgccgcgtacacggtgcccgtgtcgccgcagagaatccgcacgcggtgaaagaaggtcttgtcctcggcgccctcaatttcgcccagcggcatgacgggctcgcgcgtgtacaacgaacgttgaaagcggcgcagcatcgaggccgagagccccagatcgcgcgccgtgcgcagcactagggaatgcttctcgggccagatgagggtcagttgcgcctcgcggtgcgcctctacgtaggcgcaacgagcggcggtgtcctcgcaggccagcaactcgcggaaagccagcagcgaacgtaggtagcggccgcgagcggaggcgcgcgagcggcggcacagctcggcccgatggtcgggatgcaccaagggcacgttaggttgcagacgcgcgcagatggattcgtgcaccgggtcgcagcggatcatgcccttggcaaaaaatccggccagatccgaggccaactcgtacaggcagtcctcttgcgcgtcgtaggcgaacacggcgccgtacgcgtccacgaacacctggtaccggcaggtggcgtgcgagaccgtgccaatgagatgcagagctcggaattcgccgaaaaagtcgttctggcagtgctccagatcgatctcggtcagcgagtgcggcgaatgctcgcccccgaccacgtagatgcactgcgagggccagcccagcgacacgcacgagccctcgaagcgccgcaagtaacgccgcaggccctcatagtcgcgtcgcacgcacaggtcggccaagtcgcgcgtgcaaaagacctcgggtaccaagcagcgtttgcgacgcggccgacgcgcgtgcccaggcagaggaggaaggcgcgacggcggcgacgacgaggaggaagacgccgtggccgccgagcagcccttgcgacggccggacatgccggcagtccgcgacgatccacaggagacaaaaaagcagaagcagcagtagcctcggcgacccgctccaccccgtcctccacacgctcagccgcgactgaacgccggggcgcgccgctacttgggtttttatagccatctgccccccgtctcgggcacccgggagcgatctacggagacctgacagcagttgggcaacacaagatagggaaatacaaagacacttttaataaaaaacgagactactttgtgtgtgtgctccgtaaactgtttattctccccctccgcttcgctctggatgggctccgggtccgtcaacacgcgactcgcgcggcaaaaggcacgctgttgacggcgcgagagcccgtcgtgatagtccatcatgccccggagatcgtgcacaaagcagctgtcgccgcgcagaaaccgacgcagcgtctccacgtgctgcagctgccggcgcgtatcaggagccgtcatcgctgatgtcgtcatcgccctgacaggcgcgtagatggcttcgcgagatcatgcgcgttttcaaccgccgtgacacatcaggtccatcttgagctggcgccgggcctcgcgcaggtgttgcacgcgttgtgagcgggaggcgagttcggcttcttgctcgaactcctgctgctcactgtccgagagggtgcgataaaaggcggcaaagtcctccaagtcggctacatgcgccctgggtctgacgctccaaagcgtacgcagtctgatgaagcggacccatcgagcgtcacggcacgccgtcttgaacgcggggcccgggaagaggttcttctccccggcgcgctcgggccggcgaggccgacgcggtttatatacaccgtctcggacggcgggacgccgagcccgcgccgcggccgctcatccggagacggcggaaaccgcgacgccggaggaaacggggaccggcaacgacggcggtggcggcgaccagattatgggggacaaacccacgcttgtgaccctgttgaccgtcgccgtgtcgtcgccgccaccgtcgtcgccgctgccgctcgtcagcttcacggagctgttgttaccgccgccgtccgtcgccgccgctgcggtggcggcgacagcgacgagcgaggtgggcgagaaaaccgcggagcaagaggtagcggctgcggatccggagaccgggaatgagagaagagaaaacagggagaacgaaggaggggagacgaggacgacaggcaccaccgcggtcaaaaggtcgcacgacggtatccctcgccaactggcagagcgcctgcggctgtgccgccacatggaccccgagcaggactatcgtctgccggcgcaggacgtggtgacctcgtggatcgaagcgctacgcgacgcggaccgcgacaactacggtcgctgcgtgcgccacgccaagattcaccgttcggcctcgcacctgacggcctacgagtcgtacttggtgtccatcaccgagcagtacaacacggcctcgaacgtgacggagaaagcttcgtacgtgcagggctgcatctttctctcgtttcccgtcatttacaacaacacgcagggctgcggctacaagtacgactggtccaacgtggtgacgcccaaggcggcgtacgccgagctcttctttctgctctgctccaccagcgagagctccgtggtgctgcaaccgctcatcaccaagggcgggctctgctcgtccatggcggtttacgacgaggaaaccatgcggcagtcgcaggcggtgcagatcggttttctgcacacacaactggtcatggtgcccttcgtgccgcacgcctgcccgcattacgccgtgcctttcacgacgccgggaaagccgggctgcggcggtgctccgagcggcgttgcggggttggaggaggcggcgccctttggacgggtcagcgtcacgcggcatggcgcgacgctgctatgtcgcgtggaccatctgacctggatcagtaagcgcgtaaccacgtacggacacaaaaaaattacgcgctacctcgcgcagttccgcggcacgatggacgacgacgaggcagcgctacccggcgaggacgaagcgtggatcgcgtccaaaaacgtgcagtacgaattcatgggtctcattttcaccgtcaacgtggattcactatgcgtggacgcggaacagcgccaactgctgggcaccgtggccacctccttctgtcaccgcgtctcggacaagatcacggcgcgcaacatgccgcgcgctttttccttctacctgctgacgagcgcgcagcgcgggtacgacctgcgattcagccgcaacccgtcactcttttttagcggcgacgcgctcaactgtccgcttctcaatgagcccaacgtgttttcgctcacggtgcacgcgccttacgatatccacttcggggtgcaaccgcggcagacggtggagttggacttgcgctacgtgcagatcacagaccggtgtttcttggtggccaacttgccacacgaggacgccttttacacggggctcagcgtgtggcgcggcggcgagccgctcaaagtcacgctgtggacgcgcacgcgttccatcgtgatcccgcagggcacccccatcgccacgttgtatcaaattaccgagggcgacggtaacgtgtactcgtacaaccaccacacggtgtttcggcagatgcacgccgccggaacaaccacgttctttctgggcgacatgcaattgcccgcggacaactttctcacgtctccccatccctgaccctccgtccgtcctcctttcccgacacgtcactatccgatggtttcattaaaaagtacgtctgcgtgtgtgtttcttaactattcctccgtgttcttaatcttctcgatcttttggaggatgttctgcacggcgtccgacggcgttttggcgccccccatgccggcagaacccggttgcggccccgtaccgctcttctggggcgacgataggtcgaaagccaccgttttcatgcccgtcgtgctcttgacgggggaacctacggcggcggtccccgtcgagcggcgtgattgcaaagccgcgctcgcccccggtttcaggatggagggggaggccacaggcggcgcattcgatacgctgcttttggccgtagacgacggtgggtaaacggtggttaccgcgggatacgtcggcgtggtcgaggcggcccggctgctgccggacaggcgacccggcgcgctaccgctcacggggaccgagggcggtcgacctaccaccgccttgccgcccaaagtaggtttcaaggaaggaacaccgacgcggctgccccggcctttcaccggagacgggggggcactcttggccggggacggagaggctgacgaaagcatggacagcggcgatgtggcgggggacacgacatcatcctccgtgggcgacaaaacggacgccgaggctgacggctgtcgagccgaagaagcggaagaggttcccgcgccagaagtcacgttccttgatgacgtcgttttagacgaagccggttgaggttgcaacagcgtggcgggtaccgtcgacggcgtgcccgacacctgtttctctagccttccctgaaccggcgtcgacgtcaccgtctgcgctcgggcggacgcgtgcggcgtcgcgactcgcttgcccagcaccggtttctggctcgtggatgtcgtcgtcattggagacgataacttagctttacgtattctggacggcgtcgactgctcgggcgtctgactgggaggcgaaatgacgtcgttgttgtaatcggacgacggtgttgtgtgtcccaggctgacgacggagccggtgtccgaggagtcgtcgtcttcctcctcgctgtcttcgaccggtgactctgcagtttggtcccttaaagcccaaacctcatcagcggcgtcccgagacgctgtttgtgtcaccgcggcgcgtggagtcgacggcctccgaggggtggtggacacggtgttttgagaagccgtggaagtcgtaggcatcctgaagggattgtaagccaggtgaggattcttgagggcccacgcgcgttcgcgcggccagttggcggggttcatatccccgggcaacggcgccgtcggagcccagggcgagttaccgttgaccggggtttgggtacccgcgaaggtaggtgtcggggccggagcgggggccgtggaaggattgacaggcgtcggcgtgaggatggcagcgccggcgccagcaggaacgttaactccggcgccgaacgtcaacgtcggttgctcgaacttgtacgcggtggtgacgggcggtttggcgctcgtctcggtatccgtgatgtccaccagcgtgtcggtgaaacgcggatcttgacggttggggggatagccatccgagctgtcggaatcctcgtcgcccgagaaaagatcccctctggtctccgtgagcggcctcacgtcccacgcgctgtcccgacggacccttcccgggctggccttggtcacctgcggggagacgagactgaaagccgcgtgacgctgttgttgttgcgggatgttcaagggaccgctggtcggtttctgactgcccgaggataacaggccgctgaaaatgctggaaacaccgccaccactagcggcgcccttgccgctagttcccggtttcttgatgggcgtaaagatgtttttctcgtcatcatcatcgtcgtcgtcctcatcggcactggagccaaagagcctccgggaggcgctcggtttacgtgccgggggcggtggttgctgctgacgttgctgcaggttctgctgcctctcctcccaagccttcagctgctgtttctcacgctgcaccacctcgtcgtccacccgtttctgccgctcgcgacgcttttcctcttcgtcgtaatagccgacggccgccgaacgggcagcgtgggcttcggcggccggtgccagagaaccatgggcctcgaagcggaacggtttgtgtcccttccagggactggcgatccagctccagccgtccagcggctgcgtggggacatgtttcttgggtaccgacgagaaggccgaaccgccgccgagcgagaggagattggcgtcatcgtcaaactccaacgacggcgagcgcgcgcccaaaaacgtgtgcgccgactgtgggaagctgtccacgtagatatcaaagtcctcgatgagcagctccaacagcgtgtcggccgagtcgccgttttccacggcgtgcttgaggatattgcgacagtagttggaatcaaaggaaaggcacatgcgcagctccttgaccagcagcttgcagcgctcctgaatgcgcgccagacatttgcgctccagctcctcccaagacctgcgcacgttcatgatgagacggcccgtgtacacgagcttgttgacggcgttgaccagcgccgtgttggcgtgccggtccaggttaaggtcgagcggtttcacgcagaacatgttacggcgcacaccctccaggttttcttcaatgcgctgcacctccgtatccttgaggtgcacaaaggcgatgggttccgtctggccgatggctgtgaccagcgtctcgcgcaccgacatcttggccagaatgaccgcgcttacgagcgcgcgttccacgatctcggcatcgtggcgcacgtccgtatcgaattcggtacggtctagcacagccaggtggtcacgcgccttaccacgatcaccgaacgggtaagtgtagccgcgacgcgccacggccacgcaacgcacctcgaactcctcgagcactgaggagaggtcggggttgtgaaaacgcagctcgcggtagtatcccaaccaaagcatgagctcgttgaacagcaccgtacgccggtgcaggcgtttttcgccacattttttcaggatcttggggtgtgcctcgagatccacgtcgggcttttgcgtgagatggcgcagaaagttgaccagggctaccacatcgcgccgctgtagaccgataaactgcaaactcatgctggcttttctccagaacccggaagcgtcgtcgccccggactgcgcccgcggtctgctattcgcccacgatggacaccatcatccacaactcggtgagcgccccacctagagggagggggggtagtttaatagcggaggcggatacgcggttttcttttaagcgccgctgacttgtttcttctgttttttcgccccgtgtgctgttccgcccagacccgcaacaacactcctccgcacatcaatgacacttgcaacatgacagggccgctattcgccattcgaaccaccgaagccgtactcaacacattcatcatcttcgtgggcggtccacttaacgccatagtgttgatcacgcagctgctcacgaatcgcgtgcttggctattcgacgcccaccatttacatgaccaacctctactctactaattttctcacgcttactgtgctaccctttatcgtactcagcaaccagtggctgttgccggccggcgtggcctcgtgtaaatttctatcggtgatctactactcaagctgcacagtgggctttgccaccgtagctctgatcgccgccgatcgttatcgcgtccttcataaacgaacatacgcacgccaatcataccgttcaacctatatgattttgctattgacatggctcgctggactaattttttccgtgcccgcagctgtttacaccacggtggtgatgcatcacgatgccaacgataccaataatactaatgggcacgccacctgtgtactgtacttcgtagctgaagaagtgcacacagtgctgctttcgtggaaagtgctgctgacgatggtatggggtgccgcacccgtgataatgatgacgtggttctacgcattcttctactcaaccgtacagcgcacgtcacagaaacaaaggagtcgtaccttaacctttgttagcgtgctactcatctccttcgtggcgctacaaactccctacgtctctctcatgatcttcaacagttatgccacaaccgcctggcccatgcagtgtgaacacctcacactgcgacgcaccattggcacgctggcgcgtgtggtgccccacctacactgcctcattaatcccatcctgtacgcgctgctgggtcatgattttctgcaacgcatgcggcagtgtttccgcggtcagttgctggaccgccgcgctttcctgagatcgcagcagaatcagcgagctacagcggagacaaatctagcggctggcaacaattcacaatcagtggctacgtcattagacaccaatagcaaaaactacaatcagcacgccaaacgcagcgtgtctttcaattttcccagcggtacgtggaaaggcggccagaaaaccgcgtccaacgacacatccacaaaaatcccccatcgactctcacaatcgcatcataacctcagcggggtatgagctttcctgttactttattcagaaagcaccagaacccgtcgccatttcccctcatatacggtacacgtccccctgatctgtcatcacggtacacagatttcgcccgactgcggacgccgacggccaatcgcgtggcgtaggagtggcgccccggcttcattataacgccacgtcggagcccctgcgcgccacaacgccgtccggcgcaacttctgtctcggcacggtacgataaaaacaacgtcccccgtcgacgttgttttctccgagcggtgatcgttcccgtccctctcctccctccgcggcccccacggcggcggcctgctcgcacggacctatactattaccgccccaccgccgtcgtcgtcatgaacttcatcatcaccacccgagacttctccaacgacgattcagtcctgcgagccgccgagatgcgtgacaacgtggcaggctcgatttccaaagcgtacaagggcacggtacgcgccgaaggcaagaagaagctgctgctgaagcacttgcccgtgccgcccggcggctgctcgcgccgcaacagcaacctcttcgttttctgcaccgagcgcgactaccgcaagttccaccagggcatcgcacagctcaagcgcgcgccggccgaactggacccccacgagatccagcaagtcacggccagtatccgctgccgcctgcagcccagtctccgcgagccgcccacgccggccgacgagctgcagacggctgtgtcgcgcgtgtgcgcgctcttcaaccagctggttttcacggcccagctgcgccactactgcgagcaccaggacaaggtggtgagctacgcgcgcgacgagctgactaaacgctgcggcgaaaaatcggcgctgggcgtggaagtgcatcaactggtagccctgctgccacacgagcgccaccgcgaactgtgccacgtcctcatcggcttgttgcaccagacgccgcacatgtgggcgcgctccatccgtctcatcggacacctgcgccactacctgcagaacagcttcctacacctgttgatgaactcaggtttggatatcgcacaagttttcgacggctgttaccacagcgaggcctaccgcatgctcttccagatcggtcatacggactcggtgtcggcggccctggaactctcacacggcgcggcggccgggccgcccgaggccgatgaaaacaacgacgagggagaggaggacgacgacgagctccgtcacagcgacccggcgccgcttcacgagtccaagaagccccgcaacgcccgtcgtccccgcacacgcgtgccgcctcacgagcaaaagcccgaagaaaacgaggaggaagaagaggagctgtttccctcctgcaaggcaaccgcagcattcctgcgggcagaaccctccgtctccaacgacgacggcaacggcggcgaacgctgcgacacgctagcgaccgccctgcggcatcgcgccgacgaagaagacggacctctagccagccagaccgctgtgcgggtcgccgcgaccccctcaccttcagtcaccccagcccttacccccgtcacgtcccccataaccccgttgtgtatttaacgtcactggagaacaataaagcgttgatttctcaagttccgctctggttttggtttcgttttcaaagggagccccatcatggcccaaggatcgcgagccccatcgggcccgccactgcccgttctccccgtggacgactggctcaactttcgggttgatctatttggggacgagcaccggcgcctgctgctcgaaatgttgacccagggctgctccaactttgtggggctgctcaactttggcgtgcccagccccgtatacgcgctggaggccctggtggacttccaggtgcgcaacgcttttatgaaggtaaagcccgtggcccaggagattatccgtatctgcatactcgctaaccactaccgcaacagccgcgacgtgttgcgggacctgcgcacgcagctcgacgtgctgtactcggatccgcttaagacgcggctgcttagagggctcatccggctctgccgcgctgcgcaaaccggcgtcaagcccgaggacatcagcgtgcacctaggcgccgacgatgtgacattcggcgtgctaaaacgagcgctggtccggctgcaccgggtacgcgacgcgctggggctgcgcgcgtctcccgaggccgaggcgcgctatccgcgcctcaccacctacaacctgctgttccacccaccgcccttcaccacggtcgaggcggtggatctgtgcgccgagaacctgtccgacgtaacacaacgtcgtaaccgaccgttgcgctgcctcacctccatcaaacgcccgggctcacgcaccctggaggacgcgctaaacgacatgtatctgttgttgacgctgcgacacttgcagctgcgacacgcgctggagctacaaatgatgcaggactgggtggtggaacgctgcaaccggctttgcgacgcgctttacttttgttacacgcaagcccccgagacgcggcagactttcgtcacgctggtgcgtgggctggaacttgcgcggcaacacagcagtccggccttccagccgatgctgtacaatctgttgcagctactgacgcaactgcacgaggccaacgtgtacctctgcccgggatatttacatttcagcgcgtacaagctgctgaaaaagatccaatcggtctcggacgcccgcgagcgcggcgagttcggggacgaggacgaagagcaggagaacgacggcgagccgcgcgaggcccagctcgatctcgaagccgatcccacggcgcgcgagggcgagctctttttcttctccaagaacctgtacggcaacggtgaggttttccgcgtgccagaacagcccagccgctacctgcgccgacgtatgttcgtggaacggcccgaaaccctgcagatcttttataacttccacgaaggcaagatcaccaccgagacgtatcacctccagcgcatctatagcatgatgatcgagggcgcctctcggcagacgggcctgacacccaagcgcttcatggaactcctcgacagagcgcctctgggccaggagtcggaacccgagatcacagaacatcgcgatttatttgccgatgtttttcgccgtcctgtgaccgacgcggcttcttcgtcgtccgcgtcttcgtcgtcgtcctcagcatctccgaattctgtttcgctgccgtctgccaggtcgtcatccacacgaaccaccacgcccgcgtccacgtacacctcggccgggacttcttctaccacaggtctcttgctctcttcttccttgtcggggtcgcacggcattagctccgcggacctggagcagccgccccggcaacgacgccgcatggtcagcgtgaccctcttttcgccctactcggtagcctacagccaccaccgacgtcaccgaaggcgacgcagcccgccacccgcaccccgagggccggcccacacacgcttccagggacccgacagcatgccgagcactagctacggcagcgacgtcgaagacccgcgggacgatctggccgaaaacctacggcatctctgaaagcggtttttcctctttttctacgtgtctgtctcaagatgagacgtcgatatcaataaaaataccgtcgacgtggtttttttaacagtgtggttttctttattgactagcgaagtacacagtttacgagtagaaaagacagggaaaggttatataaaatgctgtattatatacaaaaacatgcacataaacaaacgggaccatcgtgctcatcatcccctccttgatcagttgttcatgtaaacgtgtggcggggtgaggggcggcatgccgttggcggcgccgggaataatgtgccgtcgaccgacgtcgcacaccttgaaacgccgtcggcgcacgcagcggtcgcaggacgggatatcccagaggaagcccatgtaggtctcggggtcctcgtcgtgaaagcggtaggagagttcaaagtggtgcaacgagcccgtccgagctcgcagcttctggcgaacaccctccacgtcatcggtgcacagcgacagtgctgggctgtcacacagggcctgaagctcctgcggccacaggtgcgtggccaggggcgagtccgtcgtcaccagtttgacgcagtgcatcaggttctcggtgatggcgtcgtacaggcgactctcagcctcctcgtgcgtcatcacgtttcgaggcagcgacagctcgtcgtcgtcatcctcgtcaaacatgatcatggggtcaggggtttttttgggatgttgacaggtgggtgtcttttccagacgcacgatggcctcacgccggccgctgaaacggtggtttcggtgtcccttctttcccatgacgcaggtgaacataaccacgtcctcggccaaacggtagacggcgtccatggcggggtcgtagccgtagacgacgccgaaagtgtccaccaagacgtactggcgtacgaggaactctttgcgttctggcacctcgtggcccagcgcgcccaacaactggtggtaacaggtgatgcgcggcacggtacggatcatgagctccatggtctggatgctgccgcccgcgcggacgacgctgaaggatgtttccttgaacttcataacctctgtgttgtgggtccagaaggcgaaatgggtgtcgggacactcatcgaaagggtcgtcgatggtgtaggaagcgtagcctcgcttggtcacctcggccgacaggctctccacgtcaccgcggtagagcatgacggcgttccagtagtcgtcgtactgcaccatgggccgctggtagtcgcgcatagtgtggaagtggtcgcggtgacgaaagccgttccgcagaaagtccttcatggtgggtgccagctcgtagacgcagtcgcgcaggtcatcgtagcagtagatgccgccgcgctgcccgatgagcacgatgagttggtagcgcataaagcccggaccctcgacgaagccaaaggggtgcaggtactcctgacagcagacgtaagcacctggtagagaatagaaaaaatccacgcacgttgaaaacacctggaaagaacgtgcccgagcgaacgtcctctttccaggtgtcttcaacgacgtggggcttaccttgcgaacagacggtgcccatcttgcccacgaagggccccagggcgctgcgcgaacggagctggatgaagcagcgttcgggccaggccacgtgcagccgggtgccgcattcctgctccagaaagtcgttgagaccgttaaagtccccggctcggatggcgatgcagccgtaggccatcagcgtgtcccgtaggtcgtccatgacggactcctctaccttcgctcgccgacgctgcgcttctccagccaccgctgcggtcgacagactcctccgtccgccttcggagaactacggcgcggcggcacggcctttatagacactatcagcgttgacgtcagacgatccgatgaacgtcgttttttgtgctggaacttccctcgtcccgacaaatgtagcggaaatcttcaagcaaatcgcgacgaagtccgatgaggaggatgcaaaagaggctgagcaacgcgatgctgcccgccgccacagtacatatgctcaacaacgcccagtgtcccaaggcgcgacttttggctcggaggagagccgaacggcggtttctccacatgacggacaacgtggtccagtacgtccatcctttgcattccggtgtccagacgggaagcgttgtcatgttatttcccgtaactgtcacgttatgttttgttttgtttctcgtgagcttaacggtcctcttgagaaatcgcgggcacatgtcttgtagaaagatataatcactttccgcgtatttcgtcagtgttgacatcacggtggtagtgttttctgaagaagtagcgttgtcagtgacgtttgtttcttcccaacgtacgtatgattcgaacggactcgtgtgcgctattgcccgcaacacgtagctgtggccggtgaagttgagcgtcagttgtcccacggtcacgttcgtgtcattcctaaaacatgctacttctccgtgaacttccgtgacgtttatctcacgactctcgttcaagacacgcaggggaaaccagccttccaggtgatactgaaaaccaaatttaagcatgacgctgtgccatttccgtcgtgattgattaaacgttacattcaagggcagtctggcttcggtcccgagacaggggccgttgtagatttgcgtgtgattgcgtgtgcagtttaggtggcagttcatgctcgtggtgttggaagtgcgattaacgtccgtaccgtggtacgtacatcggaccgaaacaccgtgtcccgtgctccaaagcagcgtcaacaacagccacacagaaacctacgtggagacgacacgggactttttattgacggagactcacgtttctaccctcccctttcccgtaggtaaaaacccacgtttatcacacacgttgtttttacctgaaacccgcgcagcccgtggacgcgacaaaaaaccgcggcactagaaagaaaatgaaacaagtatgtttattaagcagcatgtggggctaataggggggataactgaggtatagcaactatgaaaaaatactacaaaaaaaaaagctgaacatggtcatctagcagcaaagttctccttctagaccacgaccaccatctgtaccacgtcgccctccccggccgtgtacacgacatccttcaccacgaccggcggcagcggcggcgacgaggacaactcgctctcgacggaggccgggacgacagaggacgggggggtggtggcggcggaggacgaaggggtggcggcggcagcgggatcttcttccgacacgggcaacggcaggctcggcggcgcggacagcacccgttgcgccggggcgtgagaaggctgagccccggtggcctggatgtgggccaacgaattggctcgcagcgagtcgcgatccacgaaggtcataggaattttcccttcgcggatccgccgctcagattccaggatggcgcgcacgtagctgttcaccgacttggcaaaagtgcgcggcccctccgtattcttgtcgcgacgcgcttccagcacctgcttttcgtagtccagctggtggaagaccatcaccaggtcgtccatagtgtgcgcgtgctgacggacgtgggagcgcacctccaccgggaacaaagcgttccaatactccagcacgatagcaccgtgccagaactgcgccatgctgggcgccaggaaaaacaggataccggagtcgtaggcgaacacgtcccacttgggcgtcatgaacaacaccagctgacgcgtgggccgcaccgaagcttcctcccaggcctcgatgaccccgaacatgatgagctcctggtccaacggggggcagtgtcgctccagccaactgatcttgctcaggttcatctgcagaaactcgtacgaagggtcgcagatgcacacgtagagacccgagtcgtgccgcagcctggctccgcgcttcatcagtttcctcaccgcgtagcgaagcgccaccttgcccaacgccgacgcctggatcagtccccccacgtccatctgcgtctgtcgccactcggcctcgtccagcaggctcatgatagcggcagtgctatgcgtggtcgtagtcatcctttctatccttctctatgaatagcagcaatagcggtaaagtcccttcttatactatcccggagtctgtggtttttttgtttacccctgcttactggtgagactgctgggggccgttgtgctgcagcatccgagctcgttgccgccgttgccacaggaaccggtgtctccgcagggcctttttgagggcttcgcaggcttctcgcgcaagtcctgagaggccctcggcgtcgatggggttcacctcgggcgtccgagcctcgttttcttcttcttcatcctccctttcctcctccgtgtcctcccgctctgtgtcctccgttacgctctcctccccggcctcggccaagagcgcggccaccaagtccacggaccgctcggtctccgagttctcaccgtcaattacgccatgttggcggcgtaaccggtgccgagaacgccgggtgagcgcacatgcttttttctttcttaaccaaggcgggagaggatcttcaaggcgttttcgctggatccagcggtagctaaagtaccaaaaggccagcaggcccacgctacctaacagattcacgtagactggagacataattaaagaaagaagtgaaacccgcgtgtgggtctcacgtcgtcttgaaacaccgtcttatatacatgaagatgccggacatgacgcgcccaagacacgtggggttttccccttaggcgacccggtttcttaagatgtttttcatcttcgcacgcgatgtactacatcaaagggtcggctgaccgaccgcattgacgcacagtttccgagtacgcgcgtctcggagcacctgacggtgagccacccaactcacgcggataggggacaacactgacgtgaggggcgattcacgtcactgacgggaataagacgggtgagggatttccacctttttcttaagtgtgactctctttacggtaaatcgcacctgtgacctcttaacccctcctccctggtacccgataaccgtgaaaaacacacaccacacgtcacgacaccgatcgattttctttattcttagtgtgatgataggtaagggcactcgtgaggatgtgcaattatcattatcaagcctttttcaaggcgtagtgatgatcgttgggcagaacccccaggctcctagcgatctgggaatagaaggaggagaacgagcccagggccagaatgcccacagtgtacatggcccaggtctccagaccgaacgtggcgggtcgcagcttcagatggtaggccacccgctccgagagttgtgaatgctcgttcaggcaacaggactgcaggtgggtgagcccaaaagcgctttcgtttacgccgcgcacgtgcaccgtctgggccgggcaatcctggtgttgcgcgcgaaagtggtcctgacaggaaattccgtctacgtggcggcgcgtgttgttacccacttcgatcaacaacgtgttatcggcaggatgatgcgagaacgcgacgacggtgttgttggaggtctggcggcaacagtacacgtcgagcgtcatgagggccatgtcgccttggtggtacacggcgtacgcccaaccctggaacacgagcggacataacggaccgtgagcggacgtcacggcggcggttgttaccgtcgtctcggcaggagaacacaataaactcctgatcctcatacacaggagtccaaccgtcagaattaaagtccgcggagccataaccgcgcaagtgaagccgatacgagtgttgctgaatttgttcattctgccgactgttgctcacgagcgttcggaggcggtgccacaggctgttggccattaaaaagtcctggcccgaatgacgacaagacagagcccgaggcgaagaaaaaggcgcccgtcatgaagacgtaggcaggggaattcccatatttttatggcttcttttaaaagtctgtatccgactccatccggcgcttttcccaaaccgtggtctcctcgtcgtccgactcggtacccaggaggtggtaagtcttttgccgcacgtagaaagctttcaacgtggagcaaaagatgagaataaagaccccgaaaacgaaacaaaccacgccgatcatgccgatgcagacgttcatgtcgacgtagccggcggtgctgttggcggtgcggcaaaagagtgtcatgtcgtgcgtgcacaaaaaacaacacacaccacaggccaggtcgtagcgtagttattattccgtagcagcaatgatggtacagtcaagcacatgctctatccccgttaccccgatgatgcttgcgtccccgttgttatattggcactgtcccggttaatcaccacggtgaacaccacggccaagaaaatgatccctaatatagcgaccactaagagagcaaaagtccatttccagccgttgtcaaagtacgcccccgtggtgggatgcatggtggcgggcatttccatcatgtccatgtcgaacgtgtgtcgcggcgacggcgaactaaccaggcagtacgggggtcgatagggcggtgggctgcagtcgggtggtggcggcggtggcgtggaaaccgtcgtcgggcacagacccatggcctgctcgtaggtggggggcgcgtcgtcgtgatcccggtcgcggagcatcggcgtgggctccatgtcggtggcagtgacggcgacggtggtaactgtggtggagacggtaccgacggcgtccgcggttcaccttcgagcaaagagccccttctttttgcgcaaacgacggcaaaacagttctctgggacaaccggtggcgcggtaagcgggtgccacgctttcagggtgggtaaaacagtcgcgggcgaagcagtagttgttgcagaaccgcaagaacccgacgcgaaagaagcccaggagtccgcgcgccagaaagtgcgcctgccgcgtctcgggatgcacgccgaagacggcgccgctctcgttcaccagtatggagatgtccaggcgctgctgcgactccaccggcacggcccgcaccacaaatacctgcagcacgttcagcgagcacgtctcttttaaccagttgccgtgggccggatcctcgtaagtctggctcccgttcaagacgaccgtcgtcagcgcctcattaccgtctcgccagctgaagatggaaccctcgcgcttcatgcacaggcgccacaaggccagcaggtcgcgcgccaacatgaactcgcgacccacgtcgccgccggtctcgaagcggacatagcccagttcttcgcgcagcggcgcgtagttgcgcaggccctcctgcacgaagccgcggaaaccggaccgcgacaccaggtacagcgattccaccacgggcgagtagacgtagacgcgaccgccttcgccgatgagtacgggtagcggtgggcggccgatggcttcgcaacgactcacagtgcccaccggcagcaggaacttgtcgcagcacaggaaggtcttctccaaacctttaatattgagatgtccaaagtaaccaacgcgtaacaggtcgcagtaggtgaagaaccaaccgtttggccagctgagacgcagcaccgtgccgctgacgcgacgaaccagcttctgcaggtccttgcgagcgtcggaggtgacagagcagcggaaggtctcgttaaccagctcgacagccagcgcgtcctccagcgtgcgttccttcatctcgtcgttgatgctctgacggcgccgccggatttcgtcgaaacgggccgcggaggcggcgaccgacgcggaggtcgtccgaacgccctctgtgacgctgccgtccggccagtcaagaaagctaaggctggcgctgcgccgcctaaagtgtccgatccgcgcgggacgtcgctgagggacggtggctggtctgctggggcgggtacggccgcgggtgtccgcggacacgttagttatacacggaattgagtcacgtggcacgttgccagctgaaaccgccgtcgtctccgccggcgttttctccatcacgggaccgcgccgtgcgcgcgttcccaggcacgcggcccgcgctctagccgcacttttgcttcttggtgttagggacgaactcgaacgttacagaatcctcgctgtcgctctcctctttcgcgtcgttgaagtaattgccggagttgcgatccaaaccgccgcctcctcctcctccgccgccgcccgatccacctttggacgtcaggtagctggtgatcttgtgctgctcgtatttttccttggaggaaagaccgtggtcgtgatcaccgccgccgccaccgctgctcattttccgcgtaccggaaccaccgccgccaccgcggtcgtgcttcttgccgccaccgccgccacctcctcccagaccgccgagacccatgggctcgttcatgagatcgttatccagacccgggccgtcgtcatgcagaccgccggcattggccagcgaagagaggctgccgccaccaccgccgccgccacgcgacttgccgctgttcccgacgtaatttttgtcgaagggatcgccacgctggaaaggttcctcggtgagaaaattctccacggcgaacagaccgttgcggctggccacgtacaacagcgtgtcgtgctccgtaactatacgcaacgtgcacggcagtttggtgacggcgcaattgagcagcgtctggtagaagttcttcagctgcacgttgatacgcatgtttttcacgccgtggaaactgacgcggttattggctgtgaattccagctcgctgccgttggtcaggataaacttgatggccggtggaccggcgtgcaccagaatctgcacggtgcccgtagggcagggcgcttttttaacgttacgcttgacgcgggtatgcggcccgatccacttaagcaggtcggccaccacgccgaaatctagatccacgtgcacggccgaattctcgctttcgcgcacaatgtcttggccgtgcacgcaggccgagctgaactccatattgaaatcgggcgcgcacatggagatcttggccgacaggtccgagatgtcctgcacgtagaacttggtcaggtccttgctggaagtcaggtacatgaaattacccagcagcggcgtggaattgttaatggtcttgggctgaaacgacttgtcagtgatgtagaggcatgagctgttaaaagtgatttttgacacgcagtgactgcgtaccgtttgcaagataagcgacggcgtgggcaagaaggtaaccgtggtgttctccttgagcgcacggatcacagatcgcagctgctggatagccgtcttgtacggcttcagccgcagcgccagcgtcggcggctccgagaggcgcgtcttgcgatccatcccggacagcgtgcaagtctcgactaaggagcgggcgcgagcgagcgaaagttttatagagagcacacacgacgaccgggaacgctgcgaagacgcccggcgtctaataatacagccgcgccgagccagcgggcccccgactaagaggcacagtacttatatactccgaccttaaagcgccagtggtaccacttgagcatcctggccagaagcacgtcgggcgtcatccccgagtcatagtagaaaaccagggccacgcactggtccacaaacacgctcaggttcacggccgccatttccacgtcgttttggatcgccggtgccgcctggaacagacactgcgtcgccttgccctcctcctggtgctgctccaaccacgcgtaattcaccacgggcacgcgcagcggcctccgcaccacggtggggaagtaacactcacggttgggcgggcacaatgaccacaccgtctcctcctcgaacacggtgccgcgcgaagcccacactgacggcgtcacgccccacagatgcgccacctcgtcgtcgggacccaccgccagaaactgacagttgcgcaatccgaactcgagcatgtcggcgcgcagcgcttcccagcgcgcgctggcgatggagagccgcggcaaccgatacaattcgaaaatgaatttgccctcttgatagatggtgcgttcgaaccactcgcagcgcggcaaacccgacttgcacaaatcgacgctagcgcgcaccgcggcaaagtacatgtgctcaaagatgcgctcgatcaagtcccaagaggcaaagtacgtgaaccctaaccgcatgagcgccgtgtgcaagccagccacgccgatgtgcagcggacgcagtttttccagcgcgctctctacccaccattcggacgccgacattagcgcgtccaagcgcgcgttgccccaaaccaccgcctcggtcaccaactcgcgcagcacgctcaaatcaaagtaacgtcgcgtgttccccaaaaccacgtcgggtagatgcagcttctgctcgtcgctacgcgcaaacacgcagcgagccacgttcaccgtcagccgctgcaccggcatgtcacactcgccaaagtggcacgacgccatatcgggactcaagcacggcggcaggcacacgctgtcggccataatcgagtacttgactacgtgatggacaaagaccaccgaggcacggcccttgagcgcgcacagcaacatctttttcagaaaatcgtccgtgttcacgaccaccttggggcacgattgctcgcagcgcgaatactctttctcgaaagccgactcctgacccaggtccgagagccgccgggagacaggccgcccaaacagcgagtagcgctgctcacgcgcacggtagcgcttcattaacacgctaggcacgttgaaagcgtagcaaacccccgtcaactccgacgtgctttctttgagaataaagttaatcacgcggatagcggccacgtcccacatgtccacaaacacacgtaccacgggtcgatgcacctccttctcgcgtatcaaatcgcagtatccccccaggcaacgaatcacgctgttcacatcggcgttaagtcgcgttacgttcaccgacacagaaacgccgcaactcaaggtactcatccacttgcacatggccgcccaactggcgtcacgcgagaaagggtcggccgagatcagaaagtcgtactgcggcacgcgatcgaaacccacggtagacatggtgaaggtggacagcgacagctgcccatcgcgacagcgcttcaacaccgattccaacacctcgccctcgaaacgcgcatccagatggaaacgatagatgcgcgagtgcctactgttctcgatagcggccgtcaacgccacggcgatgcgcaaaaacacgccgcccgggctctcgtcctgtccgtgcagttggcgacacaccttatccaaacacaaaatggccgcgtacaagccccagcaaccggccaattccacaaaacgcgccgtctcctcggccagcttgggtagatcctccatgtgacgcagcacaaaacggcgcaccgactcatcgcacagctccgaagcgtaacacagtggcgtgcggctttcacgcgcccagttggctttgaaataaaagcgacccaacagcaggtcgcaacgcggcgagtgacgaatcagacagggaccgtggcgcataatgagctgaaatagcctgaaactgcccaaaccggcactgtgccgcgacacggtgtccatctcgcgccacagcgcgttcctgtcggacggcagctcccgcgccggctcctgtacgccgcaaaagcgaaacttgccccagtagccgtgacaatgacactttttgcccatcaacatgcgcgtagcttgtatcggcggcgatactttgcagagcgaagccccgaaatcgtcctcctcctcgacactgtccagctccatcctggtcgcgccggccggattaaaggtgctcagaccgctactcacgcgtccaccgcgactgggcacggcgggaccgctgtcacgcgtcaacgacagcacagacggcgtgccgtcgggagacggcgactcgggacgccaactgacgacgccgccaccactcgtaaaacccgctacacatgctacaccgctcgatacgttggtatttccagcggacgcttccttgtcacccccgggcagcggcccctcctcgagctcgctgtcatctcccccgatagtatcagcggcgacctctgccgacgattcctccgtctcggtttccgcgctgcggcttggaatcctacctggccggcaccgatgtgcgggcaccgaggacacccgctgttcctcgtccgcgtcagccggattcataagtttacgaggaaaataacaaagaaatcaggtagatttcaataaagtgagtctagatggcgccgacaactacggtttataaagtctgtgtgcgatgtgtttttttcttctgtgtctcctccccgtatgctgtcagcgccgctcagacgaattctcgaaagtctcccaattcgacgctaaagttgtccaaacggacgacggacagtttgagttctttgtgtaccaggaacgaggtgtgaatgtcgtcagccaggcaccagcccagcttttgtatgaccccggtacacagagggatctggcgcgggcgcgtgatgcgacggttgacaaagctacagcgctcgcgggcgaactttccgcgtgcaacgtcgaccaaggtctgccagtgtgcgatgctggaggtgagcacgtagatgccgggacgtgtttcgggcccgtcatagtcatagacgatgattaaatacacgtattgcagccgtccccgggtctcttcccacgtcaggtacatgtctttcggtatcatcaacgcgaacacctccgttttgagcgtgttgtaaaggtagccgcgcatgacgcaggtgagcaacgaggtgatgcccagcgagacggtcttgacgcagcccagcgtctcgaggcggcggtgcagcagatgcgggcccaggtccagccactgcagcgcggcgcgcgcggccgaggccgtgtacacgctttcgagcaggcagcgcgtgctggccgagacgttggaggcgcgaatgcctaacaggtaaaggctaatgtagaggtgtcgcggcgagtcgcaacccgtctccatgcggatgagcagcgcgcccggctgcgcctcgaactctaccaggccctcgggcacgaagaaacgcgccgtgagcgcctggtgatcggcgtggtagagatagcgaaccgatatagtatttacctcgcgtttggctttgagcgccgtcactagttcattgtcctcgtcggccgggtcgcgcggccgtttggccaccgcgcgcgcgtccatgatggcgaggcgcacggtagatttcaaaaagttgatagagcagctgcgggcacgggccacggacaaagcggaggcgttaaataccgtgagccaattggagatcggcgcggtggatgcccaggacgtgaccgcgagcgccgtgcgcgccttcgtgggtgcgttgccgagctcgggctaccactttggcttcgtgcgtcagaacgtggtcttttacctcctaagccacgccacggtacagacggcgcgcgacccgctgtacgccgccgagcagttgcacgaacagctggaccgcttcctgcgacaccagcacgacggcggcggagacgaggaccggttgccgttctaccacaacggggccacgctgacggctttccagaagctgttgcagaccctgcgcgagatccagaccgtaatagccgaacagagcggcggcaccgcggcggcggcggacttgatcgccagtaacaacgcgtcgaccgagcgccgcggcaagaagggcggttcgagttccgggggccagcagccgctggtccgccgggtgatcacgcagctggaaacggctgccacggaggcgcggccctacgtcaattgtcgcgccgtggccgaactcctggacctgacctaccagcggctcatctactgggcctgcacgctcatgccctacgtgttgtttcggcgcgacaccgacaccgaactggacacggtgcttctgatgcattttttttacacacactaccgttcggttaacggcgatttggccgtggagtttcaaaactacgtcaagaacagcgtgcggcacatgagctctttcgtcagttccgatatcgacggcgaccagaagcccggtgccgaacacatgcgtgacgtcagctacaagctgttcgtgggtaatctgcaagcgcgtgacgccagcggcctcatgtttcccatcattagcacgcgcatctccaccgtgaacctttacctgtcgcccgaacgtatgtttttccacccgggtctgatctcgcgtctgttgagtgaggaagtttcgccgcgcgccaacctagacgcttacgcgcgcgtgtgcgatcgcgtgctggaagaccacttgcatacgccgcgacgcgtgcaacggctactagatctgacgcagatggtaatgcgactggtggaactgggtttcaatcacgatacctgcgcggcctacgcacaaatggcgctgatccagccggccagtcagaagagctcgctctttgtcagcgagattcgcgagaaactcatacagatcatctacaatttttacacgtttttcatgtgcctctatgtgtacagccccacgttcctgttcgaccaccggcggcggttgattttggagcagcatcgatccacgttgatcggctccaaggaggaactacagcacgtctggagcaacgtgacactgaacgtcaatacgcactttgcggttcagtacacggaagaagactttgaggcacatacgaagggtgccacggaggcggagcgcgagtacctgtatcgggacctgcacagcaagtggggcgtgcacctgtttaccttgcgtccgtctcgcggcgcggccggcgcggcctcgcctttgcctccgcttgacggcgtcacacgctccgacatcttacgcgaatgcgcgctcgttaatctgaacgaaggccgcgtcaactacgcctccctgctagccttcagccatcatcccgagttccccagcatcttcgcgcagttggtggtggtaactgagttctcggagatctttggtatcccgcagggcctgtttcaagccgtgggttcgccgcgtcttttcgcactcattcagctgtgccgtgtattgttgcccgagcaggtgacgctgtaccagaacctggtctccatctacaacctgaccaccttcgtcaagcacatcgacgccgcggtttttaagacggtacgcgattgcgtcttcgacatcgccacgactctcgagcacctcagcggtgtacccgtcacgcccaatgtggacctgctggccgagctcatggcgcgctccgtagcgcataacctgtacaccaccgtcaacccgctgatcgaggacgtgatgcgcagcagcgccggcagtctgagaaactatctgcgacatacgcgactctgtttcggtctggcgcgtggccgggcgcgcctctcggaggacggcgtgacggtgtacgtggaggtacaaggtcaatacggactacgcgtacccaccacgcgtttcgtagaacagttgcgcgagctggttcgccgcgatcggctgttggccgagaatctgcgcggcttgaacgagcgcctgctgagtgttcgcgtgcgcgtacgtcagatcagcagcgacacagaggaagtaagccgacacgccaagggtcaccgcacggtggcccagatgagcaaggcgctcaaaaagacggcctccaaaatcaaagtgttggaaacacgcgtgacattggcgctcgagcaggcgcaacgttccaatggcgccgtcgttaccgcggtgcaacgcgcgctagccgtctttgacgtactaagtcgcgagaacttggaacgccgcggcgcacagctctgtctgacggaagcgacgagcctactgcaccgacatcgcgcgctagcgccgatgacctggcccgcgggcacgggcgttgcggcggcggccgaagcggatcgcgccttacgcgagttcttggaggcgccctgggaatcggcgccccaaccgccgcgactccgcatgacgcccgacaccgatcacgaagaatcaacggcaggcgcgacgtccgtaccggaggtcctgggtgcgcgctacgaacccgcacacctggccgcgagcgacctattaaactggtacatcgtccccgtaagccaggcgcagcaggacatcttgtcttcgatcgacccgcccgccggctcgacatcggtgtccctgccgccggcctcgccatgaaagtcacacaggccagctgccaccagggcgacatcgctcgctttggagcgcgagcgggcaatcaatgcgtctgcaacggcatcatgttcctacacgccttgcacctgggtggaacgagcgccgtcctgcagaccgaggcgctggacgccatcatggaagagggcgcgcgtctggacgcgcggctagagcgcgagttgcaaaagaagctgcccgccggcgggcggctgccggtctacagactgggcgacgaagtgccgcgccgcctggagtcgcggttcggccggaccgtgcacgcgctctcgcggcccttcaacggcaccaccgagacgtgcgacctggacggctacatgtgtccgggcatcttcgactttctgcggtacgcgcacgccaaaccgcgtcccacctacgtactcgtcaccgtcaactcgttggcgcgcgccgtggtcttcaccgaggaccacatgttggtctttgatccgcacagctccgcggaatgtcacaacgccgccgtgtatcactgcgagggtctccatcaggtgctgatggtgctcacgggcttcggcgtgcagctgtcgcccgctttctactatgaggccctttttctctacatgctggatgtggcgaccgtaccagaggctgagatcgccgcgcgtttggtctccacctatcgcgaccgcgatatcgacctcaccggcgtcgtccgagaaagcgcggacacggcagcgacaacgaccaccgccgcaccttccttacctccgctgcccgaccccatcgtcgacccgggttgccctcctggcgtggcgcccagcattcccgtctacgatccctcgtcctcacccaaaaaaacacccgagaaacgccgcaaggacctcagcggtagcaaacacggaggcaaaaagaaacccccgtccacgacgtccaaaacactggccaccgcctcctcctccccctcagcgatagcggcggcctcttcttcgtccgcggtaccaccgtcctacagctgcggcgaaggggccctgccggccctgggccgctaccaacagctggtcgacgaggtagagcaggagttgaaggctctgacgctgccgccgttgcctgccaacaccagcgcctggacgttgcacgcggcgggtaccgaaagcggcgctaacgcggcaacggccacggcgccgtccttcgacgaagctttcctcaccgatcgtctccagcagctcatcatccatgccgtcaatcagcgctcgtgtctgcgtcgcccctgcggtccgcaatcggcggcgcagcaggcggtacgcgcctatctgggcctatccaagaaactggatgcctttctgctcaactggctgcaccacggcctggatctgcagcgcatgcacgactacctgagccacaagaccaccaaaggcacgtactcgacgctggatcgcgcactgctggagaaaatgcaagtcgtcttcgatccctacggacgtcagcacggcccggcgctcatcgcctgggtggaggagatgctgcgctacgtggaaagcaagcccactaacgaactgtctcaacgactgcaacgtttcgtaaccaagcgaccgatgcccgttagcgacagcttcgtctgcctgcgacccgtagactttcagcgtctgacgcaggtcatcgaacagcgacgtcgggtgttgcaacgtcaacgcgaggaataccacggcgtttacgagcacttggccggcctcatcaccagcatcgacattcacgacctagacgccagcgatctgaaccgacgcgaaattctgaaagcgctgcagccgttggacgacaacgccaagcaggaactctttcgcctgggcaacgccaaaatgctagagttgcagatggacctggaccgtctgagcacgcagctgctgacgcgcgtgcacaatcacatccttaacggctttttgccggtagaggacctgaagcagatggaacgcgtcgtcgagcaggtactgagactcttttacgacctgcgcgacctgaaactgtgtgacggcagctacgaagagggattcgtcgtcatacgcgaacaactgagctacctcatgacgggcactgtgcgcgacaacgtaccgctactgcaagagatcctgcagctgcgacacgcgtaccagcaagccacgcagcaaaacgagggtcgcctcacgcagatccacgacctgcttcatgtcatcgagacgctggtgcgcgacccgggcagccgcggctcggcgctgacactggccttggtacaggagcagctagctcagctggaagcgctaggcggcctgcagctacccgaagtgcagcagcgcctacagaacgcgcaactcgcgctaagccgcctctacgaagaggaagaggaaacgcagcgtttcctcgacggactctcgtacgacgatccgcccaacgaacagaccatcaagcgacacccacaattacgcgagatgttacgtcgcgacgaacagacgcgtctgcgactcatcaacgccgtactgagcatgttccacacattagtgatgcgactggcgcgcgacgagtcgccgcgaccgacgttttttgacgccgtcagtttgttgttgcagcaactgccacccgactcgcacgaacgtgaggatctgcgtgccgccaacgccacgtacgcgcagatggtcaagaaactggagcagatcgagaaagccggtaccggcgcatccgaaaaacgtttccaagcgttacgggagttggtttactttttccgtaatcatgaatatttctttcaacatatggtcggacgactgggcgtcggacctcaggtaacggaactctacgagcgatatcaacacgagatggaagaacagcacctggaacggctagaacgtgaatggcaagaagaggccggcaagctcacggtaacttctgtggaggacgtgcagcgtgtcttggcccgggcaccgagccatcgtgtcatgcatcaaatgcaacaaacgttaaccaccaagatgcaagactttttagacaaggagaaacgtaaacaggaagaacagcaacggcagctactggacggctaccaaaaaaaggtgcagcaggatttgcaacgcgtggtggacgccgttaagggcgagatgctctccaccatcccgcaccaaccactggaggccacactcgagctgctcttgggcctagatcaacgcgcccaaccgctactagacaagttcaaccaggacttgctgtcggcgctgcagcagctgagcaaaaaactagacgggcgaatcaacgagtgtctgcacggcgtgctgacgggtgatgtagagcggcgctgtcacccgcaccgagaagcggctatgcaaacccaagcctcgctaaaccacttggaccaaattttgggtccgcaacttctgatccatgagacgcagcaggccctgcaacacgccgtccatcaagcgcagttcatcgagaagtgtcaacagggcgatccaactacagccatcacgggcagcgagttcgagggcgactttgcacgctaccgcagcagtcaacagaagatggaggaacaattacaagagactagacaacagatgaccgagactagcgagcggctagatcgctcgctgcgccaggatcccgggagcagctccgtcacgcgtgtacccgagaaacccttcaagggtcaggagctggcgggtcggatcacgcccccgcccgccgacttccagcagcccgttttcaaaacgctgctagatcagcaggccgacgcggcccggaaagcgctcagcgacgaggccgatctgctgaatcagaaagtacagacgcagttgcgacaacgcgacgagcagctgagcacggcgcagaacctgtggactgatctggtcacgcgccacaaaatgagcggcggactggacgtgaccacccccgacgccaaggcgctgatggaaaagccgctggagacacttcgcgagctgttgggcaaagccacgcaacaactgccgtacctgtcggcggaacgcacagtgcgctggatgctggcctttctggaggaagcccttgcgcaaatcaccgcggaccctacgcacccgcatcacggaagcaggacccactaccggaacctgcaacagcaagctgtcgagagcgccgtgacgctagcgcatcaaatcgaacaaaacgcggcctgtgaaaattttattgcacagcatcaagaggcgactgccaacggcgcgtccacgccgcgggtcgacatggtccaggcggtggaagcggtctggcagcgactggaacccggacgcgtagccggcggcgccgcgcgtcatcaaaaagtgcaggaactgttgcagcgcttgggtcagacgctaggcgacctagaactgcaggaaacgttggcgacggaatactttgcgctgttacacggaatccagaccttcagctacgggctggactttcggtcgcagttggaaaagatccgcgatctgcggactcgttttgcggaactggccaagcgacgcggcacgcgtctctccaacgagggagtcctgcccaacccccggaaaccgcaggcgacgacttcactgggcgcctttacacgcgggttgaacgcgctggaacgacacgtccagctgggtcaccagtatctgctcaacaagctcaacggctcatcgctagtctataggctggaagacattcctagcgtgcttccggcaacacacgagaccgaccccgcgctgataatgcgcgaccgcctgcgtcgcctatgcttcgcgcgtcaccacgacaccttccttgaagtggtagacgtcttcggcatgcggcaaatcgtcacgcaggccggcgaacccattcacctggtcaccgattatggcaacgtagcctttaagtacttggcgctgcgagacgatggtcggcccctggcatggcggcgccgctgtagcggcggaggactcaagaacgtcgtcaccacacgttataaagccatcacggtagccgtggccgtctgtcagacattgcgcactttctggccacagatctcgcagtacgacctacgaccctacctcacgcagcatcagagccacacgcaccccgcggagactcacacgttgcataaccttaagctcttttgttatctggtgagcaccgcctggcaccagcgcatcgacacgcagcaggagctgacggccgccgatcgcgtaggcagcggcgagggtggtgacgtaggggaacagagaccgggccgcggtaccgtgctgcgcctgagtctgcaagagttttgtgtactcatagcggctctgtaccccgagtacatctacaccgtcctcaaatacccggtgcagatgtcactaccctccctcacagctcacctacatcaggatgtgatacacgcggtagtcaataacacacacaaaatgccccccgaccacctccccgaacaggtcaaggccttctgtatcacccccacccaatggcccgccatgcagctcaataaactgttttgggaaaataaactggtacagcaactgtgccaggtaggcccgcaaaaaagcacaccgcccttaggcaagctatggctctacgccatggccacgctggtctttccacaagacatgctgcagtgtctgtggctagaactgaaaccccagtacgccgagacatacgcctcggtgtccgaattggtacagacgttgtttcagattttcacgcaacaatgcgaaatggtgaccgaggggtacacgcaaccgcagctccccaccggagagccggtgcttcagatgatccgcgtgccacgtcaggacacaaccaccacagacacaaacacgaccacggagccgggacttttagatgtttttattcaaacagaaaccgccctagactacgcgctgggctcctggcttttcggcatacccgtgtgtctcggcgtgcatgtagccgacctgctgaaaggccaacgtatactagtagcgcgccacctcgaatacacgtcgcgagaccgcgacttcctccgcatccaacgctcccgggatctcaatctcagtcaactgctccaggacacgtggaccgaaacgccgctggagcactgctggctacaagcccaaatcagacggctacgcgattacctgcgtttccccacccgcttagagtttattcccctagtcatttacaacgcacaggaccacaccgtcgtacgcgtgctgcgaccgccctccacgttcgaacaggaccacagtcggctggtgttggacgaggccttccccaccttcccgctgtatgaccaagatgataactcatccgcggacaacatcgctgcgtctggcgccgctccaacaccgccggtacctttcaaccgcgtgccagtcaatattcagtttctgcgtgaaaacccgccacccatcgcgcgagttcagcagccgccgcgccgacatcgtcatcgagcggccgcggccgcagacgacgacggacagatagatcacgtacaagacgatacatcaaggacagccgactctgcattagtctctaccgcctttggcgggtccgtctttcaagaaaaccgattgggagaaacaccactatgccgagatgaacttgtggccgtggcgcccggcgccgccagcaccagtttcgcctcgccgcctatcacggtgcttacgcagaacgtcctcagtgctctagaaatactgcggctagtgcgattggacctgcgacaactggcgcaatccgtacaggacactattcaacacatgcggtttctctatcttttgtaaccgacactgacagtagcgggtaataaaaacaataggatttttatcgtttttttatgttacaaaacaacgtatcactttcacggtgatttattcttgctattccttttccccttgggctgtcagcgccgggtgcgcgacacggctaccatgcgcaacaggtccagcttaaaggcgcacttgtcattaaacaggctggacatgcgcgtgtacttgctcagcatggtggccaacaccgggtgggtggcctctgatatctcggtcggcagctccaaaacgacgttaacgacgtgacggtgtttttcgtcccgcttgttggccaccgtgggtcccggcgcggtgttagacatggggcaggccgtggggggaggacgaagaggaagccgctgctaaaccgccgcgcgcctgctgcacaatgtggccgccgacgtggcaggcggtctgtttaaccagcgcgcagccccgacacagcggggcgccgtcctcgctttccaaacagctgtcgcggtactcgcccgtctgacagcgcgcgcacagcaggccgtgcccgtgcgaagtgaggcgcaggagacgcgggaccgtcacgtcgcgtaccaccacagtggagtcgcaggtgcgtgccgcgcagggcagaatgacgtcgaaagccagccggtgatcgtacacggcacaagccgcgttgaggcccagcacggctttccagcccacgcgtacgcagcgctgtccaaagagcgtctcggagacgagctcgtagacgcgctgccgcaccacccgctgactgccgcagagcgagcagtgcacgagctcggcgtgcgtgttgaagatgacgctcttttcttgacggtcccgataatagaacatcgagttgagcggaaagttttgctggcagtgtagcttttccttacccaggttgaggcagtgtccgcactgccgacagaccacggccaccagcgagcgcgcgtccagatggcgctcgcacttgagtcgacacagacaccagagcggcaggtcgatgacgctgccgatgaggccgccgcgcagcgcggcgctgagtgcaaagaggacgatcttggtgggctctacgtgacgcgcctgctgtccggcgcccgcgtgtcctaccgccgcagctgccgccgtcgagcctcctccgcgcgtctcgtcgtgcagacccagtgcccgcaacggcaccaggtatcgcggacacgtgtcgcaaaacgtctgcaccgcttgtcgggccagtacgtagagcgggtttccgcagggtaccttcccggcgtaccggcgcaaggctgcgatgaggccccgcaactgcggcgaccgcggctgccgttggtgacaccactggttacggtggtatacggccaaatcagcgcgggcgtcgaagcgcttggcgcgtagtaatgctaggcacggcgagctggtggggtgaagcacgggcagccgaaggtccaccccgaaaaggaaacggtgaaggtcacctagcagcgaggcggtgacaccgtccaacaacgcgtgcagccgctcgggcgggtagagccgcagacggcgcagcaggtagtcggtgtcgtagcgttcgaaacgcagaaaggccatcgtgcggacggccacggtgtgcagacagtccatgctgtagacgtaagcgagaaacacaaagtagggcttggtcataaccatacgctgaaagagcgccgtcaccgcctcccgctcggcttgccgacacaccagccattcgcgcaggaagcgttggtagagacggtcgcccagctcgcgattcagaaagcgcttatccgtcacgaagagatgaaggacgcaagaacgtggcacgtgatgcaccagctgctgctggaggaccgccgacgtctgcgccgcaaactgcgccggtggctgcgacgtttctaccgccgcttcctccggctgcagcgcaccgcggccgatcaccagctgcacatggaaatggtcctcgtgaacgcagaggggcgcgaagagacggcgcagagcctggtggaactcatcagtcgcggtgtgcggagcgtgtcggagacgacgactggccatgaccgcgccacagcagagccagcaccagcagaagagccagcaccagcgggcccagagtcgcaaagcgcgcgggcagccacggcccagactgcggtcgcgatggcccggagcgcgctcgccaccacgatgacggtgcccaacgataaccagtccgctccaaggacggcgcgcacggcggagacggcggatgacggtgatgggtcgacacccctcgccgacgactcacgtgctcctccagaggccgacgcgcggaccctccgacgtcctggcccgccgctgccgctgccgccttcccttctcccgccagagccagcaactcctcctcctcttcatcagcgtctccctcgcttgcgcatccgcatcgtcccatacaggcctcacaacgacacagccgccacgaccccgccgccatgggtggcggcggcggccgaggcccggcagcggcgccgccagcggcgaccatggtgggagagcaactcggatgacgaggaggaggagggggagatgcggtccgagaggaccgctttcccgccgttcgcgtaagcgcggccgacatgcgggcgcgccacagggacggaccgctgccgctgtgactgcttacggtgacgtggttccggaccgccaacgacgtcgacgcggctttcttggcgtacagctcgcgcagcagattctcgtactcgccctcgttttcgggtccgaaggcgatgagctcgatgttgaagaccgacgccgaattggatttgcgcaccacgcacttcgtcagcactccgtaggccgagggcttgatctcctcgatgtccttgagcgtgacgatgagcgactcgttcaccttaagcacattgaactcacctacgtggcgcgccggcgaaacgagcttgacgggcgctcgtacaaaacagcagagggagacggcgcagccagtgtttttaaagataaaacaaggcacgtggtctgtgcggctctcccagtagctgagtagatactcgacacaatagaccgtgtctgtcttgagcatggcgtcgcacaccgagtaattggggtttttacagatgaggccggcatcggtgacgcgcagctcgctgggacccaacttgaggatacgccgcgtggcctgcaccagatcctgatggagaaccttgttcatctccatcgcaccgacgccaccgccgatttatttacccggcgccgactcgtcttttccctccaggattccgttaatgtccatgagcttgctgacgatcgccgttaatagttgcgtcttctcacggaggatctctccgtgactgcaggtcgcgcagtcgccgtgcacgtacttgaggaaggcggcgtacttctgacccgcgttcacgaaatttaagcgcgcgtccagagagggcagcaacagatcgtagacgcgcggcagcatcggctcgaactgtaatagcagatcgtcgtcaagatcgggtagcgcgtgtccgtcttcaccgtcctcgtcgtcaccacctcccccctcgagcccaccgctcgtaccagccgcgggctccgcgtcctcgtcgatcaccagcggtcgcgtcggcaccggagaatccacgtcatcctgcacgtcgttttcctcctctccgtcgtcatcgtccagaaacggcacccgctgcttagcccaggattccggttttagaagctccacatcgaagacgagagtggcatgtggtgggatgatgcctgggtgcccagtggcaccataggcataatctggagatatagtcagtttggctctctgacccacactcatctgggcaaccccttcttcccagcctcggatcacctcctgcttgcctagcataaacttaaagggcttgtttctgtcccgggaggaatcgactttctttccatcttcaagcatcccggtgtagtgcaccacacaggtctggccgcgcttggggaaggtgcgcccgtctcctggggagatggtttccacctgcactcccattcttttttccgcgtcctcaatcagcggcgccgatcgccatgaatccgagtacccacgtgagcagtaacggcccaacgactccccctcacgggccccacaccacgtttcttcccccgaccagcccggccccgtccaccagctccgtcgccgccgctaccttgtgcagtccgcaacgacaggccgtttcgcgttacagcggctggagcaccgagtacacccagtggcactcggacttgacaactgagctgctatggcacgcgcacccgcgtcaagtacctatggacgaagcgctggccgccgcggcggccgcctcataccaggtaaatcctcaacaccccgccaaccgttaccgtcattacgaattccagacgctcagcctcggcacctcggaggtagacgaactgctcaactgttgtgcggaagaaaccacgtgcggcggcacgcaatccaccgtactcaccaatgcgaccaacaccactagctgcggcggagccgtcgccggcagtagcaacgtaggacccgccggcgcttcggccgcctgcgacctagatgcagaactggccggcctcgaaacctcggcggccgactttgaacaactgcggcgactgtgcgcgccgctggccatcgacacgcgctgtaacctatgcgccatcatcagcatctgcctcaaacaggactgcgaccagagctggctcctcgagtacagcttgctgtgcttcaaatgcagttacgcgccccgtgcggcgctcagcacgctcatcatcatgtccgagtttacgcatctgctgcagcagcacttttccgatctgcgcatcgacgacctgttccgacaccacgttctcacggtcttcgatttccacctgcactttttcatcaatcgttgctttgaaaaacaagtgggcgacgcggttgataacgagaatgtcaccctgaaccatctggccgtggtgcgggccatggtcatgggtgaagacacggtgccttacaacaagcctcggcgccacccgcaacagaagcaaaaaaacaacccttatcacgtcgaagtgccgcaagaactgatcgacaactttctagaacacagctcacctagccgcgaccgcttcgtgcagctgcttttctatatgtgggccggcaccggcgtcatgagcaccacgccactcacggaactcacgcacactaagttcgcgcgactagacgcgttatccacggcctcggaaagagaagacgcaaggatgatgatagaagaagaggaggatgaagaaggaggagaaaaaggaggagacgatccgggccgtcacaacggcggtggcaccagcggggggttcagcgagagcacgctaaaaaaaaacgtgggtcccatttacctatgtcccgtacccgctttttttaccaagaaccaaaccagtaccgtgtgtctgctgtgcgaactcatggcctgctcctattacgataacgtcgtcctgcgcgagctgtaccgccgcgtcgtctcgtattgtcagaacaatgtgaagatggtggaccgcattcagctggtattggccgatctgttgcgcgaatgcacgtcgccgctcggcgcggcacacgaggacgtggcgcgctgtggactcgaagcacccacctcgcccggaggcgactcggactaccacggcctgagcggcgtcgacggcgcactggcgcgacccgacccggtattttgccacgtcctgcgtcaggcaggcgtcacgggcatctacaagcactttttctgcgacccgcagtgcgccggcaacatccgcgtcaccaacgaggccgtgctcttcggacgcctgcacccccaccacgtccaggaggtgaaactggccatctgtcacgacaattactatataagtcgacttccgcgacgtgtgtggctctgcatcacactcttcaaggcctttcagattacaaaacgcacctacaaaggcaaagtgcacctggcggactttatgcgcgatttcacgcagctgttggagagttgcgacatcaagctggtggaccccacgtacgtgatagacaagtatgtctagcgtgagcggcgtgcgcacgccgcgcgaacgacgctcggccttgcgctccctgctccgcaagcgccgccaacgcgagctggccagcaaagtggcgtcgacggtgaacggcgctacgtcggccaacaaccacggcgaaccgccgtcgccggccgacgcgcgcccgcgcctcacgctgcacgacctgcacgacatcttccgcgagcaccccgaactggagctcaagtaccttaacatgatgaagatggccatcacgggcaaagagtccatctgcttacccttcaatttccactcgcaccggcagcacacctgcctcgacatctcgccgtacggcaacgagcaggtctcgcgcatcgcctgcacctcgtgcgaggacaaccgcatcctgcccaccgcctccgacgccatggtggccttcatcaatcagacgtccaacatcatgaaaaatagaaacttttattacgggttctgtaagagcagcgagctactcaagctctccaccaaccagccgcccatcttccaaatttattacctgctgcacgccgccaaccacgacatcgtgccctttatgcacgccgaggacggccggttgcacatgcacgtcatcttcgaaaaccccgacgtgcacatcccctgcgactgcatcacgcagatgctcacggcggcgcgcgaagactacagcgtcacgctcaacatcgtgcgcgaccacgtcgttatcagcgtgctgtgtcacgccgtctcggccagcagcgtcaagatcgacgtgactattttgcaacgcaagattgacgagatggacattcccaacgacgtgagcgagtcctttgagcgctacaaagagctcattcaggagctgtgtcagtccagcggcaacaacctatacgaggaggccacgtcgtcctacgcgatacggtctcccttaaccgcgtcgccgttgcacgtagtttccaccaacggctgcggcccctcctcctcgtcccagtccacgccgcctcatctccacccgccgtcgcaggcgacgcagccccaccactactctcaccaccagtctcagtctcagcagcatcatcaccgtccccagtcaccaccgccgccgctgtttctcaacagcattcgtgcgccttgacactgtacggcagaaaagccggctccaagtgcaagcgccgcggcagcaccatgtgcaaaaacttgtccttgcgcgcggtttcgccgccgggaaagacgggcgacagcacgttagttacagccttgagaacctgctcaaagtacttgtcggcgtgaatgggcacgccgtgctcgcgcacgtagctcggatcttcggctacctcgtagttgcacacggccgacggtggtttccgcgccctcttctttgccggctctcctcctctcctgttgctctcctctaccccgccgccgtcagcgtcgtcgtccgtgccatcaatcgcgtccgaccgggaaaccacgccggcggttacagaatcaccgttgtcggaggaaccctgcggcgccgtccggacaccgggcgccgtcagaacgtaaaagacccgatccccgaccgagggtagctcctcagaacgggccgccaatcgcttaatgacggcaatgtgcggcaggttagattgacggtacagcgagatgtccttagagagcaccgacgaaagcaccaggtcctcgacacgcacacggtgcaggtacagatcgtcgcgggcctgcaccaagcggcgtaagatacgccagaaaccgcgtggcacgccgtacttcttgacttcatcgagtgagaggcgcgacaggcgcacggctgcttccgagacctcgcgatcctcaaagagcagcgagaggacgtcacgcgtgacgcccttgacgaactcgcaggccgtcttgcgcaccagatccacgcccttcatgctcagacccgaggcgccctccactttgccgatgtaacgtttcttgcagatcatcataagagagacgaagaccttttcaaactccagcttgacgggctccacaaaaagacaggccgtcacgtagtgcgccaggctgggcccacgcgccaccagagcctgcggcgtcaggccacgaaagcggacaaacacgctgtccgtgtccccgtagatgacccgcgcctccacccgccgttcgttcgagccccctgacgatgtttcgagcccctccggtaacgcgctgctctcctccgaatccccctcccgcgttcccactacatagtcttcctgattaaaaaaattgtgcaaaaaacacggctctgaaaagttgtctttgatgaaccgcgccgtgcgctctagcatgtcgcgaccgatgcgcgtgatgctggcggcgatgggcagacacggcatcataccgttgaccacgccggtaaaaccgtagaaagcgttgcacgttactttgagcgccatctgttccttgtcgagcagcatacggcgcacagggtcttgacactcgcgcatgcattcgcgcacggcacgccgctgcgaaacccacttgttgagcagttccgagagcaccgagacgcgcaccgaagcacgcacaaagcggtgggtcacgccgttctctagcgtgacgctgtatacgtcggcggggtccacagggtactcgccacccggcaccagcagggtggagtagcagaggttgtgggccatgatgatggaagggtagaggctggcaaagtcgaacacggccacggggtcgttgtagtaacccacctcgggctcaaacaccgtggcgccctggtacgaaaccgccgcagtaccgccggcgccgtgattgtcgttggaaacgccgacgccgccactactgccggagccgacgctgaaaacgccgacgctgctactactgttactgccggagccgggtgaaacgccgtcctgactggacggcgcagattgcaagggcggcgacatctgaaacatagccgccacagaacccgcgtcgccgggcacagcggcggtagagatgatagcagcgttaggtgacacagcaacgctattcgtttcgggcaccgtcgtacctttgctgtagtggttgggcaggataaaatcgcggcaggcgcactcgtccagcagcgaggtgtagatacggatctgctgtccgtcaaagatgacacgccgcaacggaattttagccagccgcgcgatggccccggcctcgtagtgaaaattaatggtgttgaacagatcgcgcaccaatacggcgtcctgcagacagtaacggcctacctgggcgcggccctcggcattagccacgaaacaacgcgggatgtccttgtaagacaggtcatccttgcgttgccgcaggtaaagctcggccatagtgttgagcttatagttgggcgagttagtcttggccatgcatacagggtacatgtcgataaccaccgaacccgcaatatacaccttggtggcggccgtgctggccggattgttgtgagaagccgagggaaaagcggcggcgtactgccgcttaaaacccacggcggggctgtgtaaaaagaaacggccgccctgcgccgtaggcaacttgcagaagcgctgcgagtccaccttatacaggtactcgagacgcgtgaggatgtacttcaagtcaaaagagttgatgttgtaaccggtcacaaaggccggcgcgtaccgttgaaagaaaagcataaagcccagcagcagctcgtattcggaagggaactcgtagacgtccacgtctgggcccacctgcccgcaggtgccgatcgtaaagagatgaagacccgagtgcccaaagatcacaccctccgaagtgcagccccgaccatcgttcccgtttgggatcccctgatccacggcggtgtttccccccgtctcgtagcacacgcacgagatctgaatgacaatgtcatcggacttctcggcgcagggaaaaccaccctcgccgctcatgcactcgatatcgaaggacaggcatcgatagcgcggccacgagctgtcgtcgggcacagccaccaggtcagagacatcgcagtctacctcgatatcacaagtcgacgcgcgaccctgctgccgccagtcgtaacgattcacggagcaccagccgaacgtggtgatccgccgatcgatgaccaaacgcgtcagcggatccacacggacctcgtacacgggaaaaccctgctccagcagatactcgccgatttttctggccatggtccagttgctgatagacacacactgcaaatcgggcacgggtcgcgtcccgtacccatagatggaggtcttggtggccggcgtgacagacacggcgtatggcgtccgcggttcgggcactagttcgcccacgctggcaatgacctcacgcagcctatcggtgtcgctgtactcacagtaaaagtagctgcgctgcccgaaaacgttgacgcagatactgtagccgtgttctgtggccccgaagaaacgcaacacgttccccgaaggcaccagatgctgacgatagcgcggcgacacgttttcgggcgagtcgaagaagagcacggcgtccgtctgatcgtaggtgtgaaaacgaataggtcccaccacgcgacccaccagggtctcgcgccaaggacacggccaaaccatgtcatgactcaacaaatgtttaatctctcgatagaacatgagaggcagccgtcccgtcttatgcttgatcaaccccgtctgaccgtcgaacatgacacctcgcggcacgatctgcaaaaactgtttctgtggcggccgcttgcccgagccctgcgcggagccgggctgcgaacgctgacgccggccacccgcgaccgcaccgccggtcacgccgccgctcagatacgggttgaaaaacatagcggaccgtgagaggctgacagcttacgaagcaaaatcacaaagaaaatacacatgcagcacctagatatccagtttaaccccgtatatcacaagtctctgtgtcaatattttttgtctagtttttttttcctcctggttcagacgttctcttcttcgtcggagtctttcaagtgtctgtagccgtttttgcgatgtcgcagccggtctagcaggttaggcttctgtcccttgtcctgcgtgccagtctgtccgtccaaagaatctgtaccgttctgctgcgctcgctgctctgcgtccagacgggccagggccagaagcatctggtaagcctgctcgttggtgtaaggcggagccgccgtggatgcatcagacgacggtggtcccggtcctttgcgaccagaattataaacactttcctcgtaggaaggcggagcctgtaacgacgtgtctttggtgctgcccgacgtcacggtggtcccgtcggcggacaccagatagggaaagaggttctgcagcggctgcgtgcacagacgccgctgtcgagtatagatcaaataagtgataatgactacggctatggccacgaggatgatggtgaaggctccgaaggggtttttgaggaaggtggcaacgccttcgaccacggaggccaccgcgccacccacggccccaatggctacgccaacggcctttcccgcggcgcccaggccgctcatgaggtcgtccagacccttgaggtagggcggtagcgggtcgactaccttgtcctccacgtactttacccgctgcttgtacgagttgaattcgcgcatgatctcttcgaggtcaaaaacgttgctggaacgcagctctttctgcgagtaaagttccagtaccctgaagtcggtattttccagcgggtcgatatccagggcgatcatgctgtcgacggtggagatactgctgaggtcaatcatgcgtttgaagaggtagtccacgtactcgtaggccgagttcccggcgatgaagatcttgaggctgggaagctgacattcctcagtgcggtggttgcccaacaggatttcgttgtcctcgcccagttgaccgtactgcacgtacgagctgttggcgaaattaaagatgaccacgggtcgtgagtagcagcgtcctggcgattccttcacgttcatatcacgcagcaccttgacgctggtttggttgatggtcacgcagctggccaggcccaagacatcacccatgaaacgcgcggcaatcggtttgttgtaaatggccgagagaatggctgacgggttgatcttgctgagttccttgaagacctctagggtgcgccgttgatccacacaccaggcttctgcgatttgcgccagcgcccggttgatgtaaccgcgcaacgtgtcataggtgaactgcagctgggcgtagaccagattgtgcaccgattccatgctggacaaatgagttgtattattgtcactcgtacttcttctggtcctatgagtgatattcagactggatcgattggccaaacgttccaattccaccaaagatttttgcttgatgccttgccagaacaccaccagaccgccgctggtttcgaagacggacacgtttccgtatttttcatatgtttgattgtatgaagtattgaaaatctgctgtaacttatttatagcctcatcacgtacgcagtccagcgcggagtcggacatgttcacttcttgtttcttagacagaaaagttgcagtcattttggcagaagaaaagtggtacgagtcttcggcttcggaacggatagtacgttccgaggcttcccagaaggtgagctggcaggtgacattcttctcgtcctgtatatcccaagagatcaccgagtcggcacgttcgagaaaagccaccaacctatgggtttctggcgcagcgttgggtcttccaaagtcggaaacgatggtgtagttcgggaaaatgaaaaacttgtcggcgttttctccaaagtagctggcattgcgattggttccgttgtagaaaggagaaatgtaaaccacatcacccgtggaagttgcaaaaaaatgataaggatacttggagcgcgcagtagtgatggtcagcatacagttcagattacaggtctcacgatagagccaggtgctgccgcggctgtgccactgatccttgaccgtcacgtaacgggtactgtgggtgttggaataatcgtcgggaattaattgcatggttttgttttcataactgtccctatgatatgccacgaaaaccgtgcctcctataacgcggctgtaggaactgtagcattgagcaaacttgttgatgtgatgaatctcccacataggaggcgccacgtattccgtattgctgcccagcagataagtggtgtagatgtaagcgtagctacgacgaaacgtcaaaaccttttggtagacccgtaccttaaaggtgtgcgccacgatgttgcgcttgtagaccaccatgatgccctcatccaagtcttcattgataggcttcatcgaggtgcagatgatattacgttcaaagcgaataagatccgtaccctgggccatagaacacacgcgataggggtacttggtagtgttgactcccaccacatctccgtacttgagggtagtgttgtagatagtctcgttggctctatgactgacggcttcagaagacgttacgtgttgagaatagactgaccgggtttgagcagacgtcgtacgagaagtatggcttccattgtgagtagaagaagttgcatgggaagtactagaagaggaaaccgcagcacccagacagacgatacacaggttaacgcagactaccaggcaccagatcctggattccatgttcgtcgcgggccaaatccagcagcgatgaggcgcgtcgtggtctcttgcgtgtcgcgcggaccctccgggaaacacccgcagtcgaggaggagggatacggacttggcagccaaggtcggtccggctccctgaagacacccgagacggccgcggcggccgtcagggtggagggcttggccacgggagctgttggcacgtcgccactctcatccggtctggacagatgcctgtagaggaggagatatagatctttggacttataaagacttccttcgtgacgaagcagcagcggccactctttgttatacgtgagaatcacatctctgtccgggtgcagttcgtcgcgcaggcacgcgatcgagagttgtttcccgaaagtttcattatatagtgcgacggagagcacgagctcccgcacgtgcatccacatctccttctgcagcacgtttaggtcctgacagtccgaaaaattgaaaaaacccatatacttcaccaccatccactcactgggatacacggtaccttccgcgcatttgaccaaatcgtccttgacgtggggtagtacgcccgcgttgtcgcaggcataggccatgtccacattgtgagagaggggataacgatcggtgcagtgggtgaagaggggcccgttacacaactcgtagatctgctgacccagtagcgggagggattccacaggcagactcttgtggatcaggttattgaccacatacaggtgctcatcgtaggtgaactgatcacccacgtccaccacgtcttggtcctggtggtattggctgcggtacagaaacccattcatgagcttagagataaagtccagacacaagggccccactagattgacatcgatgagcttgctagtcagacgctcctgcgttttgatgcaacggatcaccttgccatagcccacctccgagaccttctgcaggtaggcgcgtttgcgcacgttcacctcgcgagtgacgttgtggatgcgggagcgcgcgtccaccaagtcgagagcctcgtgttcgtcgcagttgcgcacccgtaagccgttctcgctgccgtcgccgtcctgcccattcacccctccccctaccactttcttgcctcctccacgagcccggccgccgccaccgttattcctctgactgtgagtactgctgttgctgctgttgctggccgtcatcaaagtcgtacccgtccccgacatcgcctcccgtccacgcaggtgaatagcctcgccctcggggccgtcgccccccgtgccatctggcagcggacgtcgaatctcctcgagaatatgcttgattttggtgtacatctcgttgctttcgtggagcttgttgaacaccgggttgtcctcgaaagcttgaatgctgagggatgtgatgaggtcgatgatcctgttgggggcggcaaagaccgaccccacgaacatgcgctcctccccgtccaacgccttttccccgagcacgaagatgtcctccacgtcctccccgtacagatggcgactgatgccgttcatgagcgcccggcacagctggtgatacacatttagctgctggatggtgatgcccacccgcttgacgataacctccgaggtacgggaccagtaggtaaaatccgacaaggaatatattcgttccggtatatccgtaaacaggttgtactccctcagcgcctcctccgcctcctggatgtagctgtggtaggccgatgaagaagagaataggcttttgagggccgaaaggactccagccaagtgggggatgcgcgttgtcaggtccagcaggtcctgctccaccgtctggatattcacatcggactggcttgacggacggtggaccgctatatggttgcacagcaagccctgcagccgcttgttcagcgagcggccctgattcgggatgatggtcagctcctcgtagcattgggcgcatgtcgtcccttcgacgtacacttcctgacgcgccaccggcgagatgccgcataggcgacggaggagctccagcaactgcgcgcagacctccaggccggcctccggcgccaggatcccgtacacgtagttcattttgcacaggaagcgctcgatgtcgttgagtgtggccagactgacgctgaaacggacgttgtccgtaaactggagctccacggtgtgatggcgatcgcagcgatccaaacggaggacggtacggtagaaggccgcccggtccggctggcgcgagtaggccatcagcgcccgatccagcaaagccgtatcctcgtgcagcgccttcagcagcatctccaggtagagcgtcagcaacgaactctgcgtacgattctgcgccaccacctccgggtagatcttccggtacagatacactatagccgccgcgtttctcttgaacggcgtggactccgccagtaacacgttcggatcgcagtactttagacactccagctccatggcgtattcgttgcatttcgaacacactacgcatagtttctgtaacaaattcatctccatgactcgactcgctcacgtacgagacgctgtcgtccggtctggcgccggccagagacatggagtcggtgcacaaataactcgcgggccgctcgctatgccgactgacgttgacgttaatatataacgacgtcgtcgacgacgcgggttctgctcccgaagctgttgccgccgcttgcggcgcaacctcctccaccaccgccgccgccggctcctccgcctcgggcgacgggggctcggagatgaccggctgtgtctgacactcctccccttcctcaggcggcccgggcgccgacgcgaatgtcggagtttgccagcgcggcggcggtctctgtctctggtgccgcggcgctaaccttcggggctgttgctgctgttgatgatgcgacgccgtctgtcgccgctgttgcggcggtagctgatacggtgtcgcctggtgctgctgtgtcggtggctgctgttgctgctgttgttgcggtctgaaaagcggccacgggggctgcgactgttgttgctgttgttgcgatgctcgtggctgcggcggccgttgtcgcggcgtttgctggcggttacaaccggctgcgtttggccggcaataacccgctgcccccgccgcccccgctgctcccgccgacgccgccagcctcgtcttcgccggcgttcacgagaaagcagccacctcccgtctcgccgggcacgccgaagcaaatggagttgcccgcgacggactcgccgagaagaagaccgccacccccgacgccggacgccgcgccgacgccactgggcgcgaagagcgccgacaggtcgtgcacctcccccccggcggcgtccgttaatcgctgggcgtcggcgtccagcacgcgtcgcaagttctccagcgaaaagtcctccacgccctgctcctgcaacgcggcaaacttgtccatcagcgacgcggccagcgcctcgcagccatccacgaagaagagcacatcgtcggacgcggggatctcctcgcgcacgctcagaatctcgtacacggccatcacttcggggtcgcaatccaagttctcggcgtccagcgccagcatgacgcggttttttataagatccgcgtcaaaaagcacgttctcgcggcgcgagcgtttgatgagcacgtcggccagacgcgtagccaagaggtagcgctggcgcatgaaacgataatcttggccgctcatagagctcacgttaaggctgcgttccacaccgttgcccgaaaagtagccgatctgcccaaactgatagatctccttgctgttgttgatacccgcatatttttccacgctcacgggcacggtcaccaaggaacgatgctcaaaaacgctccgtaccaacgattcacgcgccacagtggcggccatgggcgccggcacgcctgcggtcttcaagcccttgacatgcaacgcaaattcggcgggcgacgagaaccgcggactagcacctaacacgtgaggaaactgcgcgtggttctgcgtcgttaagcgcgtcgtcaacccgtgcagcgagccgatgtagtctttgaagccataatagcagaggaatttgttatggaaacggctttccacgtaactcagcacacagtctggcgccacatccagcagatcgtgctcctgatagtcagccgtcacagccaccagaaatttgacgaaagcattgaactcgcccatgtcacctatgggcacattcttgggcaacgcgttggaacagaccttctgccaaaactgtaagcaggggagaccacattcaggaaagagtcgctcgtgatgtcgatacagcagaaatcccaagcagcccttagccggattacgacgcggaacgtgatcgcggcgaaaaaacacgctacccgcgttgcccttgcccgcgcggtagatgggtcggtttttcacccgcaccatgatcaacgtgggtaccgacagccgcgagagcttgatctccatgggcaccacggcgtacgtgccctgcgcgtacagcctaaagtccagcaggcggtcgtgatccgaattcttggacgacttgatctgcttggtgaagagaaagcccttgcgcgacgacgtggtggagaacgcgccgtgaatggattgaaaatgctgcgtcatccatttggataccaagttggtggtcaacggattgtccacaatgtatgaggtagcggtaataagcgccacgttctggatcacgtaaaagacggatctgaaataggcgtaggctagcagcggctggaaggccacggcgtagggattcagatccaggttgaaggcctgcgtggcgcccgccacctcgtcgcggctgctcttgaggcgcacctccgaaacgaaacccagggcctcgtcgtccacaaacttgttgagcgccgaaaagacggccacaaagtcgcttttgccgtgcgcgctaaaggtatcctcgcccgtcacggggtcgatgagccgcatcttgcggcagtaatccaagatgcgattgagccgataggtacggtccacgctagcgcccaacatgcgaccgccgcgccccatcattcccccggaatccccaccacccccaccaccacgaccgccacccagaccgtcgctcgggcccccgctcacgtctcgtccaccacccccgccagcaccgccgcccggaaccccgtcgtcacctttgccgtccaaacccccgtccttggcgtcgacgttgtaacgccgaccgaagctgcccaaaatatccacgtcgttgagaaaacgcgactgcacggtgatcacgcagggctccttcttgggctgcttgggcaccacgggcaagcgggtgcgcacccgcacgaaggccgtctgataacacgtgtggcaacaagtacccccacaggcctcgcacagccccgcggcgcagcccaccaggtgattcgtgagcgtcgacgaacccgacaagcccgtgttgtacaccgagacacgattcagataccagacgaagcccgaaactagctgcggacacgtgccacacaccaacgccaaatgctgcggcccatagcgttcgtccttgagcggcgcgccctgaaacttgagcaccttgcgcgcgtcgttgtagacgtcttcgcaggccgccgacaacccgttggtgaactgaatagccttgagcaacgtctcctgactggccgtaccgccggcgctgggatgccgcgccgacgactggagatacaccagcctgtgctggtagagcaccgaattagcgctgaagaccaaggcggccacgtgcgtcgagagatgcaacttgagctcggtcagcgcgcggatcagatcgcggtgatcggttgcgttggtcactaaaggccactcggaaaagagcatagattcggcaggttggtaagccgaatcgaaaaataccgaggcaaaactgaaggccaactcgcaaaccaccgcgtcactcagcatcagatgatccttttccagactgctgagtcgctggctcatgtaccccaagtagcgcttatgtggcgccagcttcaccgactgctgactgtcgtgcacaaactgccgcaacgccgcctcgatcagcacacgcggctccgagaagcgcagcgattgacaccatgacgtgtacacgtagtagaaaagcgtctcgcttacggccggcacgtagagccctcgcgcctccacaaaagcgctgcgcgcatccagcgagacctcgtcggcttcggcgtcaagctgcaacgaattaaagagcgtaggcgggtacaacggcacgcgcaccgcctcgccgccgtgcagtcgcaccgtggtcgcctcctccacgcatggaatcagctgaccggcaaagagaaactccttcaagccgttgcccaccaccacgtgcacagtcgtctcggacgcctgacagcccaccgccgcgcacaacgccgccagatcggtaggcacgcgatccgcctcgggcatgtaagcctccaacgcgtacttctggcgggcgtcctcgcacagccgatgcacgtctccgtgatcctcggtaaaagccacgatgccttgcgtatgatgaaagtagagcgcaaaaggacagaaggacgtgactttcgtgagcaccccgccgtcgtaacaaagcacaggcgtgcgcacagagacgccgaaatccgcctccaccgtgagccccgccaacaaaggagcgatcaccacgctcgaggaacggtcgcatagcgagagagtggccagaatctcctgcgtttctgcgttcaacctgctgaagtagagaaaagccgcgggccccaccggcgctagcgcggttagttcctcgtggctcatggtggatgaacggaagacaatggctacgccgccactgagtgaattttataccaaggaaaagttcagcacgtcatgtttgacgcacgacgtctgagacaccaccgtggccaccactgcggtctggctgcggttgcggaccaccaaaggcgacaaccgcaacgatcccagcaattcgtaagaaaagctaaccgctacggtcaggtagcctctcgcagccagaccgctagccgacgcacccgcccgcgaaaatagcgtgatgttcgggacggctttgcgtcaccgccaactaacgtcggtagtcgagcacgtcgtttatcctcagcacaccgtccgatcacaatccgttttcccactcagtcgcacaagcagcacataaaaaccccacacagggcacgtgaaaacaccgtccctagaaaacggcgttttctgtcctaccgtcacccgtatacacaggcaaatcccaatcccgatccccgaaaacaccgtacggtgtttgtgacctccaaaatcacatcagctaacaaaccgtgaaaagtcacgtttcacgaacacggtgtttttaaatcacaaagaaccgcctgacggtttacaagcagaaacaccgcaccacggtggtacaagcgcgatgaatctggtctcgcaacctcaatcgccgctatcaccaccgattttcgctgcgctccgccgacaaaacgccgtacaagctacacaccccaaaaacccgcgcgcctacgggcgccaaacctgtgtgttatctcaacgtcacaacacgacacaaaccgcgtaacgtggtttcccgaacacgtacgcggcacagacccccgacacgtactcgaagaccttacagtttacgagtcaataaaacaggaaaagatccgaactttaaaattgtgtgtttttattttcccatccccctctttttaccaaaaaacacatttttcgtcttgtaaaaagtaactttcgcccattgccatgaaacaccgtgatggggaacggtgttgtgtgtcgactgacgtcactacggcgatcagtatcgacgtcgtgtatacataacggtgcccggtgtttttattcggggcgttgtcgcgtcttgatgtaatgtaacctgaaaccgccgtgcccaagaatgcggaagccagcgtgtaatcataacggggttttgggtacaatctgacgacatctggcggcgagcgtacaccatcgaatgtggcgatcgccggctctacgtcacaatgacgcaaaaacacactgtaaaacccgcgtagacagctttcctggtcaacgagcgccatctggtgtcggcataagaacaggcatcaaccccgtggccggcgaggcggtgagcacttttgttggtcacgtgaccatcagcgcaggaagcgaggcccgtagaaccgcccaagaggcggtgccagatgccaacgtcataatcacaaggtgatttgttacgtcacgcgcgcgcacgcacgcgcgcggtagaatacagcgatccctagtgaagccacacccattacgtgtagccatatccgcttacgtatacagccacacccctaggtacgccaccttatctaccaatcacagaaacggatatacaatgacccctccctagactccaccccttgtacggaaatttcagataggtggaacccgttagggttccaccgtcctcggtgtacgtacaggcttctccgtctaccggaaatatacacctgctgacgtagacgctactcccggatacgcgtcataagctactggaccctaggggggagtgtctacagggctacgtgcacgcccccttacctagggtatccgcccccttcctctgttttggcctagtaaacttaacgccgccgcttctcacgtgacccctgacaagcctacgtcacactcgcgtgaccacacccactccggatatacgtcatcctgtggaattccggacatacggtgacgtagcgagcgtagcgagctacgtcacgtatgcgtgcgtcatctccggcggaaatcatctctgatgacgtagcgagcgaagcgagctacgtcatcagtccgttttacgtataccggatgctaggcgacgccccgtaggggcggagcctagcttccacccctaggatgcataccctatatagcataattcttctaacgaaacgttctacgaaaacggactggcggaacgggaaccaccgtaacccccccccctcacccccccccttctcctccggaaccggggggggcaaatttttaccaaatttgggcaaccatgatttccaatgggacggcgtttccgtgcgcatgcgcagtcggggcgagtttttggttgtcagggcgttgccacgcggattatgggatggtgactcgagtgcgcatgcgccggggatgccgcatggaaaacctatatataaggaggggtgaaccaggggccccggggcgcatgcgcgggccagggcccgcgggagggtcgccctgcgcatgcgccggtaaaattccactgtgtgtgtcgtgcgcatgcgccagtatttttccactagaggcggtcagtgcgcatgcgtcggtaaaattccactagatgtgcgccgtgcgcatgcgccggtatttttccactgggcggccgcacctagggagcgcgagccccgtgccgggcatgggccgcggcggtggaaaattaccgctccgcccacctaggcggggcatctgaaaacctataaaacccggcgtgcccgccgccccccggcgcagtccgcggcagggttccggccgtgctgcggtccgcacgctgcgcccgctcccgcctgcctcccgccctaccccccaccctccccggccgaggcccggcgccggtccgtccgcgggcccgtcccaccgccctggagcaccatccggggccgtgggccgggcaccgggcgcggcccgctccggacctcggccgggggtccctcccctccccccgctcgacccccccatccgacggcccggccgggctgggacccccgcaccggggtcccggttcccgtccgtggcccggggggacccgagcgggggcttcccacccccaccccgctcctccccgggctccggcccgggatccctcgctgctcccggcgacctccgccggcttcccggtccacccgccgcggaatggacgggacccggggtccgcgcccttcccctccccccacggggggctgggtcgcggaccccggttcctaggctcgttccgcggtgggcgaccggggatcccccacccagctccccttcccggcccgccttgctggcttttgggcccctgcgggctttttttttccggctgggggtcgcggcggtcggccgacgacgacggtaggtgggccgggtggacggtggtggggacgggcgacgccccggctcgacggcaatcggtcccggaaggttgggggctgggggcccggtcaggagctccgggagcggggtcgaccgcgacggcttccgggtctcgcggcggctccctctcggcggctccggttgggctcccctcccccctctcgagggtccggccgccagtcgtgaccgggggtccctcggcctagccgccggctctcggtccgccttatcctgggcgttggcctgtcccgtgacgctcccctcccccgctgctccccaaaaaaactccgcccgaaccgtcgcggcttgctggccctgggcgtggtcccccactcccctccccccatcggccgcccagccggggtcggcgcctcggaccccaccaggctgtggcgtgtgtgctggccgatgcggcggcgaggttgggtgtggccggaagcgctcggggtcgacggtgggccgccatgacacctcaattgtcgtcagtacgcccctccacaatcaccgtccccacacgatgggcccggcaggtcacccaacgttggttcaggcccagtcgggttttttccccggcacgaacgcacgtccccgtgggctccacgcgttttccaccctttcctggaggggtccggaacaccgtgaatccacggggagggtcccggcacgggccgaggagaccacgaccgtcccacccggcgtgtcgactcgtccgagacccgggaagggaacaggccccacctttttttcccttctccgattttgccgtggaaaacccgtgaaccgatacgggtgcagacggccgaaaaaaatcgagacgacaatatgacggcagggcgcgatcttctcccccatccgacaaaaccgtgtcccttaaaattccccacctttctctgttcaaatggccccgaaactgtaaaacaccgtttgaccgcaccccaaccggcgccatcttggtgaccttctcgacggttctctcgctcgtcatgccgttctgagctccgacatggcggacgagagaaaatggcgtcgagagcctaggagcgtttttgctccaggcgggtaaaaaaatagcacgataacttttctgtgcttttttttgagacgttttagaagagcttttttctgctcagagcgaaaaaatgatagccctgaaaatctcgacgagtctggccgagcggcgccatcttggaggaggggcgagtcgcgggcaccgcctcggtaccccctggccgaggcgagtccgcggtcgccgcctgttccgtgatgctacctagagggcgctgtcgaggcgactcttcctgttttcgccctgagggctaacggtcgctgacgtcaaaccatctcgtgctcgctgagtcacatccggttgttgacaagcgatggaggaccgcacccaaagtgcgccctctagtcatcgcgcctgaccccttttataaactgctcgaagaaaagaacaccttatgtgaaaaaatacagaatgatgacaagttcatccaacacaaccgctcaacaacgccatatctatcagtgtccaaaaactatcttctatcctttgaaactataaatgctgcctatatacatatttagtatccaagactcttaccacgtagacgaaaagaagtgatacaatgatcttgacgtgtatcgtctatatcgtgctagatatattcagataagacgcgcaaaccatagatttctcatcagtatcatgaaagacctatagctctatatacgaacctagtcattttaggacagccgccggagaagccgacgagggatcgggcgggtgcagccagaacctcacgcccgatcccgcctccggtaggcgatttgcatctgtttggtaaaaagctcataagtctgtatgtgacctatatatattatacgctatgtacaccgaactgtcgctgttgtataagaagaaaaaactctccatatttatatcgtctgaatttttgcttgatagacacgtgtttggaactctgtccccccacgttttcactgtgtataacaaaaatatgtgtttctcaaaagatcttgaggtgtttgaaaacgggggaaacctgcgtttgggtgctctaagccccggactgggacgtagccggcgtccggcacctatatttttctatttttttttacaaaatatatgatgaaccaagaataaaactctagctctcgtctatttttaatatgctctacttagaacctttttaatgacagaatgaactccatgttatacgctctttatatagtttctctgcactaacctttaaaaccgtatccttccctgttgtacaaatcatcttttgatacacaatgatgacctgatatccctccatatatatgatcggatattattccgttagacttgtcctccttttttttcctcatctcctgtatctggagatatatgttgaccaccaccgccatgaccaccaaaaagctagccgtcacgactagaaatgtgtaggattcggactttccgttcgagaagaaaaagagaccgcgtctctggacgctctttttgtcagtctgaatcgacccgggatacgtaagagagcggccctacatcggggggcgctcgagaccgacgacgttccatctgaccagaaaaaaaaaggcacccctcggtggcgacctctcaccatcgtttgcccgtccgcccgtccttcgtagccatcatcatctcaggctctatcggtaccatcgttgtcatctgaaaaaaaaactgcctcacccacctgcgtaaaaacaccatctttccggaggtgcggtaagacgggcaaatacggtcgtgccgaggcaaaaaaaacgcaccatcgacaccacaccctcatgagcaccacctgtcggtgttggtcgtcctccatcgttctctacgaacatctcgacgcccgggtgacggacgacggcaagacgtcccggagaagacggtgttctctcgggcggtacgctctctggatctataatatctatagtagctaaacgagactgtgagtacgacgaaccacatcatcttttttttatgttgcttctttagaaaatgacttatgtcgacgacactcggcatcagccatctcgtgaaacacgctcgcttttcgtctctccaaggaacactgggtccgctgaaagggaccgtgtaccgaccaaagcaaaaaacacacacgtagtaacatgatcaaccacgtctgaatgacacgaaaacacaatcgtataacgctctattcatggaacgaacttggaataaaaaaaccatcgcaggccagaggctaagccgaaaccgtccggggaagcgggcgcgagttttccgacttagcctttggtgctcgttgagcctcttttttttttctgattctctgaagaatcaccgtcacagccctatgacgcgaaatcaattgctagaacataaacgttctcaacaggtatgaaatgaacaaactagatgatgctataaccttatattgtgtgtatatagataggtgtgaaatttgtaggataaaaagtgtcgttgtatgatgcacaacgatcgtgaaactggagactgtagctctctaccgaatgcaaatacacaaatgacatcgattcccgtccccacataaagaaatgtgctttactgtgaaagaatgaagaagattcttgttcctcgtacgacggggccctcgctcgtcgtgcctcttcccccctccgggagaggggacgtcggggccctccgtcgcaccgggccgaagccagtgaaatgtttactacactgtcatcagaatatatgatgtatattatttcctccaaactcctcaccatagccaccaattcgcatcacttaagaaagtagtagcaaccgcggcggcggcgaccggccggtcgtcgtctcctcgtcctcaaatgttgtacatgtgcagaaaaatgtgtaaatacgtgttatttatcccatgcgtcttgtacatagatatatgtttttatatacgctatttatactttatatatccttttgcataaccatagacagtcaaggattttaatgatttgctcatccgcctttgagccatcgcttaggagttagttcctctatgttctcggcccaccttttcgactacagtagcaaacccttgtactaccaccccgataaaaaccacatcatcatcgtcaccacgacctggaaacgacacacgttcccccccaatcttgggcatgtgtatatataaaaagaatgggagggagaggacgtggggctcgagaagaaataaacgccaagctcgattcgaaccaaaaaaccacatgtgtattgtgctttgtttttttttttacggtgggggaaaaggagggggccgtcattaacggaaaccgtgtatggggtccggacacgaacagtacacagcttatggggaaaaaagctcacagagagaaaaaacaccaagctcaggcacgcgtacatcattattatcatcatcggatatctcaccacgggtcatagtagtaccaaggagtgtgtaacaccattttttcttttctttgtaacgggataagggacagcaatcatcacgcacaacacccttcactctctttttagtcatccatatcatcgctgtaacacagcatgtcctcgtaatcgggcgtctggcagcgcattaccaccgagtcgtcttcttgcggtaccggtggtggtggtggtggcggcggcggctgctgctgctgggttgccgtcgtactgtgattaccgttggcggactgcaccgggatgatgggctgcttgtggggaacctggggtggactgccgccgtgagaaggcgacggcgtcatcaagttaagctcaccacggtgactccggacaccggcgaggggcgccgggggactgggagggaccgcggtcgtcttgtagacgacggtgtccccgtgtcgatccgtggctcgtaccagatcttgactgctagcgtcgtcactgtcttcgtcctcttccagctcgccctcagagtagtgctgctgtggttgcgacggtggctgggcgggaggagcggcggcgatcattggagagggatgtcgatgactcccttctctgtcctttttatcgtaggctgtcagcgttgctgggtccgtcctgctttccatatttgcgtattgctcatcggtgggatgaatttggtctcctccccgctgttgtccgccggcagtggcgtggttgctggcggttgtcgttgtcgtaccggcaaagacggtgagatccaatagcgactgctcgtcgaagggacagtacgctatcatgaaacgatagggtgccaacgcgcgttggatgcgcagttcgcacatctcgttctgacactcgtggcactgcagggcgcctaggatcaggtccgagacagcgccgcagcggtaggtacccatggcgttgttagtatcgaactggtcaaaaaattggggcgtaccggtgacttgcaacgcgcgacggcgtagcgagacggccacgcgcgagaaagagcacacataggccatggcgcggtgcatgggttgcgagaaggtctcgggcggacgcttctgcagatcgcagacgtcgtcgcgtagccaggcgctcatttgaccgggcttcttgactagccgtttgagcgtgctgcaatggtcgccccagccgtcctggtggtccaggatgcagcccaggtccaggttgttgagtttgttgaagagtagctgacgcatgccgcccaccgtctccagatagggatcgtgcgggttgacgggtagcccgtgcaggtggtggtacttcatgtagctgagcgtttcgtcgatgatggccagcaacgtgtgcaagttgggagcgttgtacacggcgaagatcttttccaccaccagcttgcgcagcaacggttcctccagccaatcgaactgttgacgaatgtgcaacaggtagtcggtgtgcatgagctcgtcgtgtgacagcaggatgcgaccgcgcggctgatgatcttgcgggaaggcggtggggaccttgagatcggcggggtagggtgccagacgtagactctcggccgtgtagcgctgaaggtcgtaaacgggcgaggtagaactcggtgaggtacccgacgaggcggcgccgcgctgcagacgcgctctttttttcttttcgatcaaacggctgagttgctgtagttcgtcctcgtccatggcgtccagttcgtcgtcaataagcgccagcatctgttgttgttgcggtccggcggacgatccgtgatgattattggctgaggaggggtgagaagaaccgaaagtcgtaggacaactgggaactcggcgacgaagatgcgtcgaatcgccgccgtgatggtgcggttcgccgtcatcgttgtcgtaagacttaccgtagtgggggttaaggggcaccgaggcggacgcggccacgcgtcgcttgaaagaggaggacgccctatgtccgccacggaagcccgcggtgcccatgatgatgtgtccgccggtgcccccgagtgcgtggcgggaggagggtggaaggggaggaggatagtggtccggatcgccttcggtatcatcgtctttgctgtagcggggtcgtcgtgcggggacgcagggtcggtgatgatgcgaggcggcgccgacggtatcttccgcgagatggtgttcgctggcggctgctccgttccgtgtcgacggcgaggttggacttcgctcgcgtcggaacttccgtggcacgggttcgtaatccagacagaagcgccgtgcgcgacgggcgcggcgttcgcgctcgctcagggaagataacgacggagcgtcgtgacggccgcgtgagtgcagctccatggccgccgtcgctaggaaggtcacgttcgggcacgctgatgtatatatagatgagaccgctgccggggggcgggtcaccggcgccgtggaaagtgaggctcagacggcggtcgccggcggcacgggcgcgtcgggcggtctgattttgatggaaatgtggacgtttttggcgttggagtgacactttttggtgaaacagcggctccagaggctggcccagagcgcgtagctgtgctcggtgcgcaggtcgatgaacacctgcacggtctcttgcgggttgcggtgcgtgtagttgagacagcgaaaatcccgcgtgcgcgcgccgtcgcgccgcttgacggccacgcagcaggcgccgtggggctgaaagaggaggacgtggggcgcggtaaactgctcgctgacgtgcggttcgtagtgttgcgtgaggtgctcgagcagcggcggccacacgcgggtgacgacgagccgctgcaagtccgtgtcggaaatcgcagcggcagtggcgccgtcgccaccgtacaggtgataggcgagcacctcggtgagaccgcggcgtcgataacgcgtcacgttaagcgagcgcgtctcgataaagttggcttcggtcgaggggcagattttgtcgcgtacgctgagaatgacgcgtggcggcggcgacaggggcaacgcgggcaggtcgtgcggcgggtggtggtgaagcaggttacgcagatccagttgggcgcgcacaaagcctagcgggtgttcgcggtaggcgtcgggcacgatgaacagcggcaacagacggcgatgcatgaaatagccgtcgtcttggtccattttatacatgtagggcagacgtacagagcgtccatggtggtagatgcctgtgtctaggctgctctcgggatgcgagatggggtccagcagcgtgtgcagttcggcgtcgagacagacggcgtgattgagcacctgcgccacggcgcgtaaaacgctggggtgtacggcgacggtgcaggcggggaacggcgtgatgatgcgcagccccagtttgcccttgcagcggcagtaagggggtgacgtgtcaacggaggacgttgttttttggaaaacgccgttatccgggacgttatttttatcctctttcccgtcttcgtcttcctctgtgtcgcgctcgtcccggtaatcgagatagtcgtcgtcatcgaaaggcgcgccggccgcgtccacgggcacgctgttgggtgggcacgcgcttttgaagaaatagaccgggtgccggtcggggtgcgtgtagccaaagaggctcgcccatacggtcatccagacgcgtcgtagtccgcgacatagctcaaagacggtgtgtcgcgccagaccggagacgccgtcgcgcagccgtaaatcaaagtcggccacaaaattgaagacgggcagacgttcgttgaagacttcgtgtcgcgtgtagtagaactgtgtctcggggctggtgctggccacgtcgtcgtcgtgtagccacacggtctcggtcagggcctcgtccgagaaacggctgtcgggtacgtgacggagcaggtcacgcggaaagaggctgcgatgccaggtttcggaggccacggcgcagaagacgtgctggtcattgggcaggtgtacgcggtagacgggcagcggtcgctccagcagcggtgccagcgcgggctcgggtagcaggtagcgacgttgcgagtaacgcgttagcgtgccggtggtgtaagtctgggctgtgcgtagcgaggcgcatagacgtaacaagccggacagggagcgttccagcggggagaagacagactcggaaagcgtgttgatgcgttcgagctggcgcgccagctgcgtggaggtgccgaagaagcccgccaggtgcgtgccgtcgatgcggccgccgtagccggccagccccaagccgtgcgggctggtcgccgagtggggggattcgtcgagacgcagtaggtgcgtctccacgtagtcgtgtagaaagttgtcgagcgagaagtatttttgcatgacgtccagcagctcggtggaaagccggcggcccagaaaacccggttcgcgcgtgcactgcgcttcgggcgccgcgtcagcgtcgtaagccaccacgcgccggtactcgagcaaccgcgcgcgtgccagcgccgtgcggtaggccaggtagacgtagtgcacgcagaccgtgtcgggcagacgcgcacgttcgcggaacgcgttgatctgcgtgtccacctgctctagctcggtgtagtcgcggcggttgcgcgcgacggcgtacgccacgaaagcggacacgcgctgacggaagggcgagcccagtagcagacgcgcgaactcgcccatggaggcgtgcgtggggatgatggtgcccaggtcgcgcgtgcagaagctgcgcacgtactcctccacggtggagatggtgctgtactggccctcgaataggtagtaggccatggtcagcagcacctggccctcggtgtgcccgaagacgctgatgaaccacgagggcgaggtggggcagaggaagacctggttgagatgacgtagcacggccgcgtggtgaaagtacaccaggtgcttgaattcgcgcacctcgccgccgtgttcgggcgagagcacgggcgtgcggaaaagatgccggtagagcggttgcgtctcggcctcgtccagactggcgatgagcgccgagagggggatgggctggcgcgcggccaggtagcgcgagagctgcagcgtttcgttgttcacggcgaagacgggcgccacccgccgcgagtccgagcacttttgcgtctgtaggcagaagtaaacacgtcgcgagacctggtgtttgaccagcagggggaagacgcagtggtccgtcggtgtctgcgagagtacgttggcgactatatgagcagaatcatactctgttgcgaacagaacgagcgtcatcgtcgcgccggcacgatgcagctggcccagcgcctgtgcgagctgctgatgtgccgtcgcaaagccgcgcctgtggccgattacgtgctgctgcagcctagcgaggacgtggagctgcgcgagctgcaggcgtttctggacgagaactttaagcagctggagatcaccccggccgacctgcgaaccttttctcgcgacacggacgtggtgaaccacctgctgaagctgctgccgctctataggcaatgccagagcaagtgcgcgttcctcaagggctatctctcggagggctgtttgcctcacacgcggccggcggccgaggtggagtgcaagaaatcgcagcgtatcctagaggccctggacattctcatcctcaaactggtggtgggcgagtttgccatgtccgaggccgacagcctggagatgttgctggacaagttctccacggatcaggcctcgctggtggaggtgcagcgcgttatgggcctggtggacatggactgcgagaaaagcgcgtacatgctcgaggccggcgcggctgcgacggttgcgccactgacgccaccggcggtcgttcagggggaaagcggcgtccgcgaggacggggaaacggttgccgccgtgtcggcctttgcctgtccctcggtttcggactcgctgatccccgaggaaacgggggtcacgcgtcctatgatgagtttggctcacattaacaccgtctcctgtcctaccgttatgaggttcgaccagcggctgctggaagagggcgacgaggaggatgaagtgaccgtgatgtcgccgtcacccgagcccgtgcaacagcagccgccggtcgagcccgtgcagcagcagccccagggacgcgggtctcaccgtcggcgctacaaggagtcggcgccgcaagagacgctgcctacgaatcacgaacgcgagattttggatctcatgcgacacagccccgacgtgcctcgggaggcggtgatgtcaccgaccatggtcaccatacctcctccccagataccctttgtgggttccgcgcgtgaactcaggggcgtgaagaaaaagaaacccacggcggcggccttgctgtcctccgcgtgaacagcctggcacgttttggaaaacgtacgtgatcacggacacgacgagtacggggtttctcatagacgtactttattaggtcagggatgacggggaggtttcgggccgacgtcaaaaataacgtcattcgtgttgacagggctttctgcgtcggagctcttttcatcttcttctgtctcgtcgacgtcatcgtctaccggcgagggtgtccgttgcagcaacgcgtgctcgggcgtgtgggtgaaaccgatgtcgggggtgggcggcacgatcatctgtcctagggggtgactgcccaccggcagataggtaaagcggtgggtggtaaaaaccgctttggctacggtggtgtgtggggagatgcagacggtggtgtgcgaagtgttgaccaccgtcacgccggccgcggtacccgggagccagatggtgggtcggatgatgagatccgattgactaaactggcgcacgcccactatgagggcgcagataccgggcgcgtgcacgtaggccgcgtcaaaatagacggtttgcgtgtgacccggaccgatcaccagcgtctgacgggtacgtaacgaaaagaaacggtgttcgttgggcggcggcaagttcatgagctgccagggttctggtacaaaacaggggaaaacgccgatatcgccttcgatggtgcccggaaagatggactgaaaagtgtcgttgaggttgacgacatccaactgcgggacttgcagcctggattccagcagctcgggcatgcaaacgaattgcgcgtccaggcatttgtaaaaggtaatgccgaaaaaaccttcggggatatagaggctgacgcccagcgaggtgggcactttgcgctcgcgtgatagccaaatgatgtgtttattgtaaaaggccagctgcgtgtggcattgtttgacgatgaaactggaaggcatccacttgtaaggaactttgagcggtgacggtaatggcgacgacgcttcatcctctcccggatgctgctctttgtcgtatttctcctcggtcgattggggcagcgtaaatgtggtttgaaaatcgctatcgctagcgaaacgcacgcagtaacgcatgttgacggatttctcggctaggatgatggagcctgatgacgatgcggactcttccttcattattaacgtaggggtctcccagaatcgctgaaaacgggagcgcggcagccgcgacagtaccagttgagagtcgattcggtcggtcaacatcgtaagcatcgtggcggtggtgtgatggagtggaacacactagtattaggtcttttagttttatcggtagtggcagagagttctggtaacaattcatccacgtcaacctctgcaactacatcaaagtcttctgctagcgtatcaactaccaaactaacaacagttgcaacaacttctgcaacaactacgacgactacgaccttatcgacaactagcactaaactcagttctaccacccacgatcctaatgtgatgagacgacatgcgaacgatgatttttacaaggcgcattgcacatcgcatatgtatgagctctcactgtccagctttgcggcctggtggactatgcttaatgctctaattctcatgggagctttttgtattgtactacgacattgctgcttccagaactttactgcaaccaccaccaaaggctattgagggtggacagatttacagcccggcggtgttccggcggggtaaggtttacatacgtgggtgaccggaggctaaagttacgaatctcatctagaaacagcagcgagtctagatagtcccacaggggatctataaatgttctctgaaaccccattgatggtgacgtaggtgtagttttgttactatcggaagctgttttgttttccacgaacatggtttcgttgtaatataaggagctcatgtcgagagtaccgtaaatagtgtacggcgtttcgttacggattagtacgtgcgtgtttttcataaattctgacacggcggttcggttgcggcttggttcacaaaaaggattttgccggtaacgtagagtggtatacacccacgttgctaggtcccttaactgtgtggccataatggacttcataaagctgctatcaggacgataagcaattgtagacgtggaaacccgccttgcggcggtagtaatactataagtcacgttagtagtgacgttgagagcggcagacgttgtataggaaaagtatggcgtagtagtactctgagttttcttagcttttttttcgaattgttccttaacgggcgcttgtttacgttttagttttcgcatagtgttttttaacttggtgccgttaatatacttggggacgcgaaatagattccggctcatggcgttaaccaggtagaaactgtgtgtacagttgcgttgtgcgtaacgtaaaagcagggcggttaaacctagaaaataaatcgtttgactatctacgttaaccttagtcggacccacgtacaatttggtgttccaacgcggtacattgaaaaacatggggttgaacgtggtgaaattaccgcaaccttgttcgccagtatcattacgtttggaaacgtttagcatttcggaaagacaagtcatggaaggcacagtaccacaaggtgggggtctgaatgttatcgttttagccgtatgattgtactgtgagtaaacgtattttgcgggttttctaagctgggtactataaaaatcaaaccacagataggttatactataattctgaatggggcccgctaaaatgtagtattgtggaaactctgtcatgttcatagtgagatttttaaccggttgtttacttacattgtattttgtagaaatagtcgtttctagttgtctcaaaatttctaacttaagctgatctaatttatatttgcctatcttagacagtaccaagcccctccaaggacgattataaagcgcttttgacataactttacagtttatgaaagaaacaagcaagaaagatatagatattagaaacaccatcttagggacgtctctcaccatcatctcttttctccccatgacagaggaggagaccccgcaccgtccgtctgccttgtggtttggcttgcctgcgtgtactcactgctgattctggtcgttttgctgctcatctaccgttgttgcatcggcttccaagacgacctagtctcccgcaccttggctgtgtaccaagcttgtatccagggcccgatatgtaaccagacccataacagtacctcgtaaataaagacgcacagacctcacgcatatagtaccatcacaccgtgtggcgtgtactttattacaacgagcaagagtgcccctaagtattggggcccgtaccgttttagaagattttgtgtgaatgtctttaacttttctgtcccttttctcataaactgtcaggttctacagtcagcatgtcttgagcatgcggtagagcagatagatgccgatgatggccgatagcgcgtagacggacatcatgaggagacgactgtcggtggcgtccacgacgacgtcagttacttctaggaccgtaccgtttttcaaaagcatgaggtagtgagttcgcggagatgagaccaccacttcgttgtagggatccagggcgaaaaggacgtcgtccgagtcgtgcatgtacatgatgttgatgacgccttgcgtgtcgtcgtattctagcagggcgctttggcaaaaggcgcagttttctagtgaaatgttgagcgccgctgtgatgctgtgtgtggtgtgcatgttgcgcgttagttcgcatttagtttgactgtccgtttgggtgatgatgaggctctggcctacgacggtggtggagacagggtaggagatacctttgatcaggtactggtttgttacgacataactgacgtgttcggagacggttagcgcggagaaggattcgccgagcggcagacaaaacaggtcggggaaggtttccagcgtgcttggttgcatggtagataggatggagagggcggcgggaacggtagcaggaacggtggcatcggggaagagacgcgtgaggcgttcgagcgagtgatcgcgtcgcccgctactggaacagggtgtgtacaggtcgctgaggtattcgtggtgcggatgagctagcaactgcgtaaagtgtgatagctcggccaatgaacagaggcccgtttctacgatgaagatttcgcgtctctccgtcgtatgtaccaacatggagtggacgaggctgcccatgaggtagagttcttggcgcgcgaaggctgaaagaaaagaggccaggtgcgttttgtgtaattgtagggcaaagtcggcgatctgtcgtagtgcccactggggaatgagatgttgctgattctgtttagaaagtatgtagaccaggcgtacgaggctggtgatgtcggtgatctggtccggcgtccagagggctcgtttggccaggtccacggctgtgggatatagcagcaatgtggtgcgtggtggtgtttgtgagaggcaggtgatcataaattcttgtatttgtaagagtgcggcctggcggtctagggctcgtgggatggagatttcggtgccggcctcttcttgtcgggctgccgcgaacagtgctaatgcgtaggcgaaggccatttctaccgtgcggcggtccaacatttgacatcgaccgcttttgagtacgtctacagcgtaacggtgaaagctgttacgtaacagtgcgctgaggtccaggtagttgaagtcgagtgcggcgtcgagaaagtccgagtctttgagataggagtgacggtttagttgagctttcttaactagtaccaggagctcgtgtttttcagtttgtcgtagtataaagttgtcgcgttgatagggcgctttgaagagtacgcgtggaagatgaccgaagataagcagcatgggtgtgtcgtcgtctatagataccgtaactacgaagaagtcctcggtcagtgtgattttaacgtaacgtagttcgtccatgaggtaaaagccctggtgcagacagggcgtaacggtgctgaaaagcagatcgtgtccatcaaagaggatacaggtctggttaaagtgtggccgatgtagtcccgaggtggtgtgcgatcccttccagtcgtgtggagtggtttggggtggcatccaaacgtgaggtattgacagatcaatgggcggtggcacggtggtgggctgctgacccaggctgtcttgtgccttcagctgctgcgaaaaagatcggtagctggccaggtctttggataccaatgcgtaggtgttaagtctctgttggtatctttctagggtttcggtcagatctacctggttcagaaactgctccgccagaggacccgcaaaaagacatcgaggcatatggaatacatagtattgattatagctttggaaaaagttgaaactgatggcgttttccctgacgaccgtgctgttacggaggctgctgttgtaggtgcactgggtggtgttttcacgcaggaagcggatgggtctcccgtaggtgttgagtagtaggtgaaatgcgtgagggtccagcgcttcggatgcggcgtccgcgccatatcgttgcgaaggtaggtgactgaggaggtagacggtgaagacagtgaggtagggggggaggccgggccgcatagcgcggctgcgccgctgggttcagcggcgtgatccaggtggtggttggcgttacacccgagagaaggagagaaaggatcccaggaaggggcacccgggtgtggcgctacgggttacaaaagtcgcgtctctgtctatttaatacgatgtcattggccgctgcgaaggaagaagaggggacacgcgggtaagccatgccgtccgggcgtggggacgacgctgattcgacggggaacgctctgcggagattgcctcacgtgcgtaagcggatcggtaagcgtaagcacctggacatctaccgtcgcctgttgcgggtctttccctcgtttgtggccctcaaccgcctgttgggaggccttttcccacccgagctgcaaaagtaccgtcgccgtcttttcatcgaagtacgattaagtcggcggattcccgactgcgtgttggtgtttttaccgccggactctgggtcgcgcggcatcgtgtattgctacgtgattgagttcaaaactacgtactcagacgccgacgatcagtccgtgcggtggcacgccacccacagcctgcagtacgccgagggcctgcgccagctcaagggcgccttggtggactttgattttctgcgtctgccgcgcggtggcggtcaagtctggagcgtagtgcccagtctggttttttttcagcaaaaggccgatcgcccatctttttaccgggcttttcgttcgggccgtttcgacttgtgtaccgattctgtcctggactatctgggacggcgtcaggatgagtctgttgcacaccttttggcggctacccgtcgccgtcttcttcgaaccgcacgaggaaaacgtgctgcgctgccccgagcgcgtgcttcggcggttgctggaggacgcggcggtgacaatgcgcggcgggggctggcgcgaggacgtgctcatggaccgggtgcgcaaacggtatctgcgtcaggagctcagggatctgggtcacagggtgcagacttactgcgaggatctcgaagggcgcgtgtccgaggcggaggcgctgttgaaccagcagtgcgagctcgacgaaggaccgtcgccgcggacgctgctacaaccaccgtgtcgtccgcgttcttcgtccccagggaccggcgtggcaggagcttctgccgtcccacacggtctttatagtcggcacgatgccatcacgggacccgccgccgccccgtctgacgtggtcgccccgtctgacgcggtcgccgcgtcagcggccgccggtgcttcttctacctggctggcgcagtgcgccgagcggccgttgcccgggaacgtacctagctactttggaatcacgcagaacgatccctttatccgctttcacaccgattttcgcggcgaggtggtcaacaccatgttcgagaatgcctctacttggactttctcctttggtatctggtactatcggctcaagcgggggttgtacacgcaaccacggtggaaacgagtgtaccatctggcgcagatggacaacttttccatttcgcaggagctgctgctcggcgtggtcaacgctttggaaaacgtgacggtgtatccgacgtacgactgtgtactctccgatttggaagccgccgcctgtctgctggccgcctacggacatgcgctttgggagggccgcgatccgccggactccgtggcgacggtgttgggtgagctccctcagctgttgccgcgtctggccgacgacgtgagtcgtgagattgccgcttgggaaggccccgtcgccgcgggtaacaactattacgcgtatcgcgactcgcccgatctacgctactacatgcccctaagcggtggtcgtcactatcacccgggcacttttgatcgtcacgtgctggtgcggcttttccacaaacgcggcgttattcagcatttgccgggctacgggacgataacggaggagctggtgcaagagcgtctgtcgggccaggtgcgcgacgacgtgctttctctctggagtcgacgtctgctggtcggcaagctgggtcgcgacgtgcccgtctttgtgcacgaacagcaatatctgcgttcgggcctgacctgcctggctggcctgctgttgttgtggaaggtgaccaacgcggatagcgtcttcgctccgcgcacgggcaaatttacgttggccgacctgctgggttcggatgccgtagccggcggcgggttgcccggggggcgcgcgggcggcgaagaggagggctacgggggacggcacgggcgggtacgtaactttgagtttctggtacggtactacatcgggccgtggtacgcgcgcgaccccgcggtcacgctgtcgcagctctttcccggcctggctctgttggccgtgaccgagagcgtgcgcagcggctgggatccctcacgtcgcgaggacagcgccggaggtggcgacggcggcggcgccgtgctcatgcagctcagcaagagcaaccccgtggccgactacatgttcgcgcagagctccaaacagtacggcgatttacgtcgcttagaggtacacgatgccctgctctttcactacgaacacgggctagggcggctgttgtcagtgaccctgccgcgtcaccgtgtgtccactctgggctcgtccctctttaacgtcaacgatatttacgaactgttgtactttttagtgttggggtttcttccgagcgtggcggtgttgtaatttccaccacgtgtcgctcgctgcataaagggcgaacgtcctcggagagggtatattcgttcggcgagagcgggcggcggtggtgggtatgtccccttctgtggaggagactacctcagtcaccgagtccatcatgttcgctattgtgagtttcaaacacatgggcccgttcgaaggctactctatgtcggccgatcgcgccgcctcggatctactcatcggcatgttcggctccgttagcctggtcaacctgctgactatcatcggttgcctctgggtgttgcgtgttacgcggccgcccgtgtccgtgatgatttttacttggaatctggtacttagtcagtttttttccatcctggccaccatgttgtccaagggtatcatgctgcgtggcgctctaaatctcagcctctgtcgcttagtgctctttgtcgacgacgtgggcctatattcgacggcgttgtttttcctctttctgatactggatcgtctgtcggccatatcttacggccgtgatctctggcatcatgagacgcgcgaaaacgccggcgtggcgctctacgcggtcgcctttgcctgggttctttccatcgtagccgctgtgcccaccgccgctacgggttcactggactaccgttggctaggctgtcagatccctatacagtatgccgcggtggacctcaccatcaagatgtggtttttgctgggggcgcccatgatcgccgtactggctaacgtggtagagttggcctacagcgatcggcgcgaccacgtctggtcctacgtgggtcgtgtctgcaccttctacgtgacgtgtctcatgctgtttgtgccctactactgcttcagagtcctacgcggtgtactgcagcccgctagcgcggccggcaccggtttcggcattatggattacgtggaattggctacgcgtacccttctcaccatgcgtcttggcattctgccgctctttatcattgcgttcttctcccgcgagcccaccaaggatctggatgactcctttgattatctggtcgagagatgtcagcaaagctgccacggtcatttcgtacgtcggttggtgcaggcgttgaagcgggctatgtatagcgtggagctggccgtgtgttacttttctacgtccgtccgagacgtcgccgaggcggtgaaaaagtcctccagccgttgttacgccgacgcgacgtcggcggccgttgtggtaacgacaaccacgtcggagaaagccacgttggtggagcacgcggaaggcatggcttccgaaatgtgtcctgggactacgatcgatgtttcggccgaaagttcctccgtcctctgcaccgacggcgaaaacaccgtcgcgtcggacgcgacggtgacggcattatgagcggcggcgctgtacggcagcggggagaaaagtggcagataaatcacgtcaggttcacacgtcgttagccagcgtcggcatatgaagggcgcgggcggccagtacggcctctgggctgagacaggacgaggcagggtgagaaagaggaggatgggggggaccggggtggtggtgctgctgctgttgtgggtgcggacggtgcgggtgccgggacagcgtgccggcgaacgttctgtaatcttccataataaaagtaaaaatgcccgtctcgtgtcgactccgctggatctcgaaggcgtcgggggtaatgcgcatcttgccggtgccgatgagataaaagtaccacattttttgacagatgatgcgaatcaagggttcgtacgcttcggcaccccagtggcgcgtgaagaaggccgccagacgaaacaagcggtgtccgtagagcgtgcctagggagaagaggatgttgccgttgcgcgccaggtcttcggggaaaacgaccggcaggccggtgtggcgctgcacaaagcgcgtcagcagtccgccgctcaagcgcgggtgacacaggcgctggctgagacgggcggcgcgcgtttcatcgaacacggccgcctcaaagtccagccccgggaaggcctggcgcagttcgcggtacagatgaggccagtagggttgcggcgtcttgcgactaagcacggcgtggtccgagacacccaggttgttcatggtttcgcgcagtagcagcgtttcgagaccgcggtgaaagaggaggacgcagatgaggcgtacgatcttgagttcttccaaacgcagcgagctcagcggctgtccgcgcgacatcttctcgctaatctgtaatattagatgattggcgcaagtaaaggagaatttgcccgtgcggacccgcgggacggcggggttctcttcgtcgcgggccatcatcgttcgctcggtgagcgggtagcgacggtgacgacaatgacgatggacgagcagcagtcgcaggctgtggcgccggtctacgtgggcggctttctcgcccgctacgaccagtctccggacgaggccgaattgctgttgccgcgggacgtagtggagcactggttgcacgcgcagggccagggacagccttcgttgtcggtcgcgctcccgctcaacatcaaccacgacgacacggccgttgtaggacacgttgcggcgatgcagagcgtccgcgacggtcttttttgcctgggctgcgtcacttcgcccaggtttctggagattgtacgccgcgcttcggaaaagtccgagctggtttcgcgcgggcccgtcagtccgctgcagccagacaaggtggtggagtttctcagcggcagctacgccggcctctcgctctccagccggcgctgcgacgacgtggaggccgcgacgtcgctttcgggctcggaaaccacgccgttcaaacacgtggctttgtgcagcgtgggtcggcgtcgcggtacgttggccgtgtacgggcgcgatcccgagtgggtcacacagcggtttccagacctcacggcggccgaccgtgacgggctacgtgcacagtggcagcgctgcggcagcactgctgtcgacgcgtcgggcgatccctttcgctcagacagctacggcctgttgggcaacagcgtggacgcgctctacatccgtgagcgactgcccaagctgcgctacgacaagcaactagtcggcgtgacggagcgcgagtcatacgtcaaggcgagcgtttcgcctgaggcggcgtgcgatattaaagcggcgtccgccgagcgttcgggcgacagccgcagtcaggccgccacgccggcggctggggcgcgcgttccctcttcgtccccgtcgcctccagtcgaaccgccatctcctgtacagccgcctgcgcttccagcgtcgccgtccgttcttcccgcggaatcaccgccgtcgctttctccctcggagccggcagaggcggcgtccatgtcgcaccctctgagtgctgcggttcccgccgctacggctcctccaggtgctaccgtggcaggtgcgtcgccggctgtgtcgtctctagcgtggcctcacgacggagtttatttacccaaagacgcttttttctcgctacttggggccagtcgctcggcagtgcccgtcatgtatcccggcgccgtagcggcccctccttctgcttcgccagcaccgctgcctttgccgtcttatcccgcgtcctacggcgcccccgtcgtgggttacgaccagttggcggcacgtcactttgcggactacgtggatccccattatcccgggtggggtcggcgttacgagcccgcgccgtctttgcatccgtcttatcccgtgccgccgccaccatcaccggcctattaccgtcggcgcgactctccgggcggtatggatgaaccaccgtccggatgggagcgttacgacggtggtcaccgtggtcagtcgcagaagcagcaccgtcacgggggcagcggcggacacaacaaacgccgtaaggaaaccgcggcggcgtcgtcgtcgtcctcggacgaagacttgagtttcccaggcgaggccgagcacggccgggcacgaaagcgtctaaaaagtcacgtcaatagcgacggtggaagtggcgggcacgcgggttccaatcagcagcagcaacaacgttacgatgaactgcgggatgccattcacgagctgaaacgcgatctgtttgctgcgcggcagagttctacgttactttcggcggctcttccctctgcggcctcttcctccccaactactactaccgtgtgtactcccaccggcgagctgacgagtggcggaggagaaacacccacggcacttctatccggaggtgccaaggtagctgagcgcgctcaggccggcgtggtgaacgccagttgccgcctcgctaccgcgtcgggttctgaggcggcaacggccgggccctcgacggcaggttcttcttcctgcccggctagtgtcgtgttagccgccgctgctgcccaagccgccgcagcttcccagagcccgcccaaagacatggtagatctgaatcggcggatttttgtggctgcgctcaataagctcgagtaagagagacgctatatttagggcttccctctcttttttttctacaccgtgataccctaataaagcacaccgcggttattatcaacgtctctgtgtttttattatttagaaataaatacagggaatgggaaaaacacgcgggggaaaaacaaagaagtctctctctagatgcggggtcgactgcgtggggtgctggaagtggaagcggtgctgatgggtgagggtcgtggcgcgggcacggaccgcaacgtgctgctgatgtctgccgcggtacgcacgtcgccgtccatgtcgctgcgcagataagaggtaggtcgtagtgcggcgtgctgcacgctcaccgttaatggtaccaagtcgtcaaggctcgcaaagacgtgccacgaggggatgacgagcgtgagagccccgttgttaccgcttcgacgtctttgtccggtcaggatcagtgccggggacagtccggcttgggtgtccgagtcctcgtcgccgctggcttcctcgaagccggcaaacatggcttcggacaggggggtcggcgtcggtgtggaggagaggtcatcttcgtcgtcctcttcctcttcttcctcctcttcctcggtgggtggtaatccgggggactgcgggagaaactcggagacggcgccgcgcatgacgttgctccgtggaaagagaccggcgcgcagctgcacctggggacgcttgattttgtccggtttaccgggtgtgagagtccaaaacccacggcggaaaaagtggatgcggcctagcggctgtcggtgttccaaatgaacggcctgatcgccggtcagcgtgacgcggagggtgattcgcacacgatcgggtagcgggccggcttctatggagacgcccgggatgttttccgggaaaaagatggtgtcgtgagtctgattggtctcgaaagcattctggatctgcacgatgtactcgggatgtatgcgcgtcagcgtaaaacttttgggaatcaacagctggaagccgttgtccggcaagcgtcgtaggtgcgggtacggattgtgtcgcgccaccacctcggcgcgatgcgtgtaaaccgaaaagtgcagaaacacgctggtcggcgggtgcggtgagtcgtgatgcagaaacagcatgatccattggcctcgttcgtccgtctccgttttgtggatgtacgtgttagggtccgaacaggccagctgctccagggcgtctaccagcgtcagcgggatggcgccggcgcgaaaggcgaactggctgacaaagatctgccctgcctccaaactgctgtcggttctgcggcgccagttcggcgttacggtcagtcgcacggcccagtggtgagccgtgcggcggatgatggcgcgcgcttccattcgcggccgattttcttcgccgccgcgccgctggctctgaaagaggtgcagtccgctaacgggcacgcggtccagcggcagcgcaaaggccagtaccgagaccgtgttgttttctgagcctggcgtcaggcgtcgtgggccaaagttgttgaggtccaccagcagtcggtcctgttcgcccaccacgcagcggcccttgatgtttaagtcggtcaggtctacggtgtcgtgcggagatttgttctcctgaaaacagcagagaaccgagggccggctcacctctatgttggtacgcaggtccaggagtcgcagacgaccggcttccagcgagccgccttccacgttggtgatgagccgaagcacctggcagtgcaggcgaccaaagcttccgctggcggcttcggcctcgctgatcgcggccgcttccgacgagggtccctcaccgggcgaggacgatgcctgagacattgcgaaggcgggatggggggagggtcaggggatgcgcaaaggtgaacgggtcttcgtgggaggtcgggaagggttccggcaactgtcgcaaatatagcagcggtgacaggtgtggcggccaaaagttgcgtgtctgagtggacgtgggtttttatagagtcgtcctaagcgcgtgcgcggcgggtggctcaacctcggtgctttttgggcgtcgaggcgatgcatggcccgggcaaggcgtcttgccggtggcggcgacgtttgggttgcgcagcgggctgccatacgccttccaattcggcgaagatgcggtagatgtcgttggcgtcccagaagaattcctggtacttcagattctgaccctgaaccgtagccaccatgggcaccaggttgcgggccaggatgccggcctgccagggcggccaggtgaacacggccggattgtggatttcgttgtcggaatcctcgtcggtgtcctcttcgggcgcgacggtggactcggccttaaggcggccgcgtgtcataacgcccgacgtgcacgccgtcgccgaggatgctgatttgcgtttgcggcccgcggaagtggaggcgcccgccatggcgccgccgccggtgacgcggggcgtcttgcgctcggtggttacgagttcttcgtcggagtccgatccgctggtccagacgtcgtcgtcgccctgggcggcaccctcgtcgtgccggtcccaggtgtgtcggtactcaagcttgccctggatgcgatactggctggtgaaggtggggtgctcgctgtactgaggcccgcgctgcagcagcaagtcgatatcgaaaaagaagagcgcagccacgggatcgtactgacgcagttccacggtctcgcgtatggcttgtacctccaggaagatctgctgcccgttcatcaacaggttacctgagatgctcaggcccgggatgctcttgggacacagcagcccaaaatgctcgtgtgaggtaaaagccacatccagcatgatgtgcgagatcttgcccggtttgattatcatatttttgggacacaacaccgtaaagccgttgcgctcgtgggggcgcatgaagggttgcgggttgcgggtcatcgtcaggtcctcttccacgtcagagcccagcgtgacgtgcataaagagcttgccggagggcacgtcctcgcagaaggactccaggtacaccttgacgtactggtcacctatcacctgcatcttggttgcgcgcgtgttctccatggagcaaaccagctcgtgcgcgcacaccacgtgccgcagtgccacgtccttggtgggaaacacgaacgctgacgtgtagtagacgtcgggctctttccactggttctgctgacgcgtccaggccagtcccgagaccgtgagacgcgcctgccacatctgcttgcccgacgcgtgaatcacagcgtcagctacgggcaggtgtcggtgtttgcgctcggccgccgacgggtagtggtgcacgttgatgctggggatgttcagcatcttgagcggcagcgcgtacacatagatcgacatgggctcctggctggggcagatgcttcggcccgtggggttgtgcacgttgaccgacacgttctccacctcgctgcccgtaaagtacgtgtgctgcacctgcagctgattgtcgccgcggtggcatggcgtcgagtcgggcgtgtactgcgataccaagatcagcgagggctggctcacgcgtacgtggatacccgtctgcaggagtcgcgtctcgtgcggcagcaccggcgtatcgccgcgactaaacacggctttcagcacgtgccccgaaatgggacccagtacggatatcatttcgggacaacggcgaccgcgcgactccatgctgcctgcgcgtacgggtgtaggcgactgagcggcgcgccctctgcggccgccgccttacataggcaggcgaccaaacgcggaacccgaaataaaaacgttctacacagagacaaccgcggattattgagtgtctttttttattacaaaaaaaagaggcgaagccccaccgtcaccacaccccatcacacaccaccaccgatttttttttgttttaatcccgtatggcgcggacgcctagtgtccgtttcccattatcagggtcctctgtttagagatcgccgcagaccatggctaaagtgacaggactcgtcttctctgtcgtattttccgtaagcttacagtcttgcggttccgtctccggggacgccagtcgcatgggcagcaggtcctccagcgcgatggaagcgcccagcaccgagagctgctgttgcgacggcgaatgggatgtggaccgcgagtgtagcgtggatttgacttggtgcgtcattgctgacaggcaaccccgattcagcgtatgctttgacgagataaaatagaggcgccccaggagcgcgtcccgtgggaacgtggcgccgttctcgtcgctcaccagtacggttaattccaaccaggagcgcggtagccagaccgtaacgggcattttgagtccctgacggttgtgtggtacaaaaacacccagataaggcccgtaaaagcggcggtagatacgtaacgtgtgcgagttcttcagcgtcaattcgtaagggacgcgcacctccagtccctcgtccgccgcgccggagcgtggcggtacaaagtaaggcagtggcgcgtccgaaaagaagggtcgtcgcaccgtttcgcgtcgcagccgcaggcgaaacgccactgggtcggctggcgcctcggtgcggtcgcaggtcacgttgaaacgtaatatgccgtcttggtatagcgtgagtgacgacagcgtcaggtccggcggtgattcgttcgggtctagctccaatcgtccaaagacggagggtcccaatgtcttggccgtggtttccgagaggcgcgccgagatacggctggtgagtccacgcggccccgagatgccgccttccactcgatgccagcacagcgcgtgtcgtacgcgcaccgtcagcgtgggcgtcagatccgcgtccgttgattccgcggtatcagcgacggaagccgcgttctccgttacgttgtttatatccagcgtcggctcgaacgtgagttctggcagatgcagcgccagacagtcgtgtaacgccgtgtgatgcgcggctttacgtcgtagcggtagccgtttcaacagcggcgtgatgatacggagcgcgaagagattgagtgataggcgcacgatggccatgcgcgtcagttgttggtcaattaccgagcgcaggatatggcagcctgggcgtgcgggaaagagagagaaggccgggcgcacgtcagaatcctcgttagagaccacgcatagaatgccgcgttcacgatcgtcgttgcggtcatcctcgtcctcttctttcttctcttgtttttccttttttttctcgggctcgtgggaagccgccgtttcttcttcttgcaacgtcgcgggggcggtttgagactcgtcgttcgcttcccccaattgcagcggcgtagagagcagaatctggaagggatcccgcaattcttcgggtcggaggtcgaggtgcaactggatcagatggtaggtgccgcggtgcacccgaggctgacggatgtcgtgtttatccgtcagtgtgaggatggtctgcggcgagccgctgtacttgtccagctcgtccggcgttttcaggaggagactgtcgtcgtcggtactggcgacgcccatcatggtcgtggtggtagtggtggcgaggaaagtgagcggcggcgccgacagagctcggcgttggcggcggcattttccgctgtgtcggctgctattgctgccaacgccaccgccgccgcctcgtctggctcgtggccggcgggcccgattccgaaggttggggtcgacgcgtggcatgcttggtgtctgcgggcgcgagagggccggctcagcctttaaatatgcaggtcgcggatttgttatcgggtgaaacgtcacacaccgtgaagacgacctgttcgcggatgaggtcatccagctgtcgcagcatgacgaaaagcgccgacagccgcgcgatctcgtcgtcgggcgacacgtgctgcggccgcgcgggcgtgcgcggctcgccgacgctgcgctcgcggtccagccgcatcagcagctcctggcacttgacgagcagcatggagctgtcctctagcgccaacttgcgcacgtaggtcatggtcagctccgaggctaggttggccaccatggacatggagaggcaggcggtcttcatgtcgatcagcaggtgctggtcgatgaccggatcggggatggtgaaggtggcgtcgcgaaaagtaatggtctgcagctgctgcacggcagcctttacctcctcgtacgaacggtcgagcgagaagaggcccatgatgagtagtcgctggttgatttccagcgccagtggcatgggtacgatccagggcagcaccagctcccactggcccagcgtcagcaggttctcgcgcgccagcggtccgtggaagagcggcggcagcacgcatagcgcgtcgcccttctcccaagtcacgggtcccgtgttgaggacggtgtagagcagtccgtgcgtgggtacgtgtaggaggatctggttgccttctacgcgccgcatcaacgtcagcgtcatattgcgcagcaggccgcgcagtcgtacgtagccgcgggtgtgatctacgaactggtgtaggcccagctggtagtgcttgatgagatgtagacgttgcggaatgggcacaacggccgctactagcttggtcagtttgcctacgtcggcgatgctgagcttgtggtcgaaagtgcagaagatgttggcctccatggccgccatagcggcggtgaaatcctggccgcgacggaggagaggcagagacgaacaacgtctgcaccgggcgcggcgtcagagcgagcgtggcgcgtccgggcccgcgtttgcgtctaggtgactcgccgctaacctgcggtcgtcgccgtcctcctcaccggacggcctcacgagttaaataacatggattgctgcagcgggatgatttcgcctacgacgtagttaccaaagtgcgtttcggacgtagcaaaagccccggcgccacccttgagtttggtctccatcagcgccagcgtggtggtgctgaggatcggtagcgcttcctgcgtcagacggcacgggttttcgatgagttgttccgtgccttcgacgcagacgtactgcgtgtccgtgtcgccgcggatgcagtccttggcgcgtagcaggtactcgtcgatggttttgaagagcgttttgttggccgcgataatctcttctgtgttaaagtactgcgcgcaagggctgtagaatttggagttgtagcctagacgttcgcgatgtcgggtgttgtagagtacgtcgctcagacagccggcttgcgaggcccaggggttgtgtgtggccgcgaaagtctgtgcgtccgcttcgcgatggtcgtagatggccttggtggcggcctccgtgtcgtacggatcgacggccagcatgcaggaggcacgcccgcgcgggttgttggggatcttaaagtaattaacgtccatcgtcaccggcgtaaggattagttcgcacgcggccttttgtccgtgcaccgtggcggcggcattgcgctcggacatgctgccgaacgtcagcatagagatggtctccgtgtctaacagttgcggccgttctacgccggccgcgtgccggatccagcggtccacctcgtcgtgccggtacacgttcatagggaagacgcgaaagaggtcctgcacgcggacgcccatgtcggttcgcacgcggtttacgtaggctacgcaggtatttgacgtgtaacccagacccatgtctacggtgttaatgttctgcgtgacgtggtacgtagtgctgatgtcgcgttcctccttggtcacgatagggttgttgatgataactgacgtgcatgatttgccgctgtagagcagcatgtccacctcgaaggtgtcggtgcgtacggccgtgagtgcgaatcccgggtggatgtgcgccttggtctgcagcaccagtgaaactggtgagattttgtataacatggcggccagcgtcatgactgagtgcaacacgttgggacaggtggccgagtaacgcgaaaagggcgagcgcagccagttgtggtactcgtgcgcgaaggctgtgggtagcgggaaaccaccgtcgtgacggtgatagtgcgggaactcggtcacgtagcgtttaatgtcgtcgctcaacgccgcgcagatggtggggtttgagtagaaacggtggaaaggtacgggtaggctgtactcgatcaacgtcttaggcgccgtcacgacgcagcagccgttgtaaagcacgtgctgacgtgagataaagtccggcaggccctgacgctgcgcgtggtccagaggcgcgcgcacttcgagcaccttgacgtgctcgcccacgaattgcacggccaaaaacagttcacgacaggcctgcagcagcggcgtatgtgcgtcggtggcgacgtcctccaccagctcggtcagcatctcgcctacggcttgacgttgcgccgctatcgagtcttcgggggtgacaccgcttgtgctctctttcgacgtcgtacctgacgtggagaccgcggtggcggccggcatcaggagaaacgccggtcggtaaaagaggtctactagcagcgtcttgaggttgagtcccaggccgcaggcccggttgttggtcatggcgggcatgaggcagagataaaagaccttttgtaacgtccattcgtcgtcggtggcacggtaatcgtccacaaacagcggctcgtcggcatccatggcgcccaaacgcggtacgtccgaaacgccgtggtgtcgcgcctcgatgttggccgggttcaacggttgccggtcggccactacctgtacgccttccatgttacgcggcaggtgcgtaacgaaggggggccacagccggtggtcgtgcagcgcgttcacgtaagccgatagcggttcctcagccagttgaccgttgttaagtcccggcagcgctgagatgcgcgttaccagacgcagcacggcgaccagattgcggtagtgaaagagcaactgcggtggtagggcgccatcagccaggtgttcggcgatcaacgtcaccagcgcgtagctgtgcgcaaaaaccagcagctgacgtgtgtgaaacatgttgacgatacaacgtgctacgaaagtgcggattagcaaaaaagcgtcgacgttgccgtgtaccagcacgtcgaccaggtagcaaagctcggggtaattggggcttgtcacggtggttttgaaaagtcgcaacgtctcttcgtagtcgggtggtggccgcagtcgcatgtgttccatgatctcccaggtgcgcagttcgtggaaggggcccggtgccagtccatctggcaaattaccgatgacgatacgcggtgtacacagcgccaccgtttcgctgttttcctggcagtgcgtaaagtcgaagaaggggtgcagctcggtgtagagcgtgatgttgcccaccttgtagaagtcggtgaccacaaagtcctgcttcatttcgttcaccgtgcgcgggacctcgcgtcgtacgcggtaaaaatgcggtatgcggcgcgccgcaccgcccatgggttcctgctgaaaacgacactcgagcagtcgttgcatggcgggttccgagggcggtccgcgttccgtgaaggtctgtagacagggcgcgggctcgtgcagcaccgggtggcacagcgtcttgagcgcgtccacaaagtctattttttgtacggcacggtcccggtttagcaggtaggccgtggtgggcaacgcgttgcgaacggtgtcgttaagcttaactttgctttccaccgtggtgtaaccgcgatcctcgggcagatacagccctacggggaagaaaaacgtcaggtccacgttacgttctagcggatctttggtatcggtgtttttgtagacgcgccgcaagttttccataatcaccgttttttcgcccagtcggatcacgtccatgctcagcggcgttaagctgtgcgccccggcctgcgaaagcgagtcgttgggcaaatgcggttggcccgaagtcagatgagccttgtacgagttgaaatcggccaggatcgagtgataggatatggcagtgacggcattttcgggactgagtacaaaattgccgtaggtggccggcgccgagaccgtttctttggtgatgtggcttgagagcagcgacatgatgatctgcataacgttggccgtgcttaccatcacgccgctgatcttggcccccgagctcgtggtgtacgtggtggggttgtctaggatgctatcggtggccgcttcggccagacgcgtgaggaacttgagcacatagtcgcgatcgcgcgtgcgattcagcaaaaagagcgtggccagcattttggccttgaagctctgcaagatgttgcttcgctggatgcggttcagtgcctgtcgcgccagtgtggcgttttctaccagcgtctgcaccacaaagtacggcggcgccttgcgtagcagtgtctgtaaaaagctgtgaatcaagccgcgctccatggcgtcggccgtgtttttaagcgcgcgcagcaccgtgtgcatggcttccacgttgaggatcttgtccaagatggtgccctcgaatgtctcgcgcagatacgtgaggcaggctgcgctgagctcgaaggggatggtgatgggggatttttcactgtatttggtgaccataatggtggtctgacgactagtgggcaaaccggcgccgctggccacacgcggcacctgcacgtggaacagcattttgcccgtagtcagtttattgaggtcgtggaacttgatggcgtgcgccgccgcggccaagccgctggtcaaaaaataaacccattccaggcgattgcagaaggtgccgaagatggcttcgaagtgaatattgtaacgctcggggtcatcgccgtagtagatgcgtaaggcctcaaacatctcctcgccggcgctggtcttgacgtgcgtcagaaagtcagtgggaatgcctactttaggcaggagctcgagcgccgaccagttctccatcgcggcggcggcgtgagcgcgaggcgtcggagctcggggaaagcagcgcgacccggagaatggccggcgctgcgccgcgccgcctcggctgtgacgctctaatagtcgttggcggctccgctatgccgcgccgggttttacacgtccccgtgcacgttcgcgcctgcaacctcacccaagagctatcgacgggcgaggacgcccgcttttgtcgtccgcgacccgttaacgtcgaacgggtgcgcgctgtttttgcggctctctaccgtgcctgtccgatacacgtgaggaccgagcccgagcgtgtcaagctggtactgggtcgtctgttactgggacccgtggccgtaccctgtttttgcgacggtgaagtggagggccacggtgaacatctggtacctacgacgcagttttgtcgcgggccgctgctctacgtgcaccgacgttgttgttgcggatccgtgaccgccgggcgcgcgctgtcctaccacgttctcgaaaaccacgtggccacgcatgtgctacgcggattgctctcgctgacggaatggaatcgagaattgccgagcctcttttgcgactgtcctggcggcggtggcgcctcgggaaccgaggaacgctacgctatggcctgcctgccgcgcgacctcagcctgcacctggacgactatccttacctgatggtggaaatcggacgcgtactcagtgtcagcgaggtagacgactacgtaaccgccgtctccggctacctgggcgaggccgcggcgccgcgcatccaggttcactacaagctgctctttggactcaacgtgcgtccgcaagcgccgtgcgcgttggacgctacacgcgacttttttctgctggagctgcaaaagctttggctgggcgttgaatatcaccacgaagtcacgtcggagtttttcggtcgcgtactggctcagctgcatcgcgaccgcgcccgcgtcatgatggcgcttcgcttgcccgagcagacggtgtgccacctgagcaccttcgttctcagtcgcttcaagcgacaggtactgtacttcaagctacaggtgagctacggcaagtgccggactggtcacgctgacagaagtgggggaggggggaacggtggaaatcagggacaccacaacctactgtgttatcgacgccttagcgtcacatttgccgacacagacacggtgtggagaaaccttttctacgtttattacgaactagctcgggatctggggtcccatgggacggaggaccgacccgtaagccgcggttacggtgtttcttgcgcttcgaggacgtcgcgactgtcaccgtcagaatcgacggtggtttcggcgaacggacacgcgctgtcttccaccgcgctcccgacgacgagcgcgggtcacaagctgtcactgccgcgcgacccggccgcagatcgcgttcgacgttacgtatgcattatctcgcgtctcatgtacgctcggtacggggagagatggcgtaaacactgtcaacggcggtcggagacgggagaagaggaggaggaagagacgctggaatcgggggagactgacgccacgccgccatttgactttacggggcagcagctgcgccgggcctatcaggaacaccgacgtcgtaaacatctagccgtgcagcgttacgcgccgtgccgtcgtaagctcatcggcgggatggagtttgccgaggtgacgggcgtgagtctagaccgcatcgccgtcaacgctttcaacaccaaccgcgttatcaatatgaaggctgcgctctcgtccatcgccgcgtcgggtctcggcgtacgcgcgccgcggcttcccaagaacatgacccacagttttgtgatgtacaagcacacctttaaggagcccgcttgcaccgtcagcacttttgtttccaacgacgccgtctacatcaactcgctcaacgtcaatattcgcggttcctaccccgagtttctgtactcgctgggcgtgtaccggctgcacgttaatatcgatcacttttttctgccggccgtggtgtgcaacagcaactcctcgctggacgtgcatgggctggaggaccaggcggtgattcgctcggagcgcagcaaggtgtactggaccaccaactttccgtgcatgatctcgcatactaacaacgtcaacgtgggctggttcaaagcggctacggccattgtgccgcgcgtctcgggcgccgacctggaagccattctgctcaaagaactctcgtgcatcaagaacatgcgcgacgtgtgcatcgattacggtctgcaccgtgttttcacgcaactagagctgcgcaattcgtaccagatccccttcctggccaagcagttagtgctgtttctgcgtgcttgcctgctcaagctgcacggtcgagagaagcggctgcagttggaccgcctagtatttgaggcggcacagcggggtctctttgactacagcaagaacctcacggcgcacaccaagatcaagcacacttgtgcgctcatcggcagtcgtctagccaacaacgtgcccaagatcctggcccggaacaaaaaagtcaaattggatcacctgggccggaacgccaacgtgctgacggtgtgtcggcacgtggaagcccacaagatccctcgcacgcgcctcaaagtgttagtcgaggtgctgggcgcgttgcagagtatcagcggtacgccgcacacgcgcgaagtgatccaccagacgttgtttcgattgtgctcggcggccgcagccacatcgggcctgtgttcatcccctcccccattgtgtgtgtcctcatcttcctccgtcccttctgtcccaacctccgtcagcgttgacggcagttctgaacccacgtcgccgcgagcgcggtttgcatcacgatgatggaagccgcggccgctgccgccgcggcgtttcgtccggaggagcgtccgacgccgggttggcacgacgcggcgttgttaatggacgacggtacggtgcgcgagcacgcgtttcgcaacggaccgctgtcgcaactgattcgccgtgtgttaccgccgccgcccgacgccgaagacgacgtggtttttgcttccgagctgtgtttttattgcagcggtcgttttaaccgcaggtcgtccgtcttctccatctattggcagaagcatagcgatctggtgtacgcgcttacgggcattacccattgcgccaagttggtggtggaatgcggtcagttggggagtagtaggctacggtggcgcgacggtgatgcgagtggtgaggagcgccggggagacgacgacagcagggacgagctgtacgacgtgccgggcatttatatgattcgcgtcaacgacggcggcagcaccggccccagacacgttatttggccgggtaccagcgtgctttgggcgccggacgttgtgatcactacggtgcagcgacgaatctcggcggcgcgcgccctggtgaacacgttccgccaatatttttttttgctggaacggcgctcgcacgaggagctggttctttgtccgcccgagatggaggagcgtctagcgccgctgttgcagagtgccacgcgcggtgattcggacatgtttgacggtgtggtggccagcgcttatcaccgtttgcgaatgagtaatattccgcgttcatccgcccgtctgctggaacactgcgtggggctggcgggtgctaagaagctgctcttgctcgacgtgccgcgtctggagaactattttctttgtcaagtctgtctttacgagctggacgaggacgagatgggcgaggagatgctgggcatgttggccggaaagcccgaggatgccgccgtctcgggcgcaagcggcggttttctgctacatcgcaagacgatgaagctggccgcctgtctgtgtttgttgctcaattcgctgcatttgcaccaggaggcgctggaggccttggatcctccgccgccgcgcgtcgaggagaacgaccttgtcaacgtggtgctgcgccgttattatcgcagtcacggcggcgtgcaggcgcggacgctggcggcggcccgggctttgttagccgactacgccgaaacgttttcgcccttggggagttttacgcgcctgggttacgatcgtctcgtttctgccgatgccggcgtcagtcgccggcacctggtggctctgctgcgtgcctagctgaccctgaaacggatggcgtgtatatcgtcacacaggtaggtggccatgatgacggcgatgataagatcgtccgagatacgattctggcgcttggccgagtaacgcgccgtcgtgccttcggccagcgtgacgcggtgcaggttctgaatctgctccagaagatactcgatggggtcgtggctcagcttgatggtgtaggagacgagctcttgcgaggctttgatgtagcccgagttgaaacgcgagatgaactgttccacggccagcgccttgtcgcggcccatgaggtagaagggctgttcgatgtggttctggtcgggcgtgtggtagaagagcacgcggatgagcgtgctgctctgcacgctctgtcggatgaggcaggcgatgcgcacggccgccgcctggttggtgttgccctccacggcgatgcgcagttcgtccaggtaagggtgcaggctcagcaccgagatgatcatgtgcgccgcgcactcggcgatggctacctcagaactctcggagaggtcgcgcaaaaagaaatgctctaggccgtaaatgagaaactggtgtcggtaggcgcctacggccgccacgcccgtgcccgaggccttgcggttggtggtgaaggccgggtccagatacacgtaaagcgtcttgccgaaataatcgtaggcgttggtgttgagcgtgctgtaacgcaaaatatcgaactcttcgcggctctggtccgtgatgagcacggtgttctgcgagattttattggtaccgccgatgatctcgtccatgaaagcgcccggcataaacatgttggccgtcttgcgcacttgcgagttgaggctgatgaaggtgggcttgtgcagtcggtagcaaggacacgccgtggcgtcgcccttctccgtgaagctgtgcaggtgctcttcgcacacgtaagagaccacgttgagcatgtcaaagggcgcattgttaaggcgcgtcaagaaacacgtggagtcactggtagtgttggtggacgatatgaagatgatcttggtggtattctgggccaggaaccccagaatggtgttgaaggcctctttcttgatgaagtgcgcctcgtccaccagcagcaagtggaagttttgtcctcggatgctctgtgtagagaggagacagaaaagggactcttatgattacgcacgctcgactggaagcctacagagtcggggtggggccggacaggtgagccaggtgagccgccaggtgaggcgggatcgccgtgtgccaaccgggctgcgacctgaaaaccggaaccaatccgccgacaccggcgccgcgtgacgcgcgcccataaaaacgaaagtgtcgtcgtcgcgacccgccacagccgccatgaactcgttgctggcggaactcaaccgactaggggtcgcgcacgccactacggaggatgtttttatctttgtcgaccgcctctttcaacacttttccttccttttccaggccgaggagtcaggcccgcgccgcttggaactggtcgcgtccgtgttcgagcacctgacggtggagtgcgttaacgacatcctggacgcctgcagtcacccggacgtgaacgtcgcggagacaagcaacacctgtcgtccctgcccttctcctgttccctccgcccccaaaactgtcagcggcgctcagacgtcatgtgcgacgcctcgggcgcctgtgacatgaggcacgtccagaacgcgtttaccgaggagatccagttacactcgctctacgcgtgcacgcgctgctttcgcacgcacctgtgtgatctgggcagcggctgcgcgctcgtctccacgctcgagggctccgtctgcgtcaagacgggcctggtatacgaggctctctatccggtggcgcgtagccacctgttggaacccatcgaggaggccgcactggacgacgtcaacatcatcagcgccgtgctcagcggcgtgtacagctacctcatgacgcacgccggccgttacgccgacgtgatccaggaggtggtcgagcgcgaccgcctcaaaaagcaggtggaggacagtatttacttcacctttaataaggttttccgttctatgcataacgtcaaccgtatttcggtgcccgtcatcagccaactttttattcagcttatcatcggtatctactcaaagcagaccaagtacgacgcgtgtgtcatcaaggttagtcgtaagaagcgcgaggacgcgcttctgaaacagatgcgttccgaatatggaaacgcacctgtattcggatctggcgtttgaagcgcggttcgctgacgatgagcaattgcctctacacttggtgctcgaccaggaggtgttgagtaacgaggaggccgagacgctgcgctacgtctactatcgtaatgtagacagcgctggccgatccacgggccgcgctccaggcggagatgaggacgacgcaccggcctccgacgacgccgaggacgccgtgggcggcgatcgcgcttttgaccgcgagcggcggacttggcagcgggcctgttttcgtgtactaccgcgcccactggagttgctcgattacctacgtcaaagcggtctcactgtgacgttagagaaagagcagcgcgtgcgcatgttctatgccgtcttcactacgttgggtctgcgctgccccgataatcggctctcaggcgcgcagacgctacacctgagactggtctggcccgacggcagctatcgtgactgggagtttttagcgcgtgacctgttacgagaagaaatggaagcgaataagcgcgaccggcagcaccagttggccacgaccacgaatcaccgtcggcggggcggactgcgtaataacttagacaatgggtcggatcgccgtttgcccgaagcggctgtggcttctctggagacggccgtcagtactccattttttgaaattccgaacggagcaggaacctcctccgcgaacggcgacggcagattcagtaacctggagcagcgggtagcgcgtttgttgcgcggcgacgaggaattcatctatcacgcgggtccattggagccgccttccaagatacgcggtcatgagttggtgcagctgcgcctggacgtaaatccagacctcatgtacgccaccgatccgcacgaccgcgacgaggtcgcgcgtacggacgagtggaagggtgccggtgtctcgcgtcttcgcgaggtctgggatgtgcagcatcgcgtgcgcctccgtgtgctgtggtacgtcaattccttttggcgcagtcgcgagctgagctacgatgaccacgaagtcgaactataccgggcgttggacgcttatcgggcgcgcatcgccgtcgagtacgtgctgattcgcgccgtgcgcgacgagatctacgctgtactacgacgggacggcggcgcgttgccacagcgtttcgcctgccacgtgtcacggaacatgtcctggcgcgttgtttgggaactttgccgtcatgccttggcgctctggatggattgggcggacgtgcgtagctgtattattaaggcgctaacgcctcgtctgagccggggtgccgccgctgccgctcagcgagctcgtcgccagcgcgagcgctcggcgcccaaaccgcaggagctgcttttcgggccgcggaacgagagcggtccgcccgccgaacagacttggtacgctgacgtggtgcgctgcgttcgcgcgcaagtggatttgggcgtggaagtgcgcgcggcgcgttgtcctcgcaccgggctttggatcgtccgtgatcgccgcggacgcctgcgacgttggctctcgcagcccgaggtgtgcgtgctgtacgtcacgccagacttggacttttactgggtgctgccgggcggctttgccgtctcttcgcgcgtcactcttcatggcttggcgcagcgggctttgcgagaccgattccagaactttgaagcagttcttgcaagaggaatgcatgtggaagctggtcggcaagagccggaaacaccgcgagtatcgggccgtcgcttgccgttcgacgatctttagtccggaggacgacagctcgtgtatcttatgccagttgctgttgctctaccgcgacggcgaatggatcatctgtttttgctgcaacggccgttatcaaggccactatggcgtgaaccacgtacatcggcgtcgtcgacgcatctgtcatctacctaccttgtaccaactgagcttcggaggtcctttgggtccagccagcatcgatttcttgccaagctttagccaggtgaccagcagtatgacgtgcgatggtattacgcccgacgtgatttacgaggtctgcatgttggtgccccaggatgaagccaagcgtatcctggtcaagggtcacggtgccatggacctgacctgtcagaaggcagtgacgctaggcggcgccggcgcctggttgctgccgcgtcccgaaggctacacgcttttcttttacattctgtgttacgacctgtttacctcatgcggcaatcggtgcgatatcccttccatgacgcgcctcatggcggcggccacggcctgcgggcaggcgggttgcagcttttgcacggatcacgagggacacgtagatcccactggcaattacgtgggttgcacccccgatatgggccgctgtctttgttacgtgccctgtgggcccatgacgcagtcgctcatccacaacgaggaacccgcgacttttttctgtgagagcgatgacgccaagtacctatgcgccgtaggttctaagaccgcggcgcaggtcacactgggagacggcctggattatcacatcggtgttaaggattctgagggccgatggctgcccgtcaagaccgatgtgtgggacctggtcaaggtagaggaacctgtgtcacgtatgatagtgtgttcctgtccggtgcttaagaacctagtgcactaacggggtctgacagttcacggggagaagaaacaagaaacaacaaaaaaaaggaggacatggactcgccacggtttgtggcaaggcgtatgttatcatcatggagctactcacgttggtgttgtagcaactggcaaaaagcgccgtgctcttggcgccgcggtggtcgatgctgatcacgttgtccttgttctcgaccacgtagtcgcgcgcgaaggtgtggcggcagcggaactcgacctctttgagcacaaactgcgacacgtgcttttggtgcgccacgtagccgatgctgatgccgatcatgtgcttaagcagaaacgagataatggggatgatgaaccaagtcttgccgtgacgtcgcggcaccaggaacacggtggctttctgcttaaagatgtcgatggaggtctgcgagaggaagtcgatctggaaggcgtggatgaggtactgcagcacgcgattggccagcacggggatcttggtcacggctataaaaaagatgacgtgtatcaataaattcttttgaaacggttcgagtcggatggcttttgcgtcgccctcgacggcggtactgaagccgccgtcgagccactttttaaagtcggtcatgaagttgttgatctgctgaaactgcggatcgcggtagagctcggtcaacgcgtccagcttctggtaggaggcgcgctgctcctcggagcacgggcgaaacgtcagtttatcgagcgcgctcttgaggcgctcgtgaaacagcagctcgcgctggctttcctcgggcgagttgtagtcgcggtggcggccgcagaaggccatgagcggcaggaaggcctcgttgcacgagtgggccagcccgagttcggggtgcatcatctggtagcgcttgcggcacagcgtcgccacattggtgaaggccgtggagatgcaggaggtggggtggctcttgcgcttctgcagctccgcgtagcgctcctggatcttggcggccgagtctccgcgcaacatgatggcggcggcggtggtgcgagcggaggttaggcggcagcggcgagaggagaggaaaaagatggcggccgcgaggacgacggaggatccacccgaaaaccacgttgttgcggacgtggctcgtgggacgggcgccgtcactcgttcgtcttcgtcgtccctagtggtgtcgtcttcctcggcgtcaggctcggacgaatcttcctccgcctctcctctcagtttccccgtctcctccccctcaactgccgtcaggtctccggggtccgccggggtttcaacgtccctgtgctcggtggaacggatggtcgagctgtcggcgcagtctccggccgccgatttctcggtctccgaggcttggcgcttcgaggaggccgtaaatatggcgctggtggcctgcgaggccgtgtcaccttacgatcgctttcgcctaattgaaacgcccgacgagaatttcttgttggtcaccaacgtaattccgcgcgaatcggccgaggtgccggtgttggatagcagtagcagcggtggcgatagcgggccggaggacaaaaagaaaaacgtcgggaataaaaccgcgggggaaaagaacggcggtgggtctcgggccaaacgccgtcgtagacgacgcgctccgaaaaacgacgccgccacgccgtcttttctacgtcgacacgacgtgctggagcgtttcgcggccgcggctaagcctttgccgtcgctttgtgtgcgtgattatgcgttacgcaatgctgaccgtgttacctacgacggcgaattaatctacggcagttacctgttgtatcgcaaggctcacgtggagctgtcactctccagcaacaaggtgcaacacgtggaagccgtgctgcgacaggtgtacacgccgggcttgttagatcatcacaacgtgtgcgacgtggaggccctgctgtggctgctgtactgtggaccgcgcagcttttgcgcgcgtgacacttgtttcggtcgcgaaaagaacggttgtcctttccccgcgttgttgcccaaactcttttacgaacccgtgcgggactatatgacctacatgaatctggctgagctgtacgtctttgtttggtatcgcggctacgaattccctgcgccgacgccgcaggcgacgacggcgggtggtggtggtggtagtggtggcggcggcggggccggcgcttgtgcggtcgagacgagcgcgtcagcaggccgggtcgatgacgccggcgacgaggtgcatttgcctttaaagcccgtctcgctggaccgtctcagagaggtgttgcaggcggtgcgcggccgcttctcggggcgcgaggtgcccgcctggccggcctcgtcgcgcacctgtttgttgtgcgcgctctacagtcagaaccgtctctgtttagatctcgcgcgtgacgaggcgcggaccgtgagttatagccccatcgttatccaagactgcgccgcggctgtcaccgacgtcactttgagccacatcttgcccggccagagcaccgtctcgcttttccccgtctaccacgtcggcaagttgctggacgctctctcgctgaacgacgcgggtctcatcacgttgaatctatgacgtcggtcaacaaacagctcttaaaggacgtgatgcgcgtcgaccttgagcgacagcagcatcagtttctgcggcgtacctacggaccgcagcaccggctcaccacgcagcaggctttgacggtgatgcgtgtggccgctcgggaacagacccgatacagtcagcgaacgacgcagtgcgtggccgcacacctgttggagcaacgggcggccgtgcagcaagagttgcaacgcgcccgacagctgcaatccggtaacgtggacgacgcgctggactctttaaccgagctgaaggacacggtagacgatgtgagagccaccttggtggactcggtttcggcgacgtgcgatttggacctggaggtcgacgacgccgtctaacaggtatagcaatctccgtcacgcctctgttcagattttattaaaaaaaaaacacaacataacgacagtgtcggtgtggtagctagtgcagccttaggaacagggaagactgtcgccactatgtcctccgcacttcggtctcgggctcgctcggcctcgctcggaacgacgactcagggctgggatccgccgccattgcgtcgtcccagcagggcgcgccggcgccagtggatgcgcgaagctgcgcaggccgccgctcaagccgcggtgcaggccgcgcaggccgccgccgctcaggtcgcccaggctcacgttgatgaaaacgaggtcgtggatctgatggccgacgaggccggcggcggcgtcaccactttgaccaccctgagttccgtcagcacaaccaccgtgcttggacacgcgactttttccgcatgcgttcgaagtgacgtgatgcgtgacggagaaaaagaggacgcggcttcggacaaggagaacctgcgtcggcccgtagtgccgtccacgtcgtctcgcggcagcgccgccagcggcgacggttaccacggcttgcgctgccgcgaaacttcggccatgtggtcgttcgagtacgatcgcgacggcgacgtgaccagcgtacgccgcgctctcttcaccggcggcagcgacccctcggacagcgtgagcggcgtccgcggtggacgcaaacgcccgttgcgtccgccgttggtgtcgctggcccgcaccccgctgtgccgacgtcgtgtgggcggtgtggacgcggtgctcgaagaaaacgacgtggagctgcgcgcggaaagtcaggacagcgccgtggcatcgggcccgggccgcattccgcagccgctcagcggtagttccggggaggaatccgccacggcggtggaggccgactccacgtcacacgacgacgtgcattgcacctgttccaacgaccagatcatcaccacgtccatccgcggccttacgtgcgacccgcgtatgttcttgcgccttacgcatcccgagctctgcgagctctctatctcctacctgctggtctacgtgcccaaagaggacgatttttgccacaagatttgttatgccgtggacatgagcgacgagagctaccgcctgggccagggctccttcggcgaggtctggccgctcgatcgctatcgcgtggtcaaggtggcgcgtaagcacagcgagacggtgctcacggtctggatgtcgggcctgatccgcacgcgcgccgctggcgagcaacagcagccgccgtcgctggtgggcacgggcgtgcaccgcggtctgctcacggccacgggctgctgtctgctgcacaacgtcacggtacatcgacgtttccacacagacatgtttcatcacgaccagtggaagctggcgtgcatcgacagctaccgacgtgccttttgcacgttggccgacgctatcaaatttctcaatcaccagtgtcgtgtatgccactttgacattacacccatgaacgtgctcatcgacgtgaacccgcacaaccccagcgagatcgtgcgcgccgcgctgtgcgattacagcctcagcgagccctatccggattacaacgagcgctgtgtggccgtctttcaggagacgggtacggcgcgccgcatccccaactgctcgcaccgtctgcgcgaatgttaccaccctgctttccgacccatgccgctgcagaagctgctcatctgcgacccgcacgcgcgtttccccgtagccggcctacggcgttattgcatgtcggagctgtcggcgctgggtaacgtgctgggcttttgcctcatgcggctgttggaccggcgcggtctggacgaggtgcgcatgggcacggaggcgttgctctttaagcacgccggcgcggcctgccgcgcgttggagaacggtaagctcacgcactgctccgacgcctgtctgctcattctggcggcgcaaatgagctacggcgcctgtctcctgggcgagcatggcgccgcgctggtgtcgcacacgctgcgctttgtggaggccaagatgtcctcgtgtcgcgtacgcgcctttcgccgcttctaccacgaatgctcgcagaccatgctgcacgaatacgtcagaaagaacgtggagcgtctgttggccacgagcgacgggctgtatttatataacgcctttcggcgcaccaccagcataatctgcgaggaggaccttgacggtgactgccgccaactgttccccgagtaaccgggacgcggaacgtgacggttgctgaggggaaaggcaacagagaaggtacaaacccaccggcggggaaaataccgaggcgccgccatcatcatgtggggcgtctcgagtttggactacgacgacgatgaggagctcacccggctgctggcggtttgggacgatgagcccctcagtctgtttctcatgaacacctttttgctgcaccaggagggcttccgtaatctgccctttacggtgctgcgtctgtcttacgcctaccgcatcttcgccaagatgctgcgggcccacggtacgccagtagccgaggactttatgacgcgcgtggccgcgctggctcgcgacgagggtctgcgcgacattttgggtcagcggcacgccgccgaagcttcgcgcgccgagatcgccgaggccctggagcgcgtggccgagcggtgcgacgaccggcacggcggctcggacgactacgtgtggctcagccggttgctggatttagcgcccaactatcggcaggtcgagctcttccagttgctggaaaaggaatcgcgcggacagtcgcgcaactcggtgtggcatctgttgcgtatggacacggtctcggccaccaagttctacgaggccttcgtcagcggctgtctgccgggcgccgcggcggcggacggttcgggtggcggcggctcgcactacacgggttcgcgcgccggcgtctcgccgggcatccagttcggtatcaaacacgagggcttagtcaaaacgctggtggaatgttacgtgatgcacggacgcgagccggtgcgcgacggcctcggtctgctcatcgaccccacgtcggggctgctgggcgcttccatggacctgtgcttcggcgtgctcaagcagggtagcggtcgcaccttgctggtggaaccgtgtgcgcgcgtctacgagatcaagtgccgctacaaatatttgcgcaaaaaggaggacccctttgtgcagaacgtgctgcggaggcacgacgcggcggccgtggcctcgctgttgcagtcacacccggtgccgggcgtggagtttcgcggtgaacgcgagaccccgtcggcacgcgagtttctgctttcgcacgacgcggcgctcttcagggccacgctcaagcgcgcgcgcccgctcaagccgcccgaaccgctgcgcgagtacctggccgatctgctgtatctcaataaggccgagtgttcggaagtgatcgtgtttgacgccaagcacctgagtgacgacaacagcgacggggacgccacgatcactattaacgcgagtctcggcctagccgcgggcgacggcgctggcggcggcgctgatcaccacctgcggggcagcccgggcgattcgccgccgccgatacctttcgaggacgaaaacacgcccgagctgctgggccggctcaacgtgtacgaggtagcgcgcttttcactgccggcttttgtcaatccgcgtcaccagtattactttcagatgctcattcagcagtacgtgctcagccaatactatataaagaagcatccggacccggagcggatcgatttccgcgacctgcctaccgtctacctggtctcggccatcttccgcgagcgcgaggaaagcgaactgggctgcgagttgctggccggcggtcgcgttttccactgcgaccacatcccgctcctgctcatcgtcacgcccgtggtctttgaccctcagtttacgcgccatgccgtctctaccgtgctagaccgttggagtcgcgacctgtcccgcaagacgaacctaccgatatgggtgccgaactctgcaaacgaatatgttgtgagttcggtaccacgcccggtgagcccctgaaagatgctctgggtcgccaggtgtctctacgctcctacgacaacatccctccgacttcctcctcggacgaaggggaggacgatgacgacggggaggatgacgataacgaggagcggcaacagaagctgcggctctgcggtagtggctgcgggggaaacgacagtagtagcggcagccaccgcgaggccacccacgacggctccaagaaaaacgcggtgcgctcgacgtttcgcgaggacaaggctccgaaaccgagcaagcagtcaaaaaagaaaaagaaaccctcaaaacatcaccaccatcagcaaagctccattatgcaggagacggacgacctagacgaagaggacacctcaatttacctgtccccgcccccggtcccccccgtccaggtggtggctaagcgactgccgcggcccgacacacccaggactccgcgccaaaagaagatttcacaacgtccacccacccccgggacaaaaaagcccgccgcctccttgcccttttaactcataaactttcaggtctcgcgtacgattcgcgagtcgggaatgggacacccgtgggtgtttctccgtgtgtatattattttttttttttgtgtgtgtttgcgcccccgtgtgtctaatgtgctgtttgaaacacgtaaagtagctggtggaagaacagataaacctttaataaaaaaaaaagtatgtgctcccgacccacggtctgcgtgtctcttttttatgtccatgtctccaagtctggtgcgggtggcggcggggttaagcgtcctcgaagtcttcatcatcgtcgtcgtcctcttcttcgcggaggcgacggctttccaagctgtcgtggtgactgagcacagcgacttcttcgccggaggctgtggccagcgcctggtacttgacactgccgctaccgcgtccgcgaaagtagcggacggcgcgacacgtcgtaaacatggcccatatgaaaaagagcatgccgaacgaccagctgatgccggtgcggtattcgttgctgaggaaggtatcgtactgcacgatggggtagatgaggccgcagagtccaaagaaggcgcccaggtggtagccgaattgcaccttgacgtattgaaaaaagacggcctcgatcagtaaaaagtagatgatggagatgatagcgtagaccacgaagacggctaacaccatgtggcctgtacgcacgaaaaagttgtttccgaagccgtagcacagggccatggctaccacggtggtgttgaaaccaagcgctacctctaccaggttgacgatgagcgtgcggaactgcaccgtacctttgagcttggggtgcagacgcgagaagaaaaagagtgagcgtttgtagctgcggtactgcgtgaccatgctcacgttgaaaatggtcaggcagaaaaagtgcacggcggccatgaaggcgatcatgctgggcagccgaaatgacatggtcagtgtgaatagttggaacgtgtccatgctgagaatgaagaggaaggctgtgaggctgtcgcccatgtacgaaatgtcgcgtgtcgactggtttaggctcatgcctttgtccttgcgcatgctgatcttgatccagcataccaggtagtagatggtcacggctaaaaagacgagctgcatgaacacggcgtagcacaccaactgcaccgagtctaagaaaagcataggcgtgtgcaggtgcattacgttgtaggccgacatgttgagcctttcaaagtccacgacgtgatagtagacgcaggggtagcccaggtgcggaaaattgctcagcactagatgcacgctgacgttgacaaaagtcagcaccatgaaaacgatagaagcgctccatgtccgtgtattcaccttatccacgtgcgagggggccatggcgatagcggcggcccgctcgctcgggaggcgatgggggcgcgccgatgacgacaggctcgcgggtcgttaaatactacgatgggagccgccgcggctcacgacgcggtttgagcacgtccgggcggtcggtgaaaaaagaccccgcgggccttcgcgactctcttctgtccgaggatgaccgctcagccgccgctgcaccaccgccaccacccgtacaccctgttcgggaccagctgtcatctcagctggtacggccttctagaggcctcggtgcctatcgtacaatgtctgtttttggatctgggtggcggccgtgccgagccgcggcttcacacgttcgtggtgcgcggtgaccgtctaccgccggctgaggtgcgtgctgtgcatcgcgccagctacgctgcgctggcctcggccgtgactacggacgccgatgagcgtcggcgcggcctagagcagcgtagcgccgtgttggcgcgcgtgttgctagaaggcagcgcgttaatccgcgtgttggcgcgcaccttcacgccggtgcagattcagacggacgctagtggcgtggagattttggaggccgcaccggcactgggcgtggaaaccgcagcgctgtcgaacgcgcttagtcttttccacgtagccaagctagtggtcatcggctcgtatcccgaagtgcacgagccgcgtgtggtcacgcataccgcggaacgcgtctccgaagagtatggcacccacgcgcacaaaaaattgcgtcgcggttactacgcctacgatttggccatgtcgtttcgcgtcggcactcacaagtatgtgctggagcgcgacgacgaggccgtcctggcacgcctctttgaggtgcgcgaggtgtgttttttgcgcacctgtctgcgtctggtcacgcctgtcggtttcgtggccgtggcagtgaccgacgagcagtgttgtttattgctgcagtcggcctggactcacctttacgacgtgcttttccgtggtttcgctgggcagccgccgctacgcgactacctggggccggacctctttgagacgggcgccgcccgttctttcttttttcccggtttcccgcccgtgcccgtctacgcggtccacggtctgcacacgttaatgcgcgagacggcgttggacgcggcggctgaggtgctctcgtggtgcggcctgcccgacatcgtgggctcggccggcaagctggaggtggaaccctgcgcgctctcgctcggcgtgcccgaggatgagtggcaggtcttcggtaccgaggccggcggcggcgccgtgcgtctcaatgccacggcttttcgcgagcgaccggccggcggcgatcgtcgctggctgttgccgccgctgccacgtgacgacggcgacggtgaaaacaacgtcgtggaagtcagcagcagcaccggcggtgcgcacccgccgagcgacgacgccactttcaccgtgcacgttcgcgacgccacgctacatcgagtgctcatcgtggatttggtcgagcgcgtgctggccaagtgtgtacgcgcgcgcgacttcaatccctacgtgcgttatagtcatcgactccacacttatgcggtttgtgaaaagtttattgagaatctgcgttttcgctcgcgacgcgctttctggcagatccagagtctgctgggctacatctccgagcacgttacgtcagcctgcgcttcggccggccttttgtgggttctgtcgcgcggccaccgcgagttttatgtctacgacggctattcgggtcacggacccgtctcggccgaagtgtgcgtgcggactgtggtcgactgttattggcgcaaactttttggcggcgacgatccgggtcccacctgtcgtgttcaagagagcgcgcccggcgtgctgttggtctggggcgacgagcggttggtgggtcccttcaacttcttctacggcaacggcggcgccggtggtagtccgctccacggggtggtgggtggtttcgcggcgggacattgcggtggcgcttgttgcgcgggctgcgtcgtcactcaccgccattctagcggcggcggtggtagtggcgtgggcgacgcggaccacgcgagtggcggcggtctagatgccgctgccgggagtggtcataacggcggtagtgatcgggtttctccctccacgccgcccgcggcgttaggtggctgttgctgcgcagccggtggcgactggctctcggccgtgggtcatgtcctgggccggctgccggcgctgttacgggagcgcgtgagcgtgtccgagctggaagccgtgtaccgcgagatcctctttcgtttcgtggctcgccgcaacgacgtggacttttggttactgcgcttccagcccggtgaaaacgaagtaaggccgcacgctggggtgattgactgcgcgcccttccacggcgtgtgggccgagcagggccagatcatcgtacagtcacgcgatacggcgttggcggccgatatcggctacggcgtctatgtggacaaggcctttgccatgctcacggcttgcgtggaggtctgggcgcgagagttattgtcgtcctccaccgcttccaccaccgcttgttcttcttcttccgttctctcctccgccttgccgtccgtcacttcgtcctcttcgggcacggcgacggtgtctcctccgtcttgttcttcttcgtcggcgacttggctcgaggagcgcgacgagtgggtgcgctcgctggcggttgacgcgcaacacgctgctaagcgggtggcttccgagggcctgcggtttttccggctcaacgcttaacgagtcacgtaggggaactacgtgggtaagtgacgtggatactagtaaaaaaagtgcgtcaaagctcttagcgtgtgacgtggatactagtaaaagggacgtcaaagctcactacgtgttgcgtgttttttttttttctatgatatgcgtgtctagttcgcttctcactcttcctctcctcgttcccagcgcggcggcagcttggggggtgagggcaaattggggtagttggcgttgagcacgtctagcaggcccaggcccacgggccaaccgtccacggtcttgcgctcggtcagcttgaggctgaacgagtgtgcctcgtcctgaccggtaaggcggaaaaagaagcgtgctaccagctgcaggcaggtatgccgcgtctgctggaagagcacgaaggtagcgggcacgtactgcacaatgtgcggctctttttcctcaaagagcaggtagagcgcgctgcagatcagccgcctggcgctgtggtgcagcagccggccgaagctttcgcgcacgttcaccgcgtccaggtactggagcaggtcgtgcaggcacttgcgcgttaagttgcaattttccacgcacgaaataacggtacagagcgcgaagtgcagcaggttgtcggctttgacgatgccgcagcggtgtttgagccgcagatccgagagcctcacctgcgtgacggcgtcttcggtctcgagcaaaaacacggcggagtagcctagaaaggccgaggtgcacagcaactcgctgcggtactcggccatggagaccagcagcccgtgctccgtgtgcagccacagcttgtcgccgcgcaccgtaaagtcgagcacttgcggctccatgatcatcacattctgtctagtgaaatccgtatggacctccagcacgccgcggatcatcagggcctccatttcgaaatcggccgacacgctctgggccgcgccgctcctcgtctgccgtgatcaagcggcgcggcgcggacctttcaagtgttcctgggccgccgctcgaggcagttcccctttctggcactccgcccgccgcttcgcggctcatttggcgccgacgcgccttctcgcggctgcaaatcagctccacgtatcggcaaaacttgctgtcgtcgtaggcggcggccacgatctcgccgaaggagagctgcaggtaggcctcgggtacggggtccagcgtgcccagcgccaggatgtgacacagatagggcagggtcacgcgctctaccgtgtaattggagtagacgatggcctcttcggccccttgatgcgtgaccagacgccgtaggcgaaaggtacggaaatactcgttttcccacaactgcgtgaggaagcgttccagcgactcggtgccgggcacgaactgcgagaagaagctgttggccaccaggcggttgtcttccaccgccagcggacggaagggcgccgcgtcgcgcgccttgcgcacggcctccaacacgggcaggtggtagagttcggcgtcgcgcgcgcccaggctcatggagtcctcgcgccgcgaggcgtagcgcgtgagcaggtcgcgcagttcgcgcacgcgattctcccaggtctggttaagcgtgcgcaggtcctggatctcgtccacctgcgactggatctgctcctccaggcacttgataacctgcttcttaaacaggtcgcggatgtcccgctcgggcgccgccgggccgggtggcggcggcagcagcccgacgtggcccgcgggtcctcccaccacggcgccgccgggtcccaccacgccgggtccgcccggaccacgcgcgggtagtagacggttttggtccaccagcgagggggtcaggtcctgcagaaaggactcgacgctgtcctcgatgccgatgcgcgatttgctgtccgagacgttaagcaaaaacttcataatggactttttggcgtcgctgccccggtcgtgctgctccatcatctccaccagcttcttgcagttgagctcgtggcggctggcggtcaccactttcacaggaaaggtattgagcaactggcagatcttttggtggcggcagagcccgtcgtagcgcagaatctcctcgtgcaggtgtgccaccggcgtggtgaacagcagcttgtcgcgctcataagccagcggttcggccgccacgtacaagcggatgtgcttgccgcgcagctgcgcctccagccgctccgagcgcaccttcttgaagacgcgtacctcgggcgcgttggctacgcgcacggcgcccaggcgctcggccacctgcagcagcagcgccaggttagcctgcagcaggtcctgcgccagcgggtgtgtctcggtggcccgctgcacggccgcgcgtacaaattgcgcccgctcggccgcctcgctcggcttggtcttcacgtccagcagcggtaccagtcccaccgttacgcaccaatccacgtagagaccatagtcgtcgttatcggcgtactgatataaaatgtcgcggagcgcgcccagcacgcccgtttgcacgctctggcgcaacgaggcgctccacaccaacagatactgctccaggtcctcttcgtccagcgcgcggtagggaaatagcgccgcgtgcaacttccactcctcggccacgcgccgcaccgtgatggtgtcaaagagcgttttgcacactccgtagagcagctgcttgcgcagcacgcacgggtcgcgcagcacctggtgcatgctttggccgcgacacgtccccagaaagccgtgcagcaaccgcaggaagctcatcgtctgccccgtggggaaaatgtcgatgacggcctcgtcatccacgccgcggcccacgcccaagtacgacgacgccttgatcctcaacctctcgtcggccgccaagatcgaacggatcgtcgacaaggtcaagtccctctcgcgcgagcgctttgcgcccgaggatttttcgttccagtggtttcgctccatcagtcgcgttgaacgaacgacagataacaacccctctgccgcaactaccgccgcggcaacgacgaccgttcactcctccgcctcctcttctgccgccgctgccgcttcgtccgaggccggcggcacgcgcgtgccctgcgtcgaccgttggcccttctttcccttccgcgcgctgctcgtcaccggcacggcgggcgccggcaagacttccagcatccaggtgctggcggccaatctagattgcgtgatcaccggtaccacggtgatcgccgcgcagaacctcagcgcgatcctcaaccgcactcgctcggcgcaggtcaagaccatctaccgcgtcttcggcttcgtcagcaagcacgtgccgctggctgacagcgccgttagccacgagacgctggaacgctaccgcgtgtgcgagccgcacgaggagaccaccatccagcgcctgcagatcaacgatctgctcgcctactggccggtcatcgccgacatcgtggacaaatgcttaaatatgtgggagcgcaaggccgcttcggcctccgccgcggccgcagccgccgcctgcgaggacctctcggagctgtgcgagagcaatatcatcgtcatcgacgagtgcggccttatgctgcgctacatgctgcaggtggtggtgtttttttactacttttacaacgccctgggcgacacgcgactttaccgcgaacgccgcgtgccctgcatcatctgcgtcggttcgcccacgcagaccgaggcgctggagagccgctacgaccactacacgcaaaacaagagcgtgcgcaagggcgttgacgtgctctcggcgctgattcagaacgaggtgctcatcaactactgcgacatcgccgacaactgggtcatgtttattcacaacaagcgttgcaccgacctggactttggcgacctgctcaagtacatggagttcggtatcccgctcaaggaggagcacgtggcctacgtggatcgcttcgtgcggccgcccagctccatccgcaacccctcgtacgccgccgagatgacgcggctttttctctcacacgtcgaggtgcaggcttacttcaagcggctgcacgagcagatccgcctgagcgagcgccaccgtctctttgatctgcccgtctactgcgtggtcaacaaccgcgcgtaccaggagctctgcgagctggccgacccgctgggcgactcgccgcagcccgtcgagctctggttccgccagaacttggcgcgcatcattaactactcgcagtttgtcgaccacaacctctccagcgagatcaccaaggaggcgctgcgccccgcggccgacgtcgttgccaccaacaactcctccgtccaggctcacggagggggaggatctgtaatcgggagcaccggcggcaacgacgagacggcgtttttccaggacgatgataccaccactgcgcccgatagccgtgagacgctgctcaccttgcgcattacctacatcaagggcagttcggtgggagtcaactctaaggtgcgggcctgtgttatcggataccagggcacggtcgaacgtttcgtggacatcttgcaaaaggacacgtttatcgaacgcacgccctgcgagcaggcggcctacgcctactcgttagtttcgggcctgctcttctcggccatgtactacttctacgtgtcgccctacacgaccgaggagatgttgcgtgagctggcgcgcgttgagctgcccgacgtgagttcgctctgcgccgctgccgccgccacggccgccgctcccgcttggagcgggggagagaatccgataaataatcacgtcgacgcggattcttctcagggcggccagagcgtgccggtatctcaacggatggaacatggccaagaggagacccacgacatcccctgcctgtccaaccaccatgacgactcggacgccatcacggacgccgaactcatggatcacaccagtctgtacgcggatcccttttttctcaaatacgtcaagccacctagcctggcgctgctttctttcgaggagacggtgcacatgtacactaccttccgcgacatttttctcaagcgctaccagctcatgcagcgtctcacgggcggtcgcttcgccacgttgccgctcgttacctacaatcgccgtaacgtggtgttcaaggccaactgtcagatcagctcgcagaccggctccttcgtgggcatgctttcgcatgtgtcgccggcgcagacgtacacgctcgagggctacaccagcgacaacgtgctcagtctgcccagtgaccgccaccgcatccaccccgaggtggtgcagcgcggcctttcgcggctggtgctacgcgatgcgcttgggttcctctttgtgctcgacgttaacgtttcgcgcttcgtcgagtcggcgcagggcaagagtctgcacgtgtgcaccaccgtggactacggcctcacttcgcgcacggccatgaccatcgccaagagtcagggcctgtcgctcgagaaggtggccgtggactttggggaccatcccaagaacctcaagatgagccacatctacgtggccatgtcgcgagtcacggaccccgaacacctcatgatgaacgttaacccgttgcgactgccctatgagaagaacaccgctatcaccccctatatctgtcgcgcgctcaaagacaaacgcaccacgcttattttttgacacaacaccgtgtaaggaaaacgtgactttattgagcagggtaaaaaccacgtacaagaaccacgttgtctatccccaaaaaaacacacaccgtcagggaacacatcgcctatagatagcggcactttacataaaaccaccgtacctgcatcacggtggctcgatacactggaaattcaataaaaaccaccgtgtctccgtgacggtacttatcgggtcagcgtcttttgagatttctgttcgtaaacttatccgtttccccggtccgcggtgtctcctcgcgaggctgacagtcgacgggtggtacctgcaagagaagaaacccgggtgggagcgacgccgtcgctgggtatcaaccccgcggctgaccgtcgtccggtaaaggaacaacccgtcgtcgcaagccgggttcgaccaagagaaaaaacccgggtgcggggggagacgggtcgtcctttggttgttcgcggacggcgtacatgccgcgtgggtcagtcgacggcgtcgctccgtgcggtcggtcatcattctgcttcacatatatgggttgtttgtgtttttttttataatgaatacgcactgatcctatccgtgactgcgcgtgtggcagagaggatgccttataacatgtattttgaaaaattgccaacagctataatttctctcatgtagcagaatagagaccttttgtcgtctttttgtttgtcattacttgttttccagggaattagagagagggaaccgcgcctccggcggcggtgcccgcggaccccggccccttctcgcgtgcgcggtgtgactggttgagcgaatgagcagctaggcttggtggtgctccgcgtgcgggggagaagacgattaacaacaaaaaataagtggaagtggccggtgggtctttgtccgcgtgcgcgcccatccgtcgccgggaccgagcagaaagtgatgtggtggtacattgattttttccttgacaggaaagaaaaaaaagagttttgttttcctatgtgagaggagaaaggtatgtgaggagatgttcgatgatcgtatgttacagttatgctgtaaggaagcttttatcgtgcgtcctgtttttcatttgatgtatatgacacaattgaaacctatcgataggcgtatatcgaggattcatcaattcttagaatcgtcgtctttttggctaattggactttgcccatgttggttgtcattcgtggcctgaggtcatcgtcgtccacgacgacgtgtctatagcgtgcggtgtgatcattgtgtcgagccagagaaagcgcgcctcgcacgacgtttgcggatcggctcgcgggtgtgtggaattcctaagaacataatcagctggtcgtctttctttgatgtgttgttgtcgtcgaggtcttgcttcgttttcttttttctttttagtcgatggaacttttcttcggtacgggttcttgttatggaagcttgtgttttcgaacatgaattcgaaaaaataaaaaggcctatcttcgtttcaaaaaaaggacagatatcaatcttcttaacttatatcatggtaaattcagaatcctatggtgtcttattatctctaaagtagtcaacattatggtctaacttgtatttccctgacgagatatatatgatccttataacctggctactatcatgaacaacaatatccttacttacagtcatcttcgtgagttaatgaagtataatatcggtcatctatcaacttatctgctatgtaacgtacccttttaggtattttgcgtttcttaacgagtgtacccgcctgtgtgaggcgaaactctgagaagtctaccgagtcgagttacaagtcactaaaacacttacacgagttatctatactaaaatcactatctatgttgtttgcttacctaattattatcctacatgacgaagctacctcccaacgtaaggtagggggagaggagacagaacaataaaaagtaactaatgtttcttagaacttacccgctaaggacttaccaaactatattcaccaaaaaacaacagctacgtgtttcatttgttttaatctaccgaagtaaaaaaaaaaagatgattagctatccagaacctacttacttcttaatgttttaactaaggatgcctatgggattggaaaaaaaatcacagcaacttgctactaatcagttgacagcgaagagactcataacaaagatttctgggtaatacggttataataatgcttatggactaaaggatacttggaaaaaaagaacgggctatgactatagagattcgtcgagatatcaaacttcaaataggcggctatcattcatggttgtggtgactatatcgtggagaaaaaatgtgatcgttagttagctaggtgagacttacagctatccatccgtctagtttttcgttgtaatgatgatagtacgtctatggtggtgatcgattttggttaacaatttgttcgtttaaaggcttaatgtacttatgctacatgatgtattattctttgattcatcgttcctcctaagggggtgtatgtatgtatgtactagtcgtatagtgttcctaacatcatgactattcagactatggcttcatctatcgtgtctaaagttcacttattctactattactatatatatgcactactatgtaactaggatatggtcctataaggtgtcttctatcacggtggcttgtttatcgcttggcggttacgagcaagagttcatcacggaccagccgtgaggcagggcacacgcgggtcggcggcgatgatgtcccccgcgaaggggacaacgaaaacaagaggccgccggccgcggccacggatgcgtagcggttacacaatgtttggttgagcgttttgtttcatcgtcgtggtggttttgttgttctctgtatatatcgtgtggtggctttatcgtcatcattattatcatcattcttgtttccatcatcacgatgagttttctccgttttcctctcctccagtggtagtcgtgtatcatcatcaatcatcgtagtgacgtcgttgctgctgctgctcttgccttcatggcggtatttctcttcctcccccctaaccccatattaactcgtgagtgtgatggttagagtggctgcttgttttttttttcttttctctttggaacaacaaaagaggataaagatggtcggtgaatgtattattattatcatcattatgatacggtcgcggtcttcttctccgatgacgaaacctgcgcacatcgaagaaaagacgagcgcgcgaaccgatagccgtccgtctgggacgaaggagaagatgatggggagaggaggagagccccagaagccagagcgagaagggagacgacagacatacgtcgtcaccgtcctctggaggaggcacggcggcgctgtttgttgtttggatgcttgattatatcctgttctatggggtagattattatcaataggcttggttttcaaaggtcagcctgtgtattgtcgtgtctttttttttcgttctcatgatcgcggagaccacacagacgtgcgcgtctcccaatggctaggcgttctttttaggtagtaattttttgatcttttttttttcttaacaagtctggcttgatttcttttatctatgatcgattcttctttttctcgggggttgcatcttccgtgaaagtaaagtgacactactctaaatggtaaccatattatctgttgattaggagaaaaaataattttttcgcacgaaatcgatcctaagtgaggtgatttacttgctatcacacgaaatgattatcttttgctgctaacgtactgaattttttaacagaattgcttctccgtaactatttccgcagattcagacagattgtcaaaaaagaatacggcacagaaatagtgggtctgtggcttttggttcgtgtacattcgcgtttgcgtgtcgagatttctacggtatgtttattcttcctgcgatgatgtagggtccttggtgtaagtaggatttcgagtatctctcttagagcgaacaaaataatcaaaaaacaacagctaggaaatcgagggttactctacgataaagtgtctctacaaagtgaagaatgttacgttgtggtggaataataagactcgcgtgatcgatgagtgatcgagagcggctcgaaccttctttaagagctttgtttagtgcaactttaaattacaaggagtagaaagctgaaatgaatctatgaaggtgctattctttgaatatcttactttgtacgcttcacattcgttatttggatagagagttgtctagagaaaatctgtgattctctatgagtgttatttttattatccttttggggactacgatttttcttcttgttctacataccactactactcgtaatcacatacatggacgaaaaaaaaattcgtcaggcagtagataccagattctccgacgttacggcgtcttttttttcttttgagagagtatctgctgagattgtccgtggtgtatctagtcgctatttttgttgttactagtagttttgcacacagtttattcagtatagtttttcttcttgccatgatcaattaagcccaccacctttttttttagagaggaggaatttcgtcttgatctccagccggagacaacggcggtggtggtggtggcgggagagacttcaaggcaatgaaaaaaaaaatttcgttttgccatcaagtggtgacgataacccgtcagattgataattggttcctacagaaactattctaaccgcggaagaaagaaattgaaaaaaaaaattgacaaaaacatcataacataaaggaccacctacctgggacgcgcagttgggcggcggactgggacggcatgctgcggcgatgctgtcggtgatggtctcttcctctctggtcctgatcgtcttttttctaggcgcttccgaggaggcgaagccggcgacgacgacgataaagaatacaaagccgcagtgtcgtccagaggattacgcgaccagattgcaagatctccgcgtcacctttcatcgagtaaaacctacgttggtaggtcacgtaggtacggtttattgcgacggtctttcttttccgcgtgtcgggtgacgtagttttcctcttgtagcaacgtgaggacgactactccgtgtggctcgacggtacggtggtcaaaggctgttggggatgcagcgtcatggactggttgttgaggcggtatctggagatcgtgtttcccgcaggcgaccacgtctatcccggactcaagacggaattgcatagtatgcgctcgacgctagaatccatctacaaagacatgcggcaatgtgtaagtgtctctgtggcggcgctgtccgcacagaggtaacaacgtgttcatagcacgctgttttacttttgtcgggctcccagcctctgttaggttgcggagataagtccgtgattagtcggctgtctcaggaggcggaaaggaaatcggataacggcacgcggaaaggtctcagcgagttggacacgttgtttagccgtctcgaagagtatctgcactcgagaaagtagcgttgcgatttgcagtccgctccggtgtcgttcacccagttactttaataaacgtactgtttaaccacgttgcgtcgtgacgttgtttgtgggtgttgctaggcgggctggaaagatgatgtataaatagagtctgcgacggggttcggcgctctgccggctgcggcggcactcgctccacggcctccgacgagcgttgcgctcgcgctttgcgccgccgcgtcatggatctccctactaccgtcgtgcgaaaatactggacttttgcgaatcctaatcgcatcctgcatcaaagcgtcaatcagactttcgacgtgcgccagttcgtcttcgataccgcgcgtctggtcaactgcgtggacggcgatggcaaggtgctgcacctcaacaagggctggctctgcgctaccattatgcagcacggcgaggcttcggccggcgccaagacgcagcagggcttcatgtctattgacattacgggcgacggggagctgcaggagcacctctttgtacgcggcggtatcgtcttcaacaaatccgtctcctcggtggtgggctccagcggacccaatgagagcgcgctgctcaccatgatttccgagaacggtaatttgcaagtgacttacgtgcggcattacctgaaaaaccacggcgaatcctccagcggaggcggtggttgcggcgccgcgtctaccgcttccgccgtctgcgtgtcctcgctgggtggcagcggcgggactcgcgacggcccttctgcggaggaacagcaacggcgaaggcaggaacagcgtcacgaagaacggcgcaaaaagtcgtcctcgtctgccggtggtggtggaggcggcggcgctggtggtggcggtggcggcggcgggagcggcggtcagcactcctcggactccgccaacggactgctgcgggatccccggttgatgaaccggcagaaggagcggcggccgcctccctcctccgagaacgacggtgagtcccggccctcctcgcgtcacggtgctttccgagtggactcgtgagcctcccgtagcgcacgagcgagcaggcgagcggtgttggtgcgctggtggttgtgtggatgataaccatgtgctttttcgtgcgctatgtgtcgtcccgtctgtaggctctcctcccctccgggaggcgaagagacaaaagaccaccgcacagcacgaaggccatggcggcggcggcaagaacgagacggagcagcagtccggtggtgctggcggtggtggtggcggcggcagcggccgcatgtcgctgccgctggacacgtctgaagcggtggcctttctcaattactcgtcctcatcctccgcggtctcttcttcctccaacaaccaccaccaccatcatcaccaccataacgccgtgacggacgtggccgccggcaccgacggtgcgttacttctacccattgagcgcggagcggtggtttcgtcgccgtcgtcgacgtcgccgtcgtcacttctttcgctccctcgacccagcagcgcccacagcgcgggcgagacggtgcaggagtccgaggcggcggcgacggcggcggctgcggggttaatgatgatgaggaggatgaggagggctccggctgaggcggcggaggcaccaccgcagtcggaggaggagaatgattccaccactccagtctctaactgccgtgttcctccgaattcgcaggaatccgcggcgcctcagcctcctcgcagtccgcgttttgatgacattatacagtcattgaccaaaatgctcaatgattgtaaggagaaaagattgtgcgatctccccctggtttccagcagactcttgccagagacgtcgggcgggactgtcgtcgtcaaccacagcagcgtcgcgaggaccgccgcagctgtctccgcagccggcgttggccccccagcagccgcatgtccgccactcgtcaccaccggtgttgtaccctcaggttccgtcgccggtgtcgcgcccgttgccgccgcaatcgaaacaccagctgctcctccccggcccgtgtgtgaaatcaagccctacgtggtaaaccccgttgtcgccaccgccgcggctgccagtaactcttcctcgtcttcttcggctccactgccgccgccgccaccaccgtcgggcggacgtcggggtcgggcccggaacaatactcgaggaggcggcggtggtggcggtggtagaaacagccggcggcaggctgcatcgtcgtcgtcctcctcctctcggagatcgcgacggagaaacaaccgccacgaggacgaggaggacaacgaccctctgctccggttgtcgcaagttgccggcaacggccgccggcgagggccctcgttcctcgaggacggactcgaaattatcgatcccagcgaggaggctgcgatcgccgccgcctcgatcgcggcgtttttcgacgattaaaaaaccgagccgagaccggaaaaaatatgaaacaggacgcgcttggacatttgggtttccaccccttttggtgtgtgtctatatatattggtcactgattttttttacaataaagagatagacatcacagttcaccaccttgtctccccggtgtgtctattatcatcaatcacccacagagtcgccagtccatggtctctcggtaatgcgtgtccagatacgcgttggccagtataaaatggtcgttgcccacgaaggcgcgggtggtgttgcgcggcgacgggtggcaggacttgagtaccaagtgccgccgtcggtcgatcaggtactcgcaggtgtgcgcgtcggcgccccacagcatgaacaccagatgctcccggcgctctgacagcctccggatcacatggttactcagcgtctgccagcctaagtgacggtgagatccaggctgtccgtgcaccacggtgaacacggtgttgagcagcagcacgccgcgtcgcgcccaggcgtccaggcaacccgaggccggacgctgaaacccgtccaccgtacgcgccagttcgcgaaacacgttgttgagggagggcggcggcggtcggcccgccagcgtgccgaaggccaggccgctggcgctgccgtcgcagtacgggtcctggcccacgatcaccacgcgcacctgctcgggcggacacagatagctccagcggtgtacgtgctcgggtgccgggtacaccatctcgagttgccgcgcgccctccaccgccgccaccgtgtcgcgcagcagcaccgtgtcgtggtcgggcaagctgaggaagcggatccagtcggcgctcagacaaaacacgcgagcctgctcgtcgggggttaacagagagcctttattatcagcaatgttagcgagcatccactgcttgagggccatagcgcgagtgagccggcaggttgacgcgcgtctgcttcagctcgggcggcagtccggcgtagtatttatctaggtggcgtagtagcggcgggtccagctggtgacgcaggcagaattccttcactgcgttgtacaggccgtaaaagagtgtgatgccctcgggcgcggcagcggtgctcacgggcagacgcacggcgcggttggtacgcgtggcttcgttgcgtatggccaccaccacgttaaagagagacggtggcaccagctcgaagcctaacacgtgttccgtgaagatgctgcgcccgtatgacagtcgcgtgaggtcgtagccgcggcacaggtcgtccacgcacgtgtacacggccggcgagccatcgccgcactcgctgtagccgcgcatcaccgtcatccagcgcggcgctgtgtccgagctcaacagcgtcagcagggcccgcaattgatccggattgttgtacagcagggccagagtgtccaggaaagcatcgtccaacagcacggagttggcggcctccggcgtaacgggacggtaacgaataagttgcgatagcgggccatcgcgtctggtaacattcaccaacgggcgcagccaactttcatacttgtcaccctgaaacacctcacccaacaggcatcgacgcgttagttcggggcactccgcggggactttctcggcggcggtaggagcgacgctgacggcgactgaggaaacaatgggcagcagaaggcaacaccacagcagtaccaccggtccaggtgagaaagagaagccgcaatccgggcggcggcacatcaagtctgcggcacgatgagagtgtgacggtaaggagccagttggcgccgaaagttggcgctcaggtcttcgatccctaaaacgttatatattgcatccagcaggtgagccaggctaaacggattcacgtaccaggtttggttacccgcgacgatgacggccagaccgtgggcgctacagttggagaggttcctgggtacgaaggtaactgagtcgatgtcgcgccacggggggaatgagacagacgactggcgcacgctgtaatcacaactgtgattgacgtgtagcgtgtaatttcggttgcactcagcctcgaagtagagggggaaccacagttcgtcgtactcgtcgtcgtcctccagttctggctcttcttcatccaccgcaatgtctacgctgctctgagattcctcttcgtacaggatgattgacaggttatggctacacaggtcctgggcgggaggacgcgtgggagcgcgggtggtggtaatgttttccagatcgtcaaaagtcggagtgtagtctgacgccgtgacgacaccgtcgacggagatggtagaagttgcggccggtgtcacggtggtaagtatggatacagaaggggagggggaagtagcgttcgtaccgatggttgtggtattattattccctgtgtttcttgttccagaaaccgttgacgttgagatgggaatcgacgtggtgctggacgtcagattgctgaccgaggaaaccgtggtgggagtggtgacggtgttactcgtggttgaagtgacgttaggggaggtagtagtggtaccggtggtggcgacggtagtgtttgtcgtggcggcggcagcggtggtactggtaacggtggtcgcgttggtttccaccgcttcacacagtaagcaaaagcacagggccgggaaaagcaaccagccccgccatcgccgccgccgcttcatgaggtgggcaggcgaaagctggtgaattcgttgtacagcggcaagtggggcgccgcgatcgaagggtacgtcaacaagctgacgttgatattaaatacgtctggctgcttttctacgatggaagcgcacagggttacggcgtcaaacaggtctttcttggtggcgcccgagacccacatctggtatacacccgtctcgtggtacgaagtagagcgcggcaccaccggacggatgcagtccagaacgcggttgggatcctggtgaaagaatttgaacgtggctacggcctgtggcgtgtgcggcatcgtctgcgtgatgagctgctggcccgctaacacggtgacgttgtgcaacttgagcagggcactcttgagggcctggaaagcgttgccgcacgaggcgctgatctgcagctgcacggccgtggagtcgtgcagccgcatgagacgtgatacctcttcgaagacgtacttgtatttgctggcaaaaagtggcgcgtaccgacagtcggccggcaaaatgtaggtggcgttaccgccgttggtggccacggcgggcgcagcggccgcggaggccggcgtaaacagcgtcagcggccggtggtggctggtaaggtcgatcatgggcggcgtggtgaccgtggcggtggcgggcatgacggggtttgcggcgacgggcactccggccacagcggcggccgcggcggccacggcggcacttgccgagcccacacccgccggcagtcctccgccacccatgacgccgccgggcagagcgtcgcccagacagacttccgcagtggcgggcgcgctctcagcggtcagtacggtttgccgatcgacctcgcgacgaaagctggtgaggaactcactgtgatccatggccgcagggcccgagatcccgggattctgcgggtgctgaccgagtgcggggcgagttatatggaagacgattagcttggagcggagttttgcgtccctagctgacctgcggatcagcgacgtgccatagggatagactgtgagcggcggccgcaacggcggggtcggccgccgctcgtcgtcacggggcggcgcgagggaggaggaggtggtgggtacgatcttgacgtggttgacgtcctgcccgtccgggggaatacgcaaaaaaccccgtcgcggcgctaccacgatggtgcgatgggtctttctcttgttggccggggccagggacttgcagatgcgtgtggagccgtagacgatctggacgtggtcctgggagaacatgaccatcgccgccaacgctcagcggggggacgtgttgggaacacagaggctgagggaaaactccgtagaagtcagcgaaataaagacaacacagcagccactcctctcgtctcgggccctaccactgcttgaagtagggcaccgggtgtttcttttcctcaacgggctcctccagtctcttataggaccagtcccgccggcgcgccagcatgtaggtcacgtacaaaagaataatcaccatgaacaccaggaaagccagcacgccgtaggccagcagccggtcctcgaacagcgggtcgctcttgataaacacgtaggtggtggtaaaacttcggcccgcaatctgaacgtggagacgcacgacagtatacgtgccgttgaggtagaagacaaactcgcgtaaccgttgtccgttatacgtcacgttactaatattccacggcggaatgagctggttgccctgatgcagatgcacggtgctgttggggtgatagaggctgctaccgttgagcaagcagtgttcgtgttcctgaagcagcacgcggacccgcatcgtggtggcgttcaggcgagtcccgtacacggcgtagatgggataggtgaaaaggtcccaagtggcgttgtgatggcggccccagctgaagaaagagcacgtgtactcagtggtctcctgcggcctgagtcccgagataagcagctcttgagcagtagcgttgtaggagagatgtagttttcctgtggataaaattcataagttgtttattttgttggcaggttggcgggggaggaaaaggggttgaacagaaaggtaggtgctacttaccttcattatcgggggggaaggcgctaagataccccacctgagtgaagggacccttgcagtctgtccgtgcataacaagtaactgataaaatgtctggatttttggtgttattcaacaggataactttgcaggtggcgtttagagacacttggtcgtggctgtagctggcttcgcaattcacagtatacaggtgcccctctttctgcgtcgtggctatcacggaagtggaggcggacgaggtagaggtttgtaccgtggtggtgacagcagaagtgacgttgttagaggtacttattgacgtagtagacgtgacggtggtattactaggggaagtgacggcgcttgtggtgctacttttcacccccgggtgcatgtcgcccaagagcgcaactacgagcgcgatcgccagtacggaacacatgttgccgtgtgacgagacggcgtgtggacgagctatatgtggcaggaggtcgcgtcacctcttgtgacgcctaaacgtccagctccagataaaagaggcgttaataatgaagaccacaaaaaccacttgcgtcagtatgacaatcataaaggctcggtgattgctacgcctaaagtacgcgggattatccaccagttcatcctgctgaacaaagtggatgattgacgtattggtgttactatccgtgttgttgaccatggatttgactaagaaagtcttggcgccaaaagtcccgttagagccccagcaggtgacgctgctattcacataagttcccggtgcccccgctagcatgtatttcagttggtgggtataattttttctgtcgttatccatgtcattgctgtagttgaccttgctggtgagaaagcgtgttttcaacggggcacttatcatcatccctgaggccaaaaagggcgaattgcaagctgtagtgttacaaaaaatagtcaagttagtgtcattgtattgataaatgtaagccatgcttacttcaggctcataccacccgattccaatcgcggccgccatcgttaccacgtcccatcttcccagacttaccaccgccaccactaacagcgtcacccccgcacggtacatagttaccctctcgacgtcgccggctgtcaatgacgtgcctgcgtcagtggctatgatttatagcttttgggcccaaccgcaacggatctgtcgtaatctaccttccacagggccgccgcgacgatgctgaacgacaggatcagacagacggcgtacaaaagtcctaggtcggcgtcgacgcggcaggtgcggatgtctcgcagggtgggtagatgggcgatgcacaactctttctccccccgcccgtacatcccatcttgtatcagcagccgtagcgtggcattgatggtcagcggggtaaccaaagaaatcacatagggatgtgtacaggaagtgcagtgacgggtatccgtgagatgtaagtcaccaccctcctcaccgtcatcatgaaagaccaggactcgggtgagacgacccgatgaatactggatctcccaccacagtctttggtccaacaccgagagggcgcaagagattctaagtctccctgggttgggggagcagatgtaagtcccgtatgtgcctctcgccatcagggccatacacatgagggggagaaggacaattatccgggaccacccgcacccccacatcacgagaccagagacggagatgtataaaaaaagctacttttattaaacagcattctcaccacacgttaatactgtcacggggaatcactatgtacaagagtccatgtctctctttccagtttttcacttactgagacttgttcctcaggtcctggatggctgcctcgatggccaggctcagggtgtccaggtcttcgggaggggtctcggtgggctgctcaaactgccccacggcgtaggccttcgcggccgtctcgtagataggcagcatgaacccaccctggttggtggagaagatgcgcaccatgacctgtttgggaaacttttgcatcaggggcaggcacaggttgagagcgcccaacaggtccacgggggtggcagcgtggatgatcatgttgcggtaatcggaggaacgggggcataattggtgggtgtgcaattctttgaggctccacgcggccttgacgccttcgttacaagcatcggctgtgcgctgcgccacttcgggtggatgtgtcacgggcatggtgtgctccatgaggaagggagtggagagggccaggttgcacatggtgcccaggcgacaccgcaccgcatccacctcactcttcacctcatgattgcgggtgtagataatctggatgcccttgttgttcacctgcatggttttgcaggctttgatggcctcatctaacacctggtgcatactgggaatcgtgaagggcaggttcttgtactcaagagagcgattggtgttgcggaacatgcggctcacctcgtcaatcttgacgcgaccccgccgagtctgcacgttgggtgtgcagaagggggtgttcttatctttcatgatattgcgcaccttctcgttgtccaactcggagatgcgtttgctcttcttcttgcggggtccggtgctcgccccgccgctgctctgatggccgcagctcagcagagaggaggaggccgcgccaccaaaaccgccgcgcccatggtggctcgaggtcacggatgctcctccgccactgctgcatttcatctcctcggactcactctccgagtccgaagccgaactgcaggaggaggaagacgaagaggaactatcttcatcgggccggcccaagggatcgggaagaggagggtggttcatctgggagagcgggtgcgtgggagaggtcactcgcggcgtgccgctgccggtggaaggggaagacgcggtagcaccgcgggtttcgacttcttcaccctgttcttcctcgctatcagagatcacgatacagccggcggtatcgataatcttgttgcggtactggatggtaaagtcgggctcgggcttgatgtcttcctgtttgatgaggggcagcatgataggcgcgggaggcacgggcggtttaataatcaccttgaaaggacgcgtggttttgcgcggtttcttacgcgggctgagctcgggagtagcggatgccccggggagaggagtgttagtaaccgcgacgctggtgggggtcggcttgttaagaggggcgctgctaacgctgcaagagtgggttgtcagcgtggggccggtgctactggaatcgataccggcatgattgacagcctgggcgaggatgtcacctgatggtgataagaagacacgggagacttagtacggtttcacaggcgtgacacgtttattgagtaggattacagagtataacatagagtataatatagagtatacaatagtgacgtgggatccataacagtaactgatatatatatacaatagtttactggtcagccttgcttctagtcaccatagggtgggtgctcttgcctccagaggcggtgggttcctcagcaccatcctcctcttcctctggggcaacttcctctatctcagacactggctcagacttgacagacacagtgtcctcccgctcctcctgagcaccctcctcctcttcctcatcactctgctcactttcttcctgatcactgttctcagccacaattactgaggacagagggatagtcgcgggtacaggggactctgggggtgacaccagagaatcagaggagctgacaccagcggtggccaaagtgtaggctacaatagcctcttcctcatctgactcctcggcgatggcccgtaggtcatccacactaggagagcagactctcagaggatcggcccccagaatgtactgggcaaagaccttcatgcagatctcctcaatgcggcgcttcattacactgataacctcaggcttggttatcagaggccgcttggccagcatcacactagtctcctctaagacatagcagcacagcacccgacagaactcacttaagagagagatgcccccgtacatggtcatcatacaagcgtcactagtgaccttgtactcattacacattgtttccacacatgtagtgaggatatccataaatatgtgatcaatgtgcgtgagcaccttgtctctctcctcatccaaaatcttaaatattttctgggcataagccataatctcatcaggggagcactgaggcaagttctgcagtgccgccatggcctgactgcagccattggtggtcttagggaaggctgagttcttggtaaagaactctatattcctgtagcacatatacatcatctttctcctaagttcatcctttttagcacgggccttagcctgcagtgcaccccccaacttgttagcggcgcccttgctcacatcatgcagctccttaatacaagccatccacatctcccgcttatcctcaggtacaatgtagttctcatacatgctctgcatagttagcccaatacacttcatctcctcgaaaggctcatgaaccttatctaagatatctaaggcattctgcaaacatcctcccatcatattaaaggcgccagtgaatttctcttccgtctgggtatattttttcagcatgtgctccttgattctatgccgcaccatgtccactcgaaccttaatctgtttgactgtagaggaggataacaacacatataagtatccgtcctcctgactcatttatcgctatctcgatgccccgctcacatgcaagagttaatctttactctatctgacatacacaagtaaatccacgtcccatgcaggttagtatacatcacatacatgtcaacagacttaccgagttctgccaggacatctttctcggggttctcgttgcaatcctcggtcacttgttcaaaagttttgagggattcttcggccaactctggaaacagcgggtctcccagactcagctgactgttaacctccttcctcaacatagtctgcaggaacgtcgtggccttggtcacgggtgtctcgggcctaaacacatgagaaatagagtcataagcacatgggtcacatacaggagatatgtatataacattaatacaattttattaaaaaaaaagggggggcacaaaccccgacacgtaccgtggcaccttggaggaagggccctcgtcaggattatcagggtccatctttctcttggcagaggactcttccggttttagaagctccacatcgaagacgagagtggcatgtggtgggatgatgcctgggtgcccagtggcaccataggcataatctggagatatagtcagtttggctctctgacccacactcatctgggcaaccccttcttcccagcctcggatcacctcctgcttgcctagcataaacttaaagggcttgtttctgtcccgggaggaatcgactttctttccatcttcaagcatcccggtgtagtgcaccacacaggtctggccgcgcttggggaaggtgcgcccgtctcctggggagatggtttccacctgcactcccatcgtgtcaaggacggtgactgcagaaaagacccatggaaaggaacagtctgttagtctgtcagctattatgtctggtggcgcgcgcggcagcaacgagtactgctcagactacactgccctccaccgttaacagcaccgcaacgggagttacctctgactcttatcagaacacaacaactcagctgcctgcatcttcttctgccgctgccttaagtcttccaaatgcgtcagcggtgcaagcccgctccccgagctcattttcagacacataccctaccgccacggccttgtgcggcacactggtggtggtgggcatcgtgctgtgcctaagtctggcctccactgttaggagcaaggagctgccgagcgaccatgagtcgctggaggcatgggagcagggctcggatgtagaagctccgccgctaccggagaagagcccatgtccggaacacgtacccgagattcgcgtggagatcccacgttatgtttaataaaaactgcgggcactggggacggtggtgttgtatatgtgaatttgtaaataataaatgagaccccatcctgtaaaaatacagagtccgtgtcagtctctgaaggacagtgtattggcatatagccaataaagagagttgtggcaaagagccatgttatggattagtaatggaaagtatcgtcaccaataggggagtggtcaataatggtcaataacccacacctataggctaagctataccatcacctataacatgaggaagcgggggtgtatagaccccaagccaaaaacagtatagcatgcataagaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatggccaatagccaatattgatttatgctatataaccaatgaataatatggctaatggccaatattgattcaatgtatagatcgatatgcattggccatgtgccagcttgatgtcgcctctatcggcgatatagcctcatatcgtctgtcacctatatcgaaactgcgatatttgcgacacacagaatcgcccaagtcaccaaagtcgtctatcgccatcccccgtaaacgatataagcgctatcgccagatatcgcgtatgcccaaaaatcacttttggaaaaatggcgatatcagttacacagaaactcacatcggcgacattttcaatatgccatattttcaaatatcgatttttccaatatcgccatctctatcggcgataaacaccactatcgcgcgacatgaatttagtcggcgacagaaatctcaaaacgcgtatttcggacaaacacacattttattattcactgcagcatatagcccattttagcgcggcacacatccagccgtttgtgttttttaacgctctccaggtactgatccaggcccacgatccgggttatcttgtcgtattccaggttgatccatcgatagggaacgctgccagcggcgcccagcaggtactgcgccttgtcgttcactttgccgcagcgtattcgcccgtcagcttcgaggtataacctacaacacggaggggaagggggggtacaaaacgtgaaattagactttttttttaatgatgttttgtccctctgtcttactttcccataggctgtaaggccctcgaggaagagacttacggattgtagttgcagctcgtcagtttgttgtgtacgacctggcgtgtcaatgaatgggtcatggtggtgacgatcccgcgaatctcagccgttttctcgggactgtagcagacttcgccgtccggacaccgcagcctgtggattcatgaaaatctactctggcattcccgaggatcgtcgatggaacatggctatcagaaacgtcgagagacaaatccagacgcaccacagaacgcagacaatcataaaaatacgtacgcgacggtgaagcgattgcacattttgaaatcgtaacagcgttccggcgggtggttgacgtttatgaattcgcaacattcttctgcgcgcacccgcggcacgcggctgtgacccaatagcagccacaacgtcgtcaagaacggcgtcaggtctttgggactcatgacgcgcggttttcaaaattccctgcgcgcgcgacgggctcaaacgatgagattgggatgggtacagaaggtgtaagtctggttattggcctcggtgaacgtcaatcgcacctgaaaagacacgctgtagtcccggaagacgtgggcccagctctccagcttcatcacacacatctgataacgcgtgccatcgttgacgacgaagcgtagcagcttggtctgcttgggcaccatgtgcgctccaaaaatcttggcgtcttccacgctgatctgcacgtttccgtcgctcggtttcgaagccgtttggggcatccgttggaggatggtctggttgcgaccgctcagataccagatcacctttttcacccaggtggagcttctctccaccaaggtctggccttcccggttgtacagcagatacagggtctcgttgcgacactcgggacccgttgatacctgctggaaccccgagaattgcaagggggaccgtgggggcgagggatagagaaaaggacagtaaaacgtcgccgcgtcatgcggtttggaatacgtcagtttagaccatggcggggacggattctggtttgccgttagcgtcgaccacggagacgccagacagggcgttgcccaaaccgcgcacagaagcaggcagtgaaagtggtgacgaagcagaagccgcagcatattatttcccgtgacgcaggctagttggcaaagagccgcacgctgaactcgaggctccgggcgtgtggcgccagcgaaccggcggcgttgaacgtggtccttttgttggtgccgccgcgacggttctgacgtctaaagtcgctgatgagcaacgacacctcggtcacgttgattctgcaagcacaggttccaaacgtcatttcataccccatgcggttacttagccgttacccgttcgcccttaccttcccgttgtcatgcacctttagcgcgtaccctcacctcttgagcacgtcaaagttgtccaagccgtggctcgcatcgtagtggtagttcaacgtgaggtccacgagctgttccacatacttgtaacgggtttggtcgggcagcgcgcgagagcacgcgtcccagtaatgcggtactcggtaataatcgtttttttccgcggtttcccgctggcactgacccagcaccacggcgcacagacaaacagacagccacacccgacacagccgcatgttgcagactgagaaagaaagctttattatgagacatcatacacatagtataggcgaggtgatggggcggggaaagagttggaaccgaaagacaaaaaaaaaagcctagtcgtactcgggatctctgagcgagacgggttgcatggcaactttcattagtttgggaatctgccagctggtgctgttcgaaggttcttccatttccgaggcggtcagttcatcgtacaccgaaacgtagtacctgatggggtcctcctcattgtccgagaggtgagattcgatggtcaaaggcgagcctctcccataattgggattcacgaacgacgtgtccaagttgccatcctttctgaaatagatgacgttctcaggatcatgtttcatgcgctcgcgggccgcggacgcctcctcctcctcgtcccagtcccgagtttccaaccgctgataagggctcgaggaacaaaatccggcggggatctgagaacctcgtcgggaaccgctgccaaacgggctgctgccgccactgtcgtccgtgtcgtccaacaggttgacggcctcttcgtcggcgaaacgaaagcggcccgggtgcttgcaacacgaggagtaaactaccgcgatcagtaccgctatgaagctgaaaatggaggtgcctgtcacgatgtagaagaggatagccagcactttcatgatttcgtcattgcgcgcgtcgtgaacggaagattcgcgggcagtggtcatgttggtttcggttgtaggttcgctactcgtggtgctctcgacggtatttctgctgctggtgctagtagggacgtttgtgctgctggtcatatttgtagcgtcgctgaagtcgatgtgaagcagcaacccgaacgcgaccaggaccaggaatgttgcgcgaaggagaccccgcggggccggcattcttgagacgtggcgacgtggatttcttgttatgtccgcgaacgacgtgtaacgaggacgtggtttccgcaagcctctaccgacgccgcgacaccaggtaggttatcaaaacgcgagcccatatcgccgccatcattgtaatcagcaatgtgttgaggtactgcacgatgaatctgtctagtgacaccagccaaccctctgcttttgcgggcaagcgcgctttcggtgacagggtgtatcgtacgtagccgcgggtcaggcgcgcgttgtagcggtacacgcagaaatctatccacaggccaacgcccggctgtagcttcggatggtggataatagcgcggtgacgtacgccgcgtggctttagaatctccacctgtaaggccatctcctccaggtagtgggtctgactgcgacgcagcgtccagttcatgtaaaagtcggtctcgccgtgtccggccacgaagaggctgcttactaatccagtctacattgtgccatttctcagtctgattgcattttttagagttatgttgccaccaacgtatcctgttacgttgattacctcgtaactgcggtcgcatcttttatggactgtataattgaaaccatcacaagtaacggtcgtggtggtgttggtacatgtggtagtctcaacgtttgtatttgtcgttgtggatatctgtgtggttgttttcgacggttttgtagaaacggtggttgctggtgcagttgcagtagagcaatttatagattctgaagtgcttttattgctgataactgttgtactgtatgttgatgtggctgtctcagtactagtggaatagttaacggtagtactacagggacattgacaggaacaggtttgattgcagctttctgataacgcggatattagtatcgtccacataaccgtaaatcgccagtccattgcaatattagttctcgctcaatgggcattaatattcctttgaacgctgagccttacagaatgttttagtttattgttcagcttcataagatgtctgcccggaaacgtagctcaatcttcatgttctgtgtgatatcgaacaatgaattctgattcactgacggtgtcttgcaacatatggtactttttgttaaaggctcgtcgtgcaaaaaacagaactatgcaggccatacaaaccaacacgatggtccatacggtgcggcttctttgcgagctatgatagagattacgtttgtggtgatgtgtattgttagtatgttgacttcctttctctctatcttcattttcgatatcggtgttgtatctagggcaaacgaaagtagcattaatagcttcagtatgattttttggtgttactaataggtagaaattttcatcttcgtgatgtcctgtgaagtaattttctttaaaacaacgtctgctgtacctgccggaattggtgatatttagatcgtacagatgtagttctgtgttgttgcacgaacgacataagtcatggtacagtgaaggtctttcatgttgagaatgacatacagtatgagaggtaaggatgcgtaaccaataccaagattgggtatgtgcgttcttacgatgacctagatgatgtccgtgtgtggatcgattgtaatgtcgtatccaggcgactgaaagacaatcccacgtagaattaccttttatggtgacattactcccttctattcctgttgtattagtttctttgaaacgtatgattgttgtctctgtgtgacaagcgttggaagagttagtacggttgtacgtggtgtacgttgtggcgctgcaatttgtaagccatggcgtgcttataagtgcagtattagtggatacgttgtgcgaagtttcatctgacgtgatagttacggtgattgttgtgttataagatgatgtagcgtttgctgttacgttggtaaaagataatatagtgttggtatttgttgaaatcaattctgtagtggcagccgtattggatatattagcatatgatgtattgaatgtagaatatacggttgtgaaagtactcaagtcggaggtaacgttggtgatgttgccaatggttgacgcttgtgaggtgacagatgtgtgtggcgtcgttgatgtgttgttattcggagtagaaaatacgctggtcacaaaggtggtagaagcagtgttgggtgatgtgatggatgcagtattggtagtagtactgttgcatgtaactctatgcagaatatagaatattatgattgtatacgccgtatgcctgtacgtgagatggtgaggtcttcggcaggcgacacgcatcttttactgtaaatccccgtccaccgtcaacaacaaaggttccgtatctaggtccgtccgcagatgttcagcgtcctgttccccgattcgttgcgatcgcaggaagcagatgaccagcgcgccaacaaagatcatcattcccgaaacccaggcgcaatggagtgagaggccggaccactggcgttttaaatccgagataattgcccggtctgcctcttgggaatccgtaaccacaactctccctggtcccggataaaagcatcgacgcgtttccaaggctcggcagaagctacgtgggtggatgatgaggtagaaagcctcgacatcgccggtatactgatcctgcaggaggtagactcccgtatctttaaccgtgagattgtacagcgtcagattttggcgcgtgcacgcgaacgccgcaccgccctgacgcgtggtttctttataggcgtctgtaatgatacaaagtggcggcatacgacgcatgtatctgctgtagatatcataacgctgccagactacgctgtgatggctagtgttaagcctggtaaccagcgtgcgtgtacggtcctcgcaggtggcacggtagttggcgagctttaggggttttttggttggttcgacggcgttcgatgaacttccctgagttgtgaacaaaaacagcgacgtgactatgacaagcgtgaggggggtgctgtaggtctgcatggtgcaaaacacgttctcgccttccttatcagacgttgtcgtcctcgtcctcttcgtcgtctgtgcccgtcggttcgatcaacggggagttatctttctgtctggagggtcggtatggaatccgttcgtagatgttctgctttttagccgcgtgttgttccagctttttgcgtgtcaggctccgataggccagacattgatctacctcggtgcccgtgttgtttttctcctcctcgcgcgcgtaaattacaaagaagaccaccagcaggactatcagcgtagccacgaacgagcccgcgccccaggccgagtatgcgcctagcatggtaatgggttctgtgatccggcatttgcacatcgcgtggcacttgctgccattgccggtattagatgatgtgttattcggactgcacttgcacgtcaaatgggtattttctgatttcacgagacagttggtggcgactttggtttcggcgcagacggccacatagcttaccaagctgagtgccagaaagcacaccgcgtgcattacacgcggatacatattaaaacaccgtgttccacaagcaccgcacacgtcaatcctccccgcacggtcttcagcccgcccatgacatgatctccctcacgttacccttcaacaccctgtagtactctgtctcggcttccggtccccatgtcctaattataacaaaacaccgtgacactgtccatctccctgtctttttgcgccgccggtcccccccaaatcatgtctctagatgccgccggccaccaaccggaggcacggcggctattggattcggcattggtgcgccgcgtcttggcctgcatgatcatcgtcatcatgattgccattagcatctggatcctgacctacgtgctgtttctctaataagaaccccggcccctgacggtaattttcctttcttctccgtttctcctcagctgccgtacgtgatgcctcacggccatctccgacaggccctctccccgacctcctggacatgtgagggcttgttgctcctcctgggattgctggtgctcttctttcaccaccacaaccagtcggccgtggagaggcgtcgccgcgtctcgttcgtcgaggccgatcgactgccgcatgagagcgggtggtattcttccgatgacgacggagaccgggacggtgatgaggaaactggagagagccacaacagaaacagcgtgggactgtccgctgtttttagctgactggcgtgcgacctgtaaaccgttactcgggtctcaagatggtttggaagttgtgactcatcttcctgtgggtgatacccaaccggacgcgagtgttccataaaagccgggcgctccggcgagaccatgccatcctcgccttcggacgccccgctcctcttctctctcctctcctccccgctgccgcggccattgccgccgccgcccataccatcggcatgtcggccgacaaatcgcagctgtcttcgccgccgcagctgtagcagttaacgtcgccggcctccaggaggagatggcgctgtgcgtcgtctcttcgtcccgtctccctctgtggtcgtgggtggtgcgagagtacacgatgggtggctctcgtctcgggggaccacagggggaggggggtaatttattattcgtattactgtaattttgtatcgcttaatttgtttagagccgcacgcttgacaacgccttgtatagccttatttatcccgatgacttttttctccgtacaagaaatggacgtcacttgagcagacacagtttcatcgaccacgacagtctcatgatctgactacctctgacccgccaacgagaaaaccgaaaagtaaaagatgaccgcgccctcggagtccttttttccttttcaatcatgaaagcaagaggcagccgagagaatgccagtaagagacgaccatcgcagacacagtacgatactcatcttagaacgaaccagcgaataaccatcacacgtacagcagaatctcatgaactagtcaaccaacgtcataaaatcttcacacaatcgtttttgcgaacttttaggaaccagcaagtcaacaaaagactaacaaagaaaaaccatcttggaattaaaaaaagtagcatcgttaccttatgaaccagcagcattcagtatatacaccagatataatatatttattaatgtatcctctctttctcctgatgtaattttgtttttgtaaattcaattgttgaaagtctctccctgggggaattgcatatcttattgatgaagaagaaatccctgccatatgtgttgtcaaactatcattatttctctatatgggtattttttttctaagaagcaaaagactagcagcagccaaaataaacctgatgaaatctttaactgaactcccagtggtctgtgtgtatatttctgttggtggtcggttgtctgaacccgggtgggttgttcggaaacggcgggacggggaaacggatggaaacagcgtcgctatatacgtgacttttgatctaaacggacgtcgctaggctgacagtttacgaattgctaaacaagataggaacaaaacaagcggggctttgcctggtaggatttcctgtggaaacaataaccggatgtgattgtggctggtacataagctggttctggctgcaagcgcttttcactgcattaggtttggcgtttgcttttgcctgggaacgctatggctataacgggaaagaaccggtttggcaacattccattgtggggggggggtacttatagcgtgcctagctatgacgttgatatatgtggatgcggataatactcgtaatgagctaaaagcgacgactggtagtaattttaccattacgcataggaaagatccgttgacaactaagtggaaaaccgtttttggtaacaatggtgatcagtggttgtgcaacgttacgggtataggtaatgctactgtgaatagtaacgcaactatttgtgtgtcgagctgtggtcataatacgttggatttatgtaatttaaagtcgggagattctggcttcttcgatctgtctcgttggttcggtgaaaacatggatgaatacagtggtgatgtgtggcacttggaagtcagctaaatgttgtatcgcttagtgaattggtgttcttacagttttcatgtaataaactacgtgtaattcgttaaatttgtgtgtttttttgttagtattctgcgtaacggtggaataaaattgcgttgacctagttagatttcctgtgtagaacaatgaccggacgtgcttggactggtacatacgcaggggctggacgtggttaccggtcactggactcggtttcgctgtagctgtggttcaacctgaacatggctcccagagctgctaggaaccggtccagtcacattttttggtgggtggggggtactaaaaaagtgtttaatatttgggtttaatgataaaatccaggttatggatatgaggaaactgaatacctcgcagggtcgaaatcttaccacagttgatgatagaagacggttttccatcgggtgggaaacatgggataacggtggtgactaataatggtacaacggtcgtcaatacaacagcctgtgtttcaagttgttcgcatacgtcgcttgtgctttgcaatatgacgcagcagactgattcgttgtacggagtgggtcatcggttgaatgacgaagaagatggtgaactgtggagagtttcggtttcttaataatcccatacgacatgtgttcatttatatctgaattttaggatgatgactatagtataactctggggaacaaatatcatacgttaatcactttaagttacgccgttaggaaaagaaaatcagtccgaatgaagcatagtcagccgaatgatacagcaatagcttgtttacaacgtgttcttttttacattatgaacgtgccttgctttttatacacacatggagacagaggtccctcagcccttgtcacgacaactccctttttctaaaccgtatgtgctccaaaccgtatctcctcatcgtcacgtgaaataccatgggaccccttttcgtcacacacgtctttccgcttacccaacgcgtcagcccgcgctcggcagagctaccatataaaaacgcaggggtttagcagcttccccagatcgctgctgccccggcgttctccagaagccccggcgggcgaatcggccggctggtcggtcggcgctcggacggatggggagaacggcggtgacttagccgcccgtggccgggagaagacggaggagccgagatgacaacagcagtcgtggaagggtcgccaagccccggtccttctcttctgtctggtcgaatcttgttttcttttttcaaccgctctttttgtcacctttttatgtgagtttctcttccgcgtctcccggccgtaccatccacccatgcagcatgcacgcgtgtatgtatgcatcgcctctcctccgtcccgactaccatcagcagtaccactgccgccacccccagcgccaccaccgctgccgtcgccaccgcgttatccgttcctcgtaggctggtcctggggaacgggtcggcggccggtcggcttctgttttattatttttttttattttttatcttctcctttccttaatctcggattatcatttccctctcctacctaccacgaatcgcagatgataaacaagagggtaaaaagaaaaaagctacagacatttgggtacctcagctttccgataactcgaagaattcaaagtcgacgattcccaacaagagaaaacagaacaaaaacaaggtcatttttatttatcctcatcgtcaacaacaactaccgacaacaacgaaacaccaccaagaatgtcaatccgcaagggtgttcctgccccctcgacgcgcctgtcgcgatcctcatggcgaggaccgcgatctccgtataggtagatgaaattatcccgtgtccggtcctgattccccgcatgccctgcacatcctgacgcgtcggtcagcagccaaacaatcataggaaatgaaccagaagaacaaaaagatcatctctctcggtgtatagcaacaccaacaacaaccgcatcgcaacatcttcatccgcaagacggaaagaaaacaacaataatgagaatgaaatcaccacaaccaagccagatttcacgtccatgagtttttattatattattatcaaaacgaaaaacagaaaaactgtcatagataaatataaaaaaaaatagaaaccacaaacgactactagtactccaatcttagatgtatatgctcctagataagatttagtattaccataatcatcgaagaatgaaagacgacgatgattccttaccgctcctgccacccggtctgtatgtagagagagaagagagaaaacggtgaatccaagatccccgggtcggcgtcggcatgccgctgatcgcagtggccccacctcggcatgccggcgccgggcgaggaattgctcatgaaaaaaagtatctttctgtaaaaaaagaaaacaatacatgattaaccgaaaagaaaccaacaaaaagaacccgagatcagtcgatttcgatcactacgataaacacatggaagatttcttgaaaaaagaaaagagaaagagaccaccttcccggcggcggacacgctcctctccgtcgccgttctgcaccatgattcgatcaataacaacatcatcatcggagaccatcttttaatcaatcagcgttgcagtagtcgactccctggacacgaaggagtcatccatttttatcctcgcacttcttcgctctcaaagccgcctttaaagttgaaatgaaaggatggaaacatggaatacagttttaattgcacgtatcaccattttactacaaaaagaaaaaaaaacaacttacacatagtattaccttaggtttacggataagtagagtgtaggcgtttttgaaacagttcagccaatgcaatcttgtctcggcataatcactctttctgcatataatagtagtagtagatttattcacatcaacacagcgaaaaactccagcatcaaagtacacctagagacagcccttaaaatatagtttgcagcttttagatgtacttacaccaaagaagattaccgtccttacgagaaaacagatactcggatataggaatcaagacagctctgcactgaaaacacactctcctgtcacgacaccgcgccacaccagaggcgtacgcgtgacttcatcgcaacgatccatcgtgatgtccctcgcagaacctaaaaagaccaaaaaaaaatcttggaccacagttgtcgatacttgaagacaatattctcgtgagaactttgagattcgcacttgaaacctcttaggatccacaaaaacaacaacctctgtatggaaaatgcgctattttatctcagcttttctcccaaacctcggtttcttcctattcttatgttttccctagtatatttgcctccttataagaaaagaagcacaagctcggtcgcacggattattccttctgctaatctattattttgttccttttttttttctttgccttcaccctcttcactccctgtagcaacacagagtagtagacacaataaatgagaagtttgcatgcatttgtcgtgtccgtggtttgttatggcgtgtggagtgctcgggatgggtggacgtggggacggattcttgaggctacaaagatacgcggagacgtcgtggcgaggggatgggtttattggatatcggtgaagcagcgtggcggcgaaagacgcgatccctgggctggtagatccccctaccccgtctaccagggacgtttatcctttggacacgtaaatgtctcggccggcatccacgcgccacgttcaccgcgttgtgcccagcgccatgtgcgggtcgtttcggcgtgaagttggacggcgtagtttcggggattgtgaaccgtggctgagggtgtagatgggacaggaaaaagcgtgtgatctgaccgaggcgaagcatgtgggtggtgcgatgcggtggatgtggcggggtgcggcggtttccgacgtggagatgtggagatgggggtgatccggatgcgtggcaagaggcctcgagcttgggcttctcccgcggatggacgttctaactgtacacggcggccgtggcctccgagtaaaaaaaccaggtgctgacgccagacagagacgccgtcctcggaatcgtgtgcgcgaaagcctgtgccgcggcagcgtacgacgttccagtcagcgaggccgtcgcgttggcgcgccaacagtaaggtgacgacaggttggcggcccatggttccgaagcgtccccacatgcaccagcagtcggcgtcaaagtcgcttgcgctgtcggcccagtcgccaccgccgcggcggatttccgcgcgggggacggggtagccgagtgctgcgccctcgccaatgttgtgaagtggatgcgtgagttgatgttgattctctgtgggaaaatgagcgctgtcctgtgggttggtgttggggtatgcgagtagtaggggttgtgtttgatcgtagaggtgttggcgggcctgtgcgcaagcagcgtagtctgcggcgtcgagctccatctgtgtgcggtgttcttcgtcggcgtgtttgtccgaggtttggacatgcggttgtgtgttgctgtggtgtaagggtaacgtgtgttgggcgtctgggtgaagcggcgtggtgtgggtgctgtttgtgtctgtggctggcatgattgtgcggcatgtgtgtgttgtagtgggtggaggttaaataggtgaggtgggttccctggtccgcgccgcaaactgtccccgtccccaacgtaacctcccctacgcggcgcgaacagccccggccccagcgcaacccccgtccccggccccaacaccgtcccgcacaccccccgtctccgcaacaccccggcatcgccggcggccagaacgctcgaaaacccccgacaagcgcagcgccgaaacgacacaggcaaggaccgtggaacgcaccggcagcgcgccgaaacaccgtcccgaagcccggtgccgacaacaaataccgtgggacgacacgcaccggcagtgcgcaggcagcgtcggacacaacacgcttacggccctcaacactccctcgaggacccaccacgcggccccgcaccggcggtgttttgggtgtgtcggggcgcggccgggtgggtgtgtgccgggtgtgtcgcgggcgtgtgttgggtgtgtcgggggtgtgttggcagggtgtgtcagggtgtgtcgcgggcgtgtgccgggtgtgtcgtgccgggtgtgtcgcgggcgtgtggcgggtgtgccggcggggtgtggtggcggggtgtgtcggcggtgtgcgcggcctcggggtgtgcggcttcgcaggaacgagtgtgtggcctcgcggccgttatttcccccgcggtccccagggccgtcgtccctcgcccccgggcgttgcttttcgtgtgtccccagggacccatgctgccgtcccccgggaacttcctcttttccccggggaatcacacagacacagacacgcgtcttcttttcgccgtgcgcgccgcacgtcgcttttattcgccgtcgccgtcctccgcaccacacgcaactagtcgccgtccacacacgcaactccaagtttcacccccccgctaaaaacacccccccgcccctcgaggacccaccacgcggcccggaatggatgtcgggcgtccacctagatgggtgcgcgcccgggaggcggctgtgcgctccagtggtacgcgcctgccgcgcgtcttccttcgggtagctgcctttcccagtccacggccttccagactgcgtggcgccaaggcggcgccagcacgcgccgtgcacgtcgctgcctataaaagccagctgcgtgtcgcccgcggcacacgggcgacgaaggcgtccgcgtgtctaaaccgcgtgctcgctgacgcgggtttgcttcctatatagtggacgtcggaggtgtccggcgcccatggcccagcgcaacggcatgtcgccgcgccccccgccccttggtcgcggccgcggggccggagggccttcgggggttggttcctctcctccttcttcttgtgtgccgatgggagcgccgtcaacagcgggcactggtgcgagtgctgcggctacgacgacgccgggccacggcgtccaccgggtagaaccccgcgggccgccgggcgcccctccgagtagcggcaacaatagcaacttttggcacggcccggagcgcctgttgctgtctcagattccggtggagcgccaggcgctgacggagctggaataccaggccatgggcgccgtgtggcgcgcggcgtttttggccaacagcacgggccgcgccatgcgcaagtggtcgcagcgcgacgcgggcacgctgctgccgctcggacggccgtacggattctacgcgcgggtgacgccgcgcagccagatgaacggcgtgggcgcgacggacctgcgtcaactgtcgccgcgggacgcgtggatcgtactggtggctaccgtggtgcacgaggtggaccccgcagccgacccgacggtgggcgacaaggccggccatcccgagggtctgtgcgcgcaggacggactgtacctggcgctgggcgccgggttccgcgtgttcgtgtacgacctggcaaacaacacgctgatcctagcggcgcgcgacgcggacgagtggtttcggcacggcgcgggcgaggtggtgcggctgtaccgctgcaaccggctgggcgtgggcaccccgcgcgcgacgctgctgcctcagccggcgctccgacagacgttgctgcgcgccgaggaggcgacggcgctcggacgggagctgcgccggcggtgggccggcacgacggtggcgctgcagacgccgggcaggcgactgcagccgatggtactgctgggcgcgtggcaggagctggcgcagtacgagccgttcgcgtcggcgccgcaccccgcgtcgctgctgacggccgtgcgtcggcacctgaaccagcgtctgtgctgcggctggctggcgctgggcgcggtgctgcccgcgcggtggctgggctgcgcggcggggccggcgacggggacggcggcggggacgacgtcgccgccagcggcgagcggcacggagacggaggccgccggcggggacgcgccgtgcgcgatagcgggagccgtggggtccgctgtacctgtgcctccgcagccgtacggcgccgccggcgggggcgcgatttgcgtgcctaacgcggacgcgcacgcggtggtcggggcggacgcggcagcagcagcggcgccgacggtgatggtgggttcgacagcgatggcgggtccggcggcgtcggggaccgtgccgcgcgccatgctggtggtgctgctggacgagctgggcgccgtgttcgggtactgcccgctggacgggcacgtgtacccgctggcggcggagctgtcgcactttctgcgcgcgggcgtgctgggcgcgctggcgctgggacgcgagtcggcgcccgccgccgaggccgcgcggcggctgctgcccgagctggaccgcgagcagtgggagcggccgcgctgggacgcgctgcacctgcacccgcgcgccgcgctgtgggcgcgcgagccgcacgggcagtgggagttcatgtttcgcgaacaacgcggtgaccccataaatgatcccctcgcatttcgtctttcggacgctcgaactctcggtctcgacctcaccaccgtcatgacagagcgtcaaagtcaattgcccgaaaagtatatcggtttctatcagattaggaaacctccttggctcatggaacaacctccacccccatctcgccaaaccaaaccggacgctgcaacgatgcccccaccgctcagtgctcaggcaagcgtcagctacgcgctccgatacgatgacgagtcctggcgcccgctcagcacagttgacgaccacaaagcctggttggatctcgacgaatcacattgggtcctcggggacagccgacccgacgatataaaacaacgcagactgctgaaggccactcaacgacgaggcgccgaaatcgacagacccatgcctgtcgtgcctgaagaatgttacgaccaacgcttcactaccgaaggccaccaggtcatcccgttgtgcgcgtccgaacccgaggatgacgacgaagatcctacctacgacgaattgccgtcgcgcccaccccagaaacataagccgccagacaaacctccgcgcttatgcaaaacgggccccggcccacctccgctgccgccaaagcaacggcacggttccaccgacggaaaagtttctgcgccccgacagtcggagcatcataaaagacagacccgaccgccaaggccgccaccgcccaaattcggggatagaaccgcggcccatctctcgcaaaatatgcgggacatgtacctcgatatgtgtacatcttcgggccacaggccacggccgccagcacctccgcggccgaaaaaatgtcaaacacacgcccctcaccacgttcatcattgaaagtctctccagtccatatgttgtcaggacgtgctgtcgttctccgcttgctgcgaagcccgttcttccgagtcgtgtcgctgcgtccagcgtcgcgcccaagatgggaatttgggtcttttcacgcgtagcctcctccaccacggctgctgatcgccgtcactaaggaccgacacggaggatgacgaggagcttctccccgactccgcggtccgcgaccggctacgtagcgcgtgtccctgccagtctccgcagttacaccacacgtcgtgagcagcgtgcacctgctgccgccactgggcctcggcgtgctcaggccacccgccggagcccggtctgagctccgacgcaggatgcgcgtactcaacgtgcgccttccagtccatacagcaacaccataggtcgtgcgagtcgtcggctacccgccgccaggccagttcccgcatgggaaggctggacacgccgaccgagaggtcaccgagcccggacgccatctcttcttcctctccgtcgctgtcattaagcagccaggtcacctcctccgctccgcggtccgccggtctcgacggaccgcgccgccgtcggcaacacggaaaacagcacgccagcccgagccgctaaggccgcatgcccctgccgcccaactgaacacgcataccccgctcaactgcgttttgccacccctgtcagtgctctcgctcgagcaccaccccgcatctcccaacctttttccaataaacgaaaccgacatgacacacgtaatgggtactcgtggctagatttattgaaataaaccgcgatcccgggcgtctcagcacacgaaaaaccgcatccacatcatagacaagttacagtccacagtcacatacacgataaacaataccaacagggtaatgtttatggagtaaaacactattgtccaggccacatgcgtgtatgacttccgcaccatcccgtactgcatgttccacatgtacgcgctagacgtgtaatccactcgcagttcggggacgcaacgcagccagatcacatccccttgcagtaccagacgcagggctagcgtctcgaagatcggcatcacatctaagttccgcacgttccactttaacgactccccgggaacgaactccacgtcgtcggcgtgtacgtacaggttctctcccacgccgccataatcggccttcggatcgaagacgaaccgactcatgttgcccacgatgctcccccgagcaaacaacttgccgttgtcaatgtagcaccggttgtcctcgatttgaaaccagggatgcttggccgtggacttccagggccggagcgcgtcttccccggctttagtgattccatcgggcaggcggatcaagggacccatggaggtccaaagacccacccaggctttccagagattgttcatggtgaaacagcgtgtggactgtacgctctttcccaatttatatcccagagtagtgacgtgagcccagccacctcccagattcctgacgttttggttgtctttcctgccaattcctcccgtaaacttatgattatcctagcccattcccgataaaaatacacggagacagtagatagagttacgaataaaccggtttatttattcaagtgtctcaggagattattgaacgagcgtggataccacgccgtcgtcagttcatggtggcattgagcagccatagcaccagagtcccggcgcccggtatcagacacgctgacctaccgggcgccttcgagtccgtaccccgcggcctgggtgttagagtccgtaccttgcagcccaggtaggtttcaggtaccagctggttcgtacctgttaaataaatcgcagacgggcgctcacccctacggtcaggagcacaagaacaaccagagagaacagatatacgagcagggttctgaacagcagaccccaattgtcgtctctcatgcttcgctgaaggtaccagttgatggtctgagagctatagtccatcctcacctgaggaacacacgcggcatatttcttggggtctccccacctcgtagacaacgtgatgtccaccatatccacggtgtgcgtcaccgggtgcccaccgatgttccactcgaaataggctccgcgctcatcatggtggtactgctcaccggacacctgcagtctgtccatgtaagattgagagacgatacccacgttcacaaagtgtttctcggtgaagttgcccgacatcctccccttgaagtacagcatgcccatatggaaccagcattggttctcctccactcgaaagtgggccgatctgatctccgataccaccacatccaggggccggggcaccgagtccgcgagtctcaggaacaagacggccaggatcgcgagcaccaacaccggcttcatggctccgaaggtccgctgctcggctccgctcaccgctccggtctggctgcagcagtgcttcgctgagaagtagcgtgtggactgaacggtgtttttgaatatatagcgtttcttggtgacgttgtttcccctacgtagtaggcaactacgtgccaaaagaggcgttacggtactttccgtactgggatttccaaaccgggactttccacacggcggtttcaacaccgggacttttcacacggtgatttcggcaccgggactttccgcacggcggtttcgccaccgctgacgttctcatcgccgcccacgtcaacggtggcgacaccgtactttcccatgcggtttataaacgtcaagagtcacgtcagtcgcccacccccattacacggcgatatcccgatagggcatgaggggacccgggtgtcgcgacatgtcgacgacaggtgcggattagtggtcgtgtcgcgacatggacgtgcagggggatgtctgtcgcgatagagttgatgtgacagcccgctacacctctctgtcgcgacatgcatacacaacgggccggcttgtcggcgattgtcgcgacatatcgttatcagttagcgaccggagttgtctatcgcgacatatcgtcgactatcgcgacagaaaaaataccgttcgtagagaatgccgtgttgaaggaacgcgcttttattgagacgataaaacagcatcaggagccacaacgtcgaatcccacgtccagtcgattcgtatgttatgctgcacagcaatgctagaataacaaccagcagggtaatcccgcaacataaatacaaagtcacagcgaagaatccgtgtcgttctatcaagcgaaacgcgttccaaacggccccgtcacagacgcagttattcataagcgttaacaaccggtggctaggatgaatatccaaatcacagggcagtagccgacggactcgttgacaggtcagcctaccctcaaggttcctatcgttcggacgggatttgtgcgttttaggcctctttttcgccgcctgcaagcattggtgcgcaaagtcctcacccagctgtttccagctatcatctgcatctgtgcagtcccctgtatcgttgtaacaaacgggtctgtgcgacttcgttctcggaacacaagcttgttgtcgcggagacagagagagaagggttttcgggtcacgcgaagaccgctcaccgggggtcggcaacgcacacatcaacagaaaaccgagacgaatcaagagatccatagtgaaggagtgatatcgacgtgcttacgaaacggcgattatatatgttctcaacaataccgccctacgttgtatgatgtaacgtgtgacgtgagtctgatccaacactgaacgctttcgtcgtgtttttcatgcagcttttacagaccatgacaagcctgacgagagcgttcatcggggcatgaagtacgcattacacaaactccatatatttgttacgatagaatacggaacggaggaggctttcgccacacctatcctgaaagcgttgcattctttatgataggtgtgacgatgtctttaccattcccacggctgctttgcgtgatgatgacattcatcatgtatttccattcacacataccttttgtgcatacggtttatatatgaccatccacgcttataacgaacctaacagtttattagcccttgacaggataggtcaaaagattatatgtaggttttccggtaaaccgaattgtgatatttctctgcaggaaatagaacagcctggtacctataaaacggacaatgcagtactgtagcagcgtaaccaagtaggtccacatgaacacgtacaaaattatggtaagccatcgtttttcataccacagcctgtagctgtcgtacatgaatgaggacggtcgaggaacccagggtagttgtaattgggggcgacattcgtactgtccagaagacaattgcacgggtttcagtgagatgagtactttagcgatgtcggcgggggcgctacgtttcaccgtgacggtgagaacttgaccgtcgttttgtatttcatgaggcacgttatacaagccactggtatcatgaaggatgacctctgatgcgatgtgaggattaaattgtccctcaaaccgccaaacgctggtcatgtttccaccgtcaattacgcagctgacggtgtgagataccacgatgttggacttaggtttgggggctaattgcctttttacaaattcccttctgtattgcaggtcctgctgccactgcttttccgtgcggaaagtcgccatgtcttccacacgtgtggcgacgatagacgccaccaaggtagctaccagaagcagctggatccgcatggcattaccgtatgtcaattagaaagttgagcggacacggttatcgttcctggcggatataagtatataaacgcgagttagcctttcccgtccgttttgtacacccgttccccacacaaatgacgaatacgacctttttttttataaaaataaaccacgtgtattatataaaaacatttacatagaaaagagacacacggatcaacataaggacttttcacacttttggggtacacaggcgtgccaccgcagatagtaagcgctggatacacggtacacagtcctggccagcacgtatcccaacagcagcaccatcgccatacagatggcgatcacgaccccgagctctaagtgtctgtattcatagtgtagtcgccgcaggttatccactgaattcccgtaactgaaataacgtatatggtaccgaggctggcaccacatgggtttgcatttggtgcacggcaccaaatgcagagtgagatggtccaagtccgtgggcacccactggcgcaaacggaatacggcttcggtggtctccacgaggcactccggggcgtgcagacggccccactttcgtccgcgacggcccgaccagccgacccgagccactatccctttctcgggatagaacgtaccctgtacacgccacacagcgtccaacacgccgtccttgacgacgcagctggcctgatagctggacacgttgttaagcggcggaaagcgaaactgacgtgccggcggagccacatagttcggttcaccgtgttgtcgcggttcgtcctccctatagtaatagtagtcgtcgtcctcataggggttgccggcgtgagccagcgttacccaacagcagcccaggccgacgaggaggcgcagccaccgcctcatggcggcttcgccagtcaatcgtctttagcctcttcttcccgtgaggtccttccggtggcgcggtgccgacctcggacccagggacgtatccacctcaggtacacacagcaggctacctggacaccgaagctgaacaaggctacgtgtttcacaaactgcaccagtaccacatagaggaatgtcaggtagcgtctctccgcaaacagccgttccaagtctgagggcgttacccgcagcggcaaccagggcagcctggacgccggccggcaatggagcacgctccggttacaggcactgcaggggtaaacggttaacatcacgtaagagagtcgtgcgtccacctgtgggagctcagtttcgtaacgtagagccccgtcattttccagctggggtgcgccgaccttgaaatgggtcgcgctccgctcgttaccccaggtgccgtaggctctcggggccgtatcggagaagttgccacgcacaagccaggcggccacgagtaccccgtgctggacgtaacattcggacacggaactggagacacggtagccggacacgtccccaaacccgcgagggtactggggcagacggacggacttgctatttgacaacggacagatacgagacgacgaggacgcagacgactcgtcgctggaccacgacaaccggagcgactccttggagcggctcgagagtacacttactgcgatcagacaccagtgccagaagaaggaacaggtggacggggaccacaggatcatagccgccggcaccgcggccggccgcaggaagccgcccggcgcgtcgtctgtgtgcgggagccgaaacaccgtgcctctttatatcgtcccgacgtgacgcgagtattacgtgtcaggggaaacccccgtcacgacgaacgtgatttgtaagtgacgcggggtgctgacggggttcggcccgagaggtgacggagcgcctcacgtcagtatgatgtccgatccgcgtcagccccgacgtggttgtggtcaccgaaacccacgtttatatggacgttgagagcagcgcctgaccacatgattcatcataccatttctcggaatcgggcccatgccgggaaagcacattccttttcagtaaacaacaatgacatcataacaaatcattttattcgcgaggtggataataaccgcatatcaggaggagggatcgggtgatgacgcaggccccgcagaacagtccgaaataaatttttagtattgccccatagtcgcctagataccagaggtacgttaagttcatcaaaacgcccatcggcgtcccggaatcgtataccgggcacacgaagcgttcataacaatcccgggaggcgagtgttagggtagcagagtagtttcggggtcggtttccttccggcgacgacagttccgtgggcagcagaatgtacagcgcctcggtagctgtcgcggtgccttccacgaggatgggctgccggtgcctttcgtgattttccccgtcgtgtagccaagccgaggcccgcaaagtcttaggcgaggggaattgtccatagagtttcaccgcacccttcagtacatggttctgaataacacagccgcacgtgaagtaggtaggttctctcgtctcctccgtggctgccgccaccactcccagccaccacaacaggcagatcgccagagggttccggaggcttccccggcgtagcatggttttgggttaaagcaaaaagtctggtgagtcgtttccgagcgactcgagatgcactccgcttcagtctatatatcaccactggtccgaaaacatccagggaaaatgtcggtgcagccaacctttcacatacagcccccaaaacacttgaatcactgccaccatcatcagcgtatactgcgccgacttaatcgtgagcgcgtagtacgccattagacggcgatcttcgaacaatagtcgttcgatgtcctctaacgagctccacaggggaacccaaggcacgaggcaccggggttcgcactctacataataagtttggcattggtggcagggggaaaagtagaacaacacgagttttgtgcgttggggaacacgatagtcccggagccagtagcgttttgcgacgaggctttcggagacgtcctccaccggcgtcggcactcgatccgcgtagccctccagcgtctggtagtacacccggggtgtcggcgtgggcacggacaggttcccgcgcagggtccacagagcctccagtcgaccgcccgatcggagcacgcagcgcgcctcggaatactctactcggtactccgaaacatcggacagaggcggtaacggctccgtctccaccaagggcggaggttcatcgaaaagagtcaaggataattcaggcatactacccgcgaccggggcccagagggctagaataagcattacaaggttcattctgtcttacaagggaaggctgttaccctgtctagactcaaaagctgtaaggctgtcttatagcatgtagtcttgcacgtcacggggaacagggtggtgatctagtgacgtcgggagaacacggtgttttagggtgcgggggacaaaggacagtacgacagattaggtgatagaaacgtttttttttatttatgaaaaagccagtgtgccgtgcggcctagggccccggcgtagtttggataccagatgggggccgtcaggggtactaccacgagcagaaacataatgacttggtccatgtatagcagcatagcggtgcgcagcaggtcgccgtccgtgtagcaatttgacggtgagcgataaagcaccgttaatgtgtcgcggataagcacgatcttgaggccgtagatgaagctcacagtcagtgctaaaatgatgcgttggtatggttcccaggactgcacggcgatgaagagccagagtatgggaagcatgaagcttagcaaacagaggatggctaaccgtcgttgcatgttccaggccatgagccaggctaggcccgtacaccagacgcagagcatggatgacaggacataggcctggattaccacggtgcgatcgaaacacagcccgatggtggacacggatatcgtagtgagggtggtatataccatgaccagcatcagggtcccgggtcggcgccgacgttccagccagtacgcgtggcaacgcagagcgcagggtagcagtgtgctccagaagggcaatgtatcgcgcaggtagggggccgtcacgcgccacggtatgagcatgaaaaggatggtagtggctatggtggcgctggtctggaacacgacagtgccgtagagacgtaccatccagagaaagtgttgaacgctccgcagggtgtcttcatctttggtgattacggtgactcgacggatcggcggtggtgacggcggcgacacgggtgggggtttctctttcttatggccgagtggctcgccttggtgaaactggatctgtaccatgacgggtgctcgacgaacagtcgtgggggctttaggtacccggcaagttttatagagaaagggggacgatgggtggtggctacgagccaccgccaccttcgcaatacgaggatctgaaggcggcaaagacggtcgtccagggcaggcgccagaggttgggactgagcacgatcagcgtgattttaaacatggtcaccagtcctacgtagatcagcagcgagccgcgtaacgtctgagcagccggcagttcgtcgcggatgtaacgcgtgccgtagaaagtcacggtcatcataaggaagacgatggcgccgtagccgtagagtagaatacgctgatgatggaacacggtctggtcgccgataacccagagcgtgatgaaaaaaacgctggtgagtacccgtgagcatatgagctcccaacgcttagcgcgaaagctgtccccaaccatgacagcgccggtgcaagctatccacagcgtgaggaccagtgtgtagtcgatgaggatggcgggcaggtcggagcaccaggtgtagaaaaccgtggtaacggagaggaggcctacgtagcccatggtcaataccacgtcgtcggggtgcctttcgccctgtatcaagaccaaacaccagagaagggagggggcaaaaaccagcagcagaggggaagattcatgttgacatatgttgtgggaatcggggatacccagccaaatcattccgcagaaagccgtactgatggcgatgtgaaagaccactagggcgtagacccggacgaggacagcaaaacggcgcagccacataaggccgtggtgcagctgcaggagggaagcccattgcggcgaatgtagcgacggtagcggcgggtccatgaggcgggtgatgcgcccgagtgaacgggtgagcgtctcggtggagtcttcttataaaccagcggagctcaggcagccttgctctggaacgtcgcagtggtggtgttgaggatgacgctgagcgtgccgttgtcaatcaggtaatgatgataggtgccgagcttggccaggtagctgaacatttggtcccagcgtgccgaccacaccacgggcgtgagcatcaggagtgtggtgtgatagattagtgtttcggtggcgtaaagtatcagcgagctgcggatgacgtggctcacgggcattttggtggcgatgtagcgcacgtcttggaaaaggacggccaggatgcagcccacgaacacggtgtagagacacagcaaagtcttatgtaaccaggtgtaagtagaagccaggacgctgaccatcaccgtcaaaagtgtggaggtaaaaagcgcgtcacgccacacggagctgagacggtgctcccaagccacgccgttgcaggccacgaacaacgtccacgttaggatgaggctagaaatgccgatgggcgctgtggcgcacaggttgagcccggcggtggtgaacgagagaagcgccacatacagcgcaaacaccaggccgttgctggggtgtctgtgatcggtgagctccagcgcgcccagaaccaatactggtgtgcagctaagcaatagcggcgagggatcgtcgctgcacttgtagcccagcgaggggtaacccagccaaaccagcgcgctaatgagtacgctgaaagcggtttccagcgtcagcaatccgtagacacgcatgacaatcgcggtccgccgtagccaacacacggcatcttcggaaactgtggacgctgtttccgaataccgggaggagatcgtgcttccctcttccaaggatcggaaagtagcgtccgtcgtttccgcggacgcggcttccctggtacgctccgtttccgacgacgcggtttcccgctgcgtggaaactgtctccatgtcgggaccgcagcgcccggcggcgtatccgcaaggtctcgaagctacagcttgtcagaggaaaagtaggtttgcaaaaaggtgcgcagggtcatgattctcagcaccatcagcagagtgaaaaccagactgagaaacaccttgacggccgccaaaagcgcgcgttccagcggcgtctcgtagcgtacagccagggccgcttcgtggaaatgcgagacggctagacaggtaatgagcacgctgaaggacaagacgatcttaaagcaccaggaccaaccacgcctcaagatgaccaccacgattgccgtgaaggtcaacgtgatcaaagcatggacgaccacgatctgacggcggacggtacgttcgggagccaacaacgctacgccggtgcagctgagaaaggccagtaaggtgaacaacgcggccgagatgaccaacgtaccgtccaggcagagacatatcacgatcaacggcggcacgtgaagcagcgtgtaaaagagcagaacgccgatattgctgggatgcgatgtttcgtaacagtgaatgaagatcactgacgtgacgggtatgacaaagacgaggctgggcgaggactccgtgagacacagacgagaatggtgaaaccacgtcgcgggcgccgcgtagcagaaggcgctcaacaacgcggtcaagccggccagctgccaacccacggcgccataggtgtgcagcgccacgcggcaacagtcgacccaagccagactgcgggtcgccagccgggtctcttggatcccggggggcacgtagatgaccgtgccatcggtgggtacttgaaaccctttttctcttctcatggtgcgctgcgttctctggaaacggctgctctgtccgaaaaccagttccgaacgaaaatctagggcgagagggtggacaacggcgtcgacgacgaagcatgggacaggtcgttcggcgttaacgtcatcgcgtcggacgacggtagttctaagagacgtagatcgctcagcaggtcctgacagttgcggattcgcaagatcagaaaaaaaagggaaatgaacgtaataaagagctgtagcgacgtatgcgccacatcgcgtggcataagaacgtgacggacgaaaaggacctgctgcgaaaagtgaccggcgaagataaggcccaccgtgctgtagaagcccaaaagcagccgcaggggccaagtccagggccgcgtgaagacgatgagaacgttgaccagaaagaccacgacccagacgccgttgatgagggtaaattgatcggacagggtgcagttgtcgcgacagatgaagactacttccgcgcagagcaaggtgatgaccaacgtgagcacaaacgacgtcaacacctcgcggggctcctggcaggcacacgtgacacctagcgccgggatgtgcgccaggaggccggcgagtaatagcaccagctgtcggaacggacgacggcagcgcgggtgccggtttcgctgagcgagaaccggtcgctcatagcggaaatacacgaagagcgcggaggccacaggcaccaggaggagcacctcgggcgcccagacaacgtgacaaggaaagcccggacgcgacttgagagtcgctgtagggaagaccagagagaagctacccaagacggccaccgccgcggagatttggaagaggagcaagccggcgattcggacgacaacctcgaagcgatgcacccagcccagcacggccaccacggccgcttcatcatagtcgtcgttgttgccgctgtcgaacagccgccgaaacacgatctgtcgctgggtcgcggtgggaaagcgcagacccatgacagccggaggctatatgaccgcgcgtctaagacgcgagatccgtggggggacttttagatgtttgggcggcccgcggttctaacaggcttgattggtggagacggccggcgcggcgggtgggggaaacgacgagtttttccgttacgccatggttcgcgtgaggtttctctgtacctcccgcaaaaggtcacagcccgaaatggaggccgcgttggtggccccggtggcgcgtgacgataaccaggtcatccaagcgatgagtttgtctaatgagtcctcggtggtgaagaggatgagaatgagcaggtacaggtacaccaggttctcatagagacacaaggtgagcaggtcagcctcggaccacgcgatctcaaacaggcgcgtggtgtcaaagaccgtgacgaccagcatgaagctgagcgccatggcgtaatagcccaaaaaaagtttgtgccccaacggtacgggctgcaggtaaagtgcgatcaagaacgcgataacgccgatcacaaacagcgtgacgatgacctgccatcgacggtgattatggccggctagacccgtgacgcagctgcagaggctaaaaagcacgcaagccaagaggcccgagaaggtcactagcgtagaggaggagcaggcgctggccacgatcaccgaaagcgtcgtgagcacgctataaatggtgagcaggccagggctcggtggcgacgtgaacgatccttcatcgcgtttgccgtgcagcagggccaaacagatggtgggcaccatcaaacttaagggcggcataaagccggtgcaacagagaaagacggtgcctttaagatgcggaaaagccagcaccaggcccagacagagcaagaaggtgcaggtgccctgcacggccacggtgctgtagacccgcatacaaagtaaaaagcgacgtacgtcgttcgtcgacacggaggaaatcataatgactccgcgcgagggtcgcgggggtgggggcgcccaggccgtcccggtggcctctgagttcggagacatgacggcggtggcgatcaaaaggcgcgtatgagaaaccgtttatagagtgtaatagaatcaccgtcattcccacacggcgttcccccataaagtcacgtaacactcgagtaagcgtgaaaaagctttattgttgaataaaaaacacgagtacaacaccgagttgcggtgtcctgtctgtctactgggtggggaaggttcatcgtctgtctctagagggaaggtggggaatgtctaagcgagcgggagcgtgtcatctcccccatctttttacaacaagctgaggagactcacgccgtcgatgcgtccgccgtgtttctcggcgtactgctgcacccagacgtggccgctaaatatggcgacgctcatgtttaggagactcatgacgatggtgtacaacacgacgctgacacagacgctgtttttagacaacgttccacgctggtagatgagatccagggtctcgtaaataagcacggccgaagcggcggtcaccaccaggacgtagagtccgctgtagatcttgctgacccacagcacgggcgaaaagtaaagcaataggtaaaagacgatgacggaccagccgtagccaatcccgatgactttccagcgcgtgggattgttgccggccaggtaggtgagaccgctgcagagaacgaaaaagaccatcaccagggcaaacgacagaccgatgacgcgcctttctccgcaaaagcccgtgcacacggtgatgccggtgttgatcagcaagcacgccaccgtgagatgagcaaaattggtggtgtgtgggcgaaactcggcgaaaccgcgtagcatagccagcgtggacacgggtacgatggaggatagggctggcactatgccgttggcgcactgtccctgcacatcggggaaggcgagccaagccagcaagcagaccgtgagggtacaagccagctgccacacgagcccgtgatagacctccatgagcagcttaaagcgtttcaaccattggaagagctgctgttcggccaccagcgcgtggctgcgatggagcggcacgatggtgaccgtcggcgactcatggtgttcggaaaccgaggcggtgtcgcccatgctgccgcttacgaccgctgtcggtctaaggtaggcgtcgatgaaacagtccgtcttatcagcacccggttaccgcggatttgattgacgtcacgagtgtggtcaaaccgtggcggcaccctgtatccgacccgtcgtcatgggctccacaaccagagcctcagaagatggtacatgccgatgaataaagccacattttcgacatagaggcgtagcgagggctgaaaactctccgggaaagaactctgacaggtgatcagggacagatcgtgaattagcatcagcgtcaccgtcaacagcgtcgtcgcgtgtaaaccgagaaagaacggggccgcggcccgcagcagccaaagtcccagcgccgtagcgcagagcagagacaggaccgacggtagccacagccgccggagagacgcgccaggatcgcaacccaaaagcgaggcccccaggcagctgagatctaccgccagggcgagaagagccgcgccgacaaaggcctgcggcgacggctggcacatcagcaaggtcagaaaggctagcgcgtgcggcaggcagtaagccaacaggagtgggagtttgcggggacaacggtcgatcgacggaccgcgtagcagcaggaacaggcagccgacgggcacgacgaggctgagatgagaaagcggcggtgggtcgtcgtcccgtccccgctcgcatagctcggccaccggtggcggcatgagccaccagctgagcacgctgagggcgacggtggcggtaagctggaaggcgacgaggacggaggcgcgcagccataccgccagcctctctaggtaggggactacctcctcgacggtccattctagcgggacgacatgaagcatggcgacaagcgcggctgctgtgaaaacgggcacggttttataggcattaggacttccccgtcgtactggcggctgtcaaagtcccgttgtccaaaggcgcgccgtccgaaagactaatccaacggggacccgagagcatgagcaacaacgtgagaaagatggccatgctgtccaggtagagacagacggcgtgacggatgcattggttaggtgggcagaaaaagatgaccataagactgtcgtaggccagaatacccaaaaagaagctgatagagaaggcgcacaacgtcaccactatcttctgcagccaatcggcgtcgcttagcagagcgagcgtgaggaacgaaagcagcattaccacgtagacgcagctgatgcatttccagcgacgtcggtcacggccacctagaaacgccagccccgtaaaggagataaacaacgccagggtcatcacgtaggaacctactagtacgcggctttcagagcacatttggaagatggccgccgtcaggctgttggccaacagatagatgaaaagcaccgtggcgttactagggtgttcgttgcccaacgtgtacgtgatgaacatgcagacgatgggcacgagcacggtgagaaagaagctgtagttctcgacgcaaaagttgcggttttgtgggaaccccaaccaaaaaacgcttcccaagccgaagctgaaagccagctgaaagatgaagatggcgtacacgcgcagccatacggtgaactttttgaaccactcgagagcctccatgcgggagagcagcagcgcgttagcctcctgcgcctgcatggtggcgacggtctcggcacaaagccgctgcggcgcacctacccttctcttatacacaagcgagcgagtggggcacggtgacgtggtcacgccgcggacacgtcgattaggagacgaactggggcgacgccgctgctgtggcagcgaccgtcgtctgagcagtgtgggcgctgccgggctcggagggcatgaagtagagcacggagacaaagaggtacatgaggtccatgtacaagcagagcgcgcccgggatataactctcatactcgatgtcgtgcaggatgtcctgcgtatcgcacaccaccgaggtcacgatgacggccaaaccggctatcatcaccaggatctcacttaccgcctcgggaaaaagagaaaatacggcgaacagtaagagaatcagcgtggatgcgcccgtcaatagggaacgctgtaattccacgtcgcgggcaaacagatacgtagcgagcgtgaggaaacaaaatagcgtcactgtggccaccatggcataaatgactgaacgatgactaaagtggaagcctgacgccgtgacagccacgctggtaagcaacgtgtacgtcagtaagatccatacgtttttgggaaagttgggctcggcccaacgcaacagacctaggcacacgatggagatcattaagcaagacagcgtcagacgcacgctggaaaagagctgctccaaccggtgcggcaacaccagccagcaaaaggcgcagacgctcataaggatgaggcattgcacccagataaggatgtagatgcgcagcaggaagaccgaccgggctatctggacctgaccgcggagcgacatggcggcaacgccggcggttatcgccgagattcgtctaaatacacgaagcgaactagaaaacgcacacacgtgatttgcaaaaagaaagcagctgccggcttattattttattaaaaatttatctgtgcagaatcataagtttatgatgaataaaaacggggaaagggaatctgcttttagggacccgggtctggtccgtcgtctcccatctggtcgggttcggggatggggacctgtttcagcgtgtgtccgcgggcgtgcatggcttttgctcgccggccgcgctgtaaccaggcctctttctctgtggtcggcgagtcttccgacgggtagggagcctgggagtccatcgcttcaggcccaccgctcgttccctcgaccgtcgtgtcgtcctcgttttcgctattacacggggtttctggagtatcgcctatacggttggcgattctccgggggcggccgctctcgtcctcgtcgctgctatcgccgcccggtaattcgacgccgcattcgttgtacggaacgcggcacatgggcggcggaaagaacttgggcatgcgaaagcagcgttgtccatccacggtctgcgtggtttcatcgttatcctcccataatcccccctgtagcgccggcagcgtttcgacgctgtgagaggggaaggcccagttctggttgtcttgcagcgcgcccgtgggcagtaggtccgtgcggccccatgcgctgctgttgttgggtaccttgtcagtgccgcgagtaggtcgcagaaaccagtccagagcgctctctagctgcgagcgtgtgatggtgcccagtgcgccgtgccagcgcagcacgtctcttttcagcgtgtggtgacagacgggcagctcctccaaccgacactcgccgcgcaatccgcggtcgaagcggcagagaccacgcagtttaagcagaccgcacttgagaaacatgtgaaaattatcggcaatgcgatataggtccgagtcctcgatcttgtgtaggtagaccacgccaaacttgtcgagcagcaccaggccgctgggcacaaaaggcccgtaggccaggtaatagcccacgaggccgacgacgtaccactcgcagcacaagcgttgacgaataaagttcagaagatcgcgaaagtccgcggccggcatgtggtcaaaaggccggcaggcgcgcaggccctcgatggagcccagcatgagcaacggctccacctcggtgcgacccggcgtgcggatgaccaggttgagaccgctcatttcgcgggccgtcttggccacggccgcagcgtcagtggggtcggtgcagaggaatttttgcacatgatagcgcggttcggtggtggcgaacggcgtttgtgggtgccgatacacatattcgcaccagagtaggccgttcttggaaaaggctttgatatcactggccacctcgtagagcccgtcggtctcccagtcgtagacgtagacggtgccgtaatgacttagcatgagcacgcagggcagttcctgcgcctgcttggtgtttcgtgttagatcgctgtcgggtggacgcacggctagtacaccgacggcttccagggtgtcatcgcagcagagatagtcggcggccagagaacgtgcgtaaatctgcgggatggcggcctgttcgcgcatcactaggaaccagttggcggggttgcgcagtgctacggtggttccttggtggcgttgcacgtaggttctcagcgccggaggatcgtactggcgcagatagaggccttgcagcatcgataacgtcttttgaaagacggtgtttctaaattgaaaaacgccgtagtcgcagcggatagcatcttcgcagcgctcgtcgcgctgtcggagataggtgccccaggcttcggcggcggctttggtgagtagggacatgccggcggagccgtctcgacagcgagtcggataaagcgcgctgcgcgaaagcttaatataggagcagcgtcagacgaatcgcggctggtggcccggggggtgggacgcgccgcctacacaaagtgctcccgaaaatcgaaactcttgacccactccggagacaaatccgtattcagattgatgcgtcgagcttccacttcggcttccgaaacctcggcctccgtccggtaggcgttaacaatacgctgacccaggtgccaacgctctttctctgccaaacgccgttgctcaaaccattcgtctacgtccttgaggtcaaagacagtgtcctcctcaaggtcaaagcctaggtcttcccactcgtcgtcatcgctctcgtggccggcggccatacgcgcggcaaccgcgtcttcccctcctcttctttcaacgttgggtaccacgttgttttcttcgggttccataggttctgcgccgctgtcgtcatcatcctctccctgctcctcatcgtccgccaaggcgtcgtggattacctccaggttctgattgtcgggtacgacgtggttatcttcgtcgtcgtcgcgtggcatgggcggcggccgacggcggacgaccggcatggcgcggccgtcgtttccttcgtcttcctcttcaccgtctcccaaggaacgcggtcgacgacgttccgcgaagtcgccgcggaccacgcgcgcctgccaaatggtaaacgcgtcccaaccgtcccagttattgagcatttcggcgcgaaaacggtcgcctcgacagagccagcgaaactgccgcgcgtagtcgcggtctacgccgctgtcgaacatggtaaagtgcagacgcgccgcctcgcccatgtgtacgcagcctccgttgcgttccagcctggccgcgcgccgtagaccgtgttcgtagcggcgacgcacgtacaccttcatgaggccggcgcgaaaaagttcctctaggctgtcggccagccggtagatttcaccggctagacgctgcaggggcggcgagcggtccagatgcgacttgacaatcaccacgtaaaaacgacagaaacggtcgaagatgatgaggaaggacgtgtcaaaaaaaccaccggcgcggtaagagcccacggcacccagcaggtaccagcggcaacgcagttgcagcgtgacgtacatttcgcactcggccaagcgggcggctggcgctacctcgaagggccagcagtccgtcaagcagccgaaactggtcaggagtttcaacgttttggcatggcgtccaggtgtatgaaagttcacgtcgcgtccgtggtgttcgccaacgcaggcggccaacgcgtcggcgtcatgaccgtgacgcagcagcatcgctaccacgtcgtgcggtacccgcgtagcaaatggcgtctgtggctgacggtatacggcttcggtgtacatcataccgtaacgcgccagctcgtccagatgacgcgcgcacagcagcagaatctcttgcgagggttcgtagatgtagaggcgcgtaccgccacccatgcagagcaccagctccgtctcttcgtagtgatcttccaccatgatcacgcacttgcctagcacgataaggcgttcggggcaacaaatcacgtcgtccagcagctggtcgcgtagctccggcatggtgctgccgggccgtacctgcaggaaccagttgtgcggaatgccgagcgacagcacctggtcgacgtggttacggacccagtcgcgaagcacgtcggcgctgtactggcactcgaagatgccctgaaagtcgcccatgacccgcagaaaagtttcgtagcgcgtgtggcaatagaggaattcatcgtttcgcgtaaacgtgggagctccgtcttcccaacgtgtacgccacatgtcaaaagaggccgccagctagacaccccagaaaagaagcagagaaagagacttctttgtgcgacacgttttattctgcgtcctccgctcgacgttcaaatctggatgtactcgcgcacacccgtcaggctctttaagggaaaagggtccgagtacgtcactaaccgcgactgatgcaccagggcggtaatcacccgctccgcgccctcgcgcgtcgacgaacgcgtcgtcaccaggcaatgcagccgcgggcccgtatcgtcctgatgaccagcggcctcgcgctcggctgcttccacaccgacaatgtcgggatccaacacgtagctctgcgagttggtgtcgtagcggtgtagcaccaacgtgttggggtccagacgctcccacgcgccctcgtgcgggtcaaaacgctccgttaaacagagccagtcatactgctgctgcagaatacgccgctcgcgctcgcgtcgctcatcgggcaacgcggcgtcttcgttgaagagaatgtcccgcttgtggtctacggcacgctcgtggtggtgcgggcacaggtgacggtgttccatacgcgtctgacgttgacgctcgcgctcaaaacgccggtgtcgaaagaccattttcagcaaccccatgcggaaaaactccgtgatggtgttggcaacgcgccgcacatagtggttggggtcgtccatctggatggcgtacacggcaccgaaccagtccagcagtaccagcacttcggccacaaaactgcgtcccggtcgcggacgtcccgtcacgcctagcacataccacggcgtggccagattagcacggacagcccaccaccaacgacggctctccacctcggtgagcgcacaaaagggccaaatgcggtgtaactgctgtaccgttttcatcaaccgcataatcaccgtaccgtaacccggtgtatgcaactttacgtcgcaacccaggattcgttcggccgtggcgtacgagccctcgggcgtggtgtcattgagaaacaaaacatgcatggtacgcgcgcccttaggatatcgtcgcggaacgggtaccgtcattctccgcagagtggtgtgaatcacgtcgcgatacgcaatctccgaacgtgacacaccgtaacgtgccagttcgtccaggttgtgcgataccaacaccatgtacttttcacgagtgtcgtaggcgtagacgcgagaaaagcgacccataaaaaccacgtacggagtagccaccatgccatcatggtgatcgcgacgtggctcgggcaacaaaataacagcgtatcccaacggcgtcaacggctcgcggcaacagatgagctttgacgccgcctgtttggcggcggtaatgatcccgtcctccgtacgtaacatcacatgccagcccttggggggacccaaggacagacagcgtccctcgttacgatgaacgtaacgcgtgatttccattggctccaggcaaaagaacagttccttaaaatcccgcaacacttgtcggtataacgccatgggatcctcggccgccacaggcagcgcggggagctccggcggcataactgcagcgccgtcagggccagaacccgcagccggatccatcattgagcgacactctcagccggacaaccggcgtcactgacagaagccgagccaaatacagagaaagcaacgctacaccgtcaccccgctcccaagcgccgcggaaagtgctccgatttttcaccgtcgttcgcgacgttgatttgcctcggtctgagaaccgacctagcgttcggaccggtgcgcagaaacagccggcggtccgagccactgagcggttcacagccccggccgccgatagttaccggagagacgttcgagctgcaggtacatcagcgcttcccgcttcgccaccccgcgcccgccccagtttatactctccgacgccccgtccaacgcgcctgtggagggccaatcggaccgcgggagctctccaagtggatgacaggcacagccgagtgcccgaccgtgaagagccctcatccacctgaacagaccgctaaccgaaggaccccgagtcgcgtccgtcggtcccgacgtccgtcgccatctggctccctgctgttggctacctctcggatttcaaaaaagagcacgtgccgatgacggtgcacaggaaagagccaaagtgtcacggcgtctttttttatttgtattcctttcctgttttgtactcgtaaactgttgatgttgtttttacatccaaaagggcaagtaagaaacaggatgaggcatggtaggtttgggcgtggggcggccctccagcacggcggcccgggccgcccggcgggtgagcacccggcgttgcgccgtgtctatcttgtgtttcttctgtgtctttttcctatcttgttccgcgacggcctctttcatcacgttcagcatgcgttcctcgacgccctccagggatcctggggaggagggagtcctagtgaggcttccaatgttgttttgtggattttcggtttccttttcttggtcgtcatcgtcggacgtgtcgtcttcctcttgatcctcttcttcgtccgagtagtagacgcatagtccttggttcatcaggctgggattcatcaggttctgacggggaatccgctgttgtagacgtttaaccgcccgttccaggcgagagctcatgccgcaccagacgctgtaacgccgcacgggcccgtagcgggctgtttgttcgcgtacatgatcgttgagctcttgccaatattgtttggcacactccagatcggaggtttgtggatagtcgggtcggatccgcggatcccaactgacatcggcggtgccggagacttcgtccagactgttacgcatagagcaccagtcgggtcggacgataaacctgtccttgcggattaaccatttataacgtagttcgtgatggcgtgtagaggcccgtacacgctccacggtcccaaagcggtcccagaagggaaagttttcgtgggggcagcgacccggcacttccagacgttcggcgtcgtccacggcgtagtgaaaacgccggccggcctggtaaattttgagcagacccactgttaacaacatatccacgctgtcagccaaccgccagatctcgcggcgagacacgtcaaaatagaaaaattcgcaggctcggtcgaccaggatcacgaaatcggcgtgaaagacgccggagggtagcgattcgcccaccacacccattatcatggtttcacagcataagcggtccacaaagaacttcaacaggtcgttgaattgctccgtctccatacagatgaagggccagacgcctttgaggttctcggcctggccgcagagcagtagcggacgtgtcatctcgcccggagtgcgcagaggcacgcattcgccgcgataacgacaggtcacacgctgtagttcgctgatgctgttgtcgtgcaggcgaaggtcgcagataatatgatccggttgcgtggttagcagcggcgtgcgcatttgctcgccgtagatggcctcgcagtgcaacagcccgtgtcgcgcaaaatcgtccaaactgtgcgccaggtagtaaagcaccccgcgatcgcggtctagacaccacacggtttcgtaacgtcctaacaggagcaccagacgggcctggctaggtggctcaatttcctctacatacacgaaaaagtcgtcatcgtccgagtcctcgtcctcagaagaggaccgcggcccgtgtactctgggcaacacggtggtagagaactgcaggacgcccagagactcgagcgattcttcgcagcagatgagctgaccccagggcgtttcgggcccgtcggtgacagccgcgctgccaaagatgtcctcaaactctacaaaatctagacgccatccgggtggcgctgaaatgggaaggctaatgttcatatcagcatagctacgaactaagtggcggatgtcctgccgcaagtcttggcagagaatgagctttcgtaaacccttgagggtcctccgaacaacggccccagacgcgtagcgataggactggcgcatggtgccgcggcgtggagcggcacttggcagcctattttatggagtttcttcagtgacgtggcttgttcacgtcgttcgtgggctgcggttggcagctccggtctgtaaaccacccgaaaagactgacatcgacgtcaaagactcacgtaatttggaacatgtgcgaccgcaaagtgcgtcagaatagcacgtggctttaggacataaaaagtaccgtgaggtctagacgtgggttttgtgattgacacttacaccaggtaagccaagggacggtgaaactgtatgtgaggaatctgggtgcttagacgactaacgtgtaatgctttttacaggactgttcgacaggtgatagtacctgtaaggtgatgaccacctctacaaataatcaaaccttaacgcaggtgagcaacatgacaaaccacaccttaaacagcaccgaaatttatcagttgttcgagtacactcggttgggggtatggttgatgtgcatcgtgggcacgtttctgaacgtgctggtgattaccaccatcctgtactaccgtcgtaagaaaaaatctccgagcgatacctacatctgcaacctggctgtagccgatctgttgattgtcgtcggcctgccgttttttctagaatatgccaagcatcaccctaaactcagccgagaggtggtttgttcgggactcaacgcttgtttctacatctgtctttttgccggcgtttgttttctcatcaacctgtcgatggatcgctactgcgtcatcgtttggggtgtagaattgaaccgcgtgcgaaataacaagcgggccacctgttgggtggtgattttttggatactagccgtgctcatggggatgccacattacctgatgtacagccataccaacaacgagtgtgttggtgaattcgctaacgagacgtcgggttggttccccgtgtttttgaacaccaaagttaacatttgcggctacctggcgcccatcgcgctgatggcgtacacgtacaaccgtatggtgcggtttatcattaactacgttggtaaatggcacatgcagacgctccacgttcttttggttgtggttgtgtcttttgccagcttttggtttcctttcaacctggcgctatttttagaatccatccgtcttctggcgggagtgtacaatgacacacttcaaaacgttattatcttctgtctatacgtcggtcagtttttggcctacgttcgcgcttgtctgaatcctgggatctacatcctagtaggcactcaaatgaggaaggacatgtggacaaccctaagggtattcgcctgttgctgcgtgaagcaggagataccttaccaggacattgatattgagctacaaaaggacatacaaagaagggccaaacacaccaaacgtacccattatgacagaaaaaatgcacctatggagtccggggaggaggaatttctgttgtaattcgatcctctctcacgcgtccgccgcacatctatttttgctaattgcacgtttcttcgtggtcacgtcggctcgaagaggttggtgtgaaaacgtcatctcgccgacgtggtgaaccgctcatatagaccaaaccggacgctgcctcagtctctcggtgcgtggaccagacggcgtccatgcaccgagggcagaactggtgctatcatgacaccgacgacgacgaccgcggaactcacgacggagtttgactacgatgaagacgcgactccttgtgttttcaccgacgtgcttaatcagtcaaagccagttacgttgtttctgtacggcgttgtctttctcttcggttccatcggcaacttcttggtgatcttcaccatcacctggcgacgtcggattcaatgctccggcgatgtttactttatcaacctcgcggccgccgatttgcttttcgtttgtacactacctctgtggatgcaatacctcctagatcacaactccctagccagcgtgccgtgtacgttactcactgcctgtttctacgtggctatgtttgccagtttgtgttttatcacggagattgcactcgatcgctactacgctattgtttacatgagatatcggcctgtaaaacaggcctgccttttcagtattttttggtggatctttgccgtgatcatcgccattccacactttatggtggtgaccaaaaaagacaatcaatgtatgaccgactacgactacttagaggtcagttacccgatcatcctcaacgtagaactcatgcttggtgctttcgtgatcccgctcagtgttatcagctactgctactaccgcatttccagaatcgttgcggtgtctcagtcgcgccacaaaggtcgcattgtacgggtacttatagcggtcgtgcttgtctttatcatcttttggctgccgtaccacctaacgctgtttgtggacacgttaaaactcctcaaatggatctccagcagctgcgagttcgaaagatcgctcaaacgtgcgctcatcttgaccgagtcgctcgccttttgtcactgttgtctcaatccgctgctgtacgtcttcgtgggcaccaagtttcggcaagaactacactgtctgctggccgagtttcgccagcgactcttttcccgcgatgtatcctggtaccacagcatgagcttttcgcgtcggagctcgccgagtcgaagagagacatcttccgacacgctgtccgacgaggtgtgtcgcgtctcacaaattataccgtaatataacttcgtatagcatacattatacgaagttattaaaaaagcgctacctcggccttttcatacaaaccccgtgtccgcccctcttttccccgtgcccgatatacacgatattaaacccacgaccatttccgtgcgattagcgaaccggaaaagtttatggggaaaaagacgtaggaaaggatcatgtagaaaaacatgcggtgtttccgatggtggctctacagtgggtggtggtggctcacgtttggatgtgctcggaccgtgacggtgggtttcgtcgcgcccacggtccgggcacaatcaaccgtggtccgctctgagccggctccgccgtcggaaacccgacgagacaacaatgacacgtcttacttcagcagcacctctttccattcttccgtgtcccctgccacctcagtggaccgtcaatttcgacggaccacgtacgaccgttgggacggtcgacgttggctgcgcacccgctacgggaacgccagcgcctgcgtgacgggcacccaatggagcaccaacttttttttctctcagtgtgagcactaccctagtttcgtgaaactcaacggggtgcagcgctggacacctgttcggagacctatgggcgaggttgcctactacgggggttgttgtatggtgggcgggggtaatcgtgcgtacgtgatactcgtgagcggttacgggaccgccagctacggcaacgctttacgcgtgaattttgggcgcggcaactgcacggcgccgaaacgcacctaccctcggcgcttggaactgcacgatggccgcacagaccctagccgttgcgatccctaccaagtgtatttctacggtctgcagtgtcctgagcaactggttatcaccgcccacggcggcgtgggtatgcgccgctgtcctaccggctctcgtcccaccccgtcccggccccaccggcatgacttggagaacgagctacatggtctgtgtgtggatcttctggtgtgcgtccttttattagctctgctgctgttggagctcgttcccatggaagccgtgcgtcacccgctgcttttctggcgacgcgtggcgttatcgccgtccacttccaaggtggatcgcgccgtcaagctgtgtcttcggcgcatgtttggtctgccgccgccaccgtcagtcgcaccacctggggaaaagaaggagctaccggctcaggcggccttgtcgccgccactgaccacctggtcactaccgccgtttccgtccacgcggatacctgacagtccgccgccaccgtaccagcttcgtcacgccacgtcactagtgacggtacccacgctgctgttatatacgtcatccgacatcggtgacacagcttcagaaacaacgtgtgtggcgcacgctacttatggggaacccccggagcccgctcgatcgacggctacggttcaggaatgtaccgttcttaccgccccgaattgcggcatcgtcaacaacgacggcgcggtctctgaaggccaagaccatggagatgcggttcaccatagcctggatgtggtttcccagtgtgctgctgatactggggttgttgacacctccgagtaacgggtgcaccgtcgatgttggacgaaacgtatccattggagaacagtgccgccttcgaaacggtgcgacgttctccaagggagacatcgaaggtaacttcagtgggcccgtcgtcgtggagttggactacgaagatatcgatattactggcgaacggcagcgacttcggttccatctcagcggactcgggtgtcctacaaaggaaaatataagaaaagacaatgaaagcgacgtcaacggtggaattcgctgggctctatatatacaaaccggcgacgccaagtacggtattcgtaaccagcatttgagtatacggttaatgtatcctggggaaaaaaatacacaacagctgttggattctgatttcagttgcgaacgtcaccggagaccgtccacgccgttgggaaagaacgccgaagtgcctcccgcgacccgcacgtcttctacatacagcgtcctcagcgcttttgtagtgtggatcggatccggcctcaatatcatctggtggaccggcatcgtgcttctggcggtggacgctctcggacttggcgagcgttggctgaggttagcactgtctcaccgggacaaacatcacgcatcgcgaaccgcggcgctccagtgtcaacgcgacatgttacttcggcaacgtcgacgggctcggcggctgcacgccgtttctgaaggcaaactgcaggaagagaagaaacgacagtctgctctggtctggaacgttgaggcgcgaccctttccgtccacacatcagctgattgtgctgccccctcctgtagcgtcagctcctcctgcggttccctcgcagccccccgagtattcgtctgtgtttccgcctgtataaaaataaagagacgggaggctgatcgcggccttcagcgtctcatttgtctttactctcgagtgcggtcggtgtctcgtcggtgagacgaggccgccgcccgacaagttcgatctcatgtcgctcttggagcgcgaagagagttggcgtcgcgtagtcgactactcgcacaacctgtggtgtacgtgcggtaactggcagagccacgttgagattcaggacgaggagccaaactgcgagcagccggagcccgcacactggctggaatacgtggcggtccagtggcaggcccgggttcgcgattctcacgatcgctggtgtctctgcaacgcctggcgtgatcacgccttgcgcggccgttggggtacggcgtattcctcgggttcctcggcctcttcctccggtttcgtcgcggagagcaagttcacctggtggaaacgactgcgccacagtacccggcgctggttgtttcgccgccggcgagctcgatacactccgtctaactgtggggaaagtagcactagcagcggccagagtagcggtgacgagagtaactgcagtctacgcacccacggcgtgtacacacggggtgaacaacactaatcgataagtcgcgtgtaggcgactggctacatcaaccggatatctgcggggatttaaaaagacgacccgttgtcatccggcttagaccaaaccgtccttttatcatcttccgtcgccatggctatgtacacatccgaatccgaacgcgactggcgtcgtgtaatccacgactcgcacggcctgtggtgcgactgcggcgactggcgagagcacctctattgtgtgtacgacagccattttcagcgacgacccacgacccgagccgaacggagggccgccaattggcggcgacagatgcggcggttacaccgtttgtggtgtttttgtcaggactggaagtgtcacgcgttatacgccgagtgggacggcaaagaatccgacgacgagtcgtcggcgtcttcctcgggcgaagcgccagagcaacaggtccccgcttggaagaccgtgcgggccttctcgcgggcctaccaccaccgcattaaccggggtctgcggggcacgcccccaccgcgcaacttgccgggatacgagcacgcctccgagggctggcggttttgcagtcgacgggaacggcgagaggacgatcttcgcacgcgggctgagccggaccgcgtggtgttccagttagggggagtaccccctcgccgtcaccgggaaacttacgtgtaagaacacggcgtgacaataaacaacatagcgtaaatccccgtgtgatgtgtgtgattgacgttcgggaaacatgtccccatcatcagcgtcacaattgacgtgggttggtcactgacgtgcaggatgttacgcgagtcagagaatcgcataagaacggagtggtgagcgggttcccacaggagtctctggcgcaaaagcaccatgagcctcaggttccccgagagggtgggttacgagaaactgggataccgcccgcatgccaaacgcgtgcgggtgcatgactcgttgggattgacgcggtttatcatgaggcaactcatgatgtacccgctggtgttgccgttcacttttccgttttacgtgccgcggtcctagcacgtcagtggtgacgctgataattgcaacatggcccatgacgaacccgcttgggacgaacgtcaataccacgtcaaaccaccgtgacttggctgaacgttgaaacataaagccaaagcgccgtcggcacttggcttcagagcagcgcctcggggcgatgcgacggcgatgaacttagagcaactcatcaacgtccttggtctgctcgtctggattgccgctcgtgctgtcagccgcgttggtccgcatggctccggactcgtttatcgtgagcttcatgatttctacgggtatctgcagctggaccttctgggaccagtggtggcggggaatcgctcagtccggacctggagagagcaggcggaccgagccagagggaccttcgcttggcgttcaggccttaatactagccgcatcttacctgtcggcagcatgtatcggggctccgacgccttacccgccggcctgtatcgtcccgaagaagaggtgttcctcctcttgaatcgctgccatgggccactgtcaacgccgaaaaatgcttgtctggctgaggttggtgtcgctaatgccacttttttgtctcgcttcaatgtcggtgattttcacggagcgtcatgggaaaacggtaccgctcccgatggagagcccggggtatgctgaaattcctcttaaaatttcgtaaacgacgtcgtccagtcgttgtgccgcgattcgtacggttcatcgtctacgtcgttttgttcaccgtcgctgtgcaacgcgtgaaacaagagcgtgatgcgcaccttcggcggtatgaagaacgattacggaaaaaccgcgcacggcgtcggcagtcttttccgtgacttggggcgatgggtccgagctgcggtatgggtcacggcggcgtgtgttttattgacgaagatgccgatgtgtgactaaaaacgtcccagccccagagcgatatgtttcaataaaaaaaatatgtagtatcatattatgcgtgtcctggtttttcatttttggatgtatgtatcgcataaagggtggcgaggtgtgaggatgaaacatatgcagatacgcagtgttgttatccgaacgaaacccgtgtaatgcgtacaacggtacttcagtatgaaagtcccgtgtgtggggggggggggcaaatagttgcgtttgccgttgggcgtacgctacgtttgtatttctggctataatatgtgcggtcatgtgtcgatgttcctattgggaagggtgtgactgtagggtgtataaagtacggtgggacgcagagggacattgatagaaacaggttgagcgctgtacgagtttcacacgctgaatcggcgccaagaataaaacagtggttattcgtaaaagtatgggggggggggggatgttgtcgacggttgttagatgcattgcgtatctgtattagtagttttgcaagccgtggtgcgtgttattgtgacgtagcaattatcgtattgtgcatgtgtcgttcatcacagagtttagtatactaatatgaagcgtcgcgagtattaaagcaattggtgtctctgtgctagtctaacaacacctgtgtaatgcgtacaacgagaaaaaagacgcgaaagcaacgtgtatgggggggggggggaataatattgctaatcatgcgtcttgcagtacagatagccgctgtatcttacgcgtattgtcgcaacagttccacatcggtgtaattggatgtctggtacttatcactggcgtcgttataacattgtaaaacaagttttcgaaacataacgacagctgcaaaagaaaaccagtttattgagcattgtaatggtagtgtgtggctatattagaaaacgtgacgcgtcgcatgtcgcggcacaatctggcagcggggtcggggtagggtacggtgggaggcatgtacacagatggaacaaaagcagaagtaacgtgagaaggagcatacagtccagtatccagcggttcctgagtagcaccacccatcaactgaatgccctcatgagtaaaagtctgcgggcgacagcccttggggaccgttggcatgggacgatcaatctccaaaccacagcgtaacaccgttttcttccaacgtcgttgatagacgtcgtttttacggttactcccaagaacccagaaagtctcgtccaagtcgtaccaggaatcttctccggggagacgcgacggtttccaatcctcgtcgtctcgtctcaaagcacgtcccaaactggcttgaggagtcaacggtggttctgtgggtcgggtgtagcgcgagtgttttcccttcatgagcgattcatcctccttgcctttaggctttttggtctttttgtgtatcatctggccgccggcctccataaccaccgtggccaagtccagtcccagagcttgagcgtcggcgcggcgtcgggcgtcttgcaggtagtcttccacatttgcacagatggccgggtgtttggtggctagggtgaggacctcagcctcgccgcgacccggacgtagcaaaaaagccaactgcccgtgcggctcgcgcgcccacagcgcggcgcgcgggtgcaggtgcagcgcgtcccagcgcggccgctcccactgctcgcggtccagctcgggcagcagccgccgcgcggcctcggcggcgggcgccgactcgcgtcccagcgccagcgcgcccagcacgcccgcgcgcagaaagtgcgacagctccgccgccagcgggtacacgtgcccgtccagcgggcagtacccgaacacggcgcccagctcgtccagcagcaccaccagcatggcgcgcggcacggtccccgacgccgccggacccgccatcgctgtcgaacccaccatcaccgtcggcgccgctgctgctgccgcgtccgccccgaccaccgcgtgcgcgtccgcgttaggcacgcaaatcgcgcccccgccggcggcgccgtacggctgcggaggcacaggtacagcggaccccacggctcccgctatcgcgcacggcgcgtccccgccggcggcctccgtctccgtgccgctcgccgctggcggcgacgtcgtccccgccgccgtccccgtcgccggccccgccgcgcagcccagccaccgcgcgggcagcaccgcgcccagcgccagccagccgcagcacagacgctggttcaggtgccgacgcacggccgtcagcagcgacgcggggtgcggcgccgacgcgaacggctcgtactgcgccagctcctgccacgcgcccagcagtaccatcggctgcagtcgcctgcccggcgtctgcagcgccaccgtcgtgccggcccaccgccggcgcagctcccgtccgagcgccgtcgcctcctcggcgcgcagcaacgtctgtcggagcgccggctgaggcagcagcgtcgcgcgcggggtgcccacgcccagccggttgcagcggtacagccgcaccacctcgcccgcgccgtgccgaaaccactcgtccgcgtcgcgcgccgctaggatcagcgtgttgtttgccaggtcgtacacgaacacgcggaacccggcgcccagcgccaggtacagtccgtcctgcgcgcacagaccctcgggatggccggccttgtcgcccaacgtcgggtcggctgcggggtccacctcgtgcaccacggtagccaccagtacgatccacgcgtcccgcggcgacagttgacgcaggtccgtcgcgcccacgccgttcatctggctgcgcggcgtcacccgcgcgtagaatccgtacggccgtccgagcggcagcagcgtgcccgcgtcgcgctgcgaccacttgcgcatggcgcggcccgtgctgttggccaaaaacgccgcgcgccacacggcgcccatggcctggtattccagctccgtcagcgcctggcgctccaccggaatctgagacagcaacaggcgctccgggccgtgccaaaagttgctattgttgccgctactcggaggggcgcccggcggcccgcggggttctacccggtggacgccgtggcccggcgtcgtcgtagccgcagcactcgcaccagtgcccgctgtggacggcgctcccatcggcacacaagaagaaggaggagaggaaccaacccccgaaggccctccggccccgcggccgcgaccaaggggcggggggcgcggcgacatgccgttgcgctgggccatgggcgccggacacctccgacgtccactatataggaagcaaacccgcgtcagcgagcacgcggtttagacacgcggacgccttcgtcgcccgtgtgccgcgggcgacacgcagctggcttttataggcagcgacgtgcacggcgcgtgctggcgccgccttggcgccacgcagtctggaaggccgtggactgggaaaggcagctacccgaaggaagacgcgcggcaggcgcgtaccactggagcgcacagccgcctcccgggcgcgcacccatctaggtggacgcccgacatccattccgggccgcgtggtgggtcctcgaggggcgggggggtgtttttagcgggggggtgaaacttggagttgcgtgtgtggacggcgactagttgcgtgtggtgcggaggacggcgacggcgaataaaagcgacgtgcggcgcgcacggcgaaaagaagacgcgtgtctgtgtctgtgtgattccccggggaaaagaggaagttcccgggggacggcagcatgggtccctggggacacacgaaaagcaacgcccgggggcgagggacgacggccctggggaccgcgggggaaataacggccgcgaggccacacactcgttcctgcgaagccgcacaccccgaggccgcgcacaccgccgacacaccccgccaccacaccccgccggcacacccgccacacgcccgcgacacacccggcacgacacacccggcacacgcccgcgacacaccctgacacaccctgccaacacacccccgacacacccaacacacgcccgcgacacacccggcacacacccacccggccgcgccccgacacacccaaaacaccgccggtgcggggccgcgtggtgggtcctcgaggg

(19) United States
(12) Patent Application Publication
(10) Pub. No.: US 2025/0011732 A1
(43) Pub. Date: Jan. 9, 2025

(54) SCALABLE CHROMATOGRAPHY PROCESS FOR PURIFICATION OF HUMAN CYTOMEGALOVIRUS

(71) Applicant: Merck Sharp & Dohme LLC, Rahway, NJ (US)

(72) Inventors: Adam Kristopeit, Lansdale, PA (US); Janelle Konietzko, Lansdale, PA (US); Wanli MA, Lansdale, PA (US); Katherine Phillips, Audubon, PA (US); Andrew Swartz, Chalfont, PA (US); Sheng-Ching Wang, Eagleville, PA (US); Marc D. Wenger, Allentown, PA (US); Matthew Woodling, Lansdale, PA (US); Tiago Matos, Philadelphia, PA (US)

(73) Assignee: Merck Sharp & Dohme LLC, Rahway, NJ (US)

(21) Appl. No.: 18/769,900

(22) Filed: Jul. 11, 2024

Related U.S. Application Data
(63) Continuation of application No. 17/046,871, filed on Oct. 12, 2020, now Pat. No. 12,060,583.
(60) Provisional application No. 62/661,928, filed on Apr. 24, 2018.

(51) Int. Cl. C12N 7/02; B01D 15/36; C12N 5/079

(52) U.S. Cl. CPC C12N 7/02; B01D 15/362; B01D 15/363; C12N 5/0621; C12N 2710/16111

(57) ABSTRACT

The present invention relates to a scalable process for the purification of human cytomegalovirus particles from cell culture medium. In particular, the process involves a two step chromatography process starting with an anion exchange chromatography step followed by a polishing chromatography step selected from mixed mode chromatography or cation exchange chromatography.

### CROSS-REFERENCE TO RELATED APPLICATIONS

This application is a continuation of U.S. patent application Ser. No. 17/046,871, filed Oct. 12, 2020, which is a National Stage Application of International Application No. PCT/US2019/028216, filed Apr. 19, 2019, which claims priority to U.S. Provisional Application No. 62/661,928 filed Apr. 24, 2018. The entire contents of these applications are hereby incorporated herein by reference in their entireties.

### REFERENCE TO SEQUENCE LISTING SUBMITTED ELECTRONICALLY

This instant application contains a Sequence Listing which has been submitted electronically in XML format and is hereby incorporated by reference in its entirety. The XML file, created on May 7, 2024, is named 24557_US_CNT_SL.XML and is 233,303 bytes in size.

### FIELD OF INVENTION

The present invention relates to a scalable chromatography process for the purification of human cytomegalovirus (HCMV) particles from cell culture medium. In particular, the process can be scaled for the production of commercial quantities of HCMV particles for use in vaccines.

### BACKGROUND OF THE INVENTION

Human cytomegalovirus (HCMV), also known as human herpesvirus 5 (HHV-5), is a herpes virus classified as being a member of the beta subfamily of herpesviridae. HCMV is an enveloped virus, having an outer lipid bilayer envelope containing a variety of glycoproteins including glycoprotein B (gB), gH, gL, gM, gN, and gO, with a double stranded linear DNA core in an icosahedral nucleocapsid. See Crough et al., 2009, Clinical Microbiology Reviews 22:76-98.

According to the Centers for Disease Control and Prevention, HCMV infection is found fairly ubiquitously in the human population, with an estimated 40-80% of the United States adult population having been infected. The virus is spread primarily through bodily fluids and is frequently passed from pregnant mothers to the fetus or newborn. In most individuals, HCMV infection is latent, although virus activation can result in high fever, chills, fatigue, headaches, nausea, and splenomegaly. HCMV infections in immunocompromised individuals (such as HIV-positive patients, allogeneic transplant patients and cancer patients) or persons whose immune system has yet to be fully developed (such as newborns) can be particularly problematic. See Mocarski et al., 2007, Cytomegalovirus, In Knipes and Howley (Eds.), Field Virology pp. 2701-2772. HCMV infection in such individuals can cause severe morbidity, including pneumonia, hepatitis, encephalitis, colitis, uveitis, retinitis, blindness, and neuropathy, among other deleterious conditions. In addition, HCMV infection during pregnancy is a leading cause of birth defects. Sec, e.g., Adler, 2008, J. Clin Virol 41:231; Arvin et al., 2004, Clin Infect Dis 39:233; and Revello et al., 2008, J Med Virol, 80:1415.

To date, there is no available HCMV vaccine. However, a promising candidate based on a live attenuated HCMV is currently in clinical trials. See U.S. Pat. No. 9,546,355 and Wang et al., 2016, Sci Transl Med 8:362ra145. This vaccine is produced using human retinal pigment epithelial cells (ARPE-19) infected with a genetically modified HCMV.

For commercial vaccine production based on a live virus, process impurities resulting from the upstream production process, including host cell contaminants and cell culture components, such as serum, endonucleases and supplements, need to be removed. Purification of HCMV has typically involved centrifugation-and filtration-based methods. See, e.g., Chambers et al., 1971, Appl Microbiol 22:914-8; Huang et al., 1973, J. Virol., 12:1473-81; Talbot et al., 1977, J. Gen. Virol. 36:345-9; Vicente et al., 2014, Eng. Life Sci. 14:318-2; and Schneider-Ohrum et al., 2016, J. Virol. 90:10133-44.

While effective at laboratory scale, centrifugation-based methods such as density gradient sedimentation are not easily applied at a manufacturing scale. In contrast, while filtration methods are amenable to manufacturing scale, their effectiveness and/or efficiency decreases with cruder feed streams having higher levels of impurities, causing fouling of the filter. Other options for the scalable purification of an enveloped virus such as HCMV are often constrained by a narrow stability range (e.g., pH, ionic strength, compatible detergents/solvents, shear stress, and temperature) and large particle size, which challenges chromatography efficiency (capacity and yield) and decreases sterile filtration yields.

Various purification methods for live viruses employing chromatography and/or filtration have been described. See U.S. Pat. No. 8,124,106 (anion exchange chromatography and size exclusion chromatography) and U.S. Pat. No. 9,663,766 (mixed mode chromatography and anion exchange chromatography for adenoviruses); International Patent Application Publication Nos. WO2003/097797 (ion exchange chromatography for adenovirus), WO2013/106398 (ion exchange chromatography and tangential flow filtration for herpes virus) and WO2016/156613 (aseptic solid-phase matrix chromatography for viruses); Puig et al., 2014, Methods Mol Biol 1089:197-210 (hydrophobic interaction chromatography and anion exchange chromatography for canine adenovirus); Langfield et al., 2011, Methods Mol Biol 737:345-66 (tangential flow filtration for measles virus); Mundle et al., 2016, Vaccine 34:3690-6 (core bead chromatography and TFF for respiratory syncytial virus); Tseng et al., 2017, Vaccine S0264-410X(17):30322-5 (anion exchange chromatography and ligand core chromatography for influenza virus); Lee et al., 2016, J. Chromatography 1445:1-9 (negative chromatography for hepatitis B virus like particle); James et al., 2016, Sci Rep 6:36826 (Capto™ Core for reovirus); and GE Healthcare Life Sciences Application Note 29-0003-34 AA, 2012 (Capto™ Core 700 for influenza virus).

### SUMMARY OF THE INVENTION

The present invention is directed to a two-step chromatography process suitable for large-scale purification of HCMV particles. Specifically, the present invention is directed to a method of purifying human cytomegalovirus particles (HCMV) from a cell culture medium, the method comprising: a) contacting the cell culture medium comprising HCMV to an anion exchange chromatography medium under conditions that allow the HCMV to bind to the anion exchange chromatography medium; b) eluting the HCMV from the anion exchange chromatography medium to obtain an eluate; c) contacting the eluate with a polishing chromatography medium; and d) collecting the HCMV from the polishing chromatography medium to obtain purified HCMV. In one embodiment, the polishing chromatography medium is selected from a mixed mode chromatography medium and a cationic exchange chromatography medium. In one aspect of the embodiment, the polishing chromatography medium is a mixed mode chromatography resin and the HCMV flows through the mixed mode chromatography resin. In another aspect of this embodiment, the mixed mode chromatography resin has size exclusion properties and the HCMV is excluded from the mixed mode chromatography resin. In one aspect, the mixed mode chromatography resin is a hydrophobic anion exchange chromatography ligand having a molecular weight exclusion of about 700 kDa. In another embodiment, the polishing chromatography resin is a cationic exchange chromatography medium, wherein the HCMV binds the cation exchange chromatography medium, and the HCMV is eluted from the cationic exchange chromatography medium.

The methods of the invention can be used where the cell culture medium comprises one or more contaminants selected from proteins and nucleic acids. In certain embodiments, the cell culture medium comprises serum proteins, host cell proteins, Shield-1, exogenous endonuclease and host cell DNA.

The methods of the invention also contemplate additional steps either before or after the two-step chromatography. In certain embodiments, the method further comprises performing tangential flow filtration on the eluate from step b), the purified HCMV from step d), or both. In certain embodiments, the method further comprises e) a sterilizing filtration step using a 0.2 μm membrane made of PVDF or cellulose acetate. In certain embodiments, the method involves a step prior to step a) wherein the cell culture medium comprising the HCMV is subject to nuclease treatment. In certain embodiments, the method involves a step prior to step a) wherein the cell culture medium is subject to clarification.

In certain embodiments, the methods of the invention provide for eluting in step b) using a salt selected from sodium chloride, potassium chloride, ammonium chloride, and sodium sulfate each at a concentration of about 0.5-2 M. In certain embodiments, the methods of the invention provide for eluting in step b) using an amino acid selected from arginine and histidine each at a concentration of about 0.25-1 M.

In one embodiment, the two-step chromatography process is performed aseptically. In one aspect of this embodiment, the two-step chromatography process is performed aseptically using an anion exchange membrane and a sterilized slurry of a mixed mode chromatography resin.

The methods of the invention can be used to purify any HCMV and can be used to obtain purified HCMV having at least 80% HCMV protein purity and/or a process yield of at least 40%.

The present invention also directed to a method for the purification of HCMV from a cell culture medium comprising the steps of: a) harvesting cell culture medium from a culture of ARPE-19 cells infected with HCMV; b) subjecting the cell culture medium to nuclease treatment using an endonuclease at a concentration of 10-160 U/mL; c) clarifying the nuclease-treated cell culture medium using a 1.2 μm glass fiber filter to obtain a clarified cell culture medium; d) contacting the clarified cell culture medium comprising HCMV with an anion exchange chromatography membrane under conditions that allow the HCMV to bind to the anion exchange chromatography membrane and then eluting the HCMV from the anion exchange chromatography membrane to obtain an eluate; e) contacting the eluate with a mixed mode chromatography resin which is a hydrophobic anion exchange chromatography resin having a molecular weight exclusion of about 700 kDa and then collecting the HCMV from the mixed mode chromatography resin to obtain purified HCMV; and f) performing tangential flow filtration on the purified HCMV collected from step e) to adjust to the desired concentration and buffer. In one embodiment, step b) is run prior to step a) by addition of nuclease to the cell culture medium prior to harvesting, i.e., during cell growth. In another embodiment, step b) is run following step a). In one embodiment, the cells are grown on microcarriers. In one embodiment, the purified HCMV has at least 80% HCMV protein purity. In one embodiment, the purified HCMV has a yield of at least 40%. In one embodiment, the purified HCMV contains 10 ng or less hcDNA per dose.

In certain embodiments, the HCMV is a recombinant genetically modified HCMV. In one aspect of this embodiment, the HCMV is a recombinant genetically modified HCMV having a genomic sequence of SEQ ID NO: 1.

The present invention also is directed to a process for sterilizing a large virus comprising: a) applying cell culture medium comprising said virus onto a 0.2 μm membrane made of PVDF or cellulose acetate; and b) collecting purified virus.

### DETAILED DESCRIPTION OF THE INVENTION

The present invention is directed to purification of HCMV using a two-step chromatography purification process (anion exchange chromatography followed by polishing chromatography) in combination with additional processing steps to yield an active, concentrated virus product, having high purity and low host cell protein and DNA levels, suitable for use in a vaccine. Vaccine preparations must be sterile (i.e., free from independently replicating organisms) and should contain no more than 10 ng of host cell DNA (hcDNA) per human dose, per World Health Organization guidance.

The purification processes described herein are readily scalable, use procedures and reagents that are compatible with current good manufacturing processes, and are sufficiently simple, rapid, and efficient to be used for the production of purified HCMV for vaccine use. With the proper choice of chromatography materials, the process can be performed aseptically. The HCMV purification process described by the current invention is capable of achieving an overall process yield>40% and a resulting protein purity of >80%.

The present invention is based, in part, on the discovery that HCMV purification using a tangential flow filtration-based scheme was not a robust purification strategy for large scale processing as host cell impurities may increase under these conditions. Applicants discovered that a chromatographic product capture step was necessary to reduce filter fouling for large scale purification of HCMV.

In one embodiment, the two-step chromatography process is performed aseptically. Due to the large size of the HCMV particles (˜100 to 400 nM), it may be desirable to perform the chromatography process aseptically and avoid a sterile filtration step (which potentially would decrease overall yield). In another embodiment, the two-step chromatography process is followed by sterilizing filtration using a 0.2 μm membrane made of PVDF (polyvinylidene difluoride). In another embodiment, the two-step chromatography process is followed by sterilizing filtration using a Pall SuporLife® 0.2 μm membrane made of PES (polyethersulfone). In another embodiment, the two-step chromatography process is followed by sterilizing filtration using a 0.2 μm membrane made of cellulose acetate.

The present invention is also directed to a method for sterile filtration of a large virus such (such as HCMV; size range of ˜100-400 nm) comprising applying a cell culture medium containing a virus preparation onto a 0.2 μm membrane made of PVDF or cellulose acetate. This invention is based on the unexpected observation that virus particles which are larger than the nominal 0.2-micron pore size of most sterilizing filters can pass through membranes made of cellulose acetate or PVDF, and in some cases, PES.

The present invention is also directed to a purification process comprising the following steps: (1) harvest of cell culture medium, (2) nuclease treatment, (3) clarification, (4) anion exchange chromatography (bind and elute), (5) polishing chromatography, and (6) tangential flow filtration (TFF). The process is scalable from laboratory scale (<3 L) to manufacturing scales (e.g., 50-2000 L) and capable of purifying a range of feed streams, including medium from static cell culture (e.g., from cell stacks) and culture in bioreactors. In the purification sequence, nuclease treatment may be performed before (added to cell culture medium) or after harvest of the cell culture medium, and the TFF may be performed before and/or after the polishing chromatography (i.e., before, after, or both).

The following purification attributes have been achieved by the processes described herein.

TABLE 1
Purity Results Demonstrated by HCMV
Vaccine Purification Process

| | | Result |
|---|---|---|
| Attribute | Analytical method | Demonstrated |
| Protein Purity | Mass Spectrometry | ≥80% HCMV protein |
| Residual BSA | BSA western or | ≤50 ng/dose |
| | ELISA method | |
| Host Cell DNA | qPCR | ≤10 ng/dose |
| Process Yield- | Flow Cytometry (Particle | ≥40% |
| Total Viral | Counts) or Viral antigen | |
| Particles | (western blot) | |
| Process Yield- | Relative Infectivity assay | ≥30% |
| Infectious Viral | | |
| Particles | | |

As used herein, purification of “Human cytomegalovirus” or “HCMV” generally refers to purification of HCMV particles.

As used herein, in connection with chromatography, a “support” refers to the base matrix without a ligand. A support can be in the form of a membrane or resin.

As used herein, in connection with chromatography, a “medium” refers to the support with a ligand.

As used herein, in connection with chromatography, a “resin” refers to a bead-based chromatography medium that can be packed into a column or used in a slurry mode.

As used herein, in connection with chromatography, a “ligand” refers to a chemical species affixed to the chromatography support media that is used to effect separation between the desired product and other species.

As used herein, “mixed-mode chromatography” or “multimodal chromatography” refers to the use of a solid phase chromatographic support that employs a combination of two or more chemical mechanisms in order to achieve separation of components such as protein, DNA, and virus in a mixture. Examples include, but are not limited to, chromatographic supports that exploit combinations of cation exchange (i.e., in which the support is anionic), anion exchange (i.e., in which the support is cationic), hydrophobic interaction, hydrophilic interaction, hydrogen bonding, pi-pi bonding, and metal affinity. Mixed-mode chromatography may also employ a size exclusion component. The solid phase can be a porous particle, nonporous particle, membrane, or monolith.

As used herein, “anion exchange chromatography” refers to the use of a solid phase chromatographic support that uses a positively charged ion exchange support that has an affinity for molecules having net negative surface charges in order to achieve separation of components. The solid phase can be a porous particle, nonporous particle, membrane, or monolith.

As used herein, “cation exchange chromatography” refers to the use of a solid phase chromatographic support that uses a negatively charged ion exchange support that has an affinity for molecules having net positive surface charges in order to achieve separation of components. The solid phase can be a porous particle, nonporous particle, membrane, or monolith.

As used herein, “cell culture medium” refers to the liquid portion of a cell culture having one or more components other than HCMV particles, such as, but not limited to, proteins, nucleic acids, lipids, various cell culture medium components and additives. HCMV particles are released by the cells into the cell culture medium without any intervention or the cells may be lysed or rendered permeable to virus through chemical agents or mechanical means. The cell culture medium can be partially purified using centrifugation, clarification or other methods. In certain embodiments, the cell culture medium contains HCMV through its release into the cell culture.

As used herein, “polishing chromatography” refers to additional chromatography operations after a capture step to remove residual impurities such as host cell protein and DNA. For purposes of the invention, polishing chromatography includes cation exchange chromatography and mixed mode chromatography.

As used herein, “process yield” refers to the fraction of product (viral particles) that is recovered after executing the purification operations. The yield of total viral particles is measured using particle counting techniques or by quantifying viral antigen. The yield of infectious particles is also measured using infectivity assays, to verify that the infectivity of the viral particles is sufficiently maintained during purification.

As used herein, “protein purity” refers to the proportion of product related proteins in the purified product, expressed as a fraction relative to total protein content.

### Cytomegalovirus

The purification processes of the present invention can be used to purify any cytomegalovirus, including wild-type cytomegalovirus, a recombinant cytomegalovirus, and a genetically modified cytomegalovirus (for example, cytomegalovirus containing a transgene). In some embodiments, the cytomegalovirus is a human cytomegalovirus (HHV-5). In some embodiments, the HCMV is AD169 strain, Toledo strain or Towne strain. In some embodiments, the cytomegalovirus is a recombinant HCMV, particularly, a conditional replication defective HCMV. In some embodiments, the cytomegalovirus is a recombinant HCMV delivering one or more transgenes. The processes of the invention can also be used to purify both infectious and noninfectious HCMV particles. See Schneider-Ohrum et al., 2016, J Virol. 90:10133-10144.

Methods of propagating cytomegalovirus are known in the art. Sec, e.g., Britt, 2010, Human Cytomegalovirus: Propagation, Quantification, and Storage; in Current Protocols in Microbiology, 18:E:14E.3:14E.3.1-14E.3.17; Chambers et al., 1971, Appl Microbiol 22:914-8; and Schneider-Ohrum et al., 2016, J Virol 90:10133-10144. In some embodiments, cell cultures are inoculated with cytomegalovirus and cultured for a period of time (e.g., 24 hours to 3 weeks) before harvesting the virus. Various methods of harvesting virus from cell culture are known in the art. In some embodiments, the cytomegalovirus is harvested from the cell culture by collecting the medium. In some embodiments, the cytomegalovirus is harvested from the cell culture by lysing the cells (e.g., by mechanical or non-mechanical lysis methods). In some embodiments, the cytomegalovirus is harvested from the cell culture by adding a cell permeation agent to release virus from host cells.

Any cytomegalovirus preparation can be used in the present invention, including unpurified or partially purified cytomegalovirus preparations from natural, synthetic, or recombinant sources. Unpurified cytomegalovirus preparations can come from various sources including, but not limited to, bacterial cell culture supernatants or lysates, yeast cell culture supernatants or lysates, mammalian cell culture supernatants or lysates, including cell culture supernatants obtained by cell harvests (e.g., by adding a cell permeation agent to release virus from host cells). Partially purified preparations can come from unpurified preparations that have been processed by at least one filtration, chromatography, clarification, precipitation, or fractionation step or any combination thereof. As used herein, “cell culture medium” is meant to encompass both unpurified and purified cytomegalovirus preparations. In some embodiments, the cytomegalovirus preparation comprises a mammalian cell lysate. In some embodiments, the cytomegalovirus preparation comprises a culture harvest of cytomegalovirus released from mammalian host cells. In some embodiments, the cytomegalovirus preparation contains one or more contaminants selected from proteins (e.g., host cell proteins, cell culture serum proteins or exogenously added proteins), and nucleic acids (e.g., host cell DNA).

Suitable host cells include, but are not limited to, epithelial cells (e.g. ARPE-19 human retinal pigment epithelial cells), endothelial cells (e.g. TIME, HUVEC), and fibroblast cells (e.g. MRC-5 human fetal lung fibroblast cells, WI-38 human fetal lung fibroblasts, HFF, etc.). In one embodiment, the cell culture medium is obtained from a cell culture of ARPE-19 cells. Suitable culture media for these cell lines is well known to those skilled in the art and includes, but is not limited to, DMEM (Dulbecco's Modified Eagle Media)/F12 medium supplemented with 10% fetal bovine serum (FBS), DMEM supplemented with 10% FBS, and EMEM (Eagle's Minimum Essential Medium) supplemented with 1% to 15% FBS.

Culturing a cell is performed to enable it to metabolize, grow, divide and/or produce virus of interest. This can be accomplished by methods well known to persons skilled in the art and includes, but is not limited to, providing nutrients for the cell, for instance, in the appropriate culture media and providing an appropriate substrate. The methods may comprise growth adhering to surfaces, growth in suspension, or combinations thereof. Culturing can be done, for instance, in dishes, roller bottles or in bioreactors, using batch, fed-batch, continuous systems, hollow fiber, and the like. High density cell culture systems, such as Corning HyperFlask® and HyperStack® systems can also be used.

In order to achieve large-scale production of virus through cell culture, it is preferred in the art to have cells capable of growing in suspension or microcarriers, and it is preferred to have cells capable of being cultured in the absence of animal- or human-derived serum or animal- or human-derived serum components. Suitable conditions for culturing cells are known. See, e.g., Tissue Culture, (1973), Kruse and Paterson (Eds.), Academic Press, and Freshney, 2000, Culture of animal cells: A manual of basic technique, fourth edition (Wiley-Liss Inc.).

In some embodiments, the cells used to propagate the HCMV are grown on microcarriers. A microcarrier is a support matrix allowing for the growth of adherent cells in spinner flasks or bioreactors (such as stirred bioreactors, rotating wall microgravity biorcactors and fluidized bed bioreactors). Microcarriers are typically 125-250 μM spheres with a density that allows them to be maintained in suspension with gentle stirring. Microcarriers can be made from a number of different materials including, but not limited to, DEAE-dextran, glass, polystyrene plastic, acrylamide, and collagen. The microcarriers can have different surface chemistries including, but not limited to, extracellular matrix proteins, recombinant proteins, peptides and charged molecules. In one embodiment, the microcarriers are Cytodex™ microcarriers (GE Healthcare Life Sciences) which are based on cross-linked dextran matrices.

In some embodiments, the processes described herein employ a nuclease to remove contaminating nucleic acids, which are mostly from the host cell. Exemplary nucleases suitable for use in the invention include BENZONASE®, PULMOZYME®, or any other DNase and/or RNase commonly used within the art. In preferred embodiments of the invention, the nuclease is BENZONASE®, which rapidly hydrolyzes nucleic acids by hydrolyzing internal phosphodiester bonds between specific nucleotides. BENZONASE® is a genetically engineered variant of an endonuclease normally expressed in Serratia marcescens, and can be commercially obtained from Millipore Sigma (Burlington, MA). The concentration at which the nuclease is employed is preferably within the range of 1-100 units/ml.

In certain embodiments, the nuclease is added to the post-harvest cell culture medium. The batch can then be incubated at temperatures ranging from the culturing temperature (e.g., about 37° C.), or at room temperature (around 20° C.) or lower (e.g., around 0° C.), wherein, in general, longer incubation times are required at lower temperatures to achieve the same result. In certain embodiments, the nuclease can be employed before the cell culture medium is harvested. It may be added just seconds prior to (or virtually concomitant with) the harvesting step, but preferably, the nuclease is added to the culture at least one minute before the harvesting step and up to several days before the harvesting step. The cell culture with the added nuclease can then be incubated above process temperature, e.g., around 40° C., or at the culturing temperature (e.g., about 37° C.), or at room temperature (around 20° C.) or lower (e.g., around 0° C.), wherein, in general, longer incubation times are required at lower temperatures to achieve the same result.

In certain embodiments, the HCMV is a recombinant HCMV vaccine. See, e.g., Lilja et al., 2012, Vaccine 30:6980. In certain embodiments, the HCMV is a recombinant vaccine vector expressing a heterologous antigen. The antigen can be from another pathogen or be a tumor antigen. See, e.g., U.S. Pat. No. 9,249,427 (pathogen antigen or tumor antigen), U.S. Patent Application Publication No. 20160354461 (HIV, SIV or TB antigens) and International Patent Application Publication No. WO1998/010311A1 (HIV or malarial antigens); Marzi et al., 2016, Sci Rep 6:21674 (Ebola virus); and Tierney et al., 2012, Vaccine 30:3047 (tetanus toxin).

In one embodiment, the HCMV is a conditional replication defective HCMV (rdHCMV). As used herein, the term “conditional replication defective virus” refers to virus particles that can replicate in certain environments but not others. In certain embodiments, a virus is made a conditional replication defective virus by destabilization of one or more proteins essential for viral replication such as IE1/2, UL51, UL52, UL79 and UL84, or a combination thereof, for example, by fusion with a destabilizing polypeptide such as a sequence of FKBP or a derivative thereof. Preferred FKBP derivatives have one or more of the following substitutions at the denoted amino acid positions F15S, V24A, H25R, F36V, E60G, M66T, R71G, D100G, D100N, E102G, K105I and L106P. The FKBP derivative having the F36V and L106P substitutions is particularly preferred. The nucleic acids encoding the wild type, non-destabilized essential proteins are modified in the conditional replication defective virus. In more preferred embodiments, one or more essential proteins are destabilized. Such fusion proteins can be stabilized by the presence of a stabilizing agent such as Shield-1, commercially available from Cheminpharma LLC (Farmington, CT) or Clontech Laboratories, Inc. (Mountain View, CA). See, e.g., U.S. Patent Application Publication No. 2009/0215169; and Clackson et al., 1998, Proc Natl Acad Sci USA 95:10437-42).

In one embodiment, the conditional replication defective HCMV is derived from AD169 strain that has a restored gH complex expression due to a repair of a mutation in the UL131 gene. As used herein, the terms “pentameric gH complex” or “gH complex” refer to a complex of five viral proteins on the surface of the HCMV virion. The complex is made up of proteins encoded by UL128, UL130, and UL131 assembled onto a gH/gL scaffold. See Wang and Shenk, 2005, Proc Natl Acad Sci USA 102:1815; and Ryckman et al., 2008, J. Virol. 82:60.

In one embodiment, the genome of the rdHCMV has a destabilized IE1/2 and UL51 and has a sequence as shown in SEQ ID NO:1. See U.S. Pat. No. 9,546,355.

### Manufacture of Conditional Replication Defective HCMV

rdHCMV can be propagated in the presence of a stabilizing agent such as Shield-1 (Shld-1) on permissive cell types, preferably human epithelial cells, and more preferably human retinal pigmented epithelial cells. In additional embodiments, the human retinal pigmented epithelial cells are ARPE-19 cells deposited with the American Type Culture Collection (ATCC) as Accession No. CRL-2302. In some embodiments, Shield-1 is present at a concentration of at least 0.5 μM in the tissue culture media. In preferred embodiments, Shield-1 is present at a concentration of at least 2.0 μM in the tissue culture media. Shield-1 can be used to control replication of the rdHCMV in conjunction with FKBP (FK506 binding protein). See, e.g., U.S. Pat. No. 9,546,355.

In certain embodimentsembodimentsembodiments, Shield-1 is one of the components to be removed during the purification steps described herein. After purification of rdHCMV from cell culture media containing Shield-1, there may be a small amount of residual Shield-1 remaining in the rdHCMV preparation. In one embodiment, the level of ShieldShieldShield-1 in the HCMV composition after purification is at least 10-fold below the level used to sustain replication in tissue culture. In another embodiment, the level of Shield-1 in the rdHCMV preparation after purification is 0.05 UM or less. In another embodiment, the level of Shield-1 in the rdHCMV preparation after purification is undetectable.

Determination of Shield-1 levels in a composition can be detected using a LC/MS (liquid chromatography-mass spectroscopy) or HPLC/MS (high performance liquid chromatography-mass spectroscopy) assays. These techniques combine the physical separation capabilities of LC or HPLC with the mass analysis capabilities of and can detect chemicals of interest in complex mixtures.

### Chromatography Media

The methods of the invention employ a combination of different chromatography technologies, including anion exchange chromatography medium and a polishing chromatography medium selected from a mixed-mode chromagraphy resin and a cation exchange chromatography medium.

Anion exchange chromatography exploits positively charged functional groups on the support to bind to negatively charged molecules. The anion exchange chromatography medium can be any weak or strong anion exchange chromatography medium. In some embodiments, the anion exchange chromatography ligand comprises a strong ionic group. Structural groups that are useful as strong anionic exchange functionalities include, for example, secondary, tertiary, and quaternary amines (e.g., diethyl aminoethyl, dimethyl aminoethyl, trimethyl aminoethyl, and quaternary aminoethyl). In certain embodiments, the anion exchange chromatography support is in the form of a column. In certain embodiments, the anion exchange chromatography support is in the form of a membrane.

Commercial examples of anion exchange chromatography media include, but are not limited to, Nuvia™ Q, UNOsphere™ Q, UNO® Q (available from Bio-Rad Laboratories, Inc., Hercules, CA), Capto™ Q (available from GE Healthcare) and POROS™ HQ (available from Applied Biosystems). Anion exchange chromatography media are also described, for example, in U.S. Pat. No. 8,138,291. Example of anion exchange chromatography membranes include, but are not limited to, those produced by Pall (e.g., Mustang™ series including Mustang™ Q) and Sartorius (e.g., Sartobind® series including Sartobind® Q) and Natrix Separations (e.g., NatriFlo® HD-Q membrane).

The cation exchange chromatography medium can be any weak or strong cation exchange chromatography medium. In some embodiments, the cation exchange chromatography ligand comprises a strong ionic group. Structural groups that are useful as strong cationic exchange functionalities include, for example, sulfonic acid ligands. In certain embodiments, the cation exchange chromatography support is in the form of a column. In certain embodiments, the cation exchange chromatography support is in the form of a membrane. Examples of cation exchange chromatography resins include POROS™ XS resin (Thermo Fisher Scientific), Capto™ S (GE Healthcare) and POROS™ HS (Applied Biosystems). Examples of cation exchange chromatography membranes include Sartorius Sartobind® S and Pall Mustang® S.

Mixed-mode chromatography resins exploit a combination of ion exchange (i.e., negatively-charged or positively charged moieties on the mixed-mode support) and hydrophobic interaction, optionally with hydrogen bonding or pi-pi bonding interactions. In some embodiments, the mixed mode ligand comprises at least one acidic moiety such as a carboxyl group, and also comprises at least one hydrophobic moiety such as, but not limited to, a phenyl ring or an aliphatic hydrocarbon chain. In some embodiments, the support is hydrophobic and weakly cationic. For example, in some embodiments, the mixed-mode ligand comprises a carboxylic acid end group in combination with at least one hydrophobic moiety, such as an aromatic hydrophobic ring. Those skilled in the art will understand the definition of “strong” and “weak” ionic groups as those groups which are, or are not, respectively, capable of maintaining their charge across a wide pH range, such as pH 2 to pH 12. In some embodiments, the mixed-mode ligand further comprises an amide bond serving as a hydrogen bond donor/acceptor. Mixed-mode chromatography supports are described in, for example, U.S. Pat. No. 7,999,085.

In certain embodiments, the mixed-mode chromatography also employs core bead chromatography. In core bead chromatography, molecules may be separated based on size. Larger molecules or complexes flow through the chromatography column, while smaller molecules or complexes flow into pores on the surface of the bead. The pore size on the surface of the bead will determine the size of the molecule that may pass through to the inner core of the bead. Accordingly, one skilled in the art may select a mixed-mode resin with an appropriate pore size smaller than the molecule or complexes (such as, for example, the HCMV virus) that is the subject of purification, such that the molecule or complexes to be purified passes through the column, while smaller molecules or complexes enter the pores of the mixed-mode resin. In certain exemplary embodiments, the mixed-mode resin comprisescomprisescomprises pores having an approximate size exclusion of 500, 600, 700, 800, 900 or 1000 nm whereby virus having a diameter greater than the size exclusion size are excluded from the pores in the mixed-mode resin and do not come into contact with the ligand which is in the interior of the resin.

Examples of mixed-mode resins include, but are not limited to, Capto™ Core 700 (multimodal anion exchange octaylamine resin; GE Healthcare), Capto™ Adhere (multimodal strong anion exchange N-benzyl-n-methyl ethanolamine resin; GE Healthcare), Capto™ MMC (multimodal weak cation exchange cross-linked resin; GE Healthcare), hydroxyapatite Type 1 (Bio-Rad), and Nuvia™ cPrime™ (Bio-Rad Laboratories, Hercules, CA). Capto™ Core 700 is a layered, bead-based matrix having a particle size of about 90 μm. The surface of the bead consists of an unliganded, inactive shell with pores that have an approximate MWCO (molecular weight cutoff) of about 700 kDa. The interior of the bead comprises an active functionalized core with multimodal octylamine ligands designed to capture impurities that are small enough to enter the bead through the pores on the surface. Smaller molecules, such as impurities and/or host cell proteins having a size smaller than about 700 kDa, pass through to the inner core octylamine ligands, where they are adsorbed, while the larger virion particles flow through. Large molecules or complexes can thereby be purified from smaller contaminants in the negative (flowthrough) purification mode.

Any solid support can be used for mixed-mode chromatography and ion exchange chromatography. The solid support can be, for example, porous or non-porous and can be in the form, for example, of a matrix, bead, particle, chip, or other conformation, e.g., a membrane or a monolith, i.e., a single block, pellet, or slab of material. Particles when used as matrices can be spheres or beads, either smooth-surfaced or with a rough or textured surface. Many, and in some cases all, of the pores are flow through-pores, extending through the particles to serve as channels large enough to permit hydrodynamic flow or fast diffusion through the pores.

The solid support can be utilized in any conventional configuration, including packed columns and fluidized or expanded-bed columns, monoliths or porous membranes, and by any conventional method, including batchwise modes for loading, washes, and elution, as well as continuous or flow-through modes. In some embodiments, the mixed-mode chromatography support and the anion exchange chromatography support are each packed in a column. In some embodiments, the mixed-mode chromatography support is packed in a column and the ion exchange chromatography supports are each in the form of a membrane. In other embodiments, the mixed mode chromatography support is a resin which is used in slurry mode, which is more amenable to batch sterilization than a packed chromatography column.

In some embodiments, the mixed-mode and/or anion exchange support is packed in a column of any dimension required to support preparative applications. Column diameter may range from less than 1 cm to more than 1 meter, and column height may range from less than 1 cm to more than 30 cm depending on the requirements of a particular application. Other column dimensions may also be used.

### Chromatography Methods

The methods of the present invention utilize a combination of forms of chromatography to purify cytomegalovirus particles from a cell culture medium, typically containing host cell proteins, host cell DNA, serum proteins, and additives. In some embodiments, the methods involve an initial mass capture of cytomegalovirus particles from the cell culture medium using an anion exchange chromatography medium under suitable conditions as known in the art to allow the cytomegalovirus particles to bind to the anion exchange chromatography medium. The cytomegalovirus particles are then eluted from the anion exchange chromatography medium and subjected to a further purification process on a polishing chromatography medium.

The ion exchange chromatography (both anionic and cationic) media that are used in the methods of the present invention are operated in a bind-elute mode. “Bind-elute mode” refers to an operational approach to chromatography in which the buffer conditions are established so that target molecules to be purified (e.g., cytomegalovirus), and optionally undesired contaminants or impurities, bind to the chromatography medium when a preparation is applied to the chromatography medium. Separation of the target molecule (e.g., cytomegalovirus) can be achieved subsequently by changing the conditions such that the target molecule is eluted from the medium. In some embodiments, contaminants or impurities remain bound following elution of the target molecule. In some embodiments, contaminants or impurities either flow through when the preparation is applied to the chromatography medium, or are bound and eluted before elution of the target molecule.

The mixed-mode chromatography resins that are used in the methods of the present invention are operated in a flow-through mode. “Flow-through mode” refers to an operational approach to chromatography in which the buffer conditions are established so that target molecules to be purified (e.g., cytomegalovirus) flow through the chromatography medium when a preparation is applied to the chromatography medium. In certain embodiments, when using mixed mode chromatography resins with a size exclusion component, the target molecules to be purified do not come into contact with ligand because the pore size prevents contact. In both cases, the target molecule does not bind to ligand. Contaminants or impurities remain bound to the chromatography medium following flow through of the target molecule.

### Anion Exchange Chromatography Step

In the methods of the invention, a cell culture medium containing HCMV (e.g., pH 6.6-7.0, conductivity ˜15 mS/cm) is first subject to anion exchange chromatograpy medium to bind HCMV to the anion exchange chromatography medium. The bound HCMV is optionally washed with one or more solutions under conditions in which the HCMV remains substantially bound to the medium, wherein the presence and amount of the solution displaces and/or removes one or more contaminants (e.g., serum proteins, host cell protein, and host cell DNA). A variety of washing agents can be used. In some embodiments, the agent is histidine and/or sodium chloride or another neutral salt (e.g., potassium chloride, ammonium chloride, or sodium sulfate) at a pH between 6 and 7. In some embodiments, the washing agent may include other amino acids such as arginine to affect separation.

Following the optional washing step or steps, the HCMV is eluted from the anion exchange chromatography medium. In some embodiments, the HCMV particles are eluted with a salt gradient (e.g., from 0 M up to 2 M salt or higher). Other elution conditions can also be applied as desired, including, e.g., elution by inclusion of mobile phase modifiers such as amino acids. In some embodiments, the HCMV particles are eluted using an elution buffer comprising sodium chloride or another neutral salt (e.g., potassium chloride, ammonium chloride, or sodium sulfate) at a concentration of about 0.5-2 M (e.g., about 0.5, 0.6, 0.7, 0.8, 0.9, 1, 1.1, 1.2, 1.3, 1.4, 1.5, 1.6, 1.7, 1.8, 1.9, or 2 M). In one embodiment, the HCMV particles are eluted using ˜1 M NaCl in a buffer of approximately pH 7. In certain embodiments, the HCMV is eluted with an amino acid with a basic side chain, e.g., arginine or histidine, at a concentration of about 0.25-1 M.

Some exemplary bind-wash-elute conditions for the anion exchange chromatography step are: binding condition: cell culture media, conductivity between 0-30 mS/cm, pH 6-8 wash condition: 50-300 mM sodium chloride, pH 6-7, in a histidine buffer containing 25-100 mM histidine elution condition: 1 M sodium chloride, pH 7, in a buffer (e.g., histidine)

In some embodiments, prior to contacting the cell culture medium comprising HCMV with the anion exchange chromatography medium (e.g., an anion exchange membrane), the chemical environment of the support is equilibrated. In some embodiments, the anion exchange medium is equilibrated to establish an appropriate pH, conductivity, and/or concentration of salts. Equilibration of the medium is accomplished, for example, by flowing an equilibration buffer containing appropriate reagents through the column. Buffering compounds may include, but are not limited to, histidine, phosphate, Tris, MES, HEPES, BICINE, and imidazole. In one embodiment, the equilibration buffer comprises histidine in an amount between 1 mM to about 50 mM, and NaCl in an amount between 50 mM and about 200 mM. The cell culture medium containing the HCMV particles can also be equilibrated to conditions compatible with the anion exchange chromatography medium equilibration buffer before applying to the anion exchange chromatography medium. In some embodiments, the cell culture medium is equilibrated by adjusting the pH, the concentration of salts, or other compound as desired. In some embodiments, the cell culture medium is equilibrated to a pH of about 6.0 to about 9.5. In some embodiments, the cell culture medium is equilibrated to a pH of about 6.0 to about 8.0 (e.g., about 6.0, about 6.5, about 7.0, about 7.5, or about 8.0). In some embodiments, the cell culture medium is equilibrated to a pH of about 7.

In some embodiments, the cell culture medium is diluted in a buffer prior to applying to the chromatography medium. In some embodiments, the cell culture medium is diluted in a buffer that is the same as or that is compatible with the anion exchange chromatography equilibration buffer. In some embodiments, the cell culture medium is diluted in an equilibration buffer that contains Tris. In some embodiments, the cell culture medium is diluted in an equilibration buffer that comprises Tris and sodium chloride or another neutral salt. In some embodiments, the eluate is diluted in buffer (e.g., equilibration buffer) at a ratio of about 1:1, about 1:2, about 1:3, about 1:4, or about 1:5 of cell culture medium to buffer.

After the chromatography medium and cell culture medium have been equilibrated, the cell culture medium can be contacted to the anion exchange chromatography medium (e.g., membrane) under conditions that allow for the cytomegalovirus particles to bind to the anion exchange column. In some embodiments, the cell culture medium can be contacted to the anion exchange medium at a flow range of, but not limited to, 0.1-5.0 chromatography bed volumes per minute.

### Polishing Chomatography Step

For polishing chromatography, a number of different options are available for removal of trace contaminants and impurities in this step. In certain embodiments, a mixed-mode chromatography step is used. In certain aspects of these embodiments, the mixed-mode chromatography is performed by flow-though (i.e., impurities bind to the mixed-mode chromatography resin while the cytomegalovirus particles do not). In certain embodiments, a cation exchange chromatography step is used using bind-elute.

In certain embodiments, the eluate from the anion exchange chromatography is processed through a suitable bioburden reduction filter prior to application to the polishing chromatography resin. The bioburden reduction filter can be, for example, a 0.2 to 0.45 micron filter.

### Mixed-Mode Chromatography Step

In some embodiments, the eluate from the anion exchange chromatography step comprising cytomegalovirus is contacted with a mixed-mode chromatography resin (e.g., a mixed-mode column, e.g., a hydrophobic cation exchange chromatography column). In some embodiments, the mixed-mode resin is equilibrated to establish an appropriate pH, conductivity, and/or concentration of salts. Equilibration of the resin is accomplished, for example, by flowing an equilibration buffer containing appropriate reagents through the column. Buffering compounds may include, but are not limited to, histidine, phosphate, Tris, MES, HEPES, BICINE, and imidazole. In some embodiments, the equilibration buffer comprises histidine. In some embodiments, the equilibration buffer comprises histidine in an amount from about 1 mM to about 50 mM (for example about 25 mM).

In some embodiments, the mixed-mode chromatography resin is equilibrated to a pH of about 5 to about 7. In some embodiments, the mixed-mode chromatography resin is equilibrated to a pH of about 5.5 to about 7.5 (e.g., about 5.5 about 6, about 6.5, about 7, or about 7.5). In some embodiments, the mixed-mode chromatography resin is equilibrated to a pH of about 6. The eluate (from the anion exchange chromatography) comprising the HCMV can also be equilibrated to conditions compatible with the mixed-mode chromatography equilibration buffer before applying the eluate to the mixed-mode chromatography resin. In some embodiments, the eluate is equilibrated by adjusting the pH, the concentration of salts, or other compound as desired. In some embodiments, the eluate is equilibrated to a pH of about 5.5 to about 7.5. In some embodiments, the eluate is equilibrated to a pH of about 5.5 to about 7.5 (e.g., about 5.5 about 6, about 6.5, about 7, or about 7.5). In some embodiments, the impure preparation is equilibrated to a pH of about 6.

In some embodiments, the eluate is diluted in a buffer prior to applying to the chromatography medium. In some embodiments, the eluate is diluted in a buffer that is the same as or that is compatible with the chromatography equilibration buffer. In some embodiments, the eluate is diluted in an equilibration buffer that comprises histidine. In some embodiments, the eluate is diluted in buffer (e.g., equilibration buffer) at a ratio of about 1:1, about 1:2, about 1:3, about 1:4, or about 1:5 of eluate to buffer.

In one embodiment, the mixed-mode chromatography support is in the form of a packed column, with the eluate (from the anion exchange chromatography step) flowed-through the column. In another embodiment, the mixed-mode chromatography support is in the form of a resin slurry, with the eluate contacted with the resin in a mixing vessel. The resin can be removed from the eluate using a sized based separation which removes the resin beads without removing the HCMV particles. For instance, the eluate may be passed through a straining mesh followed by a clarifying filter with a pore size between approximately 0.45 μm and approximately 10 μm. In one embodiment, a 50 μm mesh followed by a 1.2 μm filter is used.

Following flow-through of the HCMV through the mixed-mode chromatography resin, the resin is optionally washed with one or more solutions under conditions in which any residual HCMV flows away from the resin. In certain embodiments, the composition of the wash buffer would match the equilibration buffer. In some embodiments, the solution contains histidine or another salt. Other wash agents can also be used.

In some embodiments, the HCMV that is recovered from the mixed-mode chromatography resin (as the second step in the process) is substantially free of impurities. As used herein, “substantially free” means that the impurities are 30% or less of the purified HCMV, e.g., less than 30%, 20%, 10%, 5%, or 1%. Various methods of assessing the purity of HCMV are known in the art. In some embodiments, the purity of the cytomegalovirus that is eluted can be analyzed by gel electrophoresis (e.g., SDS-PAGE) and bands corresponding to viral structural proteins may be visualized by staining (e.g., Coomassie staining or SYPRO Ruby staining). The purity of the HCMV that is purified can also be assessed by measuring the amount of host cell protein and/or host cell DNA contaminants or serum proteins such as BSA, if any, that are present in the final purified product. The amount of host cell protein can be measured, for example, by immunoassay (e.g., ELISA) and the amount of host cell DNA can be measured, for example, by PCR (e.g., quantitative PCR against a housekeeping gene or ribosomal RNA gene). The purity of the HCMV can also be assessed by a mass spectrometry method, by quantifying the relative contributions of HCMV proteins to the total protein content of the product.

In some embodiments, the final purified product (after the two chromatography steps) comprising HCMV has a process yield of at least 40%, or at least 50% or more of the HCMV particles of the starting cell culture medium (e.g., cell lysate or cell harvest). Methods of measuring HCMV particles are known in the art (e.g.,quantitation of viral particles by a flow cytometry (e.g. Apogee Flow Systems), quantitation of viral antigen by ELISA or western blot techniques, or quantitation of infectious particles by infectivity assays (e.g., plaque)). In some embodiments, the activity of the final purified product comprising HCMV can be determined by comparing the infectivity before and after the two chromatography steps (e.g., comparing the infectivity of the original cell culture medium to the infectivity of the purified product). In some embodiments, HCMV activity is measured using plaque or relative infectivity assays.

### Cation Exchange Chromatograpy Step

In alternative embodiments for the polishing chromatography step, the eluate from the anion exchange chromatography step is subject to a cation exchange chromatography medium to bind HCMV to the cation exchange chromatography support. In these embodiments, the cationic exchange chromatography is operated in bind and elute mode.

The bound HCMV is optionally washed with one or more agents under conditions in which the HCMV particles remain substantially bound to the chromatography medium, wherein the presence and amount of the agent or agents displaces and/or removes one or more remaining contaminants (e.g., host cell protein, host cell DNA, etc.). A variety of washing agents can be used. In some embodiments, the agent is sodium chloride or another neutral salt (e.g., potassium chloride, ammonium chloride, or sodium sulfate), in a buffered solution. Following the optional washing step or steps, the HCMV is eluted from the cation exchange chromatography medium. In some embodiments, the cation is eluted with a salt gradient (e.g., from 0 M up to 2 M salt or higher). Other elution conditions can also be applied as desired, including, e.g., elution by inclusion of secondary modifiers, e.g., an amino acid. In some embodiments, the HCMV particles are eluted using an elution buffer comprising sodium chloride or another neutral salt (e.g., potassium chloride, ammonium chloride, or sodium sulfate) at a concentration of about 0.3-1 M (e.g., about 0.3, 0.4, 0.5, 0.6, 0.7, 0.8, 0.9, or 1.0 M).

Some exemplary bind-wash-elute conditions for the cation exchange chromatography step are: binding condition: 1-50 mM histidine, 1-100 mM NaCl, pH 5.5
washing condition: 1-50 mM histidine, 50-300 mM NaCl, pH 5.5
elution condition: 1-50 mM histidine, 0.3-0.6 M sodium chloride, pH 5.5,

In some embodiments, prior to contacting the eluate comprising HCMV with the cation exchange chromatography medium (e.g., a cation exchange membrane), the chemical environment of the chromatography medium is equilibrated. In some embodiments, the cation exchange medium is equilibrated to establish an appropriate pH, conductivity, and/or concentration of salts. Equilibration of the chromatography medium is accomplished, for example, by flowing an equilibration buffer containing appropriate reagents through the chromatographic support. Buffering compounds may include, but are not limited to, histidine, acetate, phosphate, Tris, MES, HEPES, BICINE, and imidazole. In some embodiments, the equilibration buffer comprises histidine or a combination of histidine and sodium chloride or another neutral salt. In some embodiments, the cation exchange chromatography medium is equilibrated to a pH of about 5.0 to about 7.0 (e.g., about 5.0, about 5.5, about 6.0, about 6.5, or about 7.0). In some embodiments, the cation chromatography medium is equilibrated to a pH of about 5.5.

The eluate (from the anion exchange chromatography step) comprising the HCMV can also be equilibrated to conditions compatible with the cation exchange chromatography equilibration buffer before applying to the cation exchange chromatography medium. In some embodiments, the eluate is equilibrated by adjusting the pH, the concentration of salts, or other compound as desired. In some embodiments, the eluate is equilibrated to a pH of about 5.0 to about 7.0, e.g., about 5.0, about 5.5, about 6.0, about 6.5, or about 7.0. In some embodiments, the eluate is equilibrated to a pH of about 5.5.

In some embodiments, the eluate from the anion exchange chromatography step is diluted in a buffer prior to applying to the cation exchange chromatography medium. In some embodiments, the eluate is diluted in a buffer that is the same as or that is compatible with the cation exchange chromatography support equilibration buffer. In some embodiments, the eluate is diluted in an equilibration buffer that comprises histidine. In some embodiments, the eluate is diluted in an equilibration buffer that comprises histidine and sodium chloride or another neutral salt. In some embodiments, the eluate is diluted in buffer (e.g., equilibration buffer) at a ratio of about 1:1, about 1:2, about 1:3, about 1:4, or about 1:5 of cell culture medium to buffer.

After the chromatography support and eluate have been equilibrated, the eluate can be contacted to the cation exchange chromatography medium (e.g., membrane) under conditions that allow for the HCMV to bind to the cation exchange medium. In some embodiments, the eluate can be contacted to the cationic exchange medium at a flow rate of, for example, 0.1-5.0 chromatography bed volumes per minute.

### Tangential Flow Filtration

Following the anion exchange chromatography step and/or the polishing chromatography step, the eluate (in the case of ion exchange chromatography) or flow-though (in the case of a mixed-mode chromatography) is optionally subject to tangential flow filtration. In some embodiments, a combination of anion exchange chromatography, optionally followed by tangential flow filtration, and mixed mode chromatography, optionally followed by tangential flow filtration, is used to purify the HCMV. In some embodiments, a combination of anion exchange chromatography, optionally followed by tangential flow filtration, and cation exchange chromatography, optionally followed by tangential flow filtration, is used to purify the HCMV.

Tangential Flow Filtration (TFF) (also referred to as Cross Flow Filtration or CFF) is well known to those of skill in the art and equipment and protocols for its implementation in a wide range of situations are commercially available from a variety of manufacturers including but not limited to the Pall Corporation (Port Washington, NY), Spectrum Labs (Rancho Dominguez, CA), and GE Healthcare Life Sciences (Malborough, MA). Generally, TFF involves the recirculation of the retentate across the surface of a membrane. This gentle cross flow feed minimizes membrane fouling, maintains a high filtration rate and provides high product recovery. In one embodiment, the TFF step may be implemented with a hollow fiber system. Hollow fiber systems are amenable to aseptic processing, if desired, with sterilization performed by gamma irradiation or steam sterilization. In one embodiment, presterilized hollow fiber units (ReadyToProcess hollow fibers from GE) are used. In another embodiment, flat sheet TFF systems can be used. In one embodiment, the Molecular Weight Cut Off (MWCO) of the TFF system is between 500-1000 kDa, preferably about 500 kDa or 750750 kDa. A method for clarification and concentration of HCMV employing TFF is described in Vicente et al., 2014, Eng. Life Sci. 14:318-326.

### Pre-Processing

In some embodiments, the cell culture medium comprising the HCMV particles is subjected to a treatment step or purification step prior to contacting the cell culture medium to the anion exchange chromatography medium. As a non-limiting example, in some embodiments, the cell culture medium is subjected to a filtration or clarification step prior to contacting the medium to the anion exchange chromatography medium. Filtration methods and reagents are known in the art; see, e.g., International Patent Application Publication No. WO2011/045381 and U.S. Patent Application Publication No. US2011/0207202. As another non-limiting example, in some embodiments, the cell culture medium is treated with a nuclease prior to contacting the preparation to the anion exchange chromatography medium. This nuclease treatment can take place through addition of nuclease during virus propagation or through a post-harvest nuclease addition. Suitable nucleases for treating the impure preparation are commercially available and methods for treating the preparation with a nuclease are known in the art. In some embodiments, the nuclease is added to the cell culture prior to or during viral propagation. For example, Benzonase® (EMD Millipore) or TurboNuclease™ (Accelagen) can be added to the cell culture (during virus propagation) for up to 8 days prior to harvest or added to the cell culture supernant (post-harvest) for at least one hour prior to the anion exchange chromatography purification step.

In some embodiments, prior to contacting the cytomegalovirus preparation to the anion exchange chromatography medium, the preparation can be subjected to a combination or two or more treatment or purification steps (e.g., a combination of a filtration or clarification step and a nuclease treatment step).

In some embodiments, the cytomegalovirus preparation comprising the cytomegalovirus particles is not subjected to a treatment or purification (e.g., filtration) step prior to contacting the cytomegalovirus preparation to the anion exchange chromatography resin.

### Aseptic Processing vs. Sterile Filtration

Preparation of sterile compositions is typically achieved by subjecting a final composition to sterile filtration, i.e., though a 0.2 μm filter membrane, prior to administration. For compositions, such as viral vaccine compositions, comprising relatively large viruses (i.e., approximately 100 nm or larger), sterile filtration may result in a substantial loss of virus due to it being retained in the membrane, reducing the viral yield from the purification process. The present invention provides two alternative methods for providing sterile compositions.

### Aseptic Process

In one embodiment, the two-step chromatography process is performed aseptically. In this embodiment, the anion exchange chromatography is an anion exchange membrane since sterilizing a packed chromatography column can be very challenging. The AEX membrane can be autoclaved, and the use of mixed mode polishing resin in “impurity binding” mode means no elution is necessary and a packed column is not required. In an impurity binding mode, the eluate is contacted with the resin whereby impurities bind to the resin and the HCMV does not bind to the resin. Because no elution step is required, this operation can occur in a mixed slurry mode rather than a packed column. The mixed-mode polishing resin can be autoclaved to achieve an aseptic resin preparation. The chromatography beads are then removed from the batch using an aseptic filtration train—for example, a 50 μm mesh followed by a filter with a nominal pore size between 0.45-10 μm.

### Sterile Filtration

In another embodiment, the two-step chromatography process is followed by a sterilizing filtration using a 0.2 μm (nominal) membrane made of cellulose acetate, PES, or PVDF (polyvinylidene difluoride). In the case where TFF is employed following the polishing chromatography step, the sterile filtration would follow TFF.

The present invention is also directed to a method for sterile filtration of a large virus such as HCMV (size range of ˜100-400 nm) comprising applying a cell culture medium containg virus or a virus preparation onto a 0.2 μm (nominal) membrane made of cellulose acetate or PVDF, and in some cases, PES. This invention is based on the surprising discovery that viruses which are larger than the nominal 0.2-micron pore size of most sterilizing filters can pass through membranes made of cellulose acetate or PVDF, and in some cases, PES.

### Exemplary Process

In one embodiment, a complete purification process involves: (1) Nuclease digestion (with Benzonase®) prior to harvest in a bioreactor containing cell culture medium and microcarrier beads having cells infected with HCMV; (2) harvest of cell culture medium from microcarrier beads; (3) clarification of harvested cell culture medium by a 1.2-micron glass-fiber filter (e.g. Sartorius Sartopore® GF Plus); (4) capture of viral particles by anion exchange (AEX) membrane chromatography (e.g., Sartorius Sartobind® Q); (5) polishing purification by Capto™ Core 700 chromatography (GE Healthcare) operated in a flow through mode; (6) concentration and buffer exchange by 750-kDa hollow fiber TFF (e.g., GE Healthcare or Spectrum). In one aspect, a sterile filtration using a 0.2-micron filter selected from Sartorius Sartobran® P, Millipore Durapore™ PVDF filter, or Pall SuporLife® is performed before or after the TFF step in (6). In an alternative aspect, no sterile filtration is performed and the process is run completely aseptically.

In one embodiment, a 750-kDa hollow fiber TFF (GE Healthcare or Spectrum) step may also be included between the AEX and polishing chromatography.

This purification can generate vaccine having ≥80% HCMV purity (as assessed by HCMV protein) and ≤10 ng DNA/dose, with a process yield≥40%.

Examples are provided below to further illustrate different features of the present invention. The examples also illustrate useful methodology for practicing the invention. These examples do not limit the claimed invention.

### EXAMPLES

### Analytical Methods

### Protein Purity by Mass Spectrometry

Fractions of HCMV, host cell protein (HCP), and bovine proteins were assessed using a proteomic approach that uses LC-MS/MS (liquid chromatography-tandem mass spectrometry) to determine the approximate percentage of these proteins in HCMV process samples. The HCMV sample was trypsin-digested and then the peptides were analyzed using LC-MS/MS. Relative quantitation at the protein level was determined by summing the MS scan intensity of all the identified peptides that met an acceptance threshold. This summed intensity of each protein is the result of the total signal arising from all the peptides (with acceptable MS/MS results) matching that protein. The reported relative abundance of each protein type (HCMV, HCP, or residual bovine protein) is reported.

### Viral Particle Concentration

A relative size distribution and total concentration of viral particles in the HCMV sample can be obtained by flow cytometry (e.g. Apogee Flow Systems). As the individual particles pass through the instrument's flow cell, they are illuminated by a 405 nm laser and the scattered light is detected. The obtained distribution is a relative size distribution of light scattering signal on a logarithmic scale. Counts in total particles per milliliter were also determined.

### Relative Infectivity Assay

This analytical procedure is a cell-based relative infectivity assay based on expression of a HCMV protein following infection. ARPE-19 cells were platedplated and incubated in 96-well micro-titer plates followed by infection with serial dilutions of HCMV samples, controls, and standards. The infected cells were incubated for approximately 1 to 5 days then fixed with a fixative (e.g. formaldehyde). After washing, the plate is incubated with a monoclonal antibody (MAb) reactive with a HCMV protein. After incubation with the anti-HCMV MAb, the plate is washed and incubated with a detection antibody. After this final incubation, the plate is washed and read on a plate reader instrument. The relative infectivity assigned to the test article is based on signal generated with an assay reference standard tested on the same plate.

### BSA Content by ELISA

This analytical procedure is a sandwich ELISA method which measures bovine serum albumin (BSA) in vaccine samples. This method is used to measure the amount of residual BSA in the purified HCMV sample. Dilutions of the purified HCMV sample are incubated on plates pre-coated with an anti-BSA capture antibody. Captured BSA is subsequently detected with an enzyme-conjugated, BSA-specific antibody. The residual BSA in HCMV sample is quantified using an optical density standard curve generated with a BSA reference standard.

### BSA Content by Simple Western

For some samples, a Capillary Electrophoresis based Simple Western™ is used to monitor BSA mass. The sample is reduced and denatured, then loaded to a capillary and separated by size, bound to the capillary using a photo-linking technology and probed with an anti-BSA monoclonal antibody. A secondary antibody, luminol, and peroxide were utilized for chemiluminescence detection of BSA. Virus concentration of test samples was determined relative to a BSA reference standard of known concentration.

Host Cell DNA content by qPCR

Total nucleic acids were isolated from the sample by proteinase K treatment, phenol-chloroform extraction, and alcohol precipitation. Host cell DNA concentration is measured by a real-time quantitative PCR (qPCR) standard curve assay (standard curve range: 200 ng/ml-20 pg/mL).

### Measuring Virus Mass Content by Simple Western™ HCMV Glycoprotein Assay:

A Capillary Electrophoresis based Simple Western™ can be used to monitor virus mass. The HCMV sample was reduced and denatured, then loaded to a capillary and separated by size, bound to the capillary using a photo-linking technology and probed with a rabbit HCMV glycoprotein monoclonal antibody. A goat anti-rabbit HRP conjugate secondary antibody, luminol, and peroxide were utilized for chemiluminescence detection of the HCMV glycoprotein. Virus concentration of test samples was determined relative to a purified HCMV reference standard of known virus concentration.

### Quantitation of Total Protein Using the Pierce® 660 nm Assay

The Pierce™ 660 nm Assay (Thermo Fisher Scientific) is a colorimetric method used to quantify total protein concentration. The assay is based on the binding of a propietary dye-metal complex to protein in acidic conditions that causes a shift in the dye's absorption maximum. A standard curve was prepared at seven concentration levels (15, 25, 50, 75, 100, 150, and 200 μg/mL) from Bovine Serum Albumin (BSA). Samples were diluted and then incubated in the presence of Benzonase® to reduce nucleic acid interference. The Pierce® 660 nm Assay Reagent was added, the plate was incubated at room temperature and the absorbance was read at 670 nm. Unknown sample concentrations were determined by interpolation from the standard curve using a linear fit.

### Protein Analysis by SDS PAGE

To measure relative purity of HCMV proteins versus impurities, SDS PAGE was used to quantify HCMV Tegument protein. The sample was reduced and denatured, then loaded to a Bis-Tris SDS PAGE gel. Staining was executed using SYPRO® RUBY gel stain (Thermo Fisher Scientific) following the manufacturer's protocol. Densitometry was used to quantify the percentage of HCMV Tegument protein present relative to other protein bands.

### Chromatography Methods

### Anion Exchange Chromatography

Cell culture medium, optionally clarified, was loaded to the Sartobind® Q AEX membrane at between 1-3 bed volumes per minute. A wash step was conducted. In some examples, the wash was conducted using 225 mM NaCl in histidine buffer at pH 7. In other examples, the wash step was conducted by increasing histidine concentration to ˜80-100 mM Histidine at pH 6. The HCMV particles were then eluted by increasing NaCl concentration to approximately 1 M NaCl, in a pH 7 histidine buffer.

### Mixed Mode Chromatography (Capto™ Core, Capto™ Adhere, CHT™ Hydroxyapatite)

AEX eluate was purified by contacting the eluate with a mixed-mode chromatography resin (Capto™ Core, Capto™ Adhere, or CHT™ Ceramic Hydroxyapatite) in either a packed column or in a mixing vessel. As described in the examples below, the AEX elutate was sometimes contacted directly with the mixed-mode chromatography resin, at a pH of 7 and a salt concentration of approximately 1 M NaCl. In other examples, a TFF step was used following the AEX step to adjust the NaCl concentration to approximately 150 mM NaCl, at a pH between 6 and 7.

### Cation Exchange Chromatography

AEX eluate was processed through a TFF step to adjust the NaCl concentration to approximately 75 mM NaCl at a pH between 5 and 6. The batch was then loaded to the Sartobind® S membrane adsorbers cation exchange (CEX) membrane at between 1-3 bed volumes per minute. A wash step was conducted at 225 mM NaCl at pH 5.5. The HCMV particles were then eluted by increasing NaCl concentration to approximately 500 mM NaCl at pH 5.5.

### Example 1: Demonstration of a Need for AEX Capture Step, Comparing to TFF-only Purification

Purification of HCMV virions or dense bodies using Tangential Flow Filtration (TFF) has been reported. See, e.g., Vicente et al., 2014, Eng. Life Sci. 14:318-326; and Schneider-Ohrum et al., 2016, J. Virol. 90:10133-10144. During development of a commercial process for HCMV vaccine purification, it was found that TFF purification alone may be capable of achieving the desired purification results for harvests with minimal host cell impurities (e.g. harvest from static cultures). However, for harvests containing more impurities, as was seen with microcarriers, TFF was found to be insufficient to achieve the desired protein purity of ≥80% HCMV protein when using similar TFF conditions as those used for static culture. Improving TFF purification by increasing membrane area or crossflow was not feasible as the TFF process developed for static culture already required feed flowrates in excess of 100 liters per minute at the desired manufacturing scale. Implementation of an anion exchange capture chromatography step was explored to reduce the amount of protein being loaded onto TFF. ARPE-19 cells (ATCC No. CRL-2302, American Type Culture Collection, Manassas, VA) were initially cultured in static vessels (i.e., T-225 Flask). Through cell expansion, cells were eventually cultured on microcarriers in a 3 L stirred tank bioreactor. Upon appropriate cell growth in the 3 L reactor, the cell culture was infected with a recombinant HCMV as described in U.S. Pat. No. 9,546,355.

On harvest day, the contents of the bioreactors were harvested, clarified using filtration across a 1.2 mm clarification filter capsule, and treated with nuclease (Benzonase® endonuclease, EMD Millipore, 80 U/mL for 2 hours).

The cultures were then purified using either (A) tangential flow filtration (750 kDa cutoff) or (B) AEX capture chromatography followed by tangential flow filtration. In each process scenario, the tangential flow filtration step was conducted by concentrating the batch to approximately 20 percent of the original harvest volume, then diafiltering against eight diavolumes of buffer. In the arm including AEX, the AEX step was conducted using a Sartobind® Q membrane adsorber. The feed material (nuclease-treated cell culture medium) was loaded on the Sartobind® Q membrane adsorber. The HCMV particles were eluted by increasing the NaCl concentration to approximately 1 M NaCl at pH 7. Protein purity was measured by mass spectometry as described in the analytical methods section. Results are shown in Table 2 alongside previous results obtained using the TFF purification process and virus harvested from static cell culture.

For the microcarrier harvest, the TFF only arm experienced membrane fouling and the purity of the resulting product, 9%, was significantly below the target value of 80% HCMV protein. Incorporation of an AEX capture step resulted in a reduced protein load to the TFF and and a significantly higher purity than the TFF-only arm. The data indicates that the AEX capture step is required to approach the goal of ≥80% HCMV protein, but also that additional polishing is required.

TABLE 2
Purifications of HCMV vaccine harvest using either TFF
or AEX capture followed by TFF. For each arm, the volumetric
loading to TFF was similar (~70 liters/m² membrane).
The feed material was nuclease treated prior to the membrane
steps in all cases. The same feed material was used for
both arms of microcarrier culture purification.

| | Static culture | | Microcarrier culture |
|---|---|---|---|
| Process | TFF | TFF | AEX/TFF |
| % HCMV | 88% | 9% | 71% |
| % Human | 9% | 18% | 24% |
| % Bovine | 3% | 74% | 6% |

### Example 2: Identification of Polishing Chromatography Resins Capable of Achieving ˜80% HCMV Process Yield

To increase purity following an AEX capture/TFF sequence, various polishing chromatography steps were evaluated. In a high throughput screening experiment, product previously purified by AEX/TFF was incubated with various chromatography resins in a plate format. A filter plate was then used to remove the resin and the products were analyzed by mass spectrometry.

Table 3 shows the protein purity results. For this purification, feed material was measured at 47.2% HCMV.

TABLE 3
Purity results for evaluation of polishing resins in a
microscale study. Resins were evaluated in a negative
purification mode (product does not bind to resin)

| Sample Name | % HCMV |
|---|---|
| Feed Material (AEX/TFF Purified) | 47.2 |
| Capto ™ Adhere | 82.3 |
| CHT ™ Hydroxyapatite Type 1 | 87.7 |
| Capto ™ Core | 77.3 |

The experiment identified three polishing chromatography resins (Capto™ Adhere, CHT™ Hydroxyapatite, and Capto™ Core) all with the ability to increase purity to near the 80% HCMV target.

Following this initial evaluation, although all three resins appeared suitable for use as a polishing chromatography, Capto™ Core was selected for the polishing step. During subsequent development experiments, HCMV polishing step yields seen with Capto™ Adhere and Capto™ Core were more consistent than with CHT™ Hydroxyapatite (data not shown). As part of comparing Capto™ Adhere and Capto™ Core as polishing resins, the two resins were evaluated for their ability to remove BSA under different salt conditions. The results are shown in Table 4.

TABLE 4
Comparison of BSA Clearance for Capto ™
Adhere and Capto ™ Core at varying salt
conditions. Batches first purified by AEX/
TFF were contacted with these polishing
resins under the conditions shown.

| | BSA per |
|---|---|
| Resin and conditions | dose (ng/dose) |
| Capto ™ Adhere, 150 mM NaCl | 63 |
| Capto ™ Adhere, 1M NaCl | 356 |
| Capto ™ Core, 150 mM NaCl | 80 |
| Capto ™ Core, 1M NaCl | 86 |

Removal of BSA was shown to be more consistent across a broad range of salt backgrounds for Capto™ Core than for Capto™ Adhere. The ability to use Capto™ Core in high salt conditions following AEX elution allows for greater process flexibility and means that there is no conductivity adjustment required following AEX elution. Reducing the conductivity of the AEX elutate requires either a significant dilution or a tangential flow filtration (TFF) step.

### Example 3: Identification of CEX Chromatography as a Polishing StepStep Capable of Achieving >80% HCMV Process Yield

A membrane CEX step (Sartorius Sartobind® S) was also identified as a polishing technology with the ability to achieve >80% HCMV.

In this experiment, cell culture harvest prepared as described in Example 1 was purified using a nuclease treatment, clarification, AEX capture, and TFF. The batch was then treated via either (a) incubation with Capto™ Core resin in batch mode or (b) pH reduction by titration, followed by bind-elute CEX chromatography (Sartorius Sartobind® S).

Table 5 shows mass spectrometry purity results following TFF and following each capture step. The purity results show that either option was capable of increasing purity to ≥80% HCMV. The data also again demonstrates that two chromatography steps (AEX followed by a polishing step) were required to achieve >80% HCMV.

TABLE 5
Mass Spectometry Purity results: Head to head polishing
comparison using Capto ™ Core or CEX Membrane
to increase purity following the AEX and TFF Steps

| Sample Name | % HCMV |
|---|---|
| TFF product (following AEX/TFF) | 50.4 |
| Final product using Capto ™ Core | 90.2 |
| Final product using CEX Membrane | 84.3 |

### Example 4: Efficiency of AEX/Capto™ Core Sequence Compared to Capto™ Core Alone

Purification of viral particles from cell culture using Capto™ Core chromatography as the primary chromatography step has been reported. See, e.g., Mundle et al., 2016, Vaccine 34:3690-6. Mundle describes using approximately 10 L of Capto™ Core media for every 100 L of cell culture harvest.

The use of an AEX capture step prior to Capto™ Core results in a more efficient use of chromatography media than that described by Mundle et al. Table 6 shows data characterizing protein content before and after the AEX capture step. In this example, viral protein makes up a small fraction (<5%) of the protein present at harvest.

As shown in Table 6, the AEX step removes >95% of the total protein load entering the process. Because most impurities flow through in the AEX step, less chromatography media is required than for a chromatography step such as Capto™ Core which binds impurities. As shown in Table 7, the AEX-Capto Core sequence uses less total chromatography media than the previously published process which uses Capto Core alone.

TABLE 6
Characterization of total protein removal by AEX capture step.

| Clarified Bulk | Volume of Batch | 500.3 | L |
|---|---|---|---|
| (AEX Feed) | [Protein], μg/mL | 1061 | |
| | Mass Protein | 530.8 | g |
| AEX Product | Volume of Batch | 100.5 | L |
| | [Protein], μg/mL | 214 | |
| | Mass Protein | 21.5 | g |

| | |
|---|---|
| Percent of Protein Removed by AEX | 95.9% |

TABLE 7
Amount of chromatography media used per 100 L
batch, for previously published Capto ™ Core
process and process described herein.

| | Previously | |
|---|---|---|
| | published | |
| | process | AEX-Capto ™ |
| | (Mundle et al.) | Core process |
| AEX Chromatography | N/A | 2 L per 100 L batch |
| Media | | |
| Capto ™ Core | 10 L per 100 L batch | 1 L per 100 L batch |
| Chromatography Media | | |

### Example 5: Demonstration of Purification Containing Two-step Chromatography Sequence (AEX→Capto™ Core), Using Aseptic Purification

Following identification of the preferred chromatography sequence (AEX membrane capture followed by Capto™ Core polishing), a series of bioreactor harvests were purified using aseptic processing unit operations. The batches below were purified as follows:
1. Benzonase® digestion by addition to bioreactor;
2. Removal of microcarriers using a 50 μm mesh;
3. Batch clarification by 1.2 μm filtration (Sartorius Sartopure® GF Plus capsules)
4. AEX membrane capture using autoclaved membrane capsules (Sartorius Sartobind® Q);
5. TFF concentration and diafiltration;
6. Capto™ Core chromatography using autoclaved resin in a batch binding mode;
7. Concentration and Buffer Exchange by hollow fiber TFF.

Table 8 summarizes the results for the purification.

TABLE 8
Aseptic purification process

| | Target/ | | | |
|---|---|---|---|---|
| | Claimed | Batch | | |
| | Value | Batch #1 | Batch #2 | Batch #3 |
| Purification Scale | N/A | 50 L | 200 L | 500 L |
| (Harvested | | | | |
| Bioreactor Volume) | | | | |
| Protein Purity | >80% HCMV | 94% | 94% | 90% |
| (Mass Spec) | | | | |
| Residual DNA | <10 ng/dose | 0.82 | 0.3 | 0.24 |
| (ng/dose) | | | | |
| (qPCR/ELISA) | | | | |
| Residual BSA | <50 ng/dose | 16 | 30 | 35.9 |
| (ng/dose) (ELISA | | | | |
| BSA/ELISA dose) | | | | |
| Product Yield | >40% | 42% | 48% | 59% |
| (Viral Particles | | (HCMV | (HCMV | (viral |
| or HCMV | | Western) | Western) | particles) |
| glycoprotein | | | | |
| Western) | | | | |
| Product Yield | >30% | 45% | 33% | 37% |
| (Infectious | | | | |
| Particles) | | | | |

The results show that at 50, 200 and 500 L scale, the process was able to achieve >80% HCMV (protein purity), <50 ng per dose BSA, <10 ng per dose residual DNA, >40% viral particle yield, and >30% infectious particle yield.

### Example 6: Ability to Sterile Filter HCMV Particles

The current regulatory expectation is that a vaccine should be sterilized by a 0.2 μm sterilizing grade filter—if the product cannot be filtered with this membrane, a fully aseptic process is typically required. The size of the HCMV particles ranges from 100-400 nm. Therefore, it is not obvious that the HCMV particles can pass through a filter with a nominal pore size of 0.2 μm, or 200 nm.

Two experiments are shown below in which filtration of purified HCMV particles was attempted with four commercially available sterile filters of different materials.

TABLE 9
HCMV sterile filtration experiment #1

| | Viral | Infectious |
|---|---|---|
| Filter | particle yield | particle yield |
| A (Millipore PES. 0.2 μm | <1% | <3% |
| nominal size) | | |
| B (Millipore PVDF. 0.2 μm, | 60% | 73% |
| nominal size) | | |
| C (Sartorius Sartobran ® P)- | 75% | 90% |
| Cellulose acetate, 0.2 μm | | |
| nominal size | | |

TABLE 10
HCMV sterile filtration experiment #2

| | Viral | Infectious |
|---|---|---|
| Filter | particle yield | particle yield |
| Sartorius Sartobran ® P | 82% | 66% |
| Cellulose acetate, 0.2 μm | | |
| nominal size | | |
| Pall SuporLife ® | 80% | 52% |
| PES, 0.2 μm nominal size | | |

The data shows that (a) HCMV particles can be sterile filtered at >70% yield with certain sterile filtration membranes, including the cellulose acetate Sartobran® P membrane and the Pall SuporLife® PES membrane; and (b) it is not obvious that HCMV particles can be sterile filtered, as some common sterile filtration membranes give very low yield, including the Millipore PES. In a wider screen of commercially available sterile filtration membranes (data not shown), approximately one third of 25 membranes screened delivered an HCMV particle yield of >70%.

### Example 7: Demonstration of Purification Process Including Sterile Filtration

The use of sterile filtration at the end of a HCMV vaccine purification process allows for the use of non-sterile technologies (e.g. packed Capto™ Core polishing chromatography columns). The following data is for a purification of HCMV bioreactor harvest (3 L bioreactor volume) using an exemplary embodiment: (1) Nuclease digestion (with Benzonase®) in the bioreactor prior to harvest; (2) harvest of cell culture medium from microcarrier beads; (3) clarification of harvested medium by a 1.2-micron glass-fiber filter (Sartorius Sartopore® GF+); (4) capture of viral particles by AEX membrane chromatography (Sartorius Sartobind® Q); (5) polishing purification by Capto™ Core 700 chromatography (GE Healthcare) operated in in flow through mode using a packed chromatography column; (6) concentration and buffer exchange by 750-kDa hollow fiber TFF (GE Healthcare), and (7) Sterile filtration using Sartobran® P. The results of the purification are shown in Table 11.

TABLE 11
Purification process using sterile filtration

| | |
|---|---|
| Process Yield (virus particles) | 46% |
| Protein Purity (Mass Spec) | 99% HCMV |
| BSA/dose | 16 ng/dose |

The results indicate that comparable yield, and purity are achieved by this process compared to the process earlier shown for 50-500 L scale harvests.

Additional data was generated from larger batches which incorporate sterile filtration and therefore use non-sterile chromatography technologies (membrane cassettes and packed chromatography columns).

Both batches (100 L and 2000 L) included the following steps:
1. Benzonase® digestion by addition to bioreactor;
2. Removal of microcarriers using a 50 μm mesh;
3. Batch clarification by 1.2 μm filtration (Sartorius Sartopure® GF Plus capsules)
4. AEX membrane capture using Sartorius Sartobind® Q membranes
5. Capto™ Core chromatography using a packed column;
6. TFF concentration and diafiltration.

A sterile filtration step with Sartobran® P was incorporated either before (2000 L batch) or after the TFF step (100 L batch).

Table 12 summarizes the results for the purification.

TABLE 12
Sterile purification process

| | Target/ | | |
|---|---|---|---|
| | Claimed | Batch | |
| | Value | Batch #1 | Batch #2 |
| Purification Scale | N/A | 100 L | 2000 L |
| (Harvested | | | |
| Bioreactor Volume) | | | |
| Location of sterile | N/A | Following | Following |
| filtration | | TFF (final | Capto ™ Core, |
| | | purification | prior to TFF |
| | | step) | |
| Protein Purity | >80% HCMV | 87% | ~90%* |
| (Mass Spec) | | | |
| Residual DNA | <10 ng/dose | 0.02 | 0.22 |
| (ng/dose) | | | |
| (qPCR/ELISA) | | | |
| Residual BSA | <50 ng/dose | 43 | 8.3 |
| (ng/dose) (ELISA | | | |
| BSA/ELISA dose) | | | |
| Product yield (Viral | >40% | 50% | 56% |
| Particles or HCMV | | | |
| glycoprotein Western) | | | |
| Product Yield | >30% | 42% | 55% |
| (infectious Particles) | | | |

*Protein purity estimated for this sample based on surrogate SDS-PAGE analysis. Mass Spec data not available.

Similar to the aseptic process, the results show that at 100 and 2000 L scale, the process was able to achieve >80% HCMV (protein purity), <50 ng per dose BSA, <10 ng per dose residual DNA, >40% viral particle yield, and >30% infectious particle yield.

### Example 8: Improved AEX Protein Purity Using Histidine Based Wash

In one version of the purification process, the AEX membrane was washed by increasing NaCl concentration at a constant histidine concentration to remove impurities. In another version, the AEX membrane was washed by increasing histidine concentration at a constant NaCl concentration and a lower pH. This was found to provide a higher purity AEX product as shown in Table 13.

TABLE 13
AEX protein purity comparison using salt based
AEX wash versus a histidine based AEX wash

| | Salt Based | Histidine based |
|---|---|---|
| Process Description | AEX Wash | AEX Wash |
| AEX Equilibration Buffer | 25 mM Histidine, | 25 mM Histidine, |
| | 150 mM NaCl, pH 7 | 150 mM NaCl, pH 7 |
| AEX Wash Buffer | 25 mM Histidine, | 100 mM Histidine, |
| | 225 mM NaCl, pH 7 | 150 mM NaCl, pH 6 |
| AEX Product protein purity | 25% | 41% |
| measure (percent HCMV | | |
| tegument by SDS-PAGE) | | |
| AEX Product BSA | 5.9 | 1.1 |
| Concentration (μg/mL) | | |

Other embodiments are within the following claims. While several embodiments have been shown and described, various modifications may be made without departing from the spirit and scope of the present invention.

1. A method of purifying human cytomegalovirus (HCMV) from a cell culture medium, the method comprising:
a) contacting the cell culture medium comprising HCMV to an anion exchange chromatography medium under conditions that allow the HCMV to bind to the anion exchange chromatography medium;
b) eluting the HCMV from the anion exchange chromatography medium to obtain an eluate;
c) contacting the eluate with a polishing chromatography medium; and
d) collecting the HCMV from the polishing chromatography medium to obtain purified HCMV.

2. The method of claim 1, wherein the polishing chromatography medium is selected from a mixed-mode chromatography resin and a cationic exchange chromatography medium.

3. The method of claim 2, wherein the polishing chromatography medium is a mixed-mode chromatography resin and the HCMV flows through the mixed-mode chromatography resin.

4. The method of claim 2, wherein the mixed-mode chromatography resin has size exclusion properties and the HCMV is excluded from the mixed-mode chromatography resin.

5. The method of claim 4, wherein the mixed-mode chromatography resin includes a hydrophobic anion exchange chromatography ligand and a molecular weight exclusion of about 700 kDa.

6. The method of claim 2, wherein the polishing chromatography medium is a cationic exchange chromatography medium, the HCMV binds the cation exchange chromatography medium, and the HCMV is eluted from the cationic exchange chromatography medium.

7. The method of claim 1, further comprising tangential flow filtration of the eluate from step b), the purified HCMV from step d), or both.

8. The method of claim 1, wherein the HCMV is a recombinant genetically modified HCMV.

9. The method of claim 8, wherein the recombinant genetically modified HCMV has a genomic sequence of SEQ ID NO: 1.

10. The method of claim 1, wherein the cell culture medium comprises one or more contaminants selected from proteins and nucleic acids.

11. The method of claim 1, wherein the cell culture medium comprises serum proteins, host cell proteins, Shield 1, exogenous endonuclease and host cell DNA.

12-14. (canceled)

15. The method of claim 1, wherein said eluting in step b) is by a salt selected from sodium chloride, potassium chloride, ammonium chloride, and sodium sulfate, each at a concentration of about 0.5-2 M.

16. The method of claim 1, wherein said eluting in step b) is by an amino acid selected from arginine and histidine, each at a concentration of about 0.25-1 M.

17-19. (canceled)

20. A method for the purification of HCMV from a cell culture medium comprising the steps of:
a) harvesting cell culture medium from a culture of ARPE-19 cells infected with HCMV;
b) subjecting the cell culture medium to nuclease treatment using an endonuclease at a concentration of 10-160 U/mL;
c) clarifying the nuclease-treated cell culture medium using a 1.2 μm glass fiber filter to obtain a clarified cell culture medium;
d) contacting the clarified cell culture medium comprising HCMV with an anion exchange chromatography membrane under conditions that allow the HCMV to bind to the anion exchange chromatography membrane and then eluting the HCMV from the anion exchange chromatography membrane to obtain an eluate;
e) contacting the eluate with a mixed-mode chromatography resin which is a hydrophobic anion exchange chromatography resin having a molecular weight exclusion of about 700 kDa and then collecting the HCMV from the mixed mode chromatography resin to obtain purified HCMV; and
f) performing tangential flow filtration on the purified HCMV collected from step e) to adjust to the desired concentration and buffer.

21. The method of claim 20, wherein step b) is run prior to step a) by addition of nuclease to the cell culture medium prior to harvesting.

22. The method of claim 20, wherein step b) is run following step a).

23. (canceled)

24. The method of claim 20, wherein the purified HCMV has at least 80% HCMV protein purity.

25. The method of claim 20, wherein the purified HCMV has a yield of at least 40%.

26. The method of claim 20, wherein the purified HCMV contains 10 ng or less hcDNA per dose.

27. (canceled) 28 A process for sterilizing a large virus comprising:
a) applying unpurified material comprising said virus onto a 0.2 μm membrane made of PVDF or cellulose acetate; and
b) collecting purified virus.
